(19) United States
(12) Patent Application Publication
(10) Pub. No.: US 2022/0290188 A1
(43) Pub. Date: Sep. 15, 2022

(54) Compositions and Methods for Modulation of Gene Expression

(71) Applicant: Altius Institute for Biomedical Sciences, Seattle, WA (US)

(72) Inventors: Fyodor Urnov, Seattle, WA (US); John A. Stamatoyannopoulos, Seattle, WA (US); Joycelynn Pearl, Seattle, WA (US); Matthew Wilken, Seattle, WA (US); Christie Ciarlo, Seattle, WA (US); Shon Green, Seattle, WA (US)

(21) Appl. No.: 17/632,365

(22) PCT Filed: Aug. 6, 2020
(86) PCT No.: PCT/US2020/045174

§ 371 Date: Feb. 2, 2022

Related U.S. Application Data
(60) Provisional application No. 62/937,011, filed on Nov. 18, 2019.
(60) Provisional application No. 62/898,343, filed on Sep. 10, 2019.
(60) Provisional application No. 62/884,028, filed on Aug. 7, 2019.

(51) Int. Cl. C12N 15/90; C07K 14/725; C07K 14/705; C12N 15/85; C12N 9/22

(52) U.S. Cl. CPC C12N 15/907; C07K 14/7051; C07K 14/70521; C12N 15/85; C07K 14/70596; C12N 9/22; C07K 2319/71; C07K 2319/81; C12N 2830/005

(57) ABSTRACT

The present disclosure provides polypeptides, compositions thereof, and methods for suppressing expression of a target gene such as PDCD1, CTLA4, LAG3, or TIM-3. The polypeptides disclosed herein include a DNA binding domain (DBD) that binds to a sequence of the target gene and a transcriptional repressor domain that suppresses expression of the target gene. The transcriptional repressor domain may be a known transcriptional repressor or may be a novel transcriptional repressor disclosed herein. Also disclosed herein are novel transcriptional repressors that are conjugated to a heterologous DNA binding domain and mediate suppression of expression of a target gene bound by the DNA binding domain.

### CROSS-REFERENCE TO RELATED APPLICATIONS

This application claims priority benefit of U.S. Provisional Application No. 62/884,028, filed Aug. 7, 2019, U.S. Provisional Application No. 62/898,434, filed Sep. 10, 2019, and U.S. Provisional Application No. 62/937,011, filed Nov. 18, 2019, the disclosures of which are incorporated herein by reference in their entirety.

### INCORPORATION OF SEQUENCE LISTING

The sequence listing named “ALTI-727WO Seq Listing_ST25” which was created on Aug. 4, 2020 and is 219 KB in size, is hereby incorporated by reference in its entirety.

### BACKGROUND

Modulating gene expression has been a strategy for enhancing the success of cancer and infectious disease therapies. In particular, cell therapies, such as CAR T cell therapies, can suffer from dampened immunogenicity by inhibition via an immune checkpoint inhibitor. Thus, there exists a need for agents that can modulate the expression of target genes, such as, immune checkpoint inhibitors. The present disclosure provides engineered polypeptides comprising DNA binding domains and repressor domains for repressing a target gene.

### SUMMARY

The present disclosure provides polypeptides, compositions thereof, and methods for suppressing expression of a target gene such as PDCD1, CTLA4, LAG3, or TIM-3. The polypeptides disclosed herein include a DNA binding domain (DBD) that binds to a sequence of the target gene and a transcriptional repressor domain that suppresses expression of the target gene. The transcriptional repressor domain may be a known transcriptional repressor or may be a novel transcriptional repressor disclosed herein.

Also disclosed herein are sequences of novel transcriptional repressor domains that are conjugated to a heterologous DNA binding domain. As shown herein, these novel transcriptional repressor domains mediate suppression of expression of a target gene bound by the heterologous DNA binding domain.

Also disclosed herein are split systems for modulating gene expression where the DBD and the functional domain are provided as separated polypeptides and are assembled using dimerization of a heterodimer pair, where the DBD and the functional domain are each fused to a member of the heterodimer pair.

### BRIEF DESCRIPTION OF THE DRAWINGS

FIGS. 1A-1C illustrate the locations in the PDCD1 gene to which the DBDs of the indicated recombinant polypeptides were designed to bind. Recombinant polypeptides that repressed expression of PDCD1 in at least 50% of cells treated with the recombinant polypeptides are indicated by clear arrows ( [symbol] or [symbol] ). Recombinant polypeptides that repressed expression of PDCD1 in less than 50% of the cells treated with the recombinant polypeptides are indicated by solid arrows ( [symbol] or [symbol] ). The orientation of the arrows indicates the DNA strand to which the recombinant polypeptide is designed to bind. Arrows having the orientation [symbol] and [symbol] are designed to bind to the anti-sense strand. Arrows having the orientation [symbol] and [symbol] are designed to bind to the sense strand.

FIG. 2 shows the fold change in number of PD-1 expressing cells 2 days after transfection of mRNA encoding the indicated recombinant polypeptides into CD3+ T cells.

FIG. 3 shows effect of dose of mRNA encoding the recombinant polypeptide, pAL040 and pAL043, on the percent of CD3+ T cells expressing PD-1 3 days after transfection.

FIG. 4 shows the fold change in number of PD-1-positive cells at the indicated number of days post-transfection of mRNA encoding the indicated recombinant polypeptide relative to control.

FIGS. 5A and 5B show that PD-1 repression with pAL043 in anti-CD19 CAR-T cells is sustained after in vivo expansion and clearance of CD19-positive NALM-6 B-ALL tumor model in NOD SCID Gamma (NSG) mice.

FIG. 6 illustrates the locations in the TIM3 gene at which the DBDs of the indicated recombinant polypeptides bind. Recombinant polypeptides that repressed expression of TIM3 in at least 50% of the cells are indicated by unfilled arrows ( [symbol] or [symbol] ). Recombinant polypeptides that repressed expression of TIM3 in less than 50% of the cells are indicated by filled arrows ( [symbol] or [symbol] ).

FIG. 7 shows the fold change in number of cells expressing TIM3 at 2 days, 5 days, 8 days, or 14 days after transfection of mRNA encoding the indicated recombinant polypeptides into CD3+ T cells.

FIG. 8 shows the fold change in number of cells expressing TIM3 at 3 days or 6 days after transfection of mRNA encoding the indicated recombinant polypeptides into CD3+ T cells.

FIG. 9 illustrates the locations in the CTLA4 gene at which the DBDs of the indicated recombinant polypeptides bind. Recombinant polypeptides that repressed expression of CTLA4 in at least 50% of the cells are indicated by unfilled arrows ( [symbol] or [symbol] ). Recombinant polypeptides that repressed expression of CTLA4 in less than 50% of the cells are indicated by filled arrows ( [symbol] or [symbol] ).

FIG. 10 shows the fold change in number of cells expressing CTLA4 at 3 days after transfection of mRNA encoding the indicated recombinant polypeptides into CD3+ T cells.

FIG. 11 illustrates the locations in the LAG3 gene at which the DBDs of the indicated recombinant polypeptides bind. Recombinant polypeptides that repressed expression of LAG3 in at least 50% of the cells are indicated by unfilled arrows ( [symbol] or [symbol] ). Recombinant polypeptides that repressed expression of LAG3 in less than 50% of the cells are indicated by filled arrows ( [symbol] or [symbol] ).

FIG. 12 shows the fold change in number of cells expressing LAG3 at 2 days, 7 days, or 12 days after transfection of mRNA encoding the indicated recombinant polypeptides into CD3+ T cells.

FIG. 13 shows the fold change in number of cells expressing LAG3 at 2 days after transfection of mRNA encoding the indicated recombinant polypeptides into CD3+ T cells.

FIGS. 14A and 14B show multiplexing of recombinant polypeptides to simultaneously suppress expression of PD-1, LAG3, and TIM3 is a single cell.

FIGS. 15A-15C illustrates specificity of the recombinant polypeptides as indicated by lack of significant off-target effect as measured by RNA-seq.

FIG. 16 shows characterization of repression of TIM3 expression by the listed candidate transcriptional repressors.

FIG. 17 shows characterization of repression of LAG3, TIM3, or PD-1 expression by the listed candidate transcriptional repressors.

FIG. 18 shows characterization of repression of TIM3 expression by the listed candidate transcriptional repressors.

FIG. 19 shows a schematic of an anti-CD19 CAR-T cell in which expression of PD1, TIM3, and LAG3 has been repressed using the engineered polypeptides (pAL043+TL8188+TL8222) described herein.

FIG. 20 shows flow cytometry data confirming repression of PD1, TIM3, and LAG3 expression in the multiplex-treated CAR-T cells.

FIG. 21 provides an overview of in vivo leukemia xenograft model and treatment using indicated CAR-T cells.

FIG. 22 demonstrates that multiplexed repression of immune checkpoint genes is sustained in vivo.

FIG. 23 demonstrates that multiplexed repression of immune checkpoint genes enhances CAR-Ts ability to resist tumor re-challenge.

FIG. 24 shows expansion of CAR-Ts in the mouse blood.

FIG. 25 TALE-KRAB split system.

FIG. 26 Large-scale analysis of functional domains enabled by split encoding of DNA targeting and functional activities.

FIG. 27 Repression of TIM3 expression using TALE-KRAB split system.

FIGS. 28 and 29 Control of gene expression using CIPHR logic gates.

### DETAILED DESCRIPTION

The present disclosure provides recombinant polypeptides, compositions and methods for suppressing target gene expression for therapeutic purposes. In particular, described herein are engineered polypeptides comprising a DNA-binding domain (DBD) and a transcription repressor. The DBD mediates binding of the disclosed polypeptides to a sequence in the target gene. The target gene may be PDCD1, LAG3, TIM3, or CTLA4.

Certain regions in these target genes have been identified that can be targeted for repression of expression of these gene when these regions are bound by the polypeptides disclosed herein. These regions may be located in the target gene within an expression control region, such as, a coding region, a non-coding region, such as, a regulatory region (e.g., promoter region) or an intron.

These regions as well as the polypeptides that bind to these regions are provided herein.

Also disclosed herein are novel transcriptional repressors that are conjugated to a heterologous DNA binding domain and mediate suppression of expression of a target gene bound by the DNA binding domain.

Also disclosed herein are split systems for modulating gene expression where the DBD and the functional domain are provided as separated polypeptides and are assembled using dimerization of a heterodimer pair, where the DBD and the functional domain are each fused to a member of the heterodimer pair.

### Definitions

As used herein, the term “derived” in the context of a polypeptide refers to a polypeptide that has a sequence that is based on that of a protein from a particular source (e.g., Xanthomonas or Legionella). A polypeptide derived from a protein from a particular source may be a variant of the protein from the particular source. For example, a polypeptide derived from a protein from a particular source may have a sequence that is modified with respect to the protein's sequence from which it is derived. A polypeptide derived from a protein from a particular source shares at least 30% sequence identity with, at least 40% sequence identity with, at least 50% sequence identity with, at least 60% sequence identity with, at least 70% sequence identity with, at least 80% sequence identity with, or at least 90% sequence identity with the protein from which it is derived.

The term “modular” as used herein in the context of a DNA binding domain, e.g., a modular animal pathogen derived nucleic acid binding domain (MAP-NBD) indicates that the plurality of repeat units present in the DBD can be rearranged and/or replaced with other repeat units and can be arranged in an order such that the DBD binds to the target nucleic acid. For example, any repeat unit in a modular nucleic acid binding domain can be switched with a different repeat unit. In some aspects, modularity of the DNA binding domains disclosed herein allows for switching the target nucleic acid base for a particular repeat unit by simply switching it out for another repeat unit. In some embodiments, modularity of the DNA binding domains disclosed herein allows for swapping out a particular repeat unit for another repeat unit to increase the affinity of the repeat unit for a particular target nucleic acid. Overall, the modular nature of the DNA binding domains disclosed herein enables the development of genome editing complexes that can precisely target any nucleic acid sequence of interest.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. In specific aspects, the terms refer to a polymeric form of amino acids of any length which include genetically coded amino acids. In particular aspects, the terms refer to a polymeric form of amino acids of any length which include genetically coded amino acids fused to a heterologous amino acid sequence.

The term “heterologous” refers to two components that are defined by structures derived from different sources. For example, in the context of a polypeptide, a “heterologous” polypeptide may include operably linked amino acid sequences that are derived from different polypeptides (e.g., a DBD and a functional domain, e.g., a transcriptional repressor, derived from different sources). Similarly, in the context of a polynucleotide encoding a chimeric polypeptide, a “heterologous” polynucleotide may include operably linked nucleic acid sequences that can be derived from different genes. Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin than the promoter, the coding sequence or both). In the context of recombinant cells, “heterologous” can refer to the presence of a nucleic acid (or gene product, such as a polypeptide) that is of a different genetic origin than the host cell in which it is present.

The term “operably linked” refers to linkage between molecules to provide a desired function. For example, “operably linked” in the context of nucleic acids refers to a functional linkage between nucleic acid sequences. By way of example, a nucleic acid expression control sequence (such as a promoter, signal sequence, or array of transcription factor binding sites) may be operably linked to a second polynucleotide, wherein the expression control sequence affects transcription and/or translation of the second polynucleotide. In the context of a polypeptide, “operably linked” refers to a functional linkage between amino acid sequences (e.g., different domains) to provide for a described activity of the polypeptide.

A “target nucleic acid,” “target sequence,” or “target site” is a nucleic acid sequence that defines a portion of a nucleic acid to which a binding molecule, such as, the DBD disclosed herein will bind. The target nucleic acid may be present in an isolated form or inside a cell. A target nucleic acid may be present in a region of interest. A “region of interest” may be any region of cellular chromatin, such as, for example, a gene or a non-coding sequence within or adjacent to a gene, in which it is desirable to bind an exogenous molecule. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, promoter sequences, leader sequences, trailer sequences or introns, or within non-transcribed regions, either upstream or downstream of the coding region. A region of interest can be as small as a five nucleotide pair or up to 200 nucleotide pairs in length, or any integral value of nucleotide pairs.

An “exogenous” molecule is a molecule that is not normally present in a cell but can be introduced into a cell by one or more genetic, biochemical or other methods. An exogenous nucleic acid can be present in an infecting viral genome, a plasmid or episome introduced into a cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, shRNA, RNAi, miRNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

The terms “conjugating,” “conjugated,” and “conjugation” refer to an association of two entities, for example, of two molecules such as two proteins, two domains (e.g., a binding domain and a transcription repressor domain), or a protein and an agent, e.g., a protein binding domain and a small molecule. The association can be, for example, via a direct or indirect (e.g., via a linker) covalent linkage or via non-covalent interactions. In some embodiments, the association is covalent. In some embodiments, two molecules are conjugated via a linker connecting both molecules. For example, in some embodiments where two proteins are conjugated to each other, e.g., a binding domain and a cleavage domain of an engineered nuclease, to form a protein fusion, the two proteins may be conjugated via a polypeptide linker, e.g., an amino acid sequence connecting the C-terminus of one protein to the N-terminus of the other protein. Such conjugated proteins may be expressed as a fusion protein.

The term “effective amount,” as used herein, refers to an amount of a biologically active agent that is sufficient to elicit a desired biological response. For example, in some aspects, an effective amount of a polypeptide comprising a transcriptional repressor may refer to the amount of the polypeptide that is sufficient to induce repression of expression from a gene specifically bound by the polypeptide. As will be appreciated by the skilled artisan, the effective amount of an agent, e.g., a recombinant polynucleotide, may vary depending on various factors as, for example, on the desired biological response, the specific allele, genome, target site, cell, or tissue being targeted, and the agent being used.

The term “strand” as used herein refers to a nucleic acid made up of nucleotides covalently linked together by covalent bonds, e.g., phosphodiester bonds. In a cell, DNA usually exists in a double-stranded form, and as such, has two complementary strands of nucleic acid referred to herein as the “top” and “bottom” strands or the “Watson” and “Crick” strands. Watson strand refers to 5′ to 3′ top strand (5′→3′), whereas Crick strand refers to 3′ to 5′ bottom strand (3′←5′). The assignment of a strand as being a top or bottom strand is arbitrary and does not imply any particular orientation, function or structure. In certain cases, complementary strands of a chromosomal DNA may be interchangeably referred to as “top” and “bottom” strands, “plus” and “minus” strands, the “first” and “second” strands, the “coding” and “noncoding” strands, the “Watson” and “Crick” strands, or the “sense” and “antisense” strands. The nucleotide sequences of the coding strand of several mammalian chromosomal regions (e.g., BACs, assemblies, chromosomes, etc.) are known, and may be found in NCBI's GenBank database, for example.

As used herein, the term, “on-target” repression refers repression of expression of a gene containing the genomic sequence that is the target of the recombinant polypeptide comprising the DBD and the transcription repressor. The DBD determines the specificity of the polypeptide for the binding the target site. An on-target repression site refers to a nucleic acid sequence that includes the DNA sequence specifically bound by the DBD of the recombinant polypeptide.

As used herein, the term, “off-target” repression refers to repression of expression of a gene containing the genomic sequence that is not the target of the recombinant polypeptide comprising the DBD and the transcription repressor but is repressed due to non-specific binding of the DBD of the recombinant polypeptide.

As used herein, the term “domain” or “protein domain” refers to a part of a protein sequence that may exist and function independently of the rest of the protein chain. In the context of the recombinant polypeptides disclosed herein, these recombinant polypeptides function as transcriptional repressors by virtue of the DBD that mediates binding to a target gene and a repressor domain that suppresses target gene expression upon binding of the polypeptide to the target gene. The recombinant polypeptides disclosed herein may also be referred to as transcriptional repressors.

The sequences provided herein may be specified to be at least 30%, 40%, 50%, 70%, 75%, 80%, 85%, 90%, 91%, 92%, 93%, 94%, 95%, 96%, 97%, 98%, or 99%, or a 100% identical to another sequence provided herein. Percent identity between a pair of sequences may be calculated by multiplying the number of matches in the pair by 100 and dividing by the length of the aligned region, including gaps.

Identity scoring only counts perfect matches and does not consider the degree of similarity of amino acids to one another. Only internal gaps are included in the length, not gaps at the sequence ends.

Percent Identity=(Matches×100)/Length of aligned region (with gaps)

The phrase “conservative amino acid substitution” refers to substitution of amino acid residues within the following groups: 1) L, I, M, V, F; 2) R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain. Guidance for substitutions, insertions, or deletions may be based on alignments of amino acid sequences of proteins from different species or from a consensus sequence based on a plurality of proteins having the same or similar function.

The terms “patient” or “subject” are used interchangeably to refer to a human or a non-human animal (e.g., a mammal).

The terms “treat”, “treating”, reatment” and the like refer to a course of action (such as administering a polypeptide comprising a DBD fused to a heterologous transcription repressor domain or a nucleic acid encoding the polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The terms “prevent”, “preventing”, “prevention” and the like refer to a course of action (such as administering a polypeptide comprising a DBD fused to a heterologous functional domain or a nucleic acid encoding the polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The phrase “therapeutically effective amount” refers to the administration of an agent to a subject, either alone or as apart of a pharmaceutical composition or as a companion therapy and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

### Recombinant Polypeptides

As noted above, the recombinant polypeptides include a DBD that mediates binding to a sequence in a target gene and a heterologous transcriptional repressor. The DBD includes a plurality of RUs ordered from N-terminus to C-terminus of the DBD to bind to a nucleic acid sequence of the target gene, where binding of the recombinant polypeptide to the nucleic acid sequence results in decreased expression of the target gene.

In certain aspects, a recombinant polypeptide disclosed herein may include from N- to C-terminus: a N-cap region, a DBD comprising a plurality of RUs ordered from N-terminus to C-terminus of the DBD to bind to a nucleic acid sequence of the target gene, a C-cap region, an optional linker, and a transcription repressor domain. In certain aspects, the transcriptional repressor domain may be at the N-terminus of the recombinant polypeptide instead of the C-terminus.

The RUs may have the sequence (X₁₋₁₁X₁₂X₁₃X_{14-33, 34, or 35})_{z} (SEQ ID NO: 453), where X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent, and wherein z=7-40, 7-35, or 7-25.

Any suitable RU such as those based upon the RUs from Xanthomonas transcription activator-like effector (TALE) systems, Ralstonia solanacearum (modular Ralstonia nucleic acid binding domain; RNBD), or an animal pathogen (e.g., Legionella quateirensis, Legionella maceachernii, Burkholderia, Paraburkholderia, or Francisella) (modular animal pathogen nucleic acid binding domain; MAP-NBD) may be used for binding to the regions of the target genes provided herein. The arrangement of the RUs in the DBD may be based upon the sequence identified in the target gene to which binding of the recombinant polypeptide results in decreased expression of the target gene. These sequences identified in PDCD-1 gene, TIM3 gene, CTLA4 gene, and LAG3 gene, and the corresponding DBDs are described in detail below.

PDCD-1 (programmed cell death 1) gene is also known as PD-1 gene and encodes a cell surface membrane protein of the immunoglobulin superfamily, which is also referred to as PDCD-1 or PD-1. PD-1 binds to the ligands PD-L1 and PD-L2. The PD-1/PD-1 ligands pathway plays a role in immunosuppression. Recent studies have shown that PD-L1 and PD-L2 are widely expressed on various cancer cells (Keir M E, et al., Annu Rev Immunol. 2008; 26 (677-704)). Expression of PD-ligands prevents cancer cells from being killed by T cells and promotes cancer progression. Targeting the PD-1 pathway has been recognized as an effective immunotherapy for different cancers (Ostrand-Rosenberg S, et al., J Immunol. 2014; 193(8):3835-41).

TIM3 (T-Cell Immunoglobulin Mucin Receptor 3) gene is also referred to as Hepatitis A Virus Cellular Receptor 2 (HAVCR2) and encodes a cell surface membrane protein of the immunoglobulin superfamily of the same name.

CTLA4 gene (Cytotoxic T-Lymphocyte Associated Protein 4) encodes an immunoglobulin superfamily protein of the same name which transmits an inhibitory signal to T cells.

LAG3 (Lymphocyte-Activation Gene 3) gene encodes the Lymphocyte Activating 3 protein which is also known as LAG3 protein.

CTLA-4, PD-1, LAG-3, and TIM3 are known immune checkpoint proteins. The pathways involving LAG3 and TIM3 are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489).

Unless stated otherwise, all nucleic acid sequences are written from 5′ to 3′ and all polypeptide sequences are from N-terminus to C-terminus. As indicated herein, a DBD may include a plurality of RUs ordered from N-terminus to C-terminus of the DBD to bind to a nucleic acid sequence of the PDCD1 gene. The plurality of RUs may be a number of repeat units sufficient to bind to a target sequence, which number may range from 7 to 40. In certain aspects, the number of RUs may range from 9 to 35. In certain aspects, the number of RUs may range from 12 to 30, 14 to 25, or 16 to 25.

In certain aspects, the recombinant polypeptides disclosed herein all reduce the expression of the target gene in at least 50% of the cells transfected with a nucleic acid encoding the recombinant polypeptides while cells not transfected with a nucleic acid encoding the recombinant polypeptides do not show a significant decrease in the expression of the target gene. In certain aspects, the recombinant polypeptides disclosed herein all reduce the expression of the target gene in at least 80% of the cells transfected with a nucleic acid encoding the recombinant polypeptides while cells not transfected with a nucleic acid encoding the recombinant polypeptides do not show a significant decrease in the expression of the target gene.

### PDCD-1 Repressors

Provided herein are recombinant polypeptides that bind to sequences in the PDCD-1 gene that have been identified to be present in regions of the gene that when bound by the recombinant polypeptides comprising a transcriptional repressor domain lead to suppression of PD-1 expression from the PDCD-1 gene.

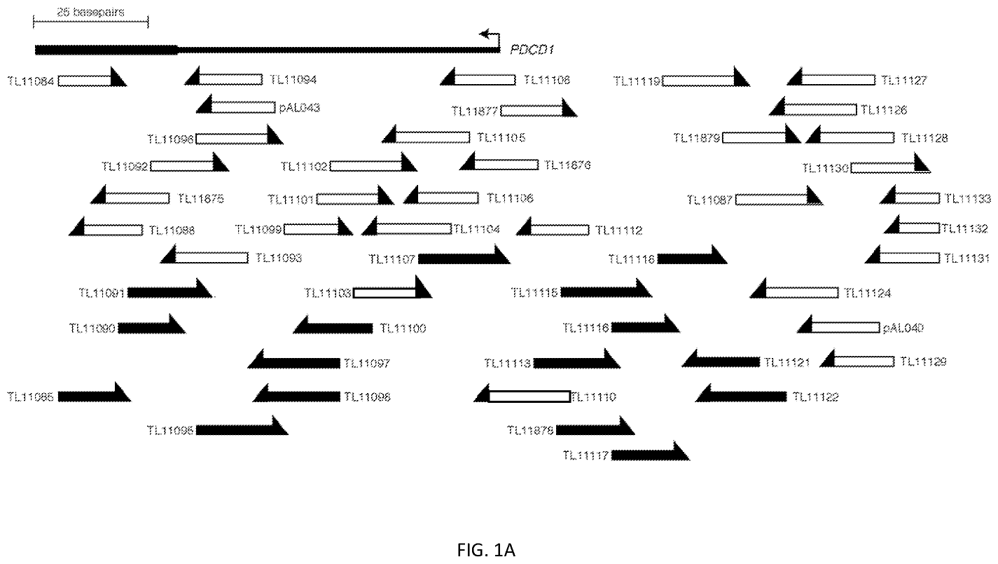

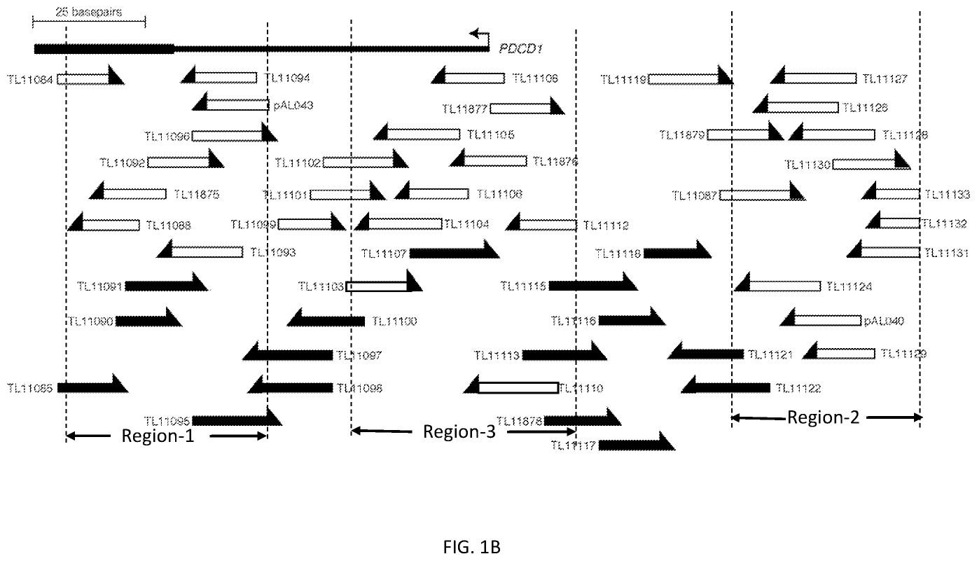

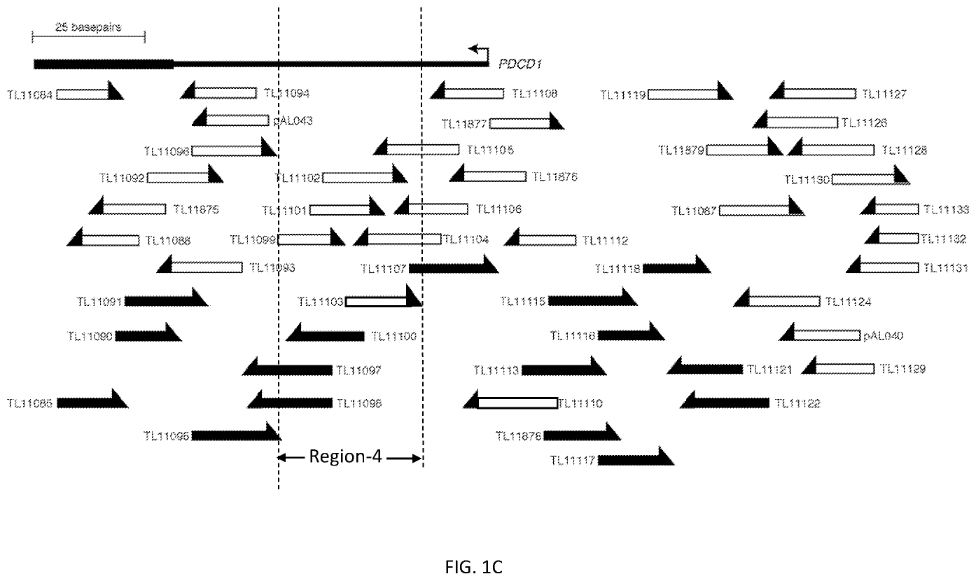

The sequences in the PDCD-1 gene that were tested to determine repression by a transcriptional repressor domain bound to the sequence are pictorially depicted in FIG. 1A. The analysis of repression by the disclosed recombinant polypeptides that are designed to bind to these sequences identified certain regions that provide repression of PDCD-1 expression in at least 50% of the cells expressing these recombinant polypeptides. These regions are depicted in FIGS. 1B-1C and include regions 1-4. In regions 1, 2, 3, the anti-sense strand of the PDCD-1 gene was successfully targeted to significantly repress expression of PD-1. In region 4, the sense strand was identified as the region of the PDCD-1 gene that can be successfully target for repression. In addition, certain sequences in the sense strand in region 1 were also identified a region that can be successfully targeted for repression.

Region 1:

Table 1 illustrates the identification of region 1 which includes sequences that can be targeted for repression. As can be seen from Table 1, the indicated recombinant polypeptides, that included RUs arranged from N-terminus to C-terminus to bind to the listed target sequence, repressed expression of PD-1 by at least 80% as compared to a negative control. The location of these target sequences when aligned reveals a region (Region 1) in minus strand of the PDCD-1 gene that may be targeted for repressing PDCD-1 expression. The alignment of the target sequences also reveals the minimal sequences within Region 1 that can be targeted for binding by the DBD for repressing PDCD-1 expression.

TABLE 1
Region 1

| TALE ID | Target Sequence | Repression |
|---|---|---|
| pAL043 (or | TGGTGGGGCTGCTCC | ≥80% |
| PD02) | (SEQ ID NO: 5) | |
| TL11094 | GGTGGGGCTGCTCCAGG | ≥80% |
| | (SEQ ID NO: 6) | |
| TL11093 | GGGGCTGCTCCAGGCATGC | ≥50% |
| | (SEQ ID NO: 9) | |
| TL11875 | GCAGATCCCACAGGCGC | ≥80% |
| | (SEQ ID NO: 7) | |
| TL11088 | CCCACAGGCGCCCTGG | ≥50% |
| | (SEQ ID NO: 8) | |
| Region 1 | TGGTGGGGCTGCTCCAGGCA | |
| | TGCAGATCCCACAGGCGCCC | |
| | TGG (SEQ ID NO: 1) | |
| Sequence | GGTGGGGCTGCTCC | |
| common to | (SEQ ID NO: 4) | |
| pAL043 and | | |
| TL11094 | | |
| Sequence | GGGGCTGCTCC (SEQ ID NO: 2) | |
| common to | | |
| pAL043, | | |
| TL11094, and | | |
| TL11093 | | |

Accordingly, in certain aspects, a recombinant polypeptide that suppresses expression of PD1 receptor encoded by the PDCD1 gene may include a DNA binding domain (DBD) and a transcriptional repressor domain. The DBD may include a plurality of RUs ordered from N-terminus to C-terminus of the DBD to bind to a nucleic acid sequence of the PDCD1 gene, wherein the nucleic acid sequence is present within the sequence: TGGTGGGGCTGCTCCAGGCATGCAGATCCCACAGGCGCCCTGG (SEQ ID NO: 1). As explained in the Examples section of the application, this sequence corresponds to Region 1 in the PDCD1 gene.

The RUs may include the sequence (X₁₋₁₁X₁₂X₁₃X_{14-33, 34, or 35})_{z} (SEQ ID NO: 453), where X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent, and wherein z=7-40, 7-35, or 7-25.

In certain aspects, the RUs are ordered from N-terminus to the C-terminus to bind to the sequence: GGGGCTGCTCC (SEQ ID NO:2), wherein the first RU at the N-terminus binds to the G at the 5′ end of the sequence and the last RU at the C-terminus binds to the C at the 3′ end of the sequence. In certain aspects, the X₁₂X₁₃ in the RUs from N-terminus to C-terminus may be NH, NH, NH, NH, HD, NG, NH, HD, NG, HD, and HD.

In certain aspects, the DBD may include at least an additional RU at the N-terminus such that the DBD binds to the nucleic acid sequence TGGGGCTGCTCC (SEQ ID NO:3), wherein X₁₂X₁₃ in the additional RU is NG, HG, KG, or RG for recognition of the T.

In certain aspects, the RUs are ordered from N-terminus to the C-terminus to bind to the sequence: GGTGGGGCTGCTCC (SEQ ID NO:4), wherein the first RU at the N-terminus binds to the G at the 5′ end of the sequence and the last RU at the C-terminus binds to the C at the 3′ end of the sequence. In certain aspects, the DBD comprises at least fourteen RUs, wherein X₁₂X₁₃ in the RUs from N-terminus to C-terminus are NH, NH, NG, NH, NH, NH, NH, HD, NG, NH, HD, NG, HD, and HD. In certain aspects, the DBD comprises three additional RU at the N-terminus such that the DBD binds to the nucleic acid sequence TGGTGGGGCTGCTCC (SEQ ID NO:5). In certain aspects, the DBD comprises three additional RUs at the C-terminus such that the DBD binds to the sequence GGTGGGGCTGCTCCAGG (SEQ ID NO:6).

In certain aspects, the RUs are arranged from N-terminus to C-terminus to bind to the sequence: GCAGATCCCACAGGCGC (SEQ ID NO:7).

In certain aspects, the RUs are arranged from N-terminus to C-terminus to bind to the sequence: CCCACAGGCGCCCTGG (SEQ ID NO:8).

In certain aspects, the RUs are arranged from N-terminus to C-terminus to bind to the sequence: GGGGCTGCTCCAGGCATGC (SEQ ID NO:9).

In certain aspects, the RUs may be arranged from N-terminus to C-terminus to bind to a sequence that is a complement of a sequence in region 1. In certain aspects, the complementary sequence may be the sequence: GGAGCAGCCCC (SEQ ID NO: 105). In certain aspects, the DBD that binds to the complementary sequence may include RUs ordered from N-terminus to C-terminus to bind to the sequence: GGAGCAGCCCCACCAGAGT (SEQ ID NO: 106).

Region 2:

Table 2 illustrates the identification of region 2 which includes sequences that can be targeted for repression. As can be seen from Table 2, the indicated recombinant polypeptides, that included RUs arranged from N-terminus to C-terminus to bind to the listed target sequence, repressed expression of PD-1 by at least 80% as compared to a negative control. The location of these target sequences when aligned reveals a region (Region 2) in the minus strand of the PDCD-1 gene that may be targeted for repressing PDCD-1 expression. The alignment of the target sequences also reveals the minimal sequence that can be targeted for binding by the DBD for repressing PDCD-1 expression.

TABLE 2
Region 2

| TALE ID | Target Sequence | Repression |
|---|---|---|
| TL11124 | CTCGCCCACGTGGATGTGG | >50% |
| | (SEQ ID NO: 345) | |
| TL11126 | CACTCTCGCCCACGTGGAT | >50% |
| | (SEQ ID NO: 346) | |
| TL11127 | CTGTCACTCTCGCCCACGT | >50% |
| | (SEQ ID NO: 347) | |
| pAL040 | TCTGTCACTCTCGCCCAC | >80% |
| | (SEQ ID NO: 14) | |
| TL11128 | GCCTCTGTCACTCTCGCCC | >80% |
| | (SEQ ID NO: 13) | |
| TL11129 | GCCTCTGTCACTCTCG | >80% |
| | (SEQ ID NO: 12) | |
| TL11131 | CCCCCAGCACTGCCTCT | >50% |
| | (SEQ ID NO: 349) | |
| TL11132 | CCTCCCCCAGCACTGC | >80% |
| | (SEQ ID NO: 16) | |
| TL11133 | CCTCCCCCAGCACTGCC | >80% |
| | (SEQ ID NO: 17) | |
| Region 2 | CCTCCCCCAGCACTGCCTCTGTC | |
| | ACTCTCGCCCACGTGGATGTGG | |
| | (SEQ ID NO: 10) | |
| Common | TCTGTCACTCTCG | |
| sequence | (SEQ ID NO: 11) | |
| bound by | | |
| pAL040, | | |
| TL111128, | | |
| TL11129 | | |
| Common | GCCTCTGTCACTCTCG | |
| sequence | (SEQ ID NO: 12) | |
| bound by | | |
| TL111128 | | |
| and | | |
| TL11129 | | |
| Common | CCCCCAGCACTGC | |
| sequence | (SEQ ID NO: 15) | |
| bound by | | |
| TL11131, | | |
| TL11132, | | |
| TL11133 | | |
| Common | CCTCCCCCAGCACTGC | |
| sequence | (SEQ ID NO: 16) | |
| bound by | | |
| TL11132 | | |
| and | | |
| TL11133 | | |

In some aspects, the DBD includes a plurality of RUs ordered from N-terminus to C-terminus of the DBD to bind to a nucleic acid sequence of the PDCD1 gene, wherein the nucleic acid sequence is present within the sequence:

(SEQ ID NO: 10)
CCTCCCCCAGCACTGCCTCTGTCACTCTCGCCCACGTGGATGTGG

As explained herein, this sequence is the sequence of Region 2.

In some aspects, the DBD includes a plurality of RUs ordered from N-terminus to C-terminus of the DBD to bind to a nucleic acid sequence of the PDCD1 gene, wherein the nucleic acid sequence is present within the sequence: CCTCCCCCAGCACTGCCTCTGTCACTCTCGCCCACGT (SEQ ID NO: 454). As shown in the Examples section of the application, all of the eight DBD-repressor domains that bound to a nucleic acid sequence within this sequence, repressed expression of PD-1 in at least 50% of the cells treated with the DBD-repressor domain as compared to mock treated cells.

In some aspects, the DBD includes a plurality of RUs ordered from N-terminus to C-terminus of the DBD to bind to a nucleic acid sequence of the PDCD1 gene, wherein the nucleic acid sequence is present within the sequence: GCCTCTGTCACTCTCGCCCAC (SEQ ID NO: 444). As shown in the Examples section of the application, all of the three DBD-repressor domains (pAL040, TL11128, and TL11129) that bound to a nucleic acid sequence within this sequence, repressed expression of PD-1 in at least 80% of the cells treated with the DBD-repressor domain as compared to mock treated cells.

In certain aspects, the RUs are ordered from N-terminus to C-terminus of the DBD to bind to the nucleic acid sequence TCTGTCACTCTCG (SEQ ID NO: 11). In certain aspects, the DBD comprises at least thirteen RUs, wherein X₁₂X₁₃ in the RUs from N-terminus to C-terminus are NG, HD, NG, NH, NG, HD, NI, HD, NG, HD, NG, HD, and NH. In certain aspects, the DBD further comprises three additional RUs at the N-terminus such that the DBD binds to the nucleic acid sequence GCCTCTGTCACTCTCG (SEQ ID NO: 12). In certain aspects, the DBD further comprises three additional RUs at the C-terminus such that the DBD binds to the nucleic acid sequence GCCTCTGTCACTCTCGCCC (SEQ ID NO: 13).

In certain aspects, the DBD comprises at least nineteen RUs, wherein X₁₂X₁₃ in the RUs from N-terminus to C-terminus are NH, HD, HD, NG, HD, NG, NH, NG, HD, NI, HD, NG, HD, NG, HD, NH, HD, HD, and HD. In certain aspects, the DBD further comprises five additional RUs at the C-terminus such that the DBD binds to the nucleic acid sequence TCTGTCACTCTCGCCCAC (SEQ ID NO: 14). In certain aspects, the DBD comprises at least eighteen RUs, wherein X₁₂X₁₃ in the RUs from N-terminus to C-terminus are NG, HD, NG, NH, NG, HD, NI, HD, NG, HD, NG, HD, NG, NH, HD, HD, HD, NI, and HD.

In certain aspects, the DBD comprises thirteen RUs ordered from N-terminus to C-terminus of the DBD to bind to the nucleic acid sequence: CCCCCAGCACTGC (SEQ ID NO: 15). In certain aspects, the DBD further comprises three additional RUs at the N-terminus such that the DBD binds to the nucleic acid sequence: CCTCCCCCAGCACTGC (SEQ ID NO: 16). In certain aspects, the DBD further comprises an additional RU at the C-terminus such that the DBD binds to the nucleic acid sequence:

(SEQ ID NO: 17)
CCTCCCCCAGCACTGCC.

Region 3:

Table 3 illustrates the identification of region 3 which includes sequences that can be targeted for repression. As can be seen from Table 3, the indicated recombinant polypeptides, that included RUs arranged from N-terminus to C-terminus to bind to the listed target sequence, repressed expression of PD-1 by at least 80% as compared to a negative control. The location of these target sequences when aligned reveals a region (Region 3) in the minus strand in the PDCD-1 gene that may be targeted for repressing PDCD-1 expression. The alignment of the target sequences also reveals the minimal sequence that can be targeted for binding by the DBD for repressing PDCD-1 expression.

TABLE 3
Region 3

| TALE ID | Target Sequence | Repression |
|---|---|---|
| TL11104 | TCCGCTCACCTCCGCCTGA | >80% |
| | (SEQ ID NO: 21) | |
| TL11105 | CCCTTCCGCTCACCTCCGC | >80% |
| | (SEQ ID NO: 23) | |
| TL11106 | TTCCCTTCCGCTCACC | >80% |
| | (SEQ ID NO: 24) | |
| TL11108 | GGGACAGTTTCCCTTC | >80% |
| | (SEQ ID NO: 26) | |
| TL11876 | GACCTGGGACAGTTTCC | >80% |
| | (SEQ ID NO: 27) | |
| TL11110 | CAACCTGACCTGGGACAGTT | >80% |
| | (SEQ ID NO: 29) | |
| TL11112 | CCCTTCAACCTGACCT | >80% |
| | (SEQ ID NO: 30) | |
| Region 3 | CCCTTCAACCTGACCTGGGACAG | |
| | TTTCCCTTCCGCTCACCTCC | |
| | GCCTGA (SEQ ID NO: 19) | |
| Common | TCCGCTCACC | |
| sequence | (SEQ ID NO: 20) | |
| bound by | | |
| TL11104, | | |
| TL1110, | | |
| TL11106 | | |
| Common | GGGACAGTTTCC | |
| sequence | (SEQ ID NO: 25) | |
| bound by | | |
| TL11108 | | |
| TL11876 | | |
| Common | CAACCTGACCT | |
| sequence | (SEQ ID NO: 28) | |
| bound by | | |
| TL11110 | | |
| TL1112 | | |

In certain aspects, the DBD includes at least nine RUs ordered from N-terminus to C-terminus of the DBD to bind to a nucleic acid sequence of the PDCD1 gene, wherein the nucleic acid sequence is present within the sequence:

CCCTTCAACCTGACCTGGGACAGTTTCCCTTCCGCTCACCTCCGCCTGA (SEQ ID NO: 19). As explained in the Examples section of the application, this sequence corresponds to region 3 of the PDCD1 gene.

In certain aspects, the DBD comprises ten RUs ordered from N-terminus to C-terminus to bind to the nucleic acid sequence: TCCGCTCACC (SEQ ID NO:20). In certain aspects, the DBD comprises nine additional RUs at the C-terminus such that the DBD binds to the nucleic acid sequence: TCCGCTCACCTCCGCCTGA (SEQ ID NO:21). In certain aspects, the DBD comprises four additional RUs at the N-terminus such that the DBD binds to the nucleic acid sequence: CCCTTCCGCTCACC (SEQ ID NO: 22). In certain aspects, the DBD comprises five additional RUs at the C-terminus such that the DBD binds to the nucleic acid sequence: CCCTTCCGCTCACCTCCGC (SEQ ID NO: 23). In certain aspects, the DBD comprises two additional RUs at the N-terminus such that the DBD binds to the nucleic acid sequence: TTCCCTTCCGCTCACC (SEQ ID NO: 24).

In certain aspects, the DBD comprises twelve RUs ordered from N-terminus to C-terminus to bind to the nucleic acid sequence: GGGACAGTTTCC (SEQ ID NO:25). In certain aspects, the DBD further comprises four additional RUs at the C-terminus such that the DBD binds to the nucleic acid sequence: GGGACAGTTTCCCTTC (SEQ ID NO:26). In certain aspects, the DBD further comprises five additional RUs at the N-terminus such that the DBD binds to the nucleic acid sequence:

(SEQ ID NO: 27)
GACCTGGGACAGTTTCC.

In certain aspects, the DBD comprises eleven RUs ordered from N-terminus to C-terminus to bind to the nucleic acid sequence: CAACCTGACCT (SEQ ID NO:28). In certain aspects, the DBD comprises nine additional RUs at the C-terminus such that the DBD binds to the nucleic acid sequence: CAACCTGACCTGGGACAGTT (SEQ ID NO:29) In certain aspects, the DBD comprises five additional RUs at the N-terminus such that the DBD binds to the nucleic acid sequence: CCCTTCAACCTGACCT (SEQ ID NO:30).

Region 4:

Table 4 illustrates the identification of region 4 which includes sequences that can be targeted for repression. As can be seen from Table 4, the indicated recombinant polypeptides, that included RUs arranged from N-terminus to C-terminus to bind to the listed target sequence, repressed expression of PD-1 by at least 80% as compared to a negative control. The location of these target sequences when aligned reveals a region (Region 4) in the plus strand of the PDCD-1 gene that may be targeted for repressing PDCD-1 expression. The alignment of the target sequences also reveals the minimal sequence that can be targeted for binding by the DBD for repressing PDCD-1 expression. PGP-46 DNA

TABLE 4
Region 4

| TALE ID | Sequence | Repression |
|---|---|---|
| TL11099 | GCCGCCTTCTCCACT | >80% |
| | (SEQ ID NO: 32) | |
| TL11101 | TCTCCACTGCTCAGGCG | >80% |
| | (SEQ ID NO: 34) | |
| TL11102 | CCACTGCTCAGGCGGAGGT | >50% |
| | (SEQ ID NO: 35) | |
| Region 4 | GCCGCCTTCTCCACTGCTCAGG | |
| | CGGAGGT (SEQ ID NO: 31) | |
| Common | TCTCCACT (SEQ ID NO: 445) | |
| sequence | | |
| bound by | | |
| TL11099 | | |
| and | | |
| TL11101 | | |

In other aspects, the DBD includes at least nine RUs ordered from N-terminus to C-terminus of the DBD to bind to a nucleic acid sequence of the PDCD1 gene, wherein the nucleic acid sequence is present within the sequence: GCCGCCTTCTCCACTGCTCAGGCGGAGGT (SEQ ID NO:31).

As explained in the Examples section of the application, this sequence corresponds to Region 4.

In certain aspects, the DBD comprises RUs arranged from N-terminus to C-terminus such that the DBD binds to the nucleic acid sequence: GCCGCCTTCTCCACT (SEQ ID NO:32).

In certain aspects, the DBD comprises RUs arranged from N-terminus to C-terminus such that the DBD binds to the nucleic acid sequence: CCACTGCTCAGGCG (SEQ ID NO:33). In certain aspects, the DBD further comprises three additional RUs at the N-terminus such that the DBD binds to the nucleic acid sequence: TCTCCACTGCTCAGGCG (SEQ ID NO:34). In certain aspects, the DBD further comprises five additional RUs at the C-terminus such that the DBD binds to the nucleic acid sequence:

(SEQ ID NO: 35)
CCACTGCTCAGGCGGAGGT.

In addition to the recombinant polypeptides that bind to a sequence in Regions 1-4 of PDCD1, the present disclosure provides additional recombinant polypeptides for repressing PDCD1 expression. In certain aspects, the DBD of the recombinant polypeptide includes at least nine RUs ordered from N-terminus to C-terminus of the DBD to bind to a nucleic acid sequence of the PDCD1 gene, wherein the nucleic acid sequence is present within the sequence: CCCAGGTCAGGTTGAAG (SEQ ID NO:63). In certain aspects, the DBD of the recombinant polypeptide includes at least nine RUs ordered from N-terminus to C-terminus of the DBD to bind to a nucleic acid sequence of the PDCD1 gene, wherein the nucleic acid sequence is present within the sequence: GGCCAGGGCGCCTGT (SEQ ID NO:36). In certain aspects, the DBD of the recombinant polypeptide includes at least nine RUs ordered from N-terminus to C-terminus of the DBD to bind to a nucleic acid sequence of the PDCD1 gene, wherein the nucleic acid sequence is present within the sequence: CTGCATGCCTGGAGCAG (SEQ ID NO:37). In certain aspects, the DBD of the recombinant polypeptide includes at least nine RUs ordered from N-terminus to C-terminus of the DBD to bind to a nucleic acid sequence of the PDCD1 gene, wherein the nucleic acid sequence is present within the sequence: GCTCCCGCCCCCTCTTCCT (SEQ ID NO:38). In certain aspects, the DBD of the recombinant polypeptide includes at least nine RUs ordered from N-terminus to C-terminus of the DBD to bind to a nucleic acid sequence of the PDCD1 gene, wherein the nucleic acid sequence is present within the sequence: CTTCCTCCACATCCACG (SEQ ID NO:39). In certain aspects, the DBD of the recombinant polypeptide includes at least nine RUs ordered from N-terminus to C-terminus of the DBD to bind to a nucleic acid sequence of the PDCD1 gene, wherein the nucleic acid sequence is present within the sequence: CCTCCACATCCACGTGGGC (SEQ ID NO:40).

In certain aspects, the RUs of the recombinant polypeptide may be arranged from N-terminus to C-terminus to bind to a sequence present in a target sequence listed in Table 9 and shown to have a PD-1 suppression of at least 50%. As noted herein the RUs may range from 7 to 40 in number. In certain aspects, the RUs of the recombinant polypeptide may be arranged from N-terminus to C-terminus to bind to the target sequence listed in Table 9 and shown to have a PD-1 suppression of at least 50%.

In certain aspects, the recombinant polypeptides disclosed herein all reduce the expression of PDCD1 gene in at least 50% of the cells transfected with a nucleic acid encoding the recombinant polypeptides while cells not transfected with a nucleic acid encoding the recombinant polypeptides do not show a significant decrease in the expression of the target gene.

In certain aspects, the recombinant polypeptides disclosed herein all reduce the expression of the PDCD1 gene in at least 80% of the cells transfected with a nucleic acid encoding the recombinant polypeptides while cells not transfected with a nucleic acid encoding the recombinant polypeptides do not show a significant decrease in the expression of the PDCD1 gene. Accordingly, in certain aspects, a recombinant polypeptide of the present disclosure may include a DBD and a transcriptional repressor domain, the DBD comprising a plurality of RUs ordered from N-terminus to C-terminus of the DBD to bind to a nucleic acid sequence of the PDCD1 gene, wherein the nucleic acid sequence is present within one of the following sequences:

(SEQ ID NO: 5)
TGGTGGGGCTGCTCC;
(SEQ ID NO: 27)
GACCTGGGACAGTTTCC;
(SEQ ID NO: 24)
TTCCCTTCCGCTCACC;
(SEQ ID NO: 32)
GCCGCCTTCTCCACT;
(SEQ ID NO: 13)
GCCTCTGTCACTCTCGCCC;
(SEQ ID NO: 63)
CCCAGGTCAGGTTGAAG;
(SEQ ID NO: 6)
GGTGGGGCTGCTCCAGG;
(SEQ ID NO: 34)
TCTCCACTGCTCAGGCG;
(SEQ ID NO: 21)
TCCGCTCACCTCCGCCTGA;
(SEQ ID NO: 23)
CCCTTCCGCTCACCTCCGC;
(SEQ ID NO: 26)
GGGACAGTTTCCCTTC;
(SEQ ID NO: 12)
GCCTCTGTCACTCTCG;
(SEQ ID NO: 7)
GCAGATCCCACAGGCGC;
(SEQ ID NO: 16)
CCTCCCCCAGCACTGC;
(SEQ ID NO: 17)
CCTCCCCCAGCACTGCC;
(SEQ ID NO: 14)
TCTGTCACTCTCGCCCAC;
and
(SEQ ID NO: 29)
CAACCTGACCTGGGACAGTT,

wherein each of the RU comprises the sequence X₁₋₁₁X₁₂X₁₃X_{14-33, 34, or 35} (SEQ ID NO: 455), wherein X₁₋₁₁ is a chain of 11 contiguous amino acids, X_{14-33 or 34 or 35} is a chain of 20, 21 or 22 contiguous amino acids, X₁₂X₁₃ is selected from (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent.

In certain aspects, the DBD comprises at least fourteen RUs, at least sixteen, or at least seventeen RUs and optionally, up to 25 RUs.

In certain aspects, DBD binds to the nucleic acid sequence selected from SEQ ID NOs.: 5, 27, 24, 32, 13, 63, 6, 34, 21, 23, 26, 12, 7, 16, 17, 14, and 29.

### TIM3 Repressors

Provided herein are recombinant polypeptides that bind to sequences in the TIM3 gene that have been identified to be present in regions of the gene that when bound by the recombinant polypeptides comprising a transcriptional repressor domain lead to suppression of TIM3 expression from the TIM3 gene.

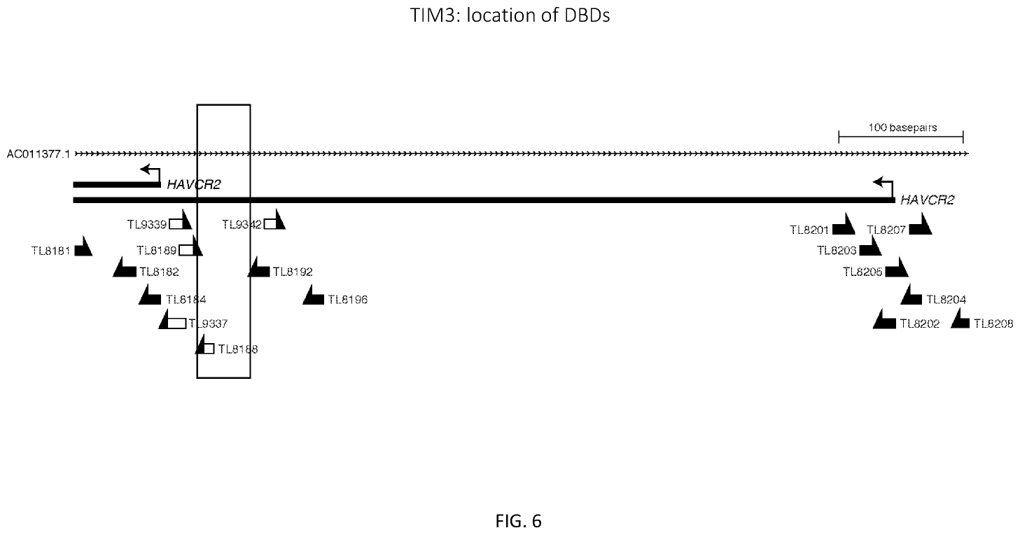

The sequences in the TIM3 gene that were tested to determine repression by a transcriptional repressor domain bound to the sequence are pictorially depicted in FIG. 6. The analysis of repression by the disclosed recombinant polypeptides that are designed bind to these sequences identified certain regions that provide repression of TIM3 expression in at least 50% of the cells expressing these recombinant polypeptides. One such region is depicted in FIG. 6. As explained in the Examples section, in this region, the anti-sense strand of the TIM3 gene as well as the sense strand was successfully targeted to significantly repress expression of TIM3.

The analysis of repression by the disclosed recombinant polypeptides that are designed bind to these sequences identified certain regions that provide repression of TIM3 expression in at least 50% of the cells expressing these recombinant polypeptides. One such region is demarcated in FIG. 6. In this region both sense and anti-sense strand of the TIM3 gene was successfully targeted to significantly repress expression of TIM3-1. The following Table illustrates the sequences present in this region of TIM3 that can be successfully targeted for repression.

TABLE 5

| TALE- | | |
|---|---|---|
| TF ID | Sequence | Repression |
| TL9337 | TGGCAATCAGACACCCGGGTG | >80% |
| | (SEQ ID NO: 48) | |
| TL8188 | GGCAGTGTTACTATAA | >80% |
| | (SEQ ID NO: 45) | |
| Anti- | GGCAGTGTTACTATAA [symbol] | |
| sense | TGGCAATCAGACACCCGGGTG | |
| | (SEQ ID NO: 41) | |
| TL8189 | TGCCAGTGATTCTTATAGT | >80% |
| | (SEQ ID NO: 51) | |
| TL9339 | TGTCTGATTGCCAGTGATT | >80% |
| | (SEQ ID NO: 53) | |
| Sense | TGTCTGATTGCCAGTGATTCTTATAGT | |
| | (SEQ ID NO: 49) | |

As evident from Table 5, the sequences to which TL9337 and TL8188 as well as the sequence between these two sequences (indicated in bold font) can be targeted for TIM3 suppression. This anti-sense sequence of TIM3 is listed in Table 5. The sequences to which TL8189 and TL9339 bind define a region in the sense strand that can be targeted for TIM3 suppression. The sequence of this sense strand is complementary to the anti-sense sequence listed in Table 5.

In certain aspects, a recombinant polypeptide that suppresses expression of TIM3 encoded by the TIM3 gene may include a DNA binding domain (DBD) and a transcriptional repressor. The DBD may include a plurality of RUs ordered from N-terminus to C-terminus of the DBD to bind to a nucleic acid sequence of the TIM3 gene, wherein the nucleic acid sequence is present within the sequence: GGCAGTGTTACTATAAGAATCACTGGCAATCAGACACCCGGGTG (SEQ ID NO:41) or a complement thereof.

In certain aspects, the DBD comprises RUs that bind to the nucleic acid sequence TGTTACTATA (SEQ ID NO:42). In certain aspects, the DBD comprises an additional RU at the C-terminus such that the DBD binds to the nucleic acid sequence TGTTACTATA (SEQ ID NO:43). In certain aspects, the DBD comprises three additional RUs at the N-terminus such that the DBD binds to the nucleic acid sequence CAGTGTTACTATAA (SEQ ID NO:44). the DBD comprises two additional RUs at the N-terminus such that the DBD binds to the nucleic acid sequence GGCAGTGTTACTATAA (SEQ ID NO:45).

In certain aspects, the DBD comprises RUs that bind to the nucleic acid sequence TCAGACACCCGGGTG (SEQ ID NO:46). In certain aspects, the DBD comprises three additional RUs at the N-terminus such that the DBD binds to the nucleic acid sequence CAATCAGACACCCGGGTG (SEQ ID NO:47). In certain aspects, the DBD comprises three additional RUs at the N-terminus such that the DBD binds to the nucleic acid sequence TGGCAATCAGACACCCGGGTG (SEQ ID NO:48).

In another aspect, a recombinant polypeptide that represses TIM3 expression may bind to a sequence that is a complement of GGCAGTGTTACTATAAGAATCACTGGCAATCAGACACCCGGGTG (SEQ ID NO:41) may bind to the sequence: TGTCTGATTGCCAGTGATTCTTATAGT (SEQ ID NO:49). In certain aspects, the DBD comprises RUs that are ordered to bind to the sequence TGCCAGTGATT (SEQ ID NO:50). In certain aspects, the DBD comprises eight additional RUs at the C-terminus such that the DBD binds to the sequence TGCCAGTGATTCTTATAGT (SEQ ID NO:51). In certain aspects, the DBD comprises RUs that are ordered to binds to the sequence TGATTGCCAGTGATT (SEQ ID NO:52). In certain aspects, the DBD comprises four additional RUs at the N-terminus such that the DBD binds to the sequence TGTCTGATTGCCAGTGATT (SEQ ID NO:53).

In addition to the recombinant polypeptides that bind to a sense or anti-sense sequence in the region of TIM3 identified herein, the present disclosure provides additional recombinant polypeptides for repressing TIM3 expression. In certain aspects, the DBD of such a recombinant polypeptide may include a plurality of RUs ordered from N-terminus to C-terminus of the DBD to bind to a nucleic acid sequence of TIM3 gene, wherein the nucleic acid sequence is: TACACACAT (SEQ ID NO:54). In certain aspects, the DBD comprises four additional RUs at the N-terminus such that the DBD binds to the sequence ACACTACACACAT (SEQ ID NO:55). In certain aspects, the DBD comprises four additional RUs at the N-terminus such that the DBD binds to the sequence TGCCACACTACACACAT (SEQ ID NO:56).

In certain aspects, the RUs of the recombinant polypeptide may be arranged from N-terminus to C-terminus to bind to a sequence present in a target sequence listed in Table 10 and shown to have a TIM3 suppression of at least 50%. As noted herein the RUs may range from 7 to 40 in number. In certain aspects, the RUs of the recombinant polypeptide may be arranged from N-terminus to C-terminus to bind to the target sequence listed in Table 10 and shown to have a TIM3 suppression of at least 50%.

In certain aspects, the recombinant polypeptides disclosed herein all reduce the expression of TIM3 gene in at least 50% of the cells transfected with a nucleic acid encoding the recombinant polypeptides while cells not transfected with a nucleic acid encoding the recombinant polypeptides do not show a significant decrease in the expression of the TIM3.

In certain aspects, the recombinant polypeptides disclosed herein all reduce the expression of the TIM3 gene in at least 80% of the cells transfected with a nucleic acid encoding the recombinant polypeptides while cells not transfected with a nucleic acid encoding the recombinant polypeptides do not show a significant decrease in the expression of the TIM3 gene. Accordingly, in certain aspects, a recombinant polypeptide of the present disclosure may include a DBD and a transcriptional repressor domain, the DBD comprising a plurality of RUs ordered from N-terminus to C-terminus of the DBD to bind to a nucleic acid sequence of the TIM3 gene, wherein the nucleic acid sequence is present within one of the following sequences:

(SEQ ID NO: 45)
GGCAGTGTTACTATAA;
(SEQ ID NO: 51)
TGCCAGTGATTCTTATAGT;
(SEQ ID NO: 48)
TGGCAATCAGACACCCGGGTG;
(SEQ ID NO: 56)
TGCCACACTACACACAT;
or
(SEQ ID NO: 53)
TGTCTGATTGCCAGTGATT,

wherein each of the RU comprises the sequence X₁₋₁₁X₁₂X₁₃X_{14-33, 34, or 35} (SEQ ID NO: 455), wherein X₁₋₁₁ is a chain of 11 contiguous amino acids, X_{14-33 or 34 or 35} is a chain of 20, 21 or 22 contiguous amino acids, X₁₂X₁₃ is selected from (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent.

In certain aspects, the DBD comprises at least fourteen RUs, at least sixteen, or at least seventeen RUs and optionally, up to 25 RUs.

In certain aspects, DBD binds to the nucleic acid sequence selected from SEQ ID NOs:45, 51, 48, 56, and 53.

### LAG3 Repressors

Provided herein are recombinant polypeptides that bind to sequences in the LAG3 gene that have been identified to be present in regions of the gene that when bound by the recombinant polypeptides comprising a transcriptional repressor domain lead to suppression of LAG3 expression from the LAG3 gene.

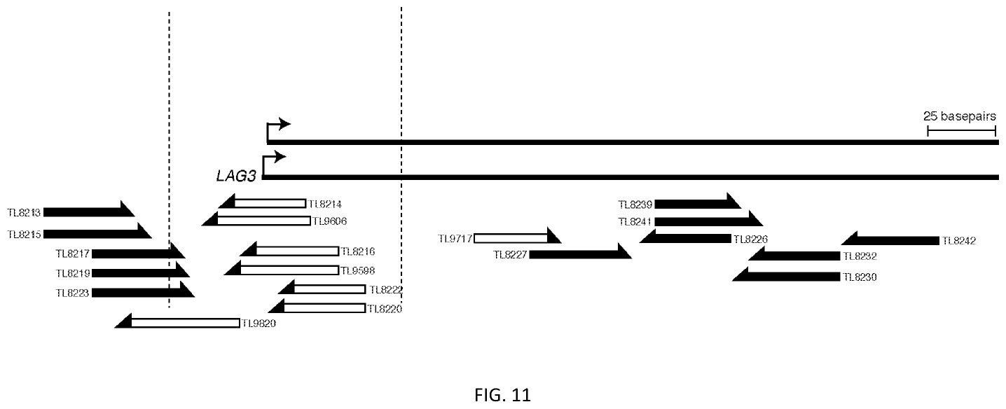

The sequences in the LAG3 gene that were tested to determine repression by a transcriptional repressor domain bound to the sequence are pictorially depicted in FIG. 11. The analysis of repression by the disclosed recombinant polypeptides that are designed bind to these sequences identified certain regions that provide repression of LAG3 expression in at least 50% of the cells expressing these recombinant polypeptides. One such region is depicted in FIG. 11. The following Table illustrates the sequences present in this region of LAG3 that can be successfully targeted for repression.

TABLE 6
LAG3 Repressors

| TALE ID | Target Sequence | Repression |
|---|---|---|
| TL8222 | GCCGTTCTGCTGGTCT | >80% |
| | (SEQ ID NO: 59) | |
| TL8220 | GCCGTTCTGCTGGTCTCT | >80% |
| | (SEQ ID NO: 60) | |
| TL9598 | TCTGCTGGTCTCTGGGCCTTC | >80% |
| | (SEQ ID NO: 450) | |
| TL8216 | TCTGCTGGTCTCTGGGCC | >80% |
| | (SEQ ID NO: 448) | |
| TL9606 | TGGTCTCTGGGCCTTCACCC | >80% |
| | (SEQ ID NO: 446) | |
| TL8214 | GGTCTCTGGGCCTTCA | >80% |
| | (SEQ ID NO: 65) | |
| TL9820 | TTCACCCCTGTGCCCGGCCTTCC | >80% |
| | (SEQ ID NO: 71) | |
| Region | GCCGTTCTGCTGGTCTCTGGGCCTTCACCC | |
| | CTGTGCCCGGCCTTCC | |
| | (SEQ ID NO: 57) | |
| Common | TCTGCTGGTCT | |
| sequence | (SEQ ID NO: 58) | |
| bound | | |
| TL8222, | | |
| TL8220, | | |
| TL9598, | | |
| TL8216 | | |

In certain aspects, the recombinant polypeptide that binds to this region may include a DBD in which the RUs are ordered from N-terminus to C-terminus of the DBD to bind to a nucleic acid sequence of the LAG3 gene, wherein the nucleic acid sequence is present within the sequence:

(SEQ ID NO: 57)
GCCGTTCTGCTGGTCTCTGGGCCTTCACCCCTGTGCCCGGCCTTCC.

In certain aspects, the DBD comprises RUs that bind to the sequence TCTGCTGGTCT (SEQ ID NO:58). In certain aspects, the DBD comprises five additional RUs at the N-terminus such that the DBD binds to the sequence GCCGTTCTGCTGGTCT (SEQ ID NO:59). In certain aspects, the DBD comprises two additional RUs at the C-terminus such that the DBD binds to the sequence GCCGTTCTGCTGGTCTCT (SEQ ID NO:60). In certain aspects, the DBD comprises four additional RUs at the C-terminus such that the DBD binds to the sequence TCTGCTGGTCTGGGC (SEQ ID NO:61). In certain aspects, the DBD comprises an additional RUs at the C-terminus such that the DBD binds to the sequence TCTGCTGGTCTGGGCC (SEQ ID NO:62). In certain aspects, the DBD comprises three additional RUs at the C-terminus such that the DBD binds to the sequence TCTGCTGGTCTGGGCCTTC (SEQ ID NO:63).

In certain aspects, the DBD comprises RUs that bind to the sequence TCTCTGGGCCTTCA (SEQ ID NO:64). In certain aspects, the DBD comprises two additional RUs at the N-terminus such that the DBD binds the sequence GGTCTCTGGGCCTTCA (SEQ ID NO:65). In certain aspects, the DBD comprises three additional RUs at the C-terminus such that the DBD binds the sequence GGTCTCTGGGCCTTCACCC (SEQ ID NO:66). In certain aspects, the DBD comprises an additional RUs at the N-terminus such that the DBD binds the sequence TGGTCTCTGGGCCTTCACC (SEQ ID NO:67).

In certain aspects, the DBD comprises RUs that bind to the sequence TTCACCCCTGTG (SEQ ID NO:68). In certain aspects, the DBD comprises four additional RUs at the C-terminus such that the DBD binds to the sequence TTCACCCCTGTGCCCG (SEQ ID NO:69). In certain aspects, the DBD comprises four additional RUs at the C-terminus such that the DBD binds to the sequence TTCACCCCTGTGCCCGGCCT (SEQ ID NO:70). In certain aspects, the DBD comprises three additional RUs at the C-terminus such that the DBD binds to the sequence TTCACCCCTGTGCCCGGCCTTCC (SEQ ID NO:71).

In addition to the recombinant polypeptides that bind to a sequence in the region of LAG3 identified herein, the present disclosure provides additional recombinant polypeptides for repressing LAG3 expression. In certain aspects, the DBD of such a recombinant polypeptide may include a plurality of RUs ordered from N-terminus to C-terminus of the DBD to bind to a nucleic acid sequence of LAG3 gene, wherein the nucleic acid sequence is: TGCTCTGTCTGC (SEQ ID NO:72). the DBD comprises two additional RUs at the C-terminus such that the DBD binds to the sequence TGCTCTGTCTGCTC (SEQ ID NO:73). In certain aspects, the DBD comprises two additional RUs at the N-terminus such that the DBD binds to the sequence TTTGCTCTGTCTGCTC (SEQ ID NO:74).

In certain aspects, the RUs of the recombinant polypeptide may be arranged from N-terminus to C-terminus to bind to a sequence present in a target sequence listed in Table 12 and shown to have a LAG3 suppression of at least 50%. As noted herein the RUs may range from 7 to 40 in number. In certain aspects, the RUs of the recombinant polypeptide may be arranged from N-terminus to C-terminus to bind to the target sequence listed in Table 12 and shown to have a LAG3 suppression of at least 50%.

In certain aspects, the recombinant polypeptides disclosed herein all reduce the expression of LAG3 gene in at least 50% of the cells transfected with a nucleic acid encoding the recombinant polypeptides while cells not transfected with a nucleic acid encoding the recombinant polypeptides do not show a significant decrease in the expression of the LAG3.

In certain aspects, the recombinant polypeptides disclosed herein reduce the expression of the LAG3 gene in at least 80% of the cells transfected with a nucleic acid encoding the recombinant polypeptides while cells not transfected with a nucleic acid encoding the recombinant polypeptides do not show a significant decrease in the expression of the TIM3 gene. Accordingly, in certain aspects, a recombinant polypeptide of the present disclosure may include a DBD and a transcriptional repressor domain, the DBD comprising a plurality of RUs ordered from N-terminus to C-terminus of the DBD to bind to a nucleic acid sequence of the TIM3 gene, wherein the nucleic acid sequence is present within one of the following sequences:

(SEQ ID NO: 65)
GGTCTCTGGGCCTTCA;
(SEQ ID NO: 448)
TCTGCTGGTCTCTGGGCC;
(SEQ ID NO: 60)
GCCGTTCTGCTGGTCTCT;
(SEQ ID NO: 59)
GCCGTTCTGCTGGTCT;
(SEQ ID NO: 71)
TTCACCCCTGTGCCCGGCCTTCC;
(SEQ ID NO: 449)
TGGTCTCTGGGCCTTCACCC;
(SEQ ID NO: 450)
TCTGCTGGTCTCTGGGCCTTC;
or
(SEQ ID NO: 74)
TTTGCTCTGTCTGCTC,

wherein each of the RU comprises the sequence X₁₋₁₁X₁₂X₁₃X_{14-33, 34, or 35} (SEQ ID NO: 455), wherein X₁₋₁₁ is a chain of 11 contiguous amino acids, X_{14-33 or 34 or 35} is a chain of 20, 21 or 22 contiguous amino acids, X₁₂X₁₃ is selected from (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent.

In certain aspects, the DBD comprises at least fourteen RUs, at least sixteen, or at least seventeen RUs and optionally, up to 25 RUs.

In certain aspects, DBD binds to the nucleic acid sequence selected from SEQ ID NOs: 65, 448, 60, 59, 71, 449, 450, and 74.

### CTLA4 Repressors

Provided herein are recombinant polypeptides that bind to sequences in the CTLA4 gene that have been identified to be present in regions of the gene that when bound by the recombinant polypeptides comprising a transcriptional repressor domain lead to suppression of CTLA4 expression from the CTLA4 gene.

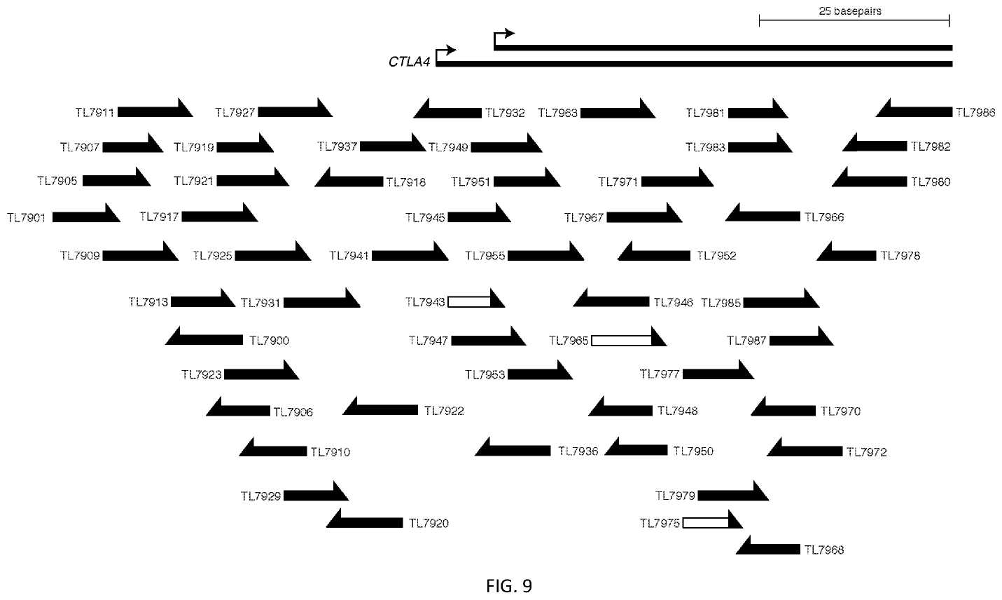

The sequences in the CTLA4 gene that were tested to determine repression by a transcriptional repressor domain bound to the sequence are pictorially depicted in FIG. 9.

In certain aspects, the DBD of the recombinant polypeptide may include at least nine RUs ordered from N-terminus to C-terminus of the DBD to bind to a nucleic acid sequence of the CTLA4 gene, wherein the nucleic acid sequence is present in the sequence: ACATATCTGGGATCAAAGCT (SEQ ID NO:75); ATATAAAGTCCTTGAT (SEQ ID NO:76); or TTCTATTCAAGTGCC (SEQ ID NO:77).

In certain aspects, the RUs of the recombinant polypeptide may be arranged from N-terminus to C-terminus to bind to a sequence present in a target sequence listed in Table 11 and shown to have a CTLA4 suppression of at least 50%. As noted herein the RUs may range from 7 to 40 in number. In certain aspects, the RUs of the recombinant polypeptide may be arranged from N-terminus to C-terminus to bind to the target sequence listed in Table 11 and shown to have a CTLA4 suppression of at least 50%.

In certain aspects, the DBD may be extended at the N-terminus, the C-terminus, or both to increase the number of RUs that contact the nucleic acid sequence is present in the sequence of SEQ ID NOs: 75-77. In certain aspects, the DBD may include at least 10, at least 12, at least 13, at least 14, at least 16, or more and up to 20, 25, 35, or 40 RUs.

### Repeat Units

As noted above, the repeat unit may have the sequence X₁₋₁₁X₁₂X₁₃X_{14-33, 34, or 35} (SEQ ID NO: 455), where X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent.

Any suitable RU such as those based upon the RUs from Xanthomonas transcription activator-like effector (TALE) systems, Ralstonia solanacearum (modular Ralstonia nucleic acid binding domain; RNBD), or an animal pathogen (e.g., Legionella quateirensis, Legionella maceachernii, Burkholderia, Paraburkholderia, or Francisella) (modular animal pathogen nucleic acid binding domain; MAP-NBD) may be arranged to bind to the nucleotide sequences in the target genes as disclosed herein.

In certain aspects, the DNA binding domains of the disclosed recombinant polypeptides may be engineered to include 5, 6, 7, 8, 9, 10, 11, 12, 13, 14, 15, 16, 17, 18, 19, 20, 21, 22, 23, 24, 25, or more, e.g., up to 30, 40 or 50 repeat units arranged in a N-terminal to C-terminal direction to bind to a predetermined 5, 6, 7, 8, 9, 10, 11, 12, 13, 14, 15, 16, 17, 18, 19, 20, 21, 22, 23, 24, 25 nucleotide length nucleic acid sequence, such as, a sequence disclosed herein. In certain aspects, DNA binding domains may be engineered to include 5, 6, 7, 8, 9, 10, 11, 12, 13, 14, 15, 16, 17, 18, 19, 20, 21, 22, 23, 24, 25, 26 or more or more, e.g., up to 30, 40 or 50 repeat units that are specifically ordered or arranged to bind to target nucleic acid sequences of length 5, 6, 7, 8, 9, 10, 11, 12, 13, 14, 15, 16, 17, 18, 19, 20, 21, 22, 23, 24, 25, 26 or more or more, e.g., up to 30, 40 or 50, respectively. In certain embodiments the RUs are contiguous. In some embodiments, half-RUs may be used in the place of one or more RUs. In some aspects, the last RU in a DBD may be a half RU.

DBD Derived from Xanthomonas TALE

In certain aspects, the RUs and the half-RU, if present, are derived from Xanthomonas TALE. In certain aspects, X₁₋₁₁ is at least 80%, at least 90%, or 100% identical to LTPEQVVAIAS (SEQ ID NO: 458), LTPAQVVAIAS (SEQ ID NO: 459), LTPDQVVAIAN (SEQ ID NO: 460), LTPDQVVAIAS (SEQ ID NO: 461), LTPYQVVAIAS (SEQ ID NO: 462), LTREQVVAIAS (SEQ ID NO: 463), or LSTAQVVAIAS (SEQ ID NO: 464). In certain aspects, X_{14-20 or 21 or 22} is at least 80%, at least 90%, at least 95%, or 100% identical to GGKQALETVQRLLPVLCQDHG (SEQ ID NO:79), GGKQALATVQRLLPVLCQDHG (SEQ ID NO: 467), GGKQALETVQRVLPVLCQDHG (SEQ ID NO: 468), or GGKQALETVQRVLPVLCQDHG (SEQ ID NO: 468). In certain aspects, the RU is at least 80%, at least 90%, at least 95%, or 100% identical to:

LTPEQVVAIASX₁₂X₁₃GGKQALETVQRLLPVLCQDHG (SEQ ID NO: 470), X₁₂X₁₃ is repeat variable diresdue (RVD) and is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent.

In certain aspects, the DBD may include a N-cap region at N-terminus of the recombinant polypeptide which N-cap region is derived from the N-cap region of a Xanthomonas TALE protein. In certain aspects, the DBD may include a N-cap region at the N-terminus which may be present immediately adjacent the first RU. In certain aspects, the N-cap region at the N-terminus which may be linked to the first RU via a linker.

An N-cap region may be any length, e.g., may comprise from about 0 to about 136 amino acid residues in length. An N-terminal cap may be about 5, about 10, about 15, about 20, about 25, about 30, about 35, about 40, about 45, about 50, about 60, about 70, about 80, about 90, about 100, about 110, about 120, or about 130 amino acid residues in length. In certain aspects, the DBD comprises a N-cap region comprising an amino acid sequence at least 80% (e.g., at least 90%, at least 95%, or 100%) identical to the amino acid sequence:

(SEQ ID NO: 339)
DYKDHDGDYKDHDIDYKDDDDKMAPKKKRKVGIHRGVPMVDLRTLGYSQQ
QQEKIKPKVRSTVAQHHEALVGHGFTHAHIVALSQHPAALGTVAVKYQDM
IAALPEATHEAIVGVGKQWSGARALEALLTVAGELRGPPLQLDTGQLLKI
AKRGGVTAVEAVHAWRNALTGAPLETPN

In certain aspects, the N-cap region is from TALE proteins like those expressed in Burkholderia, Paraburkholderia, or Xanthomonas. In certain aspects, the N-cap regions may be derived from N-cap domain used in conjunction with DNA binding domains disclosed in US20180010152. In certain aspects, the N-cap regions may be derived from the N-terminal regions disclosed in US20150225465, e.g., SEQ ID NOs.:7, 8, or 9 disclosed therein.

In some aspects, the N-cap region may include the amino acid residues from position 1 (N) through position 137 (M) of the naturally occurring Xanthomonas TALE protein (numbered backwards with N(1) being the residue immediately adjacent the first RU:

(SEQ ID NO: 107)
MVDLRTLGYSQQQQEKIKPKVRSTVAQHHEALVGHGFTHAHIVALSQHPA
ALGTVAVKYQDMIAALPEATHEAIVGVGKQWSGARALEALLTVAGELRGP
PLQLDTGQLLKIAKRGGVTAVEAVHAWRNALTGAPLN.

This amino acid sequence includes a M added to the N-terminus which is not present in the wild type N-cap region of a Xanthomonas TALE protein. This amino acid sequence is generated by deleting amino acids N+288 through N+137 of the N-terminus region of a TALE protein, adding a M, such that amino acids N+136 through N+1 of the N-terminus region of the TALE protein are present.

In some embodiments, the N-terminus can be truncated such that the fragment of the N-terminus includes amino acids from position 1 (N) through position 120 (K) of the naturally occurring Xanthomonas spp.-derived protein as follows:

(SEQ ID NO: 301)
KPKVRSTVAQHHEALVGHGFTHAHIVALSQHPAALGTVAVKYQDMIAALP
EATHEAIVGVGKQWSGARALEALLTVAGELRGPPLQLDTGQLLKIAKRGG
VTAVEAVHAWRNALTGAPLN.

In some aspects, the N-cap region can be truncated such that the fragment of the N-terminus includes amino acids from position 1 (N) through position 115 (S) of the naturally occurring Xanthomonas spp.-derived protein as follows:

(SEQ ID NO: 321)
STVAQHHEALVGHGFTHAHIVALSQHPAALGTVAVKYQDMIAALPEATHE
AIVGVGKQWSGARALEALLTVAGELRGPPLQLDTGQLLKIAKRGGVTAVE
AVHAWRNALTGAPLN.

In some aspects, the N-cap region can be truncated may include amino acids from position 1 (N) through position 110 (H) of the naturally occurring Xanthomonas spp.-derived protein as follows:

(SEQ ID NO: 447)
HHEALVGHGFTHAHIVALSQHPAALGTVAVKYQDMIAALPEATHEAIVGV
GKQWSGARALEALLTVAGELRGPPLQLDTGQLLKIAKRGGVTAVEAVHAW
RNALTGAPLN.

In certain aspects, the DBD may include a C-cap region at C-terminus of the recombinant polypeptide which C-cap region is derived from the C-cap region of a Xanthomonas TALE protein. In certain aspects, the C-cap region at the C-terminus which may be present immediately adjacent the last RU or the last half-RU, if present. In certain aspects, the C-cap region at the C-terminus which may be linked to the last RU or the last half-RU, if present, via a linker.

A C-cap may be any length and may comprise from about 0 to about 278 amino acid residues in length. A C-terminal cap may be about 5, about 10, about 15, about 20, about 25, about 30, about 35, about 40, about 45, about 50, about 60, about 80, about 100, about 150, about 200, or about 250 amino acid residues in length. In certain aspects, the DBD comprises a C-cap region comprising an amino acid sequence at least 80% (e.g., at least 90%, at least 95%, or 100%) identical to the amino acid sequence:

(SEQ ID NO: 452)
SIVAQLSRPDPALAALTNDHLVALACLGGRPALDAVKKGLPHAPALIKRT
NRRIPERTSHRVA.

In certain aspects, the C-cap region is from TALE proteins like those expressed in Burkholderia, Paraburkholderia, or Xanthomonas.

In some aspects, the C-Cap region can be positions 1 (S) through position 278 (Q) of the naturally occurring Xanthomonas spp.-derived protein as follows:

(SEQ ID NO: 108)
SIVAQLSRPDPALAALTNDHLVALACLGGRPALDAVKKGLPHAPALIKRT
NRRIPERTSHRVADHAQVVRVLGFFQCHSHPAQAFDDAMTQFGMSRHGLL
QLFRRVGVTELEARSGTLPPASQRWDRILQASGMKRAKPSPTSTQTPDQA
SLHAFADSLERDLDAPSPTHEGDQRRASSRKRSRSDRAVTGPSAQQSFEV
RAPEQRDALHLPLSWRVKRPRTSIGGGLPDPGTPTAADLAASSTVMREQD
EDPFAGAADDFPAFNEEELAWLMELLPQ.

In certain aspects, the predetermined N-terminus to C-terminus order of the plurality of RUs of the DNA binding domain determines the corresponding predetermined target nucleic acid sequence to which the recombinant polypeptides may bind. As used herein the RUs and at least one or more half RU are specifically ordered to target the genomic locus or gene of interest. In plant genomes, such as Xanthomonas, the natural TALE-binding sites always begin with a thymine (T), which may be specified by a cryptic signal within the non-repetitive N-cap region of the TALE polypeptide; in some cases this region may be referred to as repeat 0. In animal genomes, TALE binding sites do not necessarily have to begin with a thymine (T) and recombinant polypeptides disclosed herein may target DNA sequences that begin with T, A, G or C. In certain aspects, the recombinant polypeptides disclosed herein may target DNA sequences that begin with T and hence include a RU that contains a RVD that mediated binding to T. The tandem repeat of TALE RUs ends with a half-length repeat or a stretch of sequence that may share identity with only the first 20 amino acids of a repetitive full length TALE RU and this half repeat may be referred to as a half-monomer, a half RU, or a half repeat. Therefore, it follows that the length of the DNA sequence being targeted by DBD derived from TALEs is equal to the number of full RUs plus two. Thus, for example, DBD may be engineered to include X number (e.g., 5, 6, 7, 8, 9, 10, 11, 12, 13, 14, 15, 16, 17, 18, 19, 20, 21, 22, 23, 24, 25, or 26) full length RUs that are specifically ordered or arranged to target nucleic acid sequences of X+2 length (e.g., 7, 8, 9, 10, 11, 12, 13, 14, 15, 16, 17, 18, 19, 20, 21, 22, 23, 24, 25, 26, 27, or 28 nucleotides, respectively), with the first RU binding “T” and the last RU being a half-repeat.

As noted herein, in certain aspects, the last RU in the DBD may be a half repeat. The half repeat may comprise the amino acid sequence X₁₋₁₁X₁₂X₁₃X_{14-19, 20, or 21} (SEQ ID NO: 471), wherein X₁₋₁₁ is a chain of 11 contiguous amino acids, X_{14-19 or 20 or 21} is a chain of 7, 8 or 9 contiguous amino acids, and X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); (e) NV or HN for recognition of A or G; and (f) H*, HA, KA, N*, NA, NC, NS, RA, or S*for recognition of A or T or G or C, wherein (*) means that the amino acid at X₁₃ is absent. In certain aspects, X₁₋₁₁ is at least 80% identical, at least 90% identical, or 100% identical to LTPEQVVAIAS (SEQ ID NO:458). In certain aspects, X_{14-20 or 21 or 22} is at least 80% identical to GGRPALE (SEQ ID NO: 472).

As noted herein, a recombinant polypeptide disclosed herein may include from N- to C-terminus, a N-cap region, a DBD comprising a plurality of RUs, a C-cap region, an optional linker, and a transcription repressor domain. In cases, where the RUs are derived from a TALE protein, the recombinant polypeptide may be referred to as TALE-TF. The recombinant polypeptides, such as, TALE-TFs, of the present disclosure can further include a linker connecting the DBD or the C-cap region, if present, to the repressor domain. The linker can serve to provide flexibility between the TALE protein and the repressor domain, allowing for the repressor domain (e.g., KRAB to efficiently inhibit transcriptional machinery). A linker used herein can be a short flexible linker comprising an amino acid sequence comprising 0 residues, 1-3 residues, 4-7 residues, 8-10 residues, 10-12 residues, 5-20 residues, 12-15 residues, or 1-15 residues. Linkers can include, but are not limited to, residues such as glycine, methionine, aspartic acid, alanine, lysine, serine, leucine, threonine, tryptophan, or any combination thereof. The linker can have the amino acid sequence of GGGGGMDAKSLTAWS (SEQ ID NO: 109).

In certain aspects, a Xanthomonas spp.-derived repeat units can have a sequence of LTPDQVVAIASNHGGKQALETVQRLLPVLCQDHG (SEQ ID NO: 438) comprising an RVD of NH, which recognizes guanine. A Xanthomonas spp.-derived repeat units can have a sequence of LTPDQVVAIASNGGGKQALETVQRLLPVLCQDHG (SEQ ID NO: 439) comprising an RVD of NG, which recognizes thymidine. A Xanthomonas spp.-derived repeat units can have a sequence of LTPDQVVAIASNIGGKQALETVQRLLPVLCQDHG (SEQ ID NO: 440) comprising an RVD of NI, which recognizes adenosine. A Xanthomonas spp.-derived repeat units can have a sequence of LTPDQVVAIASHDGGKQALETVQRLLPVLCQDHG (SEQ ID NO: 441) comprising an RVD of HD, which recognizes cytosine.

DBD Derived from Ralstonia

In certain aspects, the RUs and one or both N-Cap and C-Cap regions may be derived from a transcription activator like effector-like protein (TALE-like protein) of Ralstonia solanacearum. Repeat units derived from Ralstonia solanacearum can be 33-35 amino acid residues in length. In some embodiments, the repeat can be derived from the naturally occurring Ralstonia solanacearum TALE-like protein.

As noted herein, the RUs may have the sequence X₁₋₁₁X₁₂X₁₃X_{14-33, 34, or 35} (SEQ ID NO: 455), where X₁₋₁₁ is a chain of 11 contiguous amino acids, X_{14-33 or 34 or 35} is a chain of 20, 21 or 22 contiguous amino acids, X₁₂X₁₃ is RVD and is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent. In certain aspects, X₁₋₁₁ may include a stretch of amino acids at least 80%, at least 90%, or a 10000 identical to the X₁₋₁₁ residues of the following RUs from Ralstonia. In certain aspects, X_{14-33, 34, or 35} may include a stretch of 20, 21, or 22 amino acids at least 80%, at least 90%, or a 100% identical to the X_{14-33, 34, or 35} residues of the following RUs from Ralstonia:

| SEQ ID NO | Sequence(X_{1-11X12X13X14-33, 34, or 35}) |
|---|---|
| 110 | LDTEQVVAIASHNGGKQALEAVKADLLDLLGAPYV |
| 111 | LDTEQVVAIASHNGGKQALEAVKADLLDLRGAPYA |
| 112 | LDTEQVVAIASHNGGKQALEAVKADLLELRGAPYA |
| 113 | LDTEQVVAIASHNGGKQALEAVKAHLLDLRGAPYA |
| 114 | LNTEQVVAIASHNGGKQALEAVKADLLDLRGAPYA |
| 115 | LNTEQVVAIASNNGGKQALEAVKTHLLDLRGARYA |
| 116 | LNTEQVVAIASNPGGKQALEAVRALFPDLRAAPYA |
| 117 | LNTEQVVAIASSHGGKQALEAVRALFPDLRAAPYA |
| 118 | LNTEQVVAVASNKGGKQALEAVGAQLLALRAVPYA |
| 119 | LNTEQVVAVASNKGGKQALEAVGAQLLALRAVPYE |
| 120 | LSAAQVVAIASHDGGKQALEAVGTQLVALRAAPYA |
| 121 | LSIAQVVAVASRSGGKQALEAVRAQLLALRAAPYG |
| 122 | LSPEQVVAIASNHGGKQALEAVRALFRGLRAAPYG |
| 123 | LSPEQVVAIASNNGGKQALEAVKAQLLELRAAPYE |
| 124 | LSTAQLVAIASNPGGKQALEAIRALFRELRAAPYA |
| 125 | LSTAQLVAIASNPGGKQALEAVRALFRELRAAPYA |
| 126 | LSTAQLVAIASNPGGKQALEAVRAPFREVRAAPYA |
| 127 | LSTAQLVSIASNPGGKQALEAVRALFRELRAAPYA |
| 128 | LSTAQVAAIASHDGGKQALEAVGTQLVVLRAAPYA |
| 129 | LSTAQVATIASSIGGRQALEALKVQLPVLRAAPYG |
| 130 | LSTAQVATIASSIGGRQALEAVKVQLPVLRAAPYG |
| 131 | LSTAQVVAIAANNGGKQALEAVRALLPVLRVAPYE |
| 132 | LSTAQVVAIAGNGGGKQALEGIGEQLLKLRTAPYG |
| 133 | LSTAQVVAIASHDGGKQALEAAGTQLVALRAAPYA |
| 134 | LSTAQVVAIASHDGGKQALEAVGAQLVELRAAPYA |
| 135 | LSTAQVVAIASHDGGKQALEAVGTQLVALRAAPYA |
| 136 | LSTAQVVAIASHDGGNQALEAVGTQLVALRAAPYA |
| 137 | LSTAQVVAIASHNGGKQALEAVKAQLLDLRGAPYA |
| 138 | LSTAQVVAIASNDGGKQALEEVEAQLLALRAAPYE |
| 139 | LSTAQVVAIASNGGGKQALEGIGEQLLKLRTAPYG |
| 140 | LSTAQVVAIASNGGGKQALEGIGEQLRKLRTAPYG |
| 141 | LSTAQVVAIASNPGGKQALEAVRALFRELRAAPYA |
| 142 | LSTAQVVAIASQNGGKQALEAVKAQLLDLRGAPYA |
| 143 | LSTAQVVAIASSHGGKQALEAVRALFRELRAAPYG |
| 144 | LSTAQVVAIASSNGGKQALEAVWALLPVLRATPYD |
| 145 | LSTAQVVAIATRSGGKQALEAVRAQLLDLRAAPYG |
| 146 | LSTAQVVAVAGRNGGKQALEAVRAQLPALRAAPYG |
| 147 | LSTAQVVAVASSNGGKQALEAVWALLPVLRATPYD |
| 148 | LSTAQVVTIASSNGGKQALEAVWALLPVLRATPYD |
| 149 | LSTEQVVAIAGHDGGKQALEAVGAQLVALRAAPYA |
| 150 | LSTEQVVAIASHDGGKQALEAVGAQLVALLAAPYA |
| 151 | LSTEQVVAIASHDGGKQALEAVGAQLVALRAAPYA |
| 152 | LSTEQVVAIASHDGGKQALEAVGGQLVALRAAPYA |
| 153 | LSTEQVVAIASHDGGKQALEAVGTQLVALRAAPYA |
| 154 | LSTEQVVAIASHDGGKQALEAVGVQLVALRAAPYA |
| 155 | LSTEQVVAIASHDGGKQALEAVVAQLVALRAAPYA |
| 156 | LSTEQVVAIASHDGGKQPLEAVGAQLVALRAAPYA |
| 157 | LSTEQVVAIASHGGGKQVLEGIGEQLLKLRAAPYG |
| 158 | LSTEQVVAIASHKGGKQALEGIGEQLLKLRAAPYG |
| 159 | LSTEQVVAIASHNGGKQALEAVKADLLDLRGAPYA |
| 160 | LSTEQVVAIASHNGGKQALEAVKADLLELRGAPYA |
| 161 | LSTEQVVAIASHNGGKQALEAVKAHLLDLRGAPYA |
| 162 | LSTEQVVAIASHNGGKQALEAVKAHLLDLRGVPYA |
| 163 | LSTEQVVAIASHNGGKQALEAVKAHLLELRGAPYA |
| 164 | LSTEQVVAIASHNGGKQALEAVKAQLLDLRGAPYA |
| 165 | LSTEQVVAIASHNGGKQALEAVKAQLLELRGAPYA |
| 166 | LSTEQVVAIASHNGGKQALEAVKAQLPVLRRAPYG |
| 167 | LSTEQVVAIASHNGGKQALEAVKTQLLELRGAPYA |
| 168 | LSTEQVVAIASHNGGKQALEAVRAQLPALRAAPYG |
| 169 | LSTEQVVAIASHNGSKQALEAVKAQLLDLRGAPYA |
| 170 | LSTEQVVAIASNGGGKQALEGIGKQLQELRAAPHG |
| 171 | LSTEQVVAIASNGGGKQALEGIGKQLQELRAAPYG |
| 172 | LSTEQVVAIASNHGGKQALEAVRALFRELRAAPYA |
| 173 | LSTEQVVAIASNHGGKQALEAVRALFRGLRAAPYG |
| 174 | LSTEQVVAIASNKGGKQALEAVKADLLDLRGAPYV |
| 175 | LSTEQVVAIASNKGGKQALEAVKAHLLDLLGAPYV |
| 176 | LSTEQVVAIASNKGGKQALEAVKAQLLALRAAPYA |
| 177 | LSTEQVVAIASNKGGKQALEAVKAQLLELRGAPYA |
| 178 | LSTEQVVAIASNNGGKQALEAVKALLLELRAAPYE |
| 179 | LSTEQVVAIASNNGGKQALEAVKAQLLALRAAPYE |
| 180 | LSTEQVVAIASNNGGKQALEAVKAQLLDLRGAPYA |
| 181 | LSTEQVVAIASNNGGKQALEAVKAQLLVLRAAPYG |
| 182 | LSTEQVVAIASNNGGKQALEAVKAQLPALRAAPYE |
| 183 | LSTEQVVAIASNNGGKQALEAVKAQLPVLRRAPCG |
| 184 | LSTEQVVAIASNNGGKQALEAVKAQLPVLRRAPYG |
| 185 | LSTEQVVAIASNNGGKQALEAVKARLLDLRGAPYA |
| 186 | LSTEQVVAIASNNGGKQALEAVKTQLLALRTAPYE |
| 187 | LSTEQVVAIASNPGGKQALEAVRALFPDLRAAPYA |
| 188 | LSTEQVVAIASSHGGKQALEAVRALFPDLRAAPYA |
| 189 | LSTEQVVAIASSHGGKQALEAVRALLPVLRATPYD |
| 190 | LSTEQVVAVASHNGGKQALEAVRAQLLDLRAAPYE |
| 191 | LSTEQVVAVASNKGGKQALAAVEAQLLRLRAAPYE |
| 192 | LSTEQVVAVASNKGGKQALEEVEAQLLRLRAAPYE |
| 193 | LSTEQVVAVASNKGGKQVLEAVGAQLLALRAVPYE |
| 194 | LSTEQVVAVASNNGGKQALKAVKAQLLALRAAPYE |
| 195 | LSTEQVVVIANSIGGKQALEAVKVQLPVLRAAPYE |
| 196 | LSTGQVVAIASNGGGRQALEAVREQLLALRAVPYE |
| 197 | LSVAQVVTIASHNGGKQALEAVRAQLLALRAAPYG |
| 198 | LTIAQVVAVASHNGGKQALEAIGAQLLALRAAPYA |
| 199 | LTIAQVVAVASHNGGKQALEVIGAQLLALRAAPYA |
| 200 | LTPQQVVAIAANTGGKQALGAITTQLPILRAAPYE |
| 201 | LTPQQVVAIASNTGGKQALEAVTVQLRVLRGARYG |
| 202 | LTPQQVVAIASNTGGKRALEAVCVQLPVLRAAPYR |
| 203 | LTPQQVVAIASNTGGKRALEAVRVQLPVLRAAPYE |
| 204 | LTTAQVVAIASNDGGKQALEAVGAQLLVLRAVPYE |
| 205 | LTTAQVVAIASNDGGKQTLEVAGAQLLALRAVPYE |
| 206 | LSTAQVVAVASGSGGKPALEAVRAQLLALRAAPYG |
| 207 | LSTAQVVAVASGSGGKPALEAVRAQLLALRAAPYG |
| 208 | LNTAQIVAIASHDGGKPALEAVWAKLPVLRGAPYA |
| 209 | LNTAQVVAIASHDGGKPALEAVRAKLPVLRGVPYA |
| 210 | LNTAQVVAIASHDGGKPALEAVWAKLPVLRGVPYA |
| 211 | LNTAQVVAIASHDGGKPALEAVWAKLPVLRGVPYE |
| 212 | LSTAQVVAIASHDGGKPALEAVWAKLPVLRGAPYA |
| 213 | LSTAQVVAVASHDGGKPALEAVRKQLPVLRGVPHQ |
| 214 | LSTAQVVAVASHDGGKPALEAVRKQLPVLRGVPHQ |
| 215 | LNTAQVVAIASHDGGKPALEAVWAKLPVLRGVPYA |
| 216 | LSTEQVVAIASHNGGKLALEAVKAHLLDLRGAPYA |
| 217 | LSTEQVVAIASHNGGKPALEAVKAHLLALRAAPYA |
| 218 | LNTAQVVAIASHYGGKPALEAVWAKLPVLRGVPYA |
| 219 | LNTEQVVAIASNNGGKPALEAVKAQLLELRAAPYE |
| 220 | LSPEQVVAIASNNGGKPALEAVKALLLALRAAPYE |
| 221 | LSPEQVVAIASNNGGKPALEAVKAQLLELRAAPYE |
| 222 | LSTEQVVAIASNNGGKPALEAVKALLLALRAAPYE |
| 223 | LSTEQVVAIASNNGGKPALEAVKALLLELRAAPYE |
| 224 | LSPEQVVAIASNNGGKPALEAVKALLLALRAAPYE |
| 225 | LSPEQVVAIASNNGGKPALEAVKAQLLELRAAPYE |
| 226 | LSTEQVVAIASNNGGKPALEAVKALLLELRAAPYE |

In certain aspects, a Ralstonia solanacearum-repeat unit can have at least 80% sequence identity with any one of the Ralstonia RUs provided herein.

In certain aspects, the DBD may include a N-cap region at the N-terminus which may be present immediately adjacent the first RU or may be linked to the first RU via a linker. In some aspects, an DBD of the present disclosure can have the full length naturally occurring N-terminus of a naturally occurring Ralstonia solanacearum-derived protein. In some aspects, any truncation of the full length naturally occurring N-terminus of a naturally occurring Ralstonia solanacearum-derived protein can be used at the N-terminus of a DBD of the present disclosure. For example, in some embodiments, amino acid residues at positions 1 (H) to position 137 (F) of the naturally occurring Ralstonia solanacearum-derived protein N-terminus can be used as the N-cap region. In particular embodiments, the truncated N-terminus from position 1 (H) to position 137 (F) can have a sequence as follows: FGKLVALGYSREQIRKLKQESLSEIAKYHTTLTGQGFTHADICRISRRRQSLRVVARNYPELAAAL PELTRAHIVDIARQRSGDLALQALLPVATALTAAPLRLSASQIATVAQYGERPAIQALYRLRRKLT RAPLH (SEQ ID NO:227). In some embodiments, the naturally occurring N-terminus of Ralstonia solanacearum can be truncated to any length and used as the N-cap of the engineered DNA binding domain. For example, the naturally occurring N-terminus of Ralstonia solanacearum can be truncated to include amino acid residues at position 1 (H) to position 120 (K) as follows: KQESLSEIAKYHTTLTGQGFTHADICRISRRRQSLRVVARNYPELAAALPELTRAHIVDIARQRSG DLALQALLPVATALTAAPLRLSASQIATVAQYGERPAIQALYRLRRKLTRAPLH (SEQ ID NO:228) and used as the N-cap of the DBD. The naturally occurring N-terminus of Ralstonia solanacearum can be truncated amino acid residues to include positions 1 to 115 and used at the N-cap of the engineered DNA binding domain. The naturally occurring N-terminus of Ralstonia solanacearum can be truncated to amino acid residues at positions 1 to 50, 1 to 70, 1 to 100, 1 to 120, 1 to 130, 10 to 40, 60 to 100, or 100 to 120 and used as the N-cap of the engineered DNA binding domain. As noted for N-cap region derived from Xanthomonas TALE, the amino acid residues are numbered backward from the first repeat unit such that the amino acid (H in this case) of the N-cap adjacent the first RU is numbered 1 while the N-terminal amino acid of the N-cap is numbered 137 (and is F in this case) or 120 (and is K in this case).

In some embodiments, the N-cap, referred to as the amino terminus or the “NH2” domain, can recognize a guanine. In some embodiments, the N-cap can be engineered to bind a cytosine, adenosine, thymidine, guanine, or uracil.

In some embodiments, an DBD of the present disclosure can include a plurality of RUs followed by a final single half-repeat also derived from Ralstonia solanacearum. The half repeat can have 15 to 23 amino acid residues, for example, the half repeat can have 19 amino acid residues. In particular embodiments, the half-repeat can have a sequence as follows: LSTAQVVAIACISGQQALE (SEQ ID NO:229).

In some embodiments, an DBD of the present disclosure can have the full length naturally occurring C-terminus of a naturally occurring Ralstonia solanacearum-derived protein as a C-cap region that is conjugated to the last RU. In some embodiments, any truncation of the full length naturally occurring C-terminus of a naturally occurring Ralstonia solanacearum-derived protein can be used as the C-cap. For example, in some embodiments, the DBD can comprise amino acid residues at position 1 (A) to position 63 (S) as follows: AIEAHMPTLRQASHSLSPERVAAIACIGGRSAVEAVRQGLPVKAIRRIRREKAPVAGPPPAS (SEQ ID NO:230) of the naturally occurring Ralstonia solanacearum-derived protein C-terminus. In some embodiments, the naturally occurring C-terminus of Ralstonia solanacearum can be truncated to any length and used as the C-cap of the DBD. For example, the naturally occurring C-terminus of Ralstonia solanacearum can be truncated to amino acid residues at positions 1 to 63 and used as the C-terminus of the DBD. The naturally occurring C-terminus of Ralstonia solanacearum can be truncated amino acid residues at positions 1 to 50 and used as the C-cap of the DBD. The naturally occurring C-terminus of Ralstonia solanacearum can be truncated to amino acid residues at positions 1 to 63, 1 to 50, 1 to 70, 1 to 100, 1 to 120, 1 to 130, 10 to 40, 60 to 100, or 100 to 120 and used as the C-cap of the DBD. TABLE 7 shows N-Cap, C-Cap, and half-repeats derived from Ralstonia.

| SEQ ID NO | Description | Sequence |
|---|---|---|
| 231 | Truncated N-terminus; | SEIAKYHTTLTGQGFTHADICRISRRRQSLRVVARNYPEL |
| | positions 1 (H) to 115 (S) | AAALPELTRAHIVDIARQRSGDLALQALLPVATALTAAPL |
| | of the naturally occurring | RLSASQIATVAQYGERPAIQALYRLRRKLTRAPLH |
| | Ralstonia solanacearum- | |
| | derived protein N-terminus | |
| 227 | Truncated N-terminus; | FGKLVALGYSREQIRKLKQESLSEIAKYHTTLTGQGFTHA |
| | positions 1 (H) to 137 (F) | DICRISRRRQSLRVVARNYPELAAALPELTRAHIVDIARQ |
| | of the naturally occurring | RSGDLALQALLPVATALTAAPLRLSASQIATVAQYGERPA |
| | Ralstonia solanacearum- | IQALYRLRRKLTRAPLH |
| | derived protein N-terminus | |
| 228 | Truncated N-terminus; | KQESLSEIAKYHTTLTGQGFTHADICRISRRRQSLRVVAR |
| | positions 1 (H) to 120 (K) | NYPELAAALPELTRAHIVDIARQRSGDLALQALLPVATAL |
| | of the naturally occurring | TAAPLRLSASQIATVAQYGERPAIQALYRLRRKLTRAPLH |
| | Ralstonia solanacearum- | |
| | derived protein N-terminus | |
| 229 | Half-repeat | LSTAQVVAIACISGQQALE |
| 230 | Truncated C-terminus; | AIEAHMPTLRQASHSLSPERVAAIACIGGRSAVEAVRQGL |
| | positions 1 (A) to 63 (S) of | PVKAIRRIRREKAPVAGPPPAS |
| | the naturally occurring | |
| | Ralstonia solanacearum- | |
| | derived protein C-terminus | |

DBD Derived from Animal Pathogens

In some embodiments, the present disclosure provides DNA binding domains in which the repeat units can be derived from a Legionellales bacterium, a species of the genus of Legionella, such as L. quateirensis or L. maceachernii, the genus of Burkholderia, the genus of Paraburkholderia, or the genus of Francisella.

As noted herein, the RUs may have the sequence X₁₋₁₁X₁₂X₁₃X_{14-33, 34, or 35} (SEQ ID NO: 455), where X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, HN, or KN for recognition of guanine (G); (b) NI, KI, RI, HI, HA, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent. In certain aspects, X₁_may include a stretch of amino acids at least 80%, at least 90%, or a 100% identical to the X₁_₁ residues of the following RUs from animal pathogens, Legionella, Burkholderia, Paraburkholderia, or Francisella. In certain aspects, X_{14-33, 34, or 35} may include a stretch of 20, 21, or 22 amino acids at least 80%, at least 90%, or a 100% identical to the X_{14-33, 34, or 35} residues of the RUs from animal pathogens, Legionella (e.g., L. quateirensis or L. maceachernii), Burkholderia, Paraburkholderia, or Francisella listed in Table 8.

TABLE 8
Repeat Unit Sequences

| SEQ | | | BCR |
|---|---|---|---|
| ID NO | Organism | Repeat Unit Sequence (X_{1-11, X12X13X14-33, 34, or 35}) | (X₁₂X₁₃) |
| 232 | L. quateirensis | FSSQQIIRMVSHAGGANNLKAVTANHDDLQNMG | HA |
| 233 | L. quateirensis | FNVEQIVRMVSHNGGSKNLKAVTDNHDDLKNMG | HN |
| 234 | L. quateirensis | FNAEQIVRMVSHGGGSKNLKAVTDNHDDLKNMG | HG |
| 235 | L. quateirensis | FNAEQIVSMVSNNGGSKNLKAVTDNHDDLKNMG | NN |
| 236 | L. quateirensis | FNAEQIVSMVSNGGGSLNLKAVKKYHDALKDRG | NG |
| 237 | L. quateirensis | FNTEQIVRMVSHDGGSLNLKAVKKYHDALRERK | HD |
| 238 | L. quateirensis | FNVEQIVSIVSHGGGSLNLKAVKKYHDVLKDRE | HG |
| 239 | L. quateirensis | FNAEQIVRMVSHDGGSLNLKAVTDNHDDLKNMG | HD |
| 240 | L. maceachernii | FSAEQIVRIAAHDGGSRNIEAVQQAQHVLKELG | HD |
| 241 | L. maceachernii | FSAEQIVSIVAHDGGSRNIEAVQQAQHILKELG | HD |
| 242 | Legionellales | LDRQQILRIASHDGGSKNIAAVQKFLPKLMNFG | HD |
| | bacterium | | |
| 243 | L. maceachernii | FSAEQIVRIAAHDGGSLNIDAVQQAQQALKELG | HD |
| 244 | L. maceachernii | FSTEQ IVCIAGHGGGSLNIKAVLLAQQALKDLG | HG |
| 245 | L. maceachernii | YSSEQIVRVAAHGGGSLNIKAVLQAHQALKELD | HG |
| 246 | L. maceachernii | FSAEQIVHIAAHGGGSLNIKAILQAHQTLKELN | HG |
| 247 | L. maceachernii | FSAEQIVRIAAHIGGSRNIEAIQQAHHALKELG | HI |
| 248 | L. maceachernii | FSAEQIVRIAAHIGGSHNLKAVLQAQQALKELD | HI |
| 249 | L. maceachernii | FSAKHIVRIAAHIGGSLNIKAVQQAQQALKELG | HI |
| 250 | L. quateirensis | FNAEQIVRMVSHKGGSKNLALVKEYFPVFSSFH | HK |
| 251 | L. maceachernii | FSADQIVRIAAHKGGSHNIVAVQQAQQALKELD | HK |
| 252 | L. maceachernii | FSAEQIVSIAAHVGGSHNIEAVQKAHQALKELD | HV |
| 253 | Burkholderia | FSSGETVGATVGAGGTETVAQGGTASNTTVSSG | GA |
| 254 | Burkholderia | FSGGMATSTTVGSGGTQDVLAGGAAVGGTVGTG | GS |
| 255 | Burkholderia | FSAADIVKIAGKIGGAQALQAFITHRAALIQAG | KI |
| 256 | Burkholderia | FNPTDIVKIAGNDGGAQALQAVLELEPALRERG | ND |
| 257 | Burkholderia | FNPTDIVRMAGNDGGAQALQAVFELEPAFRERS | ND |
| 258 | Burkholderia | FNPTDIVRMAGNDGGAQALQAVLELEPAFRERG | ND |
| 259 | Burkholderia | FSQVDIVKIASNDGGAQALYSVLDVEPTFRERG | ND |
| 260 | Burkholderia | FSRADIVKIAGNDGGAQALYSVLDVEPPLRERG | ND |
| 261 | Burkholderia | FSRGDIVKIAGNDGGAQALYSVLDVEPPLRERG | ND |
| 262 | Burkholderia | FNRADIVRIAGNGGGAQALYSVRDAGPTLGKRG | NG |
| 263 | Burkholderia | FRQADIVKIASNGGSAQALNAVIKLGPTLRQRG | NG |
| 264 | Burkholderia | FRQADIVKMASNGGSAQALNAVIKLGPTLRQRG | NG |
| 265 | Burkholderia | FSRADIVKIAGNGGGAQALQAVLELEPTFRERG | NG |
| 266 | Burkholderia | FSRADIVRIAGNGGGAQALYSVLDVGPTLGKRG | NG |
| 267 | Burkholderia | FSRGDIVRIAGNGGGAQALQAVLELEPTLGERG | NG |
| 268 | Burkholderia | FSRADIVKIAGNGGGAQALQAVITHRAALTQAG | NG |
| 269 | Burkholderia | FSRGDTVKIAGNIGGAQALQAVLELEPTLRERG | NI |
| 270 | Burkholderia | FNPTDIVKIAGNIGGAQALQAVLELEPAFRERG | NI |
| 271 | Burkholderia | FSAADIVKIAGNIGGAQALQAIFTHRAALIQAG | NI |
| 272 | Burkholderia | FSAADIVKIAGNIGGAQALQAVITHRATLTQAG | NI |
| 273 | Burkholderia | FSATDIVKIASNIGGAQALQAVISRRAALIQAG | NI |
| 274 | Burkholderia | FSQPDIVKIAGNIGGAQALQAVLELEPAFRERG | NI |
| 275 | Burkholderia | FSRADIVKIAGNIGGAQALQAVLELESTFRERS | NI |
| 276 | Burkholderia | FSRADIVKIAGNIGGAQALQAVLELESTLRERS | NI |
| 277 | Burkholderia | FSRGDIVKMAGNIGGAQALQAGLELEPAFRERG | NI |
| 278 | Burkholderia | FSRGDIVKMAGNIGGAQALQAVLELEPAFHERS | NI |
| 279 | Burkholderia | FTLTDIVKMAGNIGGAQALKAVLEHGPTLRQRD | NI |
| 280 | Burkholderia | FTLTDIVKMAGNIGGAQALKVVLEHGPTLRQRD | NI |
| 281 | Burkholderia | FNPTDIVKIAGNNGGAQALQAVLELEPALRERG | NN |
| 282 | Burkholderia | FNPTDIVKIAGNNGGAQALQAVLELEPALRERS | NN |
| 283 | Burkholderia | FNPTDMVKIAGNNGGAQALQAVLELEPALRERG | NN |
| 284 | Burkholderia | FSAADIVKIASNNGGAQALQALIDHWSTLSGKT | NN |
| 285 | Burkholderia | FSAADIVKIASNNGGAQALQAVISRRAALIQAG | NN |
| 286 | Burkholderia | FSAADIVKIASNNGGAQALQAVITHRAALAQAG | NN |
| 287 | Burkholderia | FSAADIVKIASNNGGARALQALIDHWSTLSGKT | NN |
| 288 | Burkholderia | FTLTDIVEMAGNNGGAQALKAVLEHGSTLDERG | NN |
| 289 | Burkholderia | FTLTDIVKMAGNNGGAQALKAVLEHGPTLDERG | NN |
| 290 | Burkholderia | FTLTDIVKMAGNNGGAQALKVVLEHGPTLRQRG | NN |
| 291 | Burkholderia | FTLTDIVKMASNNGGAQALKAVLEHGPTLDERG | NN |
| 292 | Burkholderia | FSAADIVKIAGNSGGAQALQAVISHRAALTQAG | NS |
| 293 | Burkholderia | FSGGDAVSTVVRSGGAQSVASGGTASGTTVSAG | RS |
| 294 | Burkholderia | FRQTDIVKMAGSGGSAQALNAVIKHGPTLRQRG | SG |
| 295 | Burkholderia | FSLIDIVEIASNGGAQALKAVLKYGPVLTQAGR | SN |
| 296 | Burkholderia | FSGGDAAGTVVSSGGAQNVTGGLASGTTVASGG | SS |
| 297 | Paraburkholderia | FNLTDIVEMAANSGGAQALKAVLEHGPTLRQRG | NS |
| 298 | Paraburkholderia | FNRASIVKIAGNSGGAQALQAVLKHGPTLDERG | NS |
| 299 | Paraburkholderia | FSQANIVKMAGNSGGAQALQAVLDLELVFRERG | NS |
| 300 | Paraburkholderia | FSQPDIVKMAGNSGGAQALQAVLDLELAFRERG | NS |
| 301 | Paraburkholderia | FSLIDIVEIASNGGAQALKAVLKYGPVLMQAGR | SN |
| 302 | Francisella | YKSEDIIRLASHDGGSVNLEAVLRLHSQLTRLG | HD |
| 303 | Francisella | YKPEDIIRLASHGGGSVNLEAVLRLNPQLIGLG | HG |
| 304 | Francisella | YKSEDIIRLASHGGGSVNLEAVLRLHSQLTRLG | HG |
| 305 | Francisella | YKSEDIIRLASHGGGSVNLEAVLRLNPQLIGLG | HG |
| 306 | L. quateirensis | LGHKELIKIAARNGGGNNLIAVLSCYAKLKEMG | RN |
| 307 | Paraburkholderia | FNLTDIVEMAGKGGGAQALKAVLEHGPTLRQRG | KG |
| 308 | Paraburkholderia | FRQADIIKIAGNDGGAQALQAVIEHGPTLRQHG | ND |
| 309 | Paraburkholderia | FSQADIVKIAGNDGGTQALHAVLDLERMLGERG | ND |
| 310 | Paraburkholderia | FSRADIVKIAGNGGGAQALKAVLEHEATLDERG | NG |
| 311 | Paraburkholderia | FSRADIVRIAGNGGGAQALYSVLDVEPTLGKRG | NG |
| 312 | Paraburkholderia | FSQPDIVKMASNIGGAQALQAVLELEPALRERG | NI |
| 313 | Paraburkholderia | FSQPDIVKMAGNIGGAQALQAVLSLGPALRERG | NI |
| 314 | Paraburkholderia | FSQPEIVKIAGNIGGAQALHTVLELEPTLHKRG | NI |
| 315 | Paraburkholderia | FSQSDIVKIAGNIGGAQALQAVLDLESMLGKRG | NI |
| 316 | Paraburkholderia | FSQSDIVKIAGNIGGAQALQAVLELEPTLRESD | NI |
| 317 | Paraburkholderia | FNPTDIVKIAGNKGGAQALQAVLELEPALRERG | NK |
| 318 | Paraburkholderia | FSPTDIIKIAGNNGGAQALQAVLDLELMLRERG | NN |
| 319 | Paraburkholderia | FSQADIVKIAGNNGGAQALYSVLDVEPTLGKRG | NN |
| 320 | Paraburkholderia | FSRGDIVTIAGNNGGAQALQAVLELEPTLRERG | NN |
| 321 | Paraburkholderia | FSRIDIVKIAANNGGAQALHAVLDLGPTLRECG | NN |
| 322 | Paraburkholderia | FSQADIVKIVGNNGGAQALQAVFELEPTLRERG | NN |
| 323 | Paraburkholderia | FSQPDIVRITGNRGGAQALQAVLALELTLRERG | NR |
| 324 | Legionellales | FKADDAVRIACRTGGSHNLKAVHKNYERLRARG | RT |
| 325 | Legionellales | FNADQVIKIVGHDGGSNNIDVVQQFFPELKAFG | HD |
| 326 | L. maceachernii | FSAEQIVRIAAHIGGSRNIEATIKHYAMLTQPP | HI |
| 327 | Francisella | YKSEDIIRLASHDGGSVNLEAVLRLNPQLIGLG | HD |
| 328 | Francisella | YKSEDIIRLASHDGGSINLEAVLRLNPQLIGLG | HD |
| 329 | Francisella | YKSEDIIRLASSNGGSVNLEAVLRLNPQLIGLG | SN |
| 330 | Francisella | YKSEDIIRLASSNGGSVNLEAVIAVHKALHSNG | SN |
| 331 | Legionellales | FSADQVVKIAGHSGGSNNIAVMLAVFPRLRDFG | HS |
| 332 | Francisella | YKINHCVNLLKLNHDGFMLKNLIPYDSKLTGLG | LN |

Residues X₁₂X₁₃ of the RU may include base contacting residues (BCR) as listed in the table 8 and may be chosen based upon the target nucleic acid sequence.

In certain aspects, the last RU in the DBD may be a half RU. In certain aspects, the half RU may include a sequence that is at least 80%, at least 90%, at least 95% or a 100% identical to the half RU from L. quateirensis (FNAEQIVRMVSX₁₂X₁₃GGSKNL) (SEQ ID NO:333). In certain aspects, the half RU may include a sequence that is at least 80%, at least 90%, at least 95% or a 100% identical to the half RU from Francisella (YNKKQIVLIASX₁₂X₁₃SGG) (SEQ ID NO:334).

In certain aspects, the polypeptide comprises an N-cap region, where the C-terminus (i.e., the last amino acid) of the N-cap region is covalently linked to the N-terminus (i.e., the first amino acid) of the first RU of the DBD either directly or via a linker. In certain aspects, the N-cap region is the N-terminus of L. quateirensis protein and may have an amino acid sequence that is at least 80% (e.g., at least 85%, at least 90%, 95%, or 99%, or a 100%) identical to the amino acid sequence:

MPDLELNFAIPLHLFDDETVFTHDATNDNSQASSSYSSKSSPASANARKRTSRKEMSGPP SKEPANTKSRRANSQNNKLSLADRLTKYNIDEEFYQTRSDSLLSLNYTKKQIERLILYKGRTSAV QQLLCKHEELLNLISPDG (SEQ ID NO:335). In certain aspects, the N-cap region comprises a fragment of SEQ ID NO:335. In certain aspects, the N-cap region is a N-terminal domain or a fragment thereof from TALE proteins like those expressed in Burkholderia, Paraburkholderia, or Xanthomonas.

In certain aspects, the polypeptide comprises a C-cap region, where the N-terminus (i.e., the first amino acid) of the C-terminal domain is covalently linked to the C-terminus (i.e., the last amino acid) of the last RU or the half-repeat unit, if present, in the DBD either directly or via a linker. In certain aspects, the C-cap region is the C-terminal domain of L. quateirensis protein and may have an amino acid sequence that is at least 80% (e.g., at least 85%, at least 90%, 95%, or 99%, or a 100%) identical to the amino acid sequence:

(SEQ ID NO: 336)
ALVKEYFPVFSSFHFTADQIVALICQSKQCFRNLKKNHQQWKNKGLSAE
QIVDLILQETPPKPNFNNTSSSTPSPSAPSFFQGPSTPIPTPVLDNSPA
PIFSNPVCFFSSRSENNTEQYLQDSTLDLDSQLGDPTKNFNVNNFWSLF
PFDDVGYHPHSNDVGYHLHSDEESPFFDF.

In certain aspects, the C-cap region comprises a fragment of SEQ ID NO:336, such as a fragment having the amino acid sequence ALVKEYFPVFSSFHFTADQIVALICQSKQCFRNLKKNHQQWKNKGLSAEQIVDLILQETPPKP (SEQ ID NO: 337). In certain aspects, the C-cap region domain is a C-terminal domain or a fragment thereof from TALE proteins like those expressed in Burkholderia, Paraburkholderia, or Xanthomonas.

### Mixed DNA Binding Domains

In some embodiments, the present disclosure provides DNA binding domains in which the repeat units, the N-cap, and the C-ap can be derived from any one of Ralstonia solanacearum, Xanthomonas spp., Legionella quateirensis, Burkholderia, Paraburkholderia, or Francisella. For example, the present disclosure provides a DNA binding domain wherein the plurality of repeat units are selected from any one of the RUs as provided herein and can further comprise an N-cap and/or C-cap as provided herein.

### Repressor Domain

The terms “repressor,” “repressor domain,” and “transcriptional repressor domain” are used herein interchangeably to refer to a portion of the recombinant polypeptide as disclosed herein which portion decreases expression of a gene when the recombinant polypeptide is bound to the target gene. In certain aspects, the repressor domain comprises Krüppel-associated box (KRAB) protein. In other aspects, the repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2. In certain aspects, the repressor domain comprises an amino acid sequence at least 80%, at least 90%, at least 95%, at least 96%, at least 97%, at least 98%, at least 99%, or a 100% to the amino acid sequence set forth in one of SEQ ID NOs:84-101. In certain aspects, the repressor domain includes a KRAB domain comprising an amino acid sequence that is at least 80%, at least 90%, at least 95%, at least 96%, at least 97%, at least 98%, at least 99%, or a 100% to the amino acid sequence set forth in SEQ ID NO:338: RTLVTFKDVFVDFTREEWKLLDTAQQIVYRNVMLENYKNLVSLGYQLTKPDVILRLEKGEEP.

### Additional Features of the DBD

In certain aspects, the N-cap region or the C-cap region included in the disclosed DBD may include a nuclear localization sequence (NLS) to facilitate entry into the nucleus of a cell, e.g., an animal cell, such as, a human cell. In certain aspects, the polypeptide may be produced in a host cell and expressed with a translocation signal at the N-terminus which translocation signal may be cleaved during translocation.

In certain aspects, the RUs may be linked C-terminus to N-terminus with no additional amino acids separating immediately adjacent RUs. In certain aspects, immediately adjacent RUs may be separated by a spacer sequence of at least one amino acid. In certain aspects, the spacer sequence includes at least 2, 3, 4, 5, 6, or 7 amino acids, or up to 5, or up to 10 amino acids. The spacer sequence may include amino acids that have small side chains. In certain aspects, the spacer sequence is a flexible linker.

In some embodiments, a DBD of the present disclosure can comprise between 2 to 50 RUs, e.g., between 5 and 36, between 9 and 36, between 9 and 40, between 12 and 30, between 5 to 10, between 10 to 15, between 15 to 20, between 20 to 25, between 25 to 30, between 30 to 35 animal pathogen-derived repeat domains, or between 35 to 40 animal pathogen-derived repeat domains. In certain aspects, a MAP-NBD described herein can comprise up to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 animal pathogen-derived repeat domains.

### Imaging Moieties

A recombinant polypeptide as disclosed herein can be linked to a fluorophore, such as Hydroxycoumarin, methoxycoumarin, Alexa fluor, aminocoumarin, Cy2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5, Rox, Alexa fluor 568, Red 613, Texas Red, Alexa fluor 594, Alexa fluor 633, Allophycocyanin, Alexa fluor 633, Cy5, Alexa fluor 660, Cy5.5, TruRed, Alexa fluor 680, Cy7, GFP, or mCHERRY. A recombinant polypeptide as disclosed herein can be linked to a biotinylation reagent. In certain aspects, a recombinant polypeptide labeled with an imaging moiety as disclosed herein may be used to image binding and/localization of the recombinant polypeptide to a site in the genome of a cell.

### Compositions

In certain aspects, the polypeptides and the nucleic acids described herein may be present in a pharmaceutical composition comprising a pharmaceutically acceptable excipient. In certain aspects, the polypeptides and the nucleic acids are present in a therapeutically effective amount in the pharmaceutical composition. A therapeutically effective amount can be determined based on an observed effectiveness of the composition. A therapeutically effective amount can be determined using assays that measure the desired effect in a cell, e.g., in a reporter cell line in which expression of a reporter is modulated in response to the polypeptides of the present disclosure. The pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Suitable pharmaceutically acceptable or physiologically acceptable diluents, carriers or excipients include, but are not limited to, nuclease inhibitors, protease inhibitors, a suitable vehicle such as physiological saline solution or citrate buffered saline.

The pharmaceutical composition may include a plurality of the polypeptides provided herein. For example, the composition may include two, three, four, or more of the polypeptides provided herein, wherein the polypeptides all bind to sequences in regulatory region of the same gene or sequences in regulatory regions of different genes. For example, the composition may include a plurality of polypeptides that bind to a sequence of a target gene as disclosed herein (e.g., PD1, TIM3, or LAG3 gene). Alternatively, the composition may include a first polypeptide that binds to regulatory region of a first gene and a second polypeptide that binds to regulatory region of a second gene, where the first and second genes are independently selected from PD1, TIM3, and LAG3. The composition may include a first polypeptide that binds to regulatory region of PD1 gene, a second polypeptide that binds to regulatory region of TIM3 gene, and a third polypeptide that binds to regulatory region of LAG3 gene. The composition may include a plurality of polypeptides that bind to regulatory region of PD1 gene, a plurality of polypeptides that bind to regulatory region of TIM3 gene, and a plurality of polypeptides that bind to regulatory region of LAG3 gene.

### Delivery

The polypeptides disclosed herein, compositions comprising the disclosed polypeptides, and nucleic acids encoding the disclosed polypeptides can be delivered into a target cell by any suitable means, including, for example, by injection, infection, transfection, and vesicle or liposome mediated delivery.

In certain aspects, a mRNA or a vector encoding the polypeptides disclosed herein may be injected, transfected, or introduced via viral infection into a target cell, where the cell is ex vivo or in vivo. Any vector systems may be used including, but not limited to, plasmid vectors, retroviral vectors, lentiviral vectors, adenovirus vectors, poxvirus vectors; herpesvirus vectors and adeno-associated virus vectors, etc. When two or more polypeptides according to present disclosure are introduced into the cell, the nucleic acids encoding the polypeptides may be carried on the same vector or on different vectors. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. Vectors suitable for introduction of polynucleotides as described herein include described herein include non-integrating lentivirus vectors (IDLV).

Non-viral vector delivery systems include electroporation, lipofection, microinjection, biolistics, virosomes, liposomes, immunoliposomes, polycation or lipid:nucleic acid conjugates, naked DNA, artificial virions, and agent-enhanced uptake of DNA.

Primary cells may be isolated and used ex vivo for reintroduction into the subject to be treated. Suitable primary cells include peripheral blood mononuclear cells (PBMC), and other blood cell subsets such as, but not limited to, CD4+ T cells or CD8+ T cells. In certain aspects, the cell may be a CART cell. Suitable cells also include stem cells such as, by way of example, embryonic stem cells, induced pluripotent stem cells, hematopoietic stem cells, neuronal stem cells, mesenchymal stem cells, muscle stem cells and skin stem cells. In certain aspects, the stem cells may be isolated from a subject to be treated or may be derived from a somatic cell of a subject to be treated using the polypeptides disclosed herein.

In certain aspects, the cells into which the polypeptides of the present disclosure or a nucleic acid encoding a polypeptide of the present disclosure may be an animal cell, e.g., from a human needing treatment.

In certain aspects, the polypeptide of the present disclosure is only transiently present in a target cell. For example, the polypeptide is expressed from a nucleic acid that expressed the polypeptide for a short period of time, e.g., for up to 1 day, 3 days, 1 week, 3 weeks, or 1 month. In applications where transient expression of the polypeptide of the present disclosure is desired, adenoviral based systems may be used. Adeno-associated virus (“AAV”) vectors can also be used to transduce cells with nucleic acids encoding the polypeptide of the present disclosure, e.g., in the in vitro production of nucleic acids and peptides, and for in vivo and ex vivo gene therapy procedures. In certain aspects, recombinant adeno-associated virus vectors (rAAV) such as replication-deficient recombinant adenoviral vectors may be used for introduction of nucleic acids encoding the polypeptides disclosed herein.

In certain aspects, nucleic acids encoding the polypeptides disclosed herein can be delivered using a gene therapy vector with a high degree of specificity to a particular tissue type or cell type. A viral vector is typically modified to have specificity for a given cell type by including a sequence encoding a ligand expressed as a fusion protein with a viral coat protein on the viruses' outer surface. The ligand is chosen to have affinity for a receptor known to be present on the cell type of interest.

In certain aspects, gene therapy vectors can be delivered in vivo by administration to an individual patient. In certain aspects, administration involves systemic administration (e.g., intravenous, intraperitoneal, intramuscular, subdermal, or intracranial infusion), direct injection (e.g., intrathecal), or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector or which have been modified by expression of the polypeptide of the present disclosure encoded by the vector.

In certain aspects, the nucleic acid encoding the polypeptides provided herein may be codon optimized to enhance expression of the polypeptide in the target cell. For example, the sequence of the nucleic acid can be varied to provide codons that are known to be highly used in animal cells, such as, human cells to enhance production of the polypeptide in a human cell. For example, silent mutations may be made in the nucleotide sequence encoding a polypeptide disclosed herein for codon optimization in mammalian cells.

### Methods for Gene Suppression in Target Cells

In some aspects, described herein is a method of suppressing expression of PDCD-1 gene in a cell, the method comprising introducing into the cell the recombinant polypeptide that comprises the DBD and the transcriptional repressor domain as provided herein, where the DBD binds to a target nucleic acid sequence present in the PDCD-1 gene and the transcriptional repressor suppresses expression of the PDCD-1 gene.

In some aspects, described herein is a method of suppressing expression of TIM3 gene in a cell, the method comprising introducing into the cell the recombinant polypeptide that comprises the DBD and the transcriptional repressor domain as provided herein, where the DBD binds to a target nucleic acid sequence present in the TIM3 gene and the transcriptional repressor suppresses expression of the TIM3 gene.

In some aspects, described herein is a method of suppressing expression of LAG3 gene in a cell, the method comprising introducing into the cell the recombinant polypeptide that comprises the DBD and the transcriptional repressor domain as provided herein, where the DBD binds to a target nucleic acid sequence present in the LAG3 gene and the transcriptional repressor suppresses expression of the LAG3 gene.

In certain aspects, the polypeptide is introduced as a nucleic acid encoding the polypeptide. In certain aspects, the nucleic acid is a deoxyribonucleic acid (DNA). In certain aspects, the nucleic acid is a ribonucleic acid (RNA). In certain aspects, the sequence of the nucleic acid is codon optimized for expression in a human cell.

In certain aspects, the cell is an animal cell. In certain aspects, the cell is a human cell. In certain aspects, the cell is a cancer cell. In certain aspects, the cell is an ex vivo cell.

In certain aspects, the introducing comprises administering the polypeptide or a nucleic acid encoding the polypeptide to a subject. In certain aspects, the administering comprises parenteral administration. In certain aspects, the administering comprises intravenous, intramuscular, intrathecal, or subcutaneous administration. In certain aspects, the administering comprises direct injection into a site in a subject. In certain aspects, the administering comprises direct injection into a tumor.

In certain aspects, the introducing may induce a repression of expression of the target gene for a period of at least 2 days, at least 3 days, at least 9 days, at least at least 15 days, at least 1 month, at least 6 months, at least 1 year to up to 5 years. In certain aspects, the introducing may suppress expression of gene expression by at least 10%, at least 20%, at least 30%, at least 40%, at least 50%, at least 60%, at least 70%, at least 80%, or more. In certain aspects, the introducing may be repeated to maintain suppression of target gene expression. In certain aspects, the introducing may be performed as a combination therapy with, for example, a cancer therapy. The combination therapy may involve introducing the recombinant polypeptide into the cell prior to, concurrently with, or after administration of cancer therapy.

An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtle, lizard or snake. A fish cell can be from a tropical fish. For example, the fish cell can be from a zebrafish (e.g., Danio rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include an epithelial cell, connective tissue cell, hormone secreting cell, a nerve cell, a skeletal muscle cell, a blood cell, an immune system cell, or a stem cell.

Exemplary mammalian cells can include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, PC12 cell line, primary cells (e.g., from a human) including primary T cells, primary hematopoietic stem cells, primary human embryonic stem cells (hESCs), and primary induced pluripotent stem cells (iPSCs).

In some cases, a target cell is a cancerous cell, e.g., in a human. Cancer can be a solid tumor or a hematologic malignancy. The solid tumor can include a sarcoma or a carcinoma. Exemplary sarcoma target cell can include, but are not limited to, cell obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma target cell can include, but are not limited to, cell obtained from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Alternatively, the cancerous cell can comprise cells obtained from a hematologic malignancy. Hematologic malignancy can comprise a leukemia, a lymphoma, a myeloma, a non-Hodgkin's lymphoma, or a Hodgkin's lymphoma. In some cases, the hematologic malignancy can be a T-cell based hematologic malignancy. Other times, the hematologic malignancy can be a B-cell based hematologic malignancy. Exemplary B-cell based hematologic malignancy can include, but are not limited to, chronic lymphocytic leukemia (CLL), small lymphocytic lymphoma (SLL), high-risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy can include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some cases, a cell can be a tumor cell line. Exemplary tumor cell line can include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

### Methods of Production of Polypeptides

In certain embodiments, the polypeptides disclosed herein are produced using a suitable method including recombinant and non-recombinant methods (e.g., chemical synthesis).

### A. Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as Fmoc and Boc, are available for synthesizing polypeptides of the present disclosure. Details of the chemical synthesis are known in the art (e.g., Ganesan A. 2006 Mini Rev. Med. Chem. 6:3-10; and Camarero J. A. et al., 2005 Protein Pept Lett. 12:723-8).

### B. Recombinant Production

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. In certain aspects, eukaryotic cells that are used as host cells for production of the polypeptides include insect cells, mammalian cells, and/or plant cells. In certain aspects, mammalian host cells are used and may include human cells (e.g., HeLa, 293, H9 and Jurkat cells); mouse cells (e.g., NIH3T3, L cells, and C127 cells); primate cells (e.g., Cos 1, Cos 7 and CV1) and hamster cells (e.g., Chinese hamster ovary (CHO) cells). In specific embodiments, the polypeptide disclosed herein are produced in CHO cells.

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences and may provide for inducible or constitutive expression where the coding region is operably-linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Also provided herein are nucleic acids encoding the polypeptides disclosed herein. In certain aspects, a nucleic acid encoding the polypeptides disclosed herein is operably linked to a promoter sequence that confers expression of the polypeptide. In certain aspects, the sequence of the nucleic acid is codon optimized for expression of the polypeptide in a human cell. In certain aspects, the nucleic acid is a deoxyribonucleic acid (DNA). In certain aspects, the nucleic acid is a ribonucleic acid (RNA). Also provided herein is a vector comprising the nucleic acid encoding the polypeptides for binding a target nucleic acid as described herein. In certain aspects, the vector is a viral vector.

In certain aspects, a host cell comprising the nucleic acid or the vector encoding the polypeptides disclosed herein is provided. In certain aspects, a host cell comprising the polypeptides disclosed herein is provided. In certain aspects, a host cell that expresses the polypeptide is also disclosed.

### Recombinant Polypeptides Comprising Novel Transcription Repressor Domains

The present disclosure also provides recombinant polypeptide comprising a DNA binding domain and a transcriptional repressor domain, wherein the DNA binding domain and the transcriptional repressor domain are heterologous, wherein the transcriptional repressor domain comprises an amino acid sequence at least 80% identical to any one of the sequences set out in SEQ ID NOs: 84-101.

In certain aspects, the transcriptional repressor domain comprises an amino acid sequence at least 85% identical, at least 90% identical, at least 95% identical, or a 100% identical to any one of the sequences set out in SEQ ID NOs: 84-101.

The DNA binding domain may be a zinc finger protein (ZFP), a transcription activator-like effector (TALE), or a guide RNA. In certain aspects, the DNA binding domain may be a DBD as disclosed herein that binds to a target sequence provided herein.

In certain aspects, the DNA binding domain may bind to a target nucleic acid sequence in a gene. The target nucleic acid sequence may be present in a PDCD1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.

The present disclosure also provides a nucleic acid encoding the recombinant polypeptide. The nucleic acid may be operably linked to a promoter sequence that confers expression of the polypeptide.

In certain aspects, the sequence of the nucleic acid is codon optimized for expression of the polypeptide in a human cell. In certain aspects, the nucleic acid is a deoxyribonucleic acid (DNA). In certain aspects, the nucleic acid is a ribonucleic acid (RNA).

The present disclosure also provides a vector comprising the nucleic acid disclosed herein. In certain aspects, the vector may be a viral vector.

The present disclosure also provides a host cell comprising the nucleic acid or the vector disclosed herein. In certain aspects, the host cell may include the polypeptide. In certain aspects, the host cell may express the polypeptide.

Also provided herein is a pharmaceutical composition comprising the polypeptide and a pharmaceutically acceptable excipient. The pharmaceutical composition may include the nucleic acid or the vector and a pharmaceutically acceptable excipient.

Also provided herein is a method of suppressing expression of an endogenous gene in a cell. The method may include introducing into the cell the recombinant polypeptide, wherein the DBD of the polypeptide binds to a target nucleic acid sequence present in the endogenous gene and the heterologous transcriptional repressor domain suppresses expression of the endogenous gene.

In certain aspects, the recombinant polypeptide is introduced as a nucleic acid encoding the polypeptide. The nucleic acid may be a deoxyribonucleic acid (DNA) or RNA. The nucleic acid may be codon optimized for expression in a human cell.

The target gene may be a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

The cell may be an animal cell. The cell may be a human cell. The cell may be a cancer cell. The cell may be an ex vivo cell or an in vivo cell.

In certain aspects, the introducing may include administering the polypeptide or a nucleic acid encoding the polypeptide to a subject. The administering may include parenteral administration. The administering may include intravenous, intramuscular, intrathecal, or subcutaneous administration. The administering may include direct injection into a site in a subject. The administering may include direct injection into a tumor.

### Split Systems for Modulating Gene Expression

Split systems for modulating gene expression are provided. In certain aspects, a DBD and a functional domain are provided as separate polypeptides instead of a single polypeptide and are assembled into a functional complex using dimerization of a heterodimer pair, where the DBD and the functional domain are each fused to a member of the heterodimer pair. In certain aspects, indirect dimerization may also be utilized by using a fused polypeptide comprising two individual members of a heterodimer pair that act as a bridge to bring a DBD and a functional domain together, as explained in detail below.

These split systems find use in screens for a DBD or a functional domain by, e.g., using a DBD fused to a first member of a heterodimer pair and screening a plurality of candidate functional domains each fused to a second member of the heterodimer pairs and vice versa.

These split systems find use in providing additional control in modulation of gene expression by a DBD: functional domain complex. In certain aspects, control of modulation of gene expression may be achieved by having the DBD and functional domain expression on board (e.g., constitutive expression) a cell as separate polypeptides and assembling a functional DBD and functional domain complex by introducing a bridging construct into the cell, when modulation of gene expression is desired. The bridging construct may be expressed transiently thereby modulating gene expression transiently. In certain aspects, control of modulation of gene expression may be achieved by disrupting the DBD and functional domain complex by introducing a disruptor comprising a heterodimer pair or an individual member of a heterodimer pair as explained below.

As would be understood by the skilled person, the individual components of a split system may be introduced into a cell as nucleic acids encoding the individual components or as polypeptides or a combination thereof.

The split systems may be used for modulating gene expression in any cell such as a mammalian cell having a target site at which the DBD binds. Examples of such cells are provided herein, e.g., in the preceding sections of the application.

The heterodimer pairs of the split system include: 37A, 37B; 13A, 13B; DHD37-BBB-A, DHD37-BBB-B; DHD150-A, DHD150-B; DHD154-A, DHD-154B; 37A, 9B; 13A, 37B; 13A, DHD150-B; 37A, DHD37-BBB-B; and DHD37-BBB-A, 37B, where each of 37A, 37B, 13A, 13B, DHD37-BBB-A, DHD37-BBB-B, DHD150-A, DHD150-B, DHD154-A, and DHD-154B, are the individual members of the listed heterodimer pairs. As used herein, the term first member and second member refers to either of the individual members of a listed heterodimer pair.

The term “37A” and the numeral “1” are used herein interchangeably and in the context of a member of a heterodimer pair refer to a polypeptide comprising an amino acid sequence that is at least 80% identical (e.g., at least 85%, at least 90%, at least 95%, at least 96%, at least 97%, at least 98%, at least 99% identical) to the amino acid sequence: DSDEHLKKLKTFLENLRRHLDRLDKHIKQLRDILSENPEDERVKDVIDLSERSVRIVKTVIKIFEDS VRKKE (SEQ ID NO: 473), and is capable of binding to 37B, 9B, and DHD37-BBB-B.

The terms “37B” and “1′” are used herein interchangeably and in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence:

GSDDKELDKLLDTLEKILQTATKIIDDANKLLEKLRRSERKDPKVVETYVELLKRHEKAV KELLEIAKTHAKKVE (SEQ ID NO: 474), and is capable of binding to 37A, 13A, and DHD37-BBB-A.

The term “13A” and the numeral “9” are used herein interchangeably and in the context of a member of a heterodimer pair refer to a polypeptide comprising an amino acid sequence that is at least 80% identical (e.g., at least 85%, at least 90%, at least 95%, at least 96%, at least 97%, at least 98%, at least 99% identical) to the amino acid sequence:

GTKEDILERQRKIIERAQEIHRRQQEILEELERIIRKPGSSEEAMKRMLKLLEESLRLLKELL ELSEESAQLLYEQR (SEQ ID NO: 475), and is capable of binding to 13B, 37B, and DHD150-B.

The terms “13B” and “9′” are used herein interchangeably and in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence:

GTEKRLLEEAERAHREQKEIIKKAQELHRRLEEIVRQSGSSEEAKKEAKKILEEIRELSKRS LELLREILYLSQEQKGSLVPR (SEQ ID NO: 476), and is capable of binding to 13A.

The term “DHD37-BBB-A” in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence: DEEDHLKKLKTHLEKLERHLKLLEDHAKKLEDILKERPEDSAVKESIDELRRSIELVRESIEIFRQS VEEEE (SEQ ID NO: 477), and is capable of binding to DHD37-BBB-B and 37B.

The term “DHD37-BBB-B” in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence: GDVKELTKILDTLTKILETATKVIKDATKLLEEHRKSDKPDPRLIETHKKLVEEHETLVRQHKELA EEHLKRTR (SEQ ID NO: 478), and is capable of binding to DHD37-BBB-A and 37A.

The term “DHD150-A” in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence: GDVKELTKILDTLTKILETATKVIKDATKLLEEHRKSDKPDPRLIETHKKLVEEHETLVRQHKELA EEHLKRTR (SEQ ID NO: 478), and is capable of binding to DHD150-B.

The term “DHD150-B” in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence: DNEEIIKEARRVVEEYKKAVDRLEELVRRAENAKHASEKELKDIVREILRISKELNKVSERLIELW ERSQERAR (SEQ ID NO: 479), and is capable of binding to DHD150-A and 13A.

The terms “DHD154-A” and “DHD-154-A” in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence: TAEELLEVHKKSDRVTKEHLRVSEEILKVVEVLTRGEVSSEVLKRVLRKLEELTDKLRRVTEEQR RVVEKLN (SEQ ID NO: 480), and is capable of binding to DHD-154-B.

The terms “DHD154-B” and “DHD-154-B” in the context of a member of a heterodimer pair refers to a polypeptide comprising an amino acid sequence that is at least 80% identical (e.g., at least 85%, at least 90%, at least 95%, at least 96%, at least 97%, at least 98%, at least 99% identical) to the amino acid sequence: DLEDLLRRLRRLVDEQRRLVEELERVSRRLEKAVRDNEDERELARLSREHSDIQDKHDKLAREIL EVLKRLLERTE (SEQ ID NO: 481), and is capable of binding to DHD-154-A.

In certain aspects, the present disclosure provides two or more nucleic acids encoding one or more of the members of the heterodimer pairs. In certain aspects, the nucleic acid encoding a fusion protein comprising a DBD and a member of a heterodimer pair and another nucleic acid encoding a fusion protein comprising a functional domain and a member of the heterodimer pair are provided.

In certain aspects, a plurality of nucleic acids are provided, where the plurality of nucleic acids encode (i) polypeptides that dimerize via direct dimerization, comprising: (A) a DBD fused to a first member of a heterodimer pair and a functional domain fused to a second member of the heterodimer pair, or (B) a DBD fused to a second member of a heterodimer pair and a functional domain fused to a first member of the heterodimer pair, wherein the first and second members of the heterodimer pair bind to each other thereby directly dimerizing the DBD and the functional domain, and wherein the heterodimer pair is selected from one of the following heterodimer pairs: 37A, 37B; 13A, 13B; DHD37-BBB-A, DHD37-BBB-B; DHD150-A, DHD150-B; DHD154-A, DHD-154B; 37A, 9B; 13A, 37B; 13A, DHD150-B; 37A, DHD37-BBB-B; and DHD37-BBB-A, 37B.

In certain aspects, the DBD in (i) (A) or (i) (B) may be fused to a first member of a first heterodimer pair and the functional domain is a first functional domain fused a second member of the first heterodimer pair and to a first member of a second heterodimer pair, and may be used with a second functional domain fused to a second member of the second heterodimer pair, wherein the members of the first heterodimer pair mediate dimerization of the DBD and the first functional domain and members of the second heterodimer pair mediate dimerization of the first functional domain and the second functional domain. In certain aspects, the DBD is fused to a first member of a first heterodimer pair and to a first member of a second heterodimer pair, and the functional domain is fused a second member of the first heterodimer pair the system further comprising a second functional domain fused to a second member of the second heterodimer pair, wherein the members of the first heterodimer pair mediate assembly of the DBD and the first functional domain and members of the second heterodimer pair mediate assembly of the DBD and the second functional domain.

In certain aspects, a plurality of nucleic acids are provided, where the plurality of nucleic acids encode (ii) polypeptides that dimerize indirectly via a bridging construct, comprising: (A) a DBD fused to a first member of a first heterodimer pair; a bridging construct comprising a second member of the first heterodimer pair fused to a first member of a second heterodimer pair; and a functional domain fused to a second member of the second heterodimer pair; or (B) a DBD fused to a second member of a first heterodimer pair; a bridging construct comprising a first member of the first heterodimer pair fused to a first member of a second heterodimer pair; and a functional domain fused to a second member of the second heterodimer pair; or (C) a DBD fused to a second member of a first heterodimer pair; a bridging construct comprising a first member of the first heterodimer pair fused to a second member of a second heterodimer pair; and a functional domain fused to a first member of the second heterodimer pair, wherein the DBD and the functional domain dimerize indirectly via the bridging construct, wherein the first and second heterodimer pairs are different and are selected from the following heterodimer pairs: 37A, 37B; 13A, 13B; DHD37-BBB-A, DHD37-BBB-B; DHD150-A, DHD150-B; DHD154-A, DHD-154B; 37A, 9B; 13A, 37B; 13A, DHD150-B; 37A, DHD37-BBB-B; and DHD37-BBB-A, 37B. For example, the DBD may be fused to 37A, the bridging construct may be a fusion of 37B and 13A, and the functional domain fused to 13B.

As described in the specification, the DBD may bind to a target nucleic acid sequence present in an endogenous gene in a cell. The functional domain may be an enzyme, a transcriptional activator, a transcriptional repressor, or a DNA nucleotide modifier. The enzyme may be a nuclease, a DNA modifying protein, or a chromatin modifying protein. The nuclease may be a cleavage domain or a half-cleavage domain. The cleavage domain or half-cleavage domain may be a type IIS restriction enzyme.

The type IIS restriction enzyme may be FokI or Bfil. The chromatin modifying protein may be lysine-specific histone demethylase 1 (LSD1). The transcriptional activator may be VP16, VP64, p65, p300 catalytic domain, TET1 catalytic domain, TDG, Ldb1 self-associated domain, SAM activator (VP64, p65, HSF1), or VPR (VP64, p65, Rta). The transcriptional repressor may be KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, MeCP2, or a novel transcriptional repressor as disclosed herein. The DNA nucleotide modifier may be an adenosine deaminase. The target nucleic acid sequence may be within a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene. The DBD may be a transcription activator-like effector (TALE). The DBD may be a novel DBD as provided herein.

Also provided herein are a DBD fused to a member of a heterodimer pair, a functional domain fused to a member of a heterodimer pair, a bridging construct comprising a member of a heterodimer pair fused to another member, such as those described in the preceding paragraphs and further described below and those encoded by the plurality of nucleic acids described above.

In certain aspects, a DBA and a functional domain is as set forth in (i)(A) or (i)(B). In certain aspects, a DBD, a bridging construct, and a functional domain is as set forth in (ii)(A), (ii)(B), or (ii)(C).

Also provided herein are host cells that include (a) nucleic acids encoding the polypeptides as set forth in (i)(A) or (i)(B); or (b) nucleic acids encoding the polypeptides as set forth in (ii)(A), (ii)(B), or (ii)(C).

Also provided herein are host cells that include host cells that include (a) the polypeptides as set forth in (i)(A) or (i)(B); or (b) the polypeptides as set forth (ii)(A), (ii)(B), or (ii)(C).

Also provided herein is a kit comprising: (a) nucleic acids encoding the polypeptides as set forth in (i)(A) or (i)(B); or (b) nucleic acids encoding the polypeptides as set forth in (ii)(A), (ii)(B), or (ii)(C).

Also provided herein is a kit comprising: (a) a first vector comprising a nucleic acid encoding the DBD set forth in (i)(A); and (b) a second vector comprising a nucleic acid encoding the functional domain set forth in (i)(A); or (a) a first vector comprising a nucleic acid encoding the DBD set forth in (i)(B); and (b) a second vector comprising a nucleic acid encoding the functional domain set forth in (i)(B).

Also provided herein is a kit comprising: a first vector comprising a nucleic acid encoding the DBD set forth in (ii)(A); a second vector comprising a nucleic acid encoding the bridging construct set forth in (ii)(A); and a third vector comprising a nucleic acid encoding the functional domain set forth in (ii)(A); or (a) a first vector comprising a nucleic acid encoding the DBD set forth in (ii)(B); (b) a second vector comprising a nucleic acid encoding the bridging construct set forth in (ii)(B); and (c) a third vector comprising a nucleic acid encoding the functional domain set forth in (ii)(B); or a first vector comprising a nucleic acid encoding the DBD set forth (ii)(C); a second vector comprising a nucleic acid encoding the bridging construct set forth in (ii)(C); and a third vector comprising a nucleic acid encoding the functional domain set forth in (ii)(C).

Also disclosed are pharmaceutical compositions comprising the nucleic acids disclosed herein or the polypeptides disclosed herein. The pharmaceutical composition may also include a pharmaceutically acceptable excipient. In certain aspects, the pharmaceutical composition may include (a) nucleic acids encoding the polypeptides as set forth in (i)(A) or (i)(B); or (b) nucleic acids encoding the polypeptides as set forth in (ii)(A), (ii)(B), or (ii)(C).

In certain aspects, the pharmaceutical composition may include (a) a first vector comprising a nucleic acid encoding the DBD set forth in (i)(A); and (b) a second vector comprising a nucleic acid encoding the functional domain set forth in (i)(A); or (a) a first vector comprising a nucleic acid encoding the DBD set forth in (i)(B); and (b) a second vector comprising a nucleic acid encoding the functional domain set forth in (i)(B).

In certain aspects, the pharmaceutical composition may include: (a) a first vector comprising a nucleic acid encoding the DBD set forth in (ii)(A); (b) a second vector comprising a nucleic acid encoding the bridging construct set forth in (ii)(A); and (c) a third vector comprising a nucleic acid encoding the functional domain set forth in (ii)(A); or (a) a first vector comprising a nucleic acid encoding the DBD set forth in (ii)(B); (b) a second vector comprising a nucleic acid encoding the bridging construct set forth in (ii)(B); and (c) a third vector comprising a nucleic acid encoding the functional domain set forth in (ii)(B); or (a) a first vector comprising a nucleic acid encoding the DBD set forth in (ii)(C); (b) a second vector comprising a nucleic acid encoding the bridging construct set forth in (ii)(C); and (c) a third vector comprising a nucleic acid encoding the functional domain set forth in (ii)(C).

In certain aspects, the pharmaceutical composition may include the DBD and a functional domain or a DNA binding domain, a functional domain and a bridging construct as provided herein and a pharmaceutically acceptable excipient. In certain aspects, the pharmaceutical composition may include the host cell as provided herein and a pharmaceutically acceptable excipient.

The split systems of DBD and functional domains and heterodimer pairs may be used in a method for modulating expression from a target gene in a cell. The method may include (i) introducing into the cell a first nucleic acid encoding a DNA binding domain fused to a first member of a heterodimer pair and a second nucleic acid encoding a functional domain fused to a second member of the heterodimer pair; or (ii) introducing into the cell a first nucleic acid encoding a DNA binding domain fused to a second member of a heterodimer pair and a second nucleic acid encoding a functional domain fused to a first member of the heterodimer pair; or (iii) introducing into the cell a DNA binding domain fused to a first member of a heterodimer pair and a functional domain fused to a second member of the heterodimer pair; or (iv) introducing into the cell a DNA binding domain fused to a second member of a heterodimer pair and a functional domain fused to a first member of the heterodimer pair. The heterodimer pair may be selected from one of the following heterodimer pairs: 37A, 37B; 13A, 13B; DHD37-BBB-A, DHD37-BBB-B; DHD150-A, DHD150-B; DHD154-A, DHD-154B; 37A, 9B; 13A, 37B; 13A, DHD150-B; 37A, DHD37-BBB-B; and DHD37-BBB-A, 37B, wherein the DBD dimerizes with the functional domain via dimerization of the members of the heterodimer pair and wherein binding of the DBD to a target nucleic acid sequence in the target gene results in modulation of expression of the target gene via the functional domain dimerized to the DBD.

In certain aspects, the method may be used for screening a candidate DBD or a candidate functional domain or for ranking DBDs or functional domains based on specificity, activity, and the like. The modulation of expression of the target gene may be assessed to determine whether a DBD is specific for the target gene and/or whether the functional domain is active in repressing or activating expression of the target gene.

The split systems of DBD and functional domains and heterodimer pairs may be used in a method for modulating expression from a target gene in a cell, where the method includes introducing into a cell expressing a DNA binding domain (DBD) fused to a first member of a first heterodimer pair and a functional domain fused to a second member of a second heterodimer pair, a bridging construct comprising a second member of the first heterodimer pair fused to a first member of the second heterodimer pair or a nucleic acid encoding the bridging construct; or introducing into a cell expressing a DNA binding domain (DBD) fused to a second member of a first heterodimer pair and a functional domain fused to a second member of a second heterodimer pair, a bridging construct comprising a first member of the first heterodimer pair fused to a first member of the second heterodimer pair or a nucleic acid encoding the bridging construct; or introducing into a cell expressing a DNA binding domain (DBD) fused to a first member of a first heterodimer pair and a functional domain fused to a first member of a second heterodimer pair, a bridging construct comprising a second member of the first heterodimer pair fused to a second member of the second heterodimer pair or a nucleic acid encoding the bridging construct, wherein the DBD and the functional domain dimerize indirectly via the bridging construct, wherein binding of the DBD to a target nucleic acid sequence in a target gene in the cell results in in modulation of expression of the target gene via the functional domain dimerized to the DBD via the bridging construct, wherein the first and second heterodimer pairs are different and are selected from the following heterodimer pairs: 37A, 37B; 13A, 13B; DHD37-BBB-A, DHD37-BBB-B; DHD150-A, DHD150-B; DHD154-A, DHD-154B; 37A, 9B; 13A, 37B; 13A, DHD150-B; 37A, DHD37-BBB-B; and DHD37-BBB-A, 37B.

Such a system may be used for fine tuning control of modulation of gene expression by controlling expression of the different components required for modulating gene expression.

Also provided is a method of reversing modulation of expression of a target gene in a cell expressing a DNA binding domain (DBD) fused to a first member of a non-cognate heterodimer pair and a functional domain fused to a second member of the non-cognate heterodimer pair, wherein the DBD binds to a target nucleic acid sequence in a target gene and the functional domain dimerized to the DBD via dimerization of the members of the heterodimer pair modulates expression of the target gene, the method comprising introducing into the cell a disruptor which binds to either the first member or the second member with a higher binding affinity than the binding affinity between the first and second members, wherein non-cognate heterodimer pairs and the corresponding disruptor are selected from one of the following combinations:

| Combination | Non-Cognate Heterodimer Pair | Disruptor |
|---|---|---|
| 1 | 37A, 9B; | 37B or 9A |
| 2 | 13A, 37B; | 13B or 37A |
| 3 | 13A, DHD150-B; | 13B or DHD150-A |
| 4 | 37A, DHD37-BBB-B; | 37B or DHD37-BBB-A |
| 5 | DHD37-BBB-A, 37B | DHD37-BBB-B or 37A |

As used herein, the term “non-cognate heterodimer pair” refers to a heterodimer pair whose members bind to each other with an affinity that is lower than the affinity with which members of a “cognate heterodimer pair” bind. For example, 37A, 37B is a cognate heterodimer pair while 37A, 9B form a non-cognate heterodimer pair, since the binding affinity between 37A and 37B is higher than that between 37A and 9B. Examples of cognate heterodimer pairs include 37A, 37B; 13A, 13B; DHD37-BBB-A, DHD37-BBB-B; DHD150-A, DHD150-B; and DHD154-A, DHD-154B. While members of a “non-cognate heterodimer” bind to each other, members that are not part of a “non-cognate heterodimer” or a “cognate heterodimer” do not significantly bind to each other and are not considered as members of a heterodimer pair.

In certain aspect, the fusion polypeptides, such as, DBD fused to a member of a heterodimer pair may be such that the C-terminus of the DBD is fused to the N-terminus of a member of a heterodimer pair and the N-terminus of the functional domain is fused to the C-terminus of a member of a heterodimer pair. In certain aspects, one or more components of the system may be expressed transiently while other component(s) are expressed stably. Stable and transient expression in a cell may be achieved by methods known in the art, such as, transient transfection, gene integration, constitutive and inducible promoters and the like.

### EXAMPLES

These examples are provided for illustrative purposes only and not to limit the scope of the claims provided herein.

### Materials and Methods

TALE backbone sequences:

N-Cap:
(SEQ ID NO: 339)
DYKDHDGDYKDHDIDYKDDDDKMAPKKKRKVGIHRGVPMVDLRTLGYSQ
QQQEKIKPKVRSTVAQHHEALVGHGFTHAHIVALSQHPAALGTVAVKYQ
DMIAALPEATHEAIVGVGKQWSGARALEALLTVAGELRGPPLQLDTGQL
LKIAKRGGVTAVEAVHAWRNALTGAPLETPN
Repeat Unit:
(SEQ ID NO: 340)
LTPDQVVAIASX₁₁X₁₂GGKQALETVQRLLPVLCQDHG
Half repeat unit:
(SEQ ID NO: 341)
LTPEQVVAIASX₁₁X₁₂GG
RVD = X₁₁X₁₂; X₁₁X₁₂ = NH for binding G; NG for
binding T; NI for binding A; and HD for binding C.
C-Cap:
(SEQ ID N: 342)
RPALESIVAQLSRPDPALAALTNDHLVALACLGGRPALDAVKKGLPHAP
ALIKRTNRRIPERTSHRVA
Flexible linker between C-Cap and KRAB:
(SEQ ID NO: 343)
GAGGGGGMDAKSLTAWS
KRAB:
(SEQ ID NO: 338)
RTLVTFKDVFVDFTREEWKLLDTAQQIVYRNVMLENYKNLVSLGYQLTK
PDVILRLEKGEEP

Anti CD19 CAR-T cell manufacturing: Primary T cells were thawed and activated with CD3/CD28 Dynabeads and cultured for 48 hours prior to electroporation with either no mRNA (control) or mRNA encoding the TALE-TFs against PD1. At 24 hours post electroporation T cells were transduced with a lentivirus vector encoding a 3^{rd} generation anti CD19 CAR construct on Retronectin at an MOI of 5 to 10. After 24 hours the virus and beads were removed and T cells expanded in RPMI+10% FBS+IL-2 for up to 5 days.

Co-culture (killing) assay: CAR-T cells and control T cells were incubated with CD19-expressing NALM-6 cells or NALM-6 cells engineered to express PDL-1 (the ligand for PD-1) or NALM-6 cells in which the target antigen CD19 was knocked out using TALENs (CD19 KO) at an effector-to-target (E:T) ratio of 1:1 in a 96-well round bottom culture plate for 16 hours at 37 degrees with 5% C02. After 16 hours of incubation, specific target cell killing was measured by release of lactate dehydrogenase (LDH) into the supernatant (Promega kit #) or by flow cytometry analysis.

Animal model: Human B-Acute Lymphoblastic Leukemia (ALL) NALM-6 cells expressing CD19 were implanted intra-venously into NOD SCID Gamma (NSG) mice at 0.5 million cells per mouse. 5 days later when tumor engraftment was detectable by in vivo imaging, mice were injected intra-venously with 2.5 million anti-CD19 CAR+ T cells either treated or untreated with the anti PD-1 TALE-TF pAL043. Mice were bled once per week after infusion and blood was processed for flow cytometry to detect human CD3+ T cells, CAR-T cells and measure expression of PD-1.

Off Target Analysis: CD3+ cells were electroporated with TALETFs (either single or multiplexed) in triplicate. Cells were harvested at 2 days post-transfection for RNA extraction and parallel analysis of expression using flow cytometry. Total RNA was extracted from these samples and from control T cells electroporated without mRNA using Qiagen miRNeasy extraction kit. Total RNA samples were constructed into libraries using Illumina's TruSeq Stranded Total RNA Plurality of nucleic acids Prep Gold kit. Libraries were then sequenced using Illumina's Hiseq 4000 platform with 2×76 bp read length to a depth of 25-50 million reads per sample. Reads were aligned using STAR paired alignment (RNA-STAR 2.3.1), mapped to the GRCh38 human genome assembly, and differential gene expression analysis was performed using edgeR.

Synthetic Repressor Design and Assembly. TAL monomers were cloned and assembled into full length TALs with modifications to established methods (T. Cermak et al., Nucleic Acids Res 39, e82 (2011); T. Sakuma et al., Genes Cells 18, 315-326 (2013) into a pVAX-based plasmid and included an N-terminal 3×-FLAG tag and SV40 nuclear localization signal. Functional domains were selected by literature search for evidence of transcriptional repressive function and annotated DNA-binding domains removed in silico before synthesis and incorporation into TAL or heterodimer constructs. Functional domains were added by Infusion cloning (Takara Bio; catalog #638909) onto the C-terminal end of the TAL. Functional domain constructs contained a 15 amino acid linker domain (GGGGGMDAKSLTAWS) (SEQ ID NO: 109) and either an epigenetic-functional domain (e.g. —KRAB) or heterodimer protein (e.g.—9′ of the 9:9′ pair).

Obligate heterodimers. Mutually orthogonal heterodimer pairs listed in Table 14 were designed and synthesized. Heterodimer sequences were appended to sequences encoding TAL-DBDs or effector domains via colinear placement in plasmids used for in vitro RNA transcription. Heterodimer epigenetic domain constructs for screening were designed with a T7 promoter, NLS (nuclear localization signal), heterodimer protein (e.g.—9′ of the 9:9′ pair), the 15 amino acid linker (see above), and the functional domain (e.g.—KRAB); and generated as double-stranded DNA (Integrated DNA Technologies; gBlocks Gene Fragments).

### Example 1

### Identification of TALE-TFs for PDCD1 Repression

This example illustrates identification of TALE-TFs that significantly repress PD-1 expression. FIG. 1 provides a pictorial map of all of the regions in the PDCD-1 gene that were tested for identifying TALE-TFs that significantly repress PD-1 expression. The results are provided in Table 9 below:

TABLE 9

| | | | SEQ | Repression |
|---|---|---|---|---|
| TALE ID | Chromosomal location | Target sequence | ID NO | at Day 2 |
| TL11094 | PDCD1_PROMOTER_- | GGTGGGGCTGCTCCAGG | 6 | ≥80% |
| | 100_+100_10_EPITF_chr2:24185883 | | | |
| | 9-241858857_MINUS | | | |
| TL11099 | PDCD1_PROMOTER_- | GCCGCCTTCTCCACT | 32 | ≥80% |
| | 100_+100_15_EPITF_chr2:24185886 | | | |
| | 0-241858876_PLUS | | | |
| TL11104 | PDCD1_PROMOTER_- | TCCGCTCACCTCCGCCTGA | 21 | ≥80% |
| | 100_+100_20_EPITF_chr2:24185887 | | | |
| | 8-241858898_MINUS | | | |
| TL11105 | PDCD1_PROMOTER_- | CCCTTCCGCTCACCTCCGC | 23 | ≥80% |
| | 100_+100_21_EPITF_chr2:24185888 | | | |
| | 2-241858902_MINUS | | | |
| TL11106 | PDCD1_PROMOTER_- | TTCCCTTCCGCTCACC | 24 | ≥80% |
| | 100_+100_22_EPITF_chr2:24185888 | | | |
| | 7-241858904_MINUS | | | |
| TL11108 | PDCD1_PROMOTER_- | GGGACAGTTTCCCTTC | 26 | ≥80% |
| | 100_+100_24_EPITF_chr2:24185889 | | | |
| | 5-241858912_MINUS | | | |
| TL11112 | PDCD1_PROMOTER_- | CCCTTCAACCTGACCT | 30 | ≥80% |
| | 100_+100_28_EPITF_chr2:24185891 | | | |
| | 1-241858928_MINUS | | | |
| TL11128 | PDCD1_PROMOTER_- | GCCTCTGTCACTCTCGCCC | 13 | ≥80% |
| | 100_+100_44_EPITF_chr2:24185897 | | | |
| | 4-241858994_MINUS | | | |
| TL11132 | PDCD1_PROMOTER_- | CCTCCCCCAGCACTGC | 16 | ≥80% |
| | 100_+100_48_EPITF_chr2:24185899 | | | |
| | 1-241859008_MINUS | | | |
| TL11133 | PDCD1_PROMOTER_- | CCTCCCCCAGCACTGCC | 17 | ≥80% |
| | 100_+100_49_EPITF_chr2:24185899 | | | |
| | 0-241859008_MINUS | | | |
| TL11876 | PDCD1_PROMOTER_- | GACCTGGGACAGTTTCC | 27 | ≥80% |
| | 100_+100_25_EPITF_chr2:24185889 | | | |
| | 9-241858917 | | | |
| TL11875 | PDCD1_PROMOTER_- | GCAGATCCCACAGGCGC | 7 | ≥80% |
| | 100_+100_5_EPITF_chr2:241858819- | | | |
| | 241858837 | | | |
| TL11877 | PDCD1_PROMOTER_- | CCCAGGTCAGGTTGAAG | 63 | ≥80% |
| | 100_+100_27_EPITF_chr2:24185890 | | | |
| | 7-241858925 | | | |
| pAL040 | chr2:241858974-241858988 | TCTGTCACTCTCGCCCAC | 14 | ≥80% |
| pAL043 | chr2:241858843-241858857 | TGGTGGGGCTGCTCC | 5 | ≥80% |
| TL11101 | PDCD1_PROMOTER_- | TCTCCACTGCTCAGGCG | 34 | ≥80% |
| | 100_+100_17_EPITF_chr2:24185886 | | | |
| | 7-241858885_MINUS | | | |
| TL11110 | PDCD1_PROMOTER_- | CAACCTGACCTGGGACAGTT | 29 | ≥80% |
| | 100_+100_26_EPITF_chr2:24185890 | | | |
| | 2-241858923_MINUS | | | |
| TL11129 | PDCD1_PROMOTER_- | GCCTCTGTCACTCTCG | 12 | ≥80% |
| | 100_+100_45_EPITF_chr2:24185897 | | | |
| | 7-241858994_MINUS | | | |
| TL11084 | PDCD1_PROMOTER_- | GGCCAGGGCGCCTGT | 36 | ≥50% |
| | 100_+100_0_EPITF_chr2:241858811- | | | |
| | 241858827_MINUS | | | |
| TL11087 | PDCD1_PROMOTER_- | CCTCCACATCCACGTGGGC | 40 | ≥50% |
| | 100_+100_3_EPITF_chr2:241858810- | | | |
| | 241858831_PLUS | | | |
| TL11088 | PDCD1_PROMOTER_- | CCCACAGGCGCCCTGG | 8 | ≥50% |
| | 100_+100_4_EPITF_chr2:241858814- | | | |
| | 241858831_MINUS | | | |
| TL11092 | PDCD1_PROMOTER_- | CTGCATGCCTGGAGCAG | 37 | ≥50% |
| | 100_+100_8_EPITF_chr2:241858831- | | | |
| | 241858849_MINUS | | | |
| TL11096 | PDCD1_PROMOTER_- | GGAGCAGCCCCACCAGAGT | 106 | ≥50% |
| | 100_+100_12_EPITF_chr2:24185884 | | | |
| | 1-241858861_PLUS | | | |
| TL11102 | PDCD1_PROMOTER_- | CCACTGCTCAGGCGGAGGT | 35 | ≥50% |
| | 100_+100_18_EPITF_chr2:24185887 | | | |
| | 0-241858890_PLUS | | | |
| TL11103 | PDCD1_PROMOTER_- | GCTCAGGCGGAGGTGAG | 344 | ≥50% |
| | 100_+100_19_EPITF_chr2:24185887 | | | |
| | 5-241858893_PLUS | | | |
| TL11119 | PDCD1_PROMOTER_- | GCTCCCGCCCCCTCTTCCT | 38 | ≥50% |
| | 100_+100_35_EPITF_chr2:24185894 | | | |
| | 1-241858957_PLUS | | | |
| TL11124 | PDCD1_PROMOTER_- | CTCGCCCACGTGGATGTGG | 345 | ≥50% |
| | 100_+100_40_EPITF_chr2:24185895 | | | |
| | 8-241858978_MINUS | | | |
| TL11126 | PDCD1_PROMOTER_- | CACTCTCGCCCACGTGGAT | 346 | ≥50% |
| | 100_+100_42_EPITF_chr2:24185896 | | | |
| | 6-241858986_MINUS | | | |
| TL11127 | PDCD1_PROMOTER_- | CTGTCACTCTCGCCCACGT | 347 | ≥50% |
| | 100_+100_43_EPITF_chr2:24185897 | | | |
| | 0-241858990_MINUS | | | |
| TL11130 | PDCD1_PROMOTER_- | GACAGAGGCAGTGCTGG | 348 | ≥50% |
| | 100_+100_46_EPITF_chr2:24185898 | | | |
| | 3-241859001_PLUS | | | |
| TL11131 | PDCD1_PROMOTER_- | CCCCCAGCACTGCCTCT | 349 | ≥50% |
| | 100_+100_47_EPITF_chr2:24185898 | | | |
| | 7-241859005_MINUS | | | |
| TL11879 | PDCD1_PROMOTER_- | CTTCCTCCACATCCACG | 39 | ≥50% |
| | 100_+100_39_EPITF_chr2:24185895 | | | |
| | 5-241858973 | | | |
| TL11093 | PDCD1_PROMOTER_- | GGGGCTGCTCCAGGCATGC | 9 | ≥50% |
| | 100_+100_9_EPITF_chr2:241858834- | | | |
| | 241858854_MINUS | | | |
| TL11085 | PDCD1_PROMOTER_- | GGCCAGGGCGCCTGTG | 350 | <50% |
| | 100_+100_1_EPITF_chr2:241858811- | | | |
| | 241858828_PLUS | | | |
| TL11090 | PDCD1_PROMOTER_- | GTGGGATCTGCATGC | 351 | <50% |
| | 100_+100_6_EPITF_chr2:241858824- | | | |
| | 241858840_PLUS | | | |
| TL11091 | PDCD1_PROMOTER_- | GGGATCTGCATGCCTGGAG | 352 | <50% |
| | 100_+100_7_EPITF_chr2:241858826- | | | |
| | 241858846_PLUS | | | |
| TL11095 | PDCD1_PROMOTER_- | GGAGCAGCCCCACCAGAGT | 353 | <50% |
| | 100_+100_11_EPITF_chr2:24185884 | G | | |
| | 1-241858862_PLUS | | | |
| TL11097 | PDCD1_PROMOTER_- | GGAGAAGGCGGCACTCTGG | 354 | <50% |
| | 100_+100_13_EPITF_chr2:24185885 | T | | |
| | 3-241858874_MINUS | | | |
| TL11098 | PDCD1_PROMOTER_- | GGAGAAGGCGGCACTCTGG | 355 | <50% |
| | 100_+100_14_EPITF_chr2:24185885 | | | |
| | 4-241858874_MINUS | | | |
| TL11100 | PDCD1_PROMOTER_- | GAGCAGTGGAGAAGGCG | 356 | <50% |
| | 100_+100_16_EPITF_chr2:24185886 | | | |
| | 3-241858881_MINUS | | | |
| TL11107 | PDCD1_PROMOTER_- | GAGCGGAAGGGAAACTGTC | 357 | <50% |
| | 100_+100_23_EPITF_chr2:24185888 | C | | |
| | 9-241858910_PLUS | | | |
| TL11113 | PDCD1_PROMOTER_- | CAGGTTGAAGGGAGGGTGC | 358 | <50% |
| | 100_+100_29_EPITF_chr2:24185891 | | | |
| | 4-241858934_PLUS | | | |
| TL11115 | PDCD1_PROMOTER_- | GAAGGGAGGGTGCCCGCCC | 359 | <50% |
| | 100_+100_31_EPITF_chr2:24185892 | C | | |
| | 0-241858941_PLUS | | | |
| TL11116 | PDCD1_PROMOTER_- | GCCCGCCCCTTGCTC | 360 | <50% |
| | 100_+100_32_EPITF_chr2:24185893 | | | |
| | 1-241858947_PLUS | | | |
| TL11117 | PDCD1_PROMOTER_- | GCCCGCCCCTTGCTCCC | 361 | <50% |
| | 100_+100_33_EPITF_chr2:24185893 | | | |
| | 1-241858949_PLUS | | | |
| TL11118 | PDCD1_PROMOTER_- | TGCTCCCGCCCCCTC | 362 | <50% |
| | 100_+100_34_EPITF_chr2:24185893 | | | |
| | 1-241858952_PLUS | | | |
| TL11121 | PDCD1_PROMOTER_- | GGAGGAAGAGGGGGCGG | 363 | <50% |
| | 100_+100_37_EPITF_chr2:24185894 | | | |
| | 7-241858965_MINUS | | | |
| TL11122 | PDCD1_PROMOTER_- | GGATGTGGAGGAAGAGGGG | 364 | <50% |
| | 100_+100_38_EPITF_chr2:24185895 | G | | |
| | 0-241858971_MINUS | | | |
| TL11878 | PDCD1_PROMOTER_- | TGAAGGGAGGGTGCCCG | 365 | <50% |
| | 100_+100_30_EPITF_chr2:24185891 | | | |
| | 9-241858937 | | | |

FIG. 1A illustrates the locations in the PDCD 1 gene to which the DBDs of the indicated recombinant polypeptides were designed to bind. Recombinant polypeptides that repressed expression of PDCD 1 in at least 50% of cells treated with the recombinant polypeptides are indicated by clear arrows ( [symbol] or [symbol] ). Recombinant polypeptides that repressed expression of PDCD1 in less than 50% of the cells treated with the recombinant polypeptides are indicated by solid arrows ( [symbol] or [symbol] ). The orientation of the arrows indicates the DNA strand to which the recombinant polypeptide is designed to bind. Arrows having the orientation [symbol] and [symbol] are designed to bind to the anti-sense strand. Arrows having the orientation [symbol] and [symbol] are designed to bind to the sense strand.

The analysis of repression by the disclosed recombinant polypeptides that are designed bind to these sequences identified certain regions that provide repression of PDCD-1 expression in at least 5000 of the cells expressing these recombinant polypeptides. These regions are depicted in FIGS. 1B-1C and include regions 1-4. In regions 1, 2, 3, the anti-sense strand of the PDCD-1 gene was successfully targeted to significantly repress expression of PD-1. In region 4, the sense strand was identified as the region of the PDCD-1 gene that can be successfully target for repression. In addition, certain sequences in the sense strand in region 1 were also identified a region that can be targeted for repression. Tables 1-4 illustrate the sequences present in each of Regions 1-4 that can be targeted for repression.

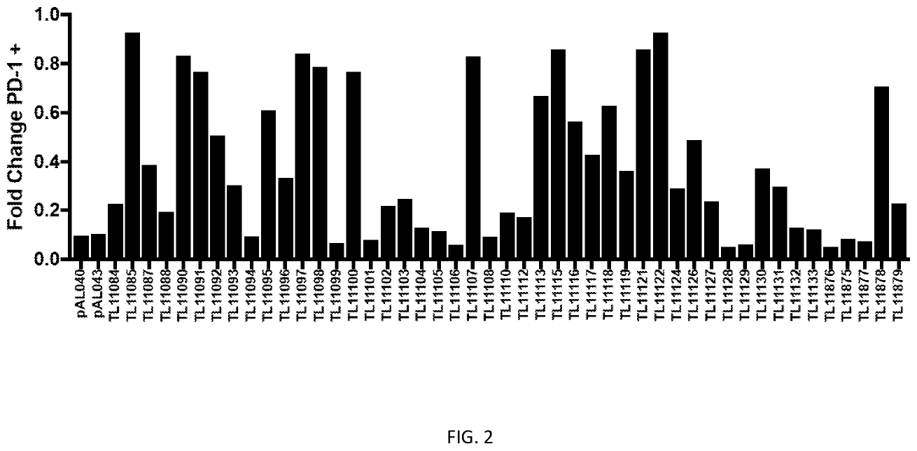

FIG. 2 shows the fold change in number of PD-1 expressing cells 2 days after transfection of mRNA encoding the indicated recombinant polypeptides into CD3+ T cells.

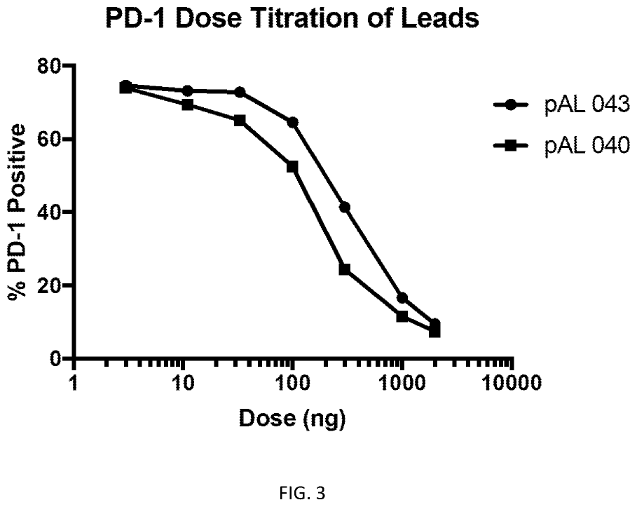

FIG. 3 shows effect of dose of mRNA encoding the recombinant polypeptide, pAL040 and pAL043, on the percent of CD3+ T cells expressing PD-1, 3 days after transfection. CD3+ T cells were activated with beads and electroporated 48 hours post activation according to standard process with varying concentration of TALE-TF mRNA from 3 ng to 2 ug per transfection (250,000 T cells per condition). PD-1 expression by flow was measured on day 3 post transfection.

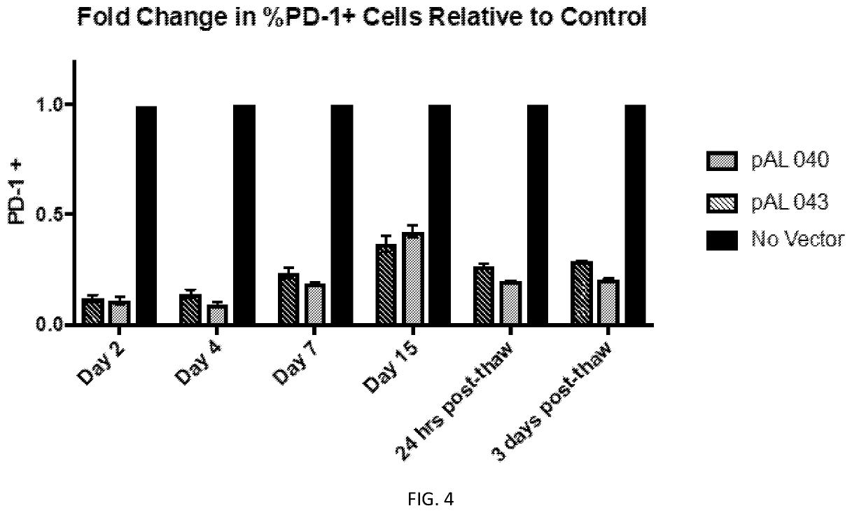

FIG. 4 shows the fold change in number of PD-1-positive cells at the indicated number of days post-transfection of mRNA encoding the indicated recombinant polypeptide relative to control, which are cells electroporated without repressor mRNA. PD-1 repression is durable for about 2 weeks in culture and after freeze-thaw.

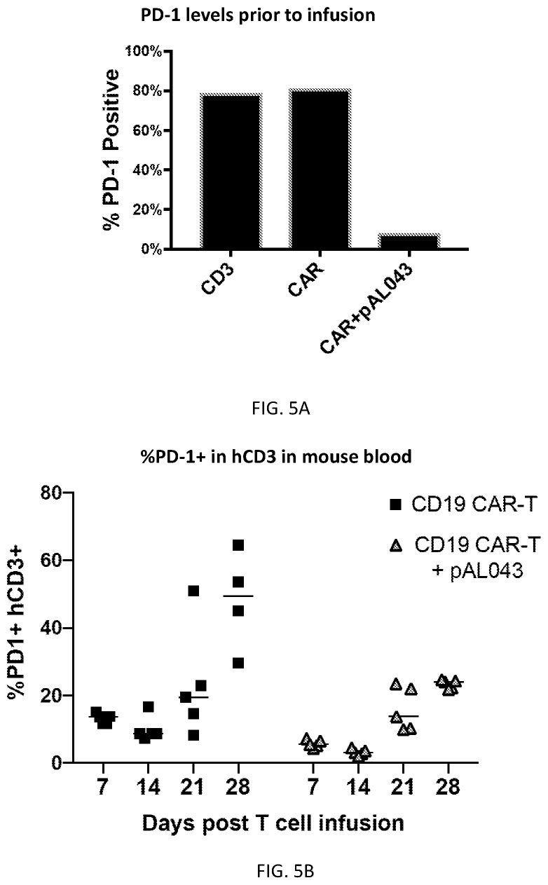

FIGS. 5A and 5B show that PD-1 repression with pAL043 in anti-CD19 CAR-T cells is sustained after in vivo expansion and clearance of CD19-positive NALM-6 B-ALL tumor model in NSG mice.

In addition to regions 1-4, targeting the sequence GGCCAGGGCGCCTGT (SEQ ID NO: 36) by TALE-TF TL11084 also significantly suppressed PD-1 expression.

### Example 2

### Identification of TALE-TFs for TIM3 Repression

This example illustrates identification of TALE-TFs that significantly repress TIM3 expression. FIG. 6 provides a pictorial map of all of the regions in the TIM3 gene that were tested for identifying TALE-TFs that significantly repress TIM3 expression. The results are provided in Table 10 below:

TABLE 10

| | | | | Re- |
|---|---|---|---|---|
| | | | | pres- |
| | Chromo- | | SEQ | sion |
| TALE | somal | | ID | at |
| ID | location | Target sequence | NO | Day 2 |
| TL8188 | chr5: | GGCAGTGTTACTATAA | 45 | ≥80% |
| | 157109141- | | | |
| | 157109142- | | | |
| | HAVCR2_ | | | |
| | +373 | | | |
| | RIGHT | | | |
| TL8189 | chr5: | TGCCAGTGATTCTTATAGT | 51 | ≥80% |
| | 157109163- | | | |
| | 157109164- | | | |
| | HAVCR2_ | | | |
| | +395 | | | |
| | LEFT | | | |
| TL9337 | chr5:chr5: | TGGCAATCAGACACCCGGGTG | 48 | ≥80% |
| | 157109125- | | | |
| | 157109146 | | | |
| | RIGHT | | | |
| TL9342 | chr5:chr5: | TGCCACACTACACACAT | 56 | ≥80% |
| | 157109206- | | | |
| | 157109223 | | | |
| | RIGHT | | | |
| TL9339 | chr5:chr5: | TGTCTGATTGCCAGTGATT | 53 | ≥80% |
| | 157109133- | | | |
| | 157109152 | | | |
| | LEFT | | | |
| TL8181 | chr5: | ACTTCTTCCAACTGT | 442 | ≥50% |
| | 157109075- | | | |
| | 157109076- | | | |
| | HAVCR2_ | | | |
| | +307 | | | |
| | LEFT | | | |
| TL8201 | chr5: | GAGAAAATTGTATTAGAT | 443 | ≥50% |
| | 157109689- | | | |
| | 157109690- | | | |
| | HAVCR2_ | | | |
| | +921 | | | |
| | LEFT | | | |
| TL8182 | chr5: | GGGGGCGGCTACTGCTCAT | 366 | <10% |
| | 157109075- | | | |
| | 157109076- | | | |
| | HAVCR2_ | | | |
| | +307 | | | |
| | RIGHT | | | |
| TL8184 | chr5: | GTGCTGAGCTAGCACTCA | 367 | <50% |
| | 157109097- | | | |
| | 157109098- | | | |
| | HAVCR2_ | | | |
| | +329 | | | |
| | RIGHT | | | |
| TL8192 | chr5 | GGCATGACAGAGAACTTT | 368 | <50% |
| | 157109184- | | | |
| | 157109185- | | | |
| | HAVCR2_ | | | |
| | +416 | | | |
| | RIGHT | | | |
| TL8196 | chr5: | ATCACAGGACAGACATCA | 369 | <50% |
| | 157109228- | | | |
| | 157109229- | | | |
| | HAVCR2_ | | | |
| | +460 | | | |
| | RIGHT | | | |
| TL8202 | chr5: | CAGAATATTAGAACAGAGA | 370 | <50% |
| | 157109689- | | | |
| | 157109690- | | | |
| | HAVCR2_ | | | |
| | +921 | | | |
| | RIGHT | | | |
| TL8203 | chr5: | ACATGCATGGCTCTCTGTT | 371 | <50% |
| | 157109711- | | | |
| | 157109712- | | | |
| | HAVCR2_ | | | |
| | +943 | | | |
| | LEFT | | | |
| TL8204 | chr5: | TGGAAGTTTGAAGGTCAA | 372 | <50% |
| | 157109711- | | | |
| | 157109712- | | | |
| | HAVCR2_ | | | |
| | +943 | | | |
| | RIGHT | | | |
| TL8205 | chr5: | AATATTCTGACTTTGACCT | 373 | <50% |
| | 157109732- | | | |
| | 157109733- | | | |
| | HAVCR2_ | | | |
| | +964 | | | |
| | LEFT | | | |
| TL8207 | chr5: | TCAAACTTCCAACTCTTCA | 374 | <50% |
| | 157109751- | | | |
| | 157109752- | | | |
| | HAVCR2_ | | | |
| | +983 | | | |
| | LEFT | | | |
| TL8208 | chr5: | GTTGCCAAAAGGAACA | 375 | <50% |
| | 157109751- | | | |
| | 157109752- | | | |
| | HAVCR2_ | | | |
| | +983 | | | |
| | RIGHT | | | |

FIG. 6 illustrates the locations in the TIM3 gene at which the DBDs of the indicated recombinant polypeptides bind. Recombinant polypeptides that repressed expression of TIM3 in at least 500% of the cells are indicated by unfilled arrows ( [symbol] or [symbol] ). Recombinant polypeptides that repressed expression of TIM3 in less than 500% of the cells are indicated by filled arrows ( [symbol] or [symbol] ). The orientation of the arrows indicates the DNA strand to which the recombinant polypeptide is designed to bind. Arrows having the orientation [symbol] and [symbol] are designed to bind to the anti-sense strand. Arrows having the orientation [symbol] and [symbol] are designed to bind to the sense strand.

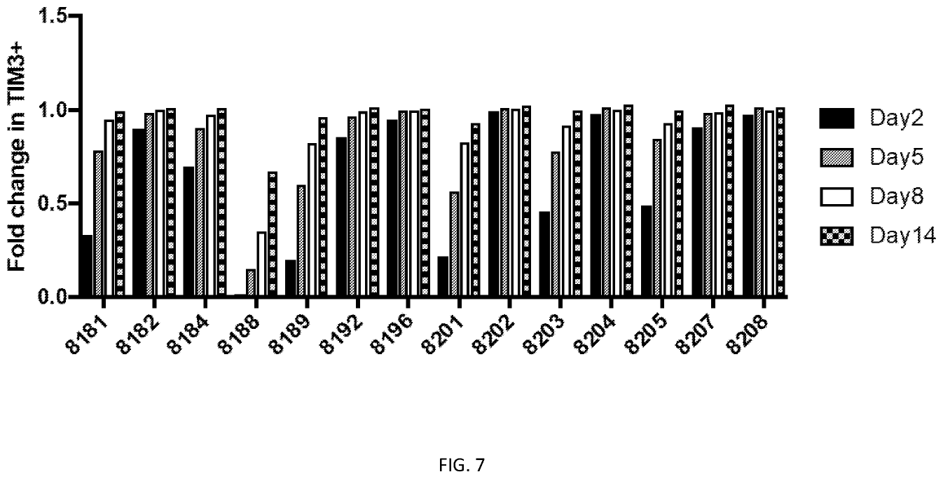

FIG. 7 shows the fold change in number of cells expressing TIM3 at 2 days, 5 days, 8 days, or 14 days after transfection of mRNA encoding the indicated recombinant polypeptides into CD3+ T cells.

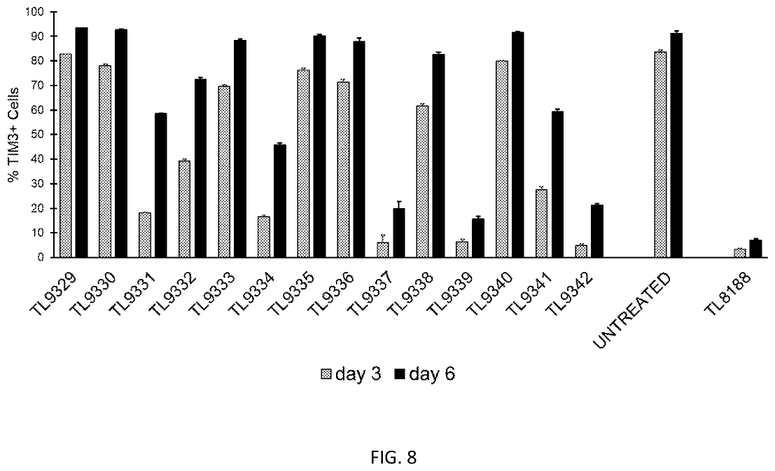

FIG. 8 shows the fold change in number of cells expressing TIM3 at 3 days or 6 days after transfection of mRNA encoding the indicated recombinant polypeptides into CD3+ T cells.

### Example 3

### Identification of TALE-TFs for CTLA4 Repression

This example illustrates identification of TALE-TFs that significantly repress CTLA4 expression. FIG. 9 provides a pictorial map of all of the regions in the CTLA4 gene that were tested for identifying TALE-TFs that significantly repress CTLA4 expression. The results are provided in Table 11 below:

TABLE 1
Region 1

| TALE ID | Target Sequence | Repression |
|---|---|---|
| pAL043 (or | TGGTGGGGCTGCTCC | ≥80% |
| PD02) | (SEQ ID NO: 5) | |
| TL11094 | GGTGGGGCTGCTCCAGG | ≥80% |
| | (SEQ ID NO: 6) | |
| TL11093 | GGGGCTGCTCCAGGCATGC | ≥50% |
| | (SEQ ID NO: 9) | |
| TL11875 | GCAGATCCCACAGGCGC | ≥80% |
| | (SEQ ID NO: 7) | |
| TL11088 | CCCACAGGCGCCCTGG | ≥50% |
| | (SEQ ID NO: 8) | |
| Region 1 | TGGTGGGGCTGCTCCAGGCA | |
| | TGCAGATCCCACAGGCGCCC | |
| | TGG (SEQ ID NO: 1) | |
| Sequence | GGTGGGGCTGCTCC | |
| common to | (SEQ ID NO: 4) | |
| pAL043 and | | |
| TL11094 | | |
| Sequence | GGGGCTGCTCC (SEQ ID NO: 2) | |
| common to | | |
| pAL043, | | |
| TL11094, and | | |
| TL11093 | | |

FIG. 9 illustrates the locations in the CTLA4 gene at which the DBDs of the indicated recombinant polypeptides bind. Recombinant polypeptides that repressed expression of CTLA4 in at least 500% of the cells are indicated by unfilled arrows ( [symbol] or [symbol] ). Recombinant polypeptides that repressed expression of CTLA4 in less than 500% of the cells are indicated by filled arrows ( [symbol] or [symbol] ). The orientation of the arrows indicates the DNA strand to which the recombinant polypeptide is designed to bind. Arrows having the orientation [symbol] and [symbol] are designed to bind to the anti-sense strand. Arrows having the orientation [symbol] and [symbol] are designed to bind to the sense strand.

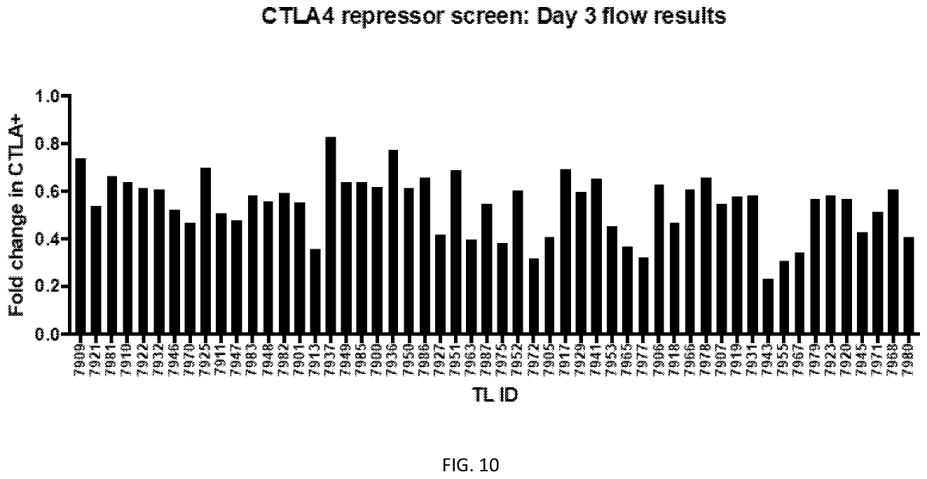

FIG. 10 shows the fold change in number of cells expressing CTLA4 at 3 days after transfection of mRNA encoding the indicated recombinant polypeptides into CD3+ T cells.

### Example 4

### Identification of TALE-TFs for LAG3 Repression

This example illustrates identification of TALE-TFs that significantly repress LAG3 expression. FIG. 11 provides a pictorial map of all of the regions in the LAG3 gene that were tested for identifying TALE-TFs that significantly repress LAG3 expression. The results are provided in Table 12 below:

TABLE 12

| | | | | Re- |
|---|---|---|---|---|
| | | | | pres- |
| | Chromo- | | SEQ | sion |
| TALE | somal | | ID | at |
| ID | location | Target sequence | NO | Day 2 |
| TL8214 | chr12: | GGTCTCTGGGCCTTCA | 65 | ≥80% |
| | 6772502- | | | |
| | 6772503- | | | |
| | LAG3_ | | | |
| | +32 | | | |
| | RIGHT | | | |
| TL8216 | chr12: | TCTGCTGGTCTCTGGGCC | 448 | ≥80% |
| | 6772506- | | | |
| | 6772507- | | | |
| | LAG3_ | | | |
| | +36 | | | |
| | RIGHT | | | |
| TL8220 | chr12: | GCCGTTCTGCTGGTCTCT | 60 | ≥80% |
| | 6772512- | | | |
| | 6772513- | | | |
| | LAG3_ | | | |
| | +42 | | | |
| | RIGHT | | | |
| TL8222 | chr12: | GCCGTTCTGCTGGTCT | 59 | ≥80% |
| | 6772513- | | | |
| | 6772514- | | | |
| | LAG3_ | | | |
| | +43 | | | |
| | RIGHT | | | |
| TL9820 | chr12: | TTCACCCCTGTGCCCGGCCTTCC | 71 | ≥80% |
| | 6772492- | | | |
| | 6772514 | | | |
| TL9606 | chr12: | TGGTCTCTGGGCCTTCACCC | 449 | ≥80% |
| | 6772508- | | | |
| | 6772527 | | | |
| TL9598 | chr12: | TCTGCTGGTCTCTGGGCCTTC | 450 | ≥80% |
| | 6772512- | | | |
| | 6772532 | | | |
| TL9717 | chr12: | TTTGCTCTGTCTGCTC | 74 | ≥80% |
| | 6772558- | | | |
| | 6772573 | | | |
| TL8241 | chr12: | CTGTTCCCTGGGACACCCCC | 451 | ≥50% |
| | 6772617- | | | |
| | 6772618- | | | |
| | LAG3_ | | | |
| | +147 | | | |
| | LEFT | | | |
| TL8213 | chr12: | GGGGAAGGTGGAGGGAA | 427 | <50% |
| | 6772502- | | | |
| | 6772503- | | | |
| | LAG3_ | | | |
| | +32 | | | |
| | LEFT | | | |
| TL8215 | chr12: | GGGGAAGGTGGAGGGAAGGC | 428 | <50% |
| | 6772506- | | | |
| | 6772507- | | | |
| | LAG3_ | | | |
| | +36 | | | |
| | LEFT | | | |
| TL8217 | chr12: | GGAGGGAAGGCCGGGCA | 429 | <50% |
| | 6772511- | | | |
| | 6772512- | | | |
| | LAG3_ | | | |
| | +41 | | | |
| | LEFT | | | |
| TL8219 | chr12: | GGAGGGAAGGCCGGGCAC | 430 | <50% |
| | 6772512- | | | |
| | 6772513- | | | |
| | LAG3_ | | | |
| | +42 | | | |
| | LEFT | | | |
| TL8223 | chr12: | GGAGGGAAGGCCGGGCACA | 431 | <50% |
| | 6772514- | | | |
| | 6772515- | | | |
| | LAG3_ | | | |
| | +44 | | | |
| | LEFT | | | |
| TL8226 | chr12: | GTCCCAGGGAACAGAGC | 432 | <50% |
| | 6772580- | | | |
| | 6772581- | | | |
| | LAG3_ | | | |
| | +110 | | | |
| | RIGHT | | | |
| TL8227 | chr12: | CTGCTCTCCGCCACGGCCC | 433 | <50% |
| | 6772593- | | | |
| | 6772594- | | | |
| | LAG3_ | | | |
| | +123 | | | |
| | LEFT | | | |
| TL8230 | chr12: | GAGGAGGTGGGGGCGGGGGT | 434 | <50% |
| | 6772596- | | | |
| | 6772597- | | | |
| | LAG3_ | | | |
| | +126 | | | |
| | RIGHT | | | |
| TL8232 | chr12: | GAGGAGGTGGGGGCGGG | 435 | <50% |
| | 6772599- | | | |
| | 6772600- | | | |
| | LAG3_ | | | |
| | +129 | | | |
| | RIGHT | | | |
| TL8239 | chr12: | CTGTTCCCTGGGACAC | 436 | <50% |
| | 6772614- | | | |
| | 6772615- | | | |
| | LAG3_ | | | |
| | +144 | | | |
| | LEFT | | | |
| TL8242 | chr12: | GGGCAGATCAGGCAGCCT | 437 | <50% |
| | 6772617- | | | |
| | 6772618- | | | |
| | LAG3_ | | | |
| | +147 | | | |
| | RIGHT | | | |

FIG. 11 illustrates the locations in the LAG3 gene at which the DBDs of the indicated recombinant polypeptides bind. Recombinant polypeptides that repressed expression of LAG3 in at least 5000 of the cells are indicated by unfilled arrows ( [symbol] or [symbol] ) Recombinant polypeptides that repressed expression of LAG3 in less than 50% of the cells are indicated by filled arrows ( [symbol] or [symbol] ). The orientation of the arrows indicates the DNA strand to which the recombinant polypeptide is designed to bind. Arrows having the orientation [symbol] and [symbol] are designed to bind to the anti-sense strand. Arrows having the orientation [symbol] and [symbol] are designed to bind to the sense strand.

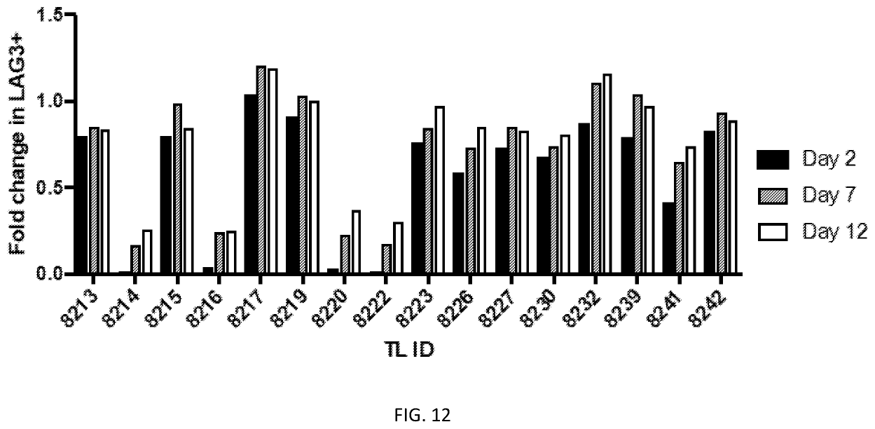

FIG. 12 shows the fold change in number of cells expressing LAG3 at 2 days, 7 days, or 12 days after transfection of mRNA encoding the indicated recombinant polypeptides into CD3+ T cells.

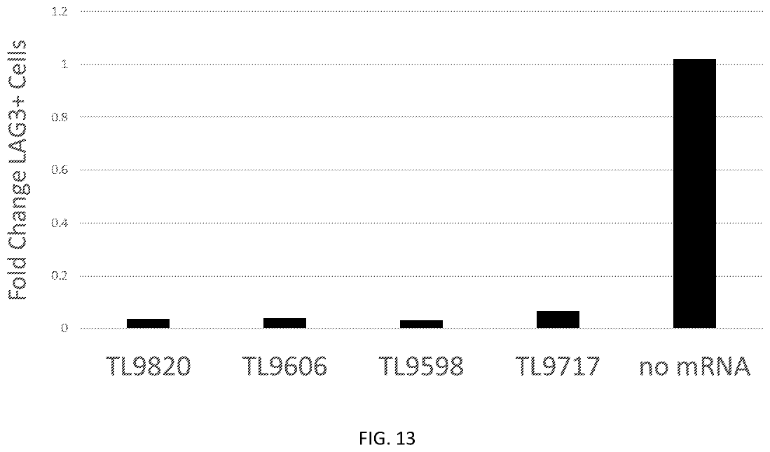

FIG. 13 shows the fold change in number of cells expressing LAG3 at 2 days after transfection of mRNA encoding the indicated recombinant polypeptides into CD3+ T cells.

### Example 5

### Multiplexing of TALE-TFs for PDCD1, TIM3, and LAG3 Repression

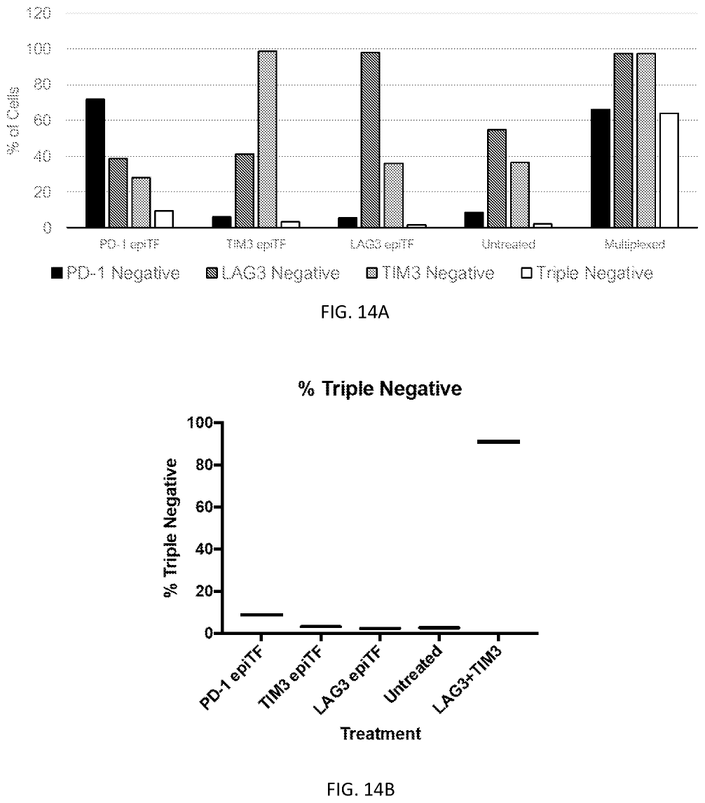

FIGS. 14A and 14B show multiplexing of recombinant polypeptides to simultaneously suppress expression of PD-1, LAG3, and TIM3 is a single cell.

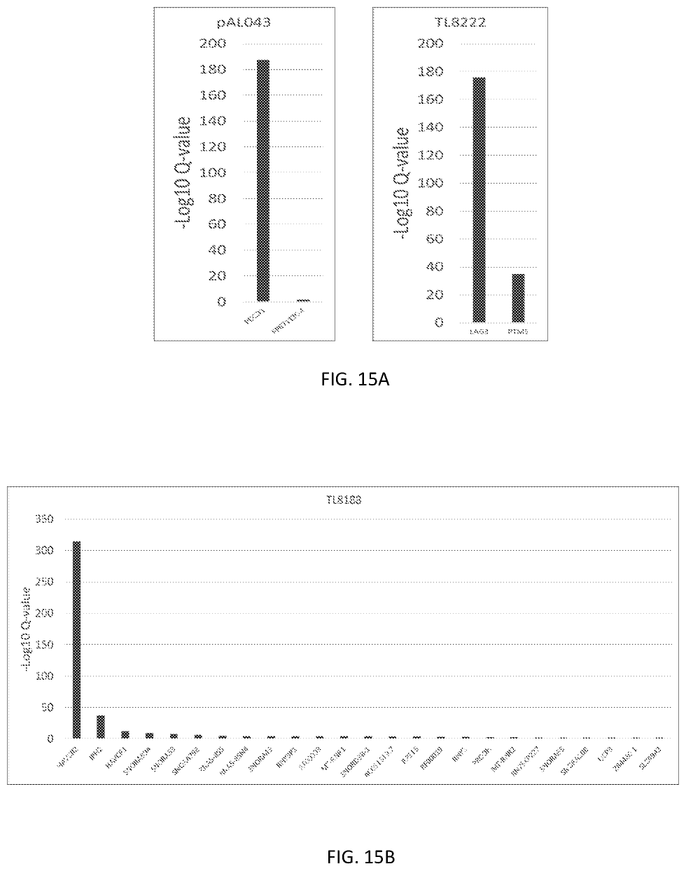

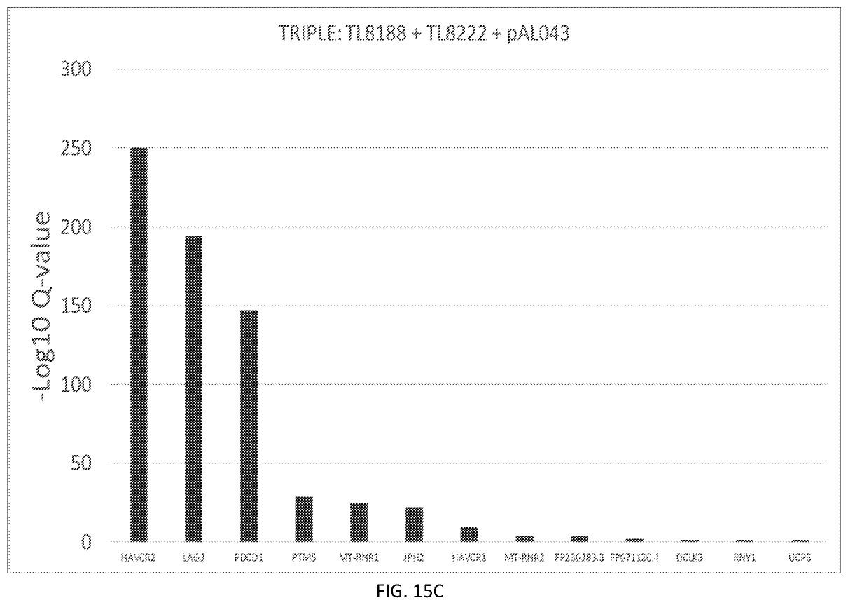

FIGS. 15A-15C illustrates specificity of the recombinant polypeptides as indicated by lack of significant off-target effect as measured by RNA-seq.

Anti-CD19 CAR-T cells were treated with epiTFs against PD-1, LAG3, and TIM3 and then used against a B-Cell Acute Lymphoblastic Leukemia (B-ALL) xenograft model in Non-obese Diabetic, NOD Scid Gamma (NSG) mice.

CAR-T cells were manufactured using lentivirus delivery of a 3rd generation anti-CD19 CAR containing FMC63 scFv, CD28 and 4-1BB co-stimulatory domains, and a truncated EGFR tag (Lenti-EF1a-CD19-EGFRt-3rd-CAR Vector, Creative Biolabs). Primary human T cells were activated with Dynabeads as previously described and transfected by electroporation with repressor mRNA at 48 hours post activation. Transfected cells along with no-mRNA transfected controls were allowed to recover for 24 hours after electroporation and then transduced with lentivirus encoding the CAR on RetroNectin (Takara Bio) according to manufacturer's protocol at an MOI of 5 and in the absence of serum. At 24 hours post transduction beads and virus were removed and CAR-T cells were allowed to expand in media with IL-2 until day 11 post activation when they were washed with PBS and administered to mice. Prior to using in animals, CAR-T cells were analyzed by flow cytometry for CAR expression (via EGFR staining) and expression of immune checkpoint genes (PD-1, LAG3, and TIM3).

Animal experiments were conducted at the Fred Hutchinson Cancer Center, Comparative Medicine department (Seattle, Wash.) according to an approved IACCUC protocol. Female NSG mice aged 6-8 weeks were implanted intravenously with 5×10⁵ NALM-6-luc-GFP tumor cells (human B-ALL cancer cells expressing CD19) and tumors were measured by total bioluminescent flux using a Xenogen Imaging System (Perkin Elmer). Each experimental arm contained 5 mice. At 4 days post tumor implantation mice were imaged and randomized into treatment arms based on baseline tumor burden. On day 5 post implantation mice were dosed intravenously with 250,000 anti-CD19 CAR-T cells either treated or untreated with repressor mRNA. Peripheral blood was collected via retroorbital bleeding at weekly intervals into EDTA-coated tubes at room temperature. Red blood cell lysis was performed using (1×RBC Lysis Buffer, eBiosciences Cat. #333-57) according to manufacturer's protocol. Flow cytometry was performed as previously described. At 3 weeks post initial dosing mice were re-challenged with 5×10⁵ NALM-6-luc-GFP tumor cells to test for persistence and activity of circulating CAR-T cells in the blood.

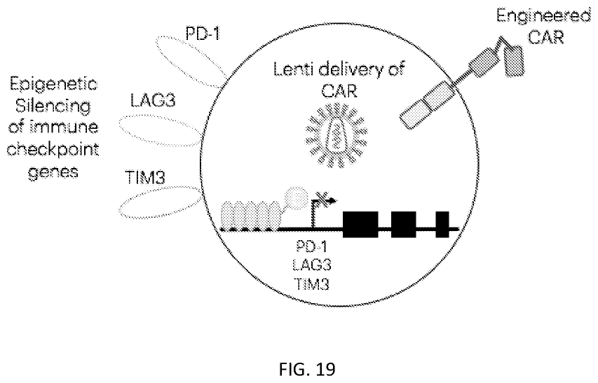

FIG. 19 shows a schematic of an anti-CD19 CAR-T cell in which expression of PD1, TIM3, and LAG3 has been repressed using the engineered polypeptides (pAL043+TL8188+TL8222) described herein.

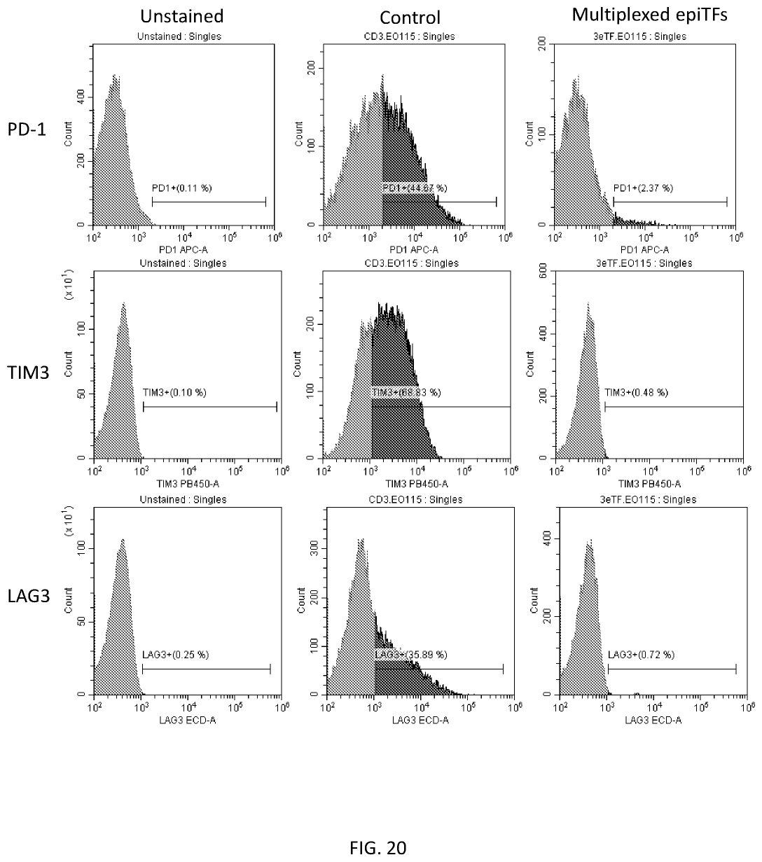

FIG. 20 shows flow cytometry data confirming repression of PD1, TIM3, and LAG3 expression in the multiplex-treated CAR-T cells. Flow cytometry, performed on CAR-T cells prior to infusion, showed repression of all three targeted immune checkpoint genes in the multiplex-treated CAR-T cells.

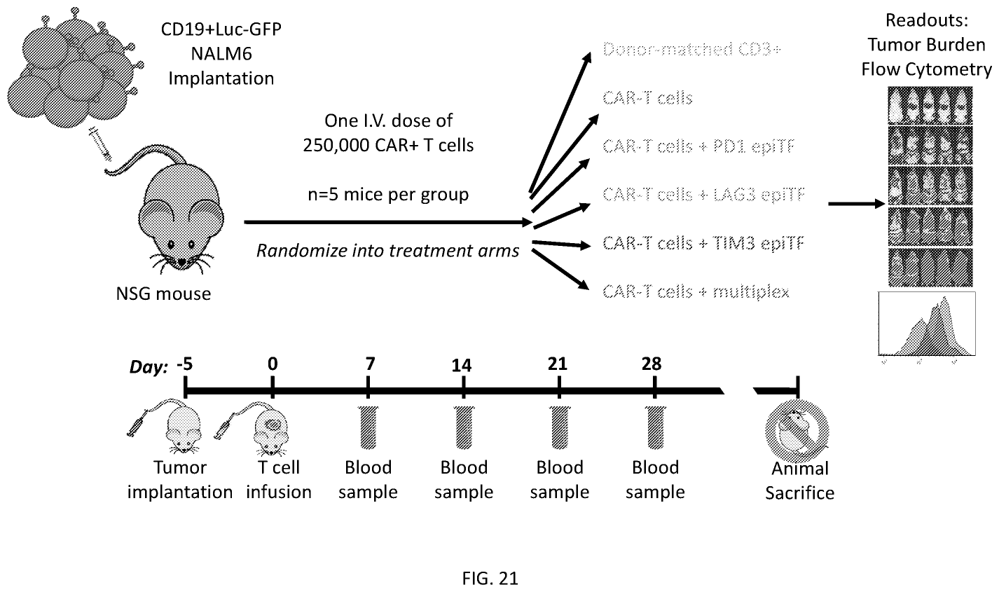

FIG. 21 provides an overview of in vivo leukemia xenograft model and treatment using indicated CAR-T cells.

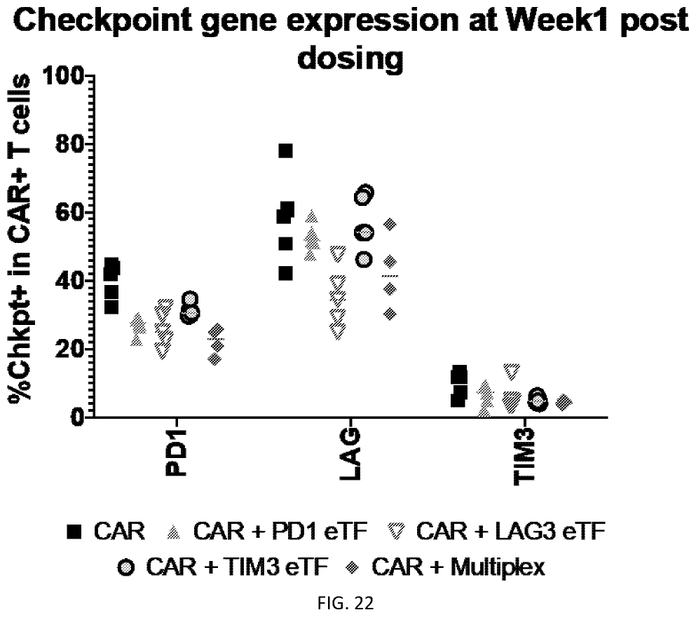

FIG. 22 demonstrates that multiplexed repression of immune checkpoint genes is sustained in vivo. Flow cytometry showed persistent repression of immune checkpoint genes at 1 week post dosing CAR-Ts into mice.

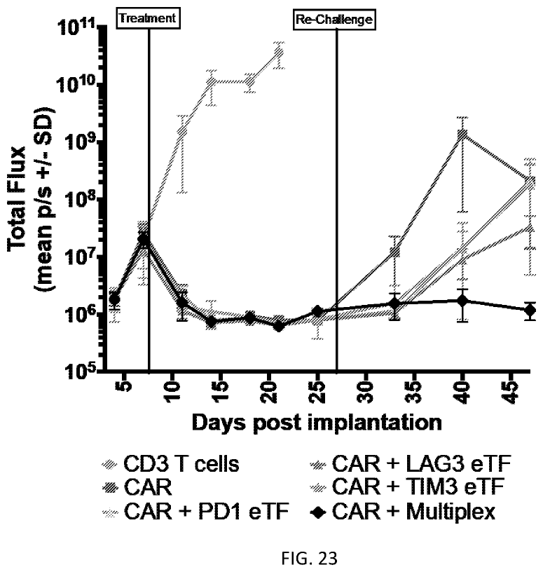

FIG. 23 demonstrates that multiplexed repression of immune checkpoint genes enhances CAR-Ts ability to resist tumor re-challenge. Tumor burden as measured by total flux (bioluminescence) showed all mice were initially “cured” of leukemia in all treatment arms, but upon re-challenge with leukemia cells only the mice treated with CAR-Ts in which all 3 immune checkpoint genes were repressed were able to completely resist tumor formation. This indicates superior persistence and resistance to exhaustion.

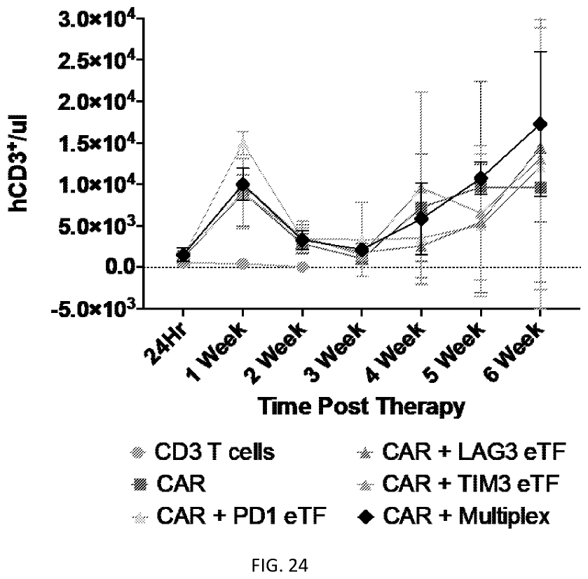

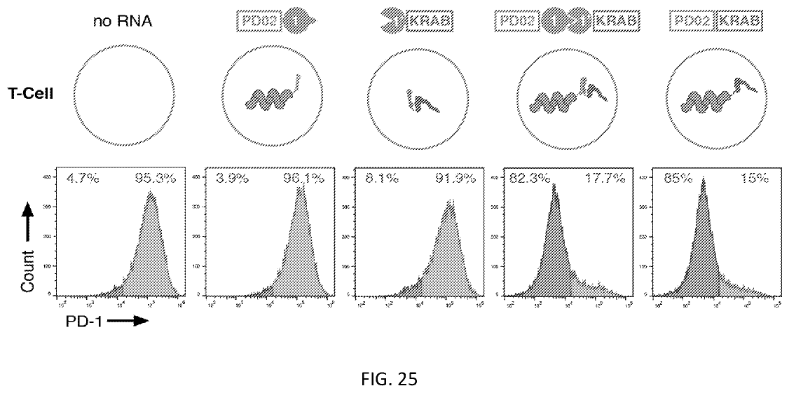

FIG. 24 shows expansion of CAR-Ts in the mouse blood. Flow cytometry data showed expansion of CAR-T cells in the mouse blood (measured as human CD3+ T cells). After the re-challenge the multiplex-treated T cells expanded the best, in line with their enhanced proliferative capacity and resistance to exhaustion.

### Example 6

### Identification of Novel Transcriptional Repressors

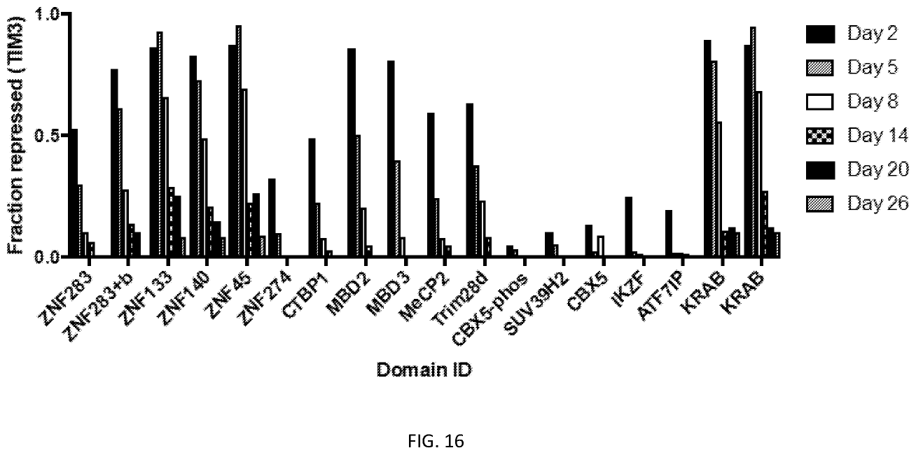

FIG. 16 shows characterization of repression of TIM3 expression by the listed candidate transcriptional repressors.

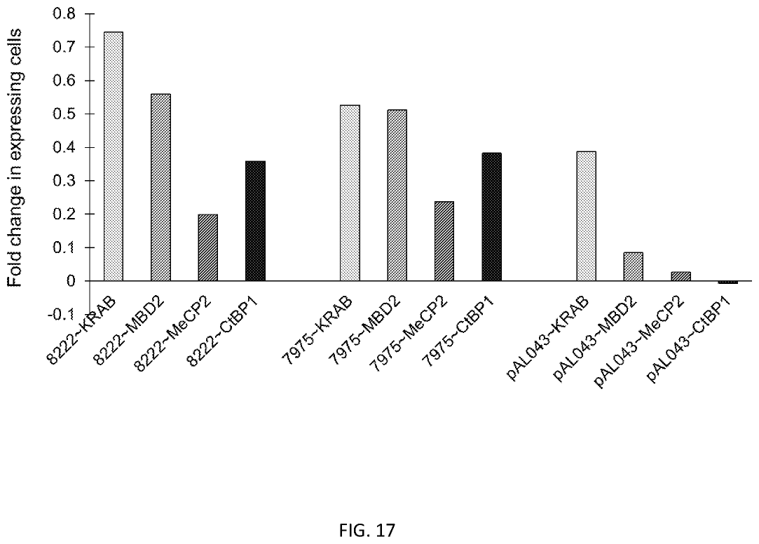

FIG. 17 shows characterization of repression of LAG3, TIM3, or PD-1 expression by the listed candidate transcriptional repressors.

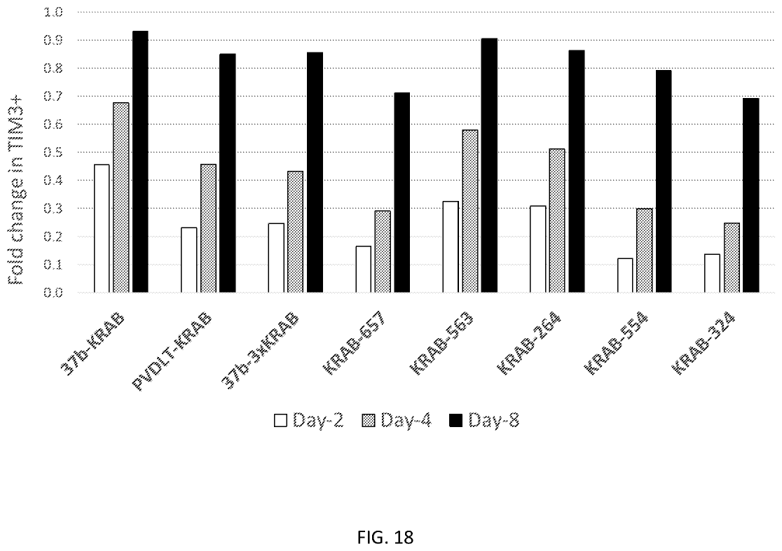

FIG. 18 shows characterization of repression of TIM3 expression by the listed candidate transcriptional repressors.

The sequences of the candidate transcriptional repressors are as follows:

MBD2:
(SEQ ID NO: 81)
MRAHPGGGRCCPEQEEGESAAGGSGAGGDSAIEQGGQGSALAPSPVSGVRREGARGGG
RGRGRWKQAGRGGGVCGRGRGRGRGRGRGRGRGRGRGRPPSGGSGLGGDGGGCGGGGSGGG
GAPRREPVPFPSGSAGPGPRGPRATESGKRMSKLQKNKQRLRNDPLNQNKGKPDLNTTLPIRQT
ASIFKQPVTKVTNHPSNKVKSDPQRMNEQPRQLFWEKRLQGLSASDVTEQIIKTMELPKGLQGV
GPGSNDETLLSAVASALHTSSAPITGQVSAAVEKNPAVWLNTSQPLCKAFIVTDEDIRKQEERVQ
QVRKKLEEALMADILSRAADTEEMDIEMDSGDEA
MBD3:
(SEQ ID NO: 82)
MRVRYDSSNQVKGKPDLNTALPVRQTASIFKQPVTKITNHPSNKVKSDPQKAVDQPRQL
FWEKKLSGLNAFDIAEELVKTMDLPKGLQGVGPGCTDETLLSAIASALHTSTMPITGQLSAAVEK
NPGVWLNTTQPLCKAFMVTDEDIRKQEELVQQVRKRLEEALMADMLAHVEELARDGEAPLDK
ACAEDDDEEDEEEEEEEPDPDPEMEHV
MeCP2:
(SEQ ID NO: 83)
MASSPKKKRKVEASVQVKRVLEKSPGKLLVKMPFQASPGGKGEGGGATTSAQVMVIKR
PGRKRKAEADPQAIPKKRGRKPGSVVAAAAAEAKKKAVKESSIRSVQETVLPIKKRKTRETVSIE
VKEVVKPLLVSTLGEKSGKGLKTCKSPGRKSKESSPKGRSSSASSPPKKEHHHHHHHAESPKAP
MPLLPPPPPPEPQSSEDPISPPEPQDLSSSICKEEKMPRAGSLESDGCPKEPAKTQPMVAAAATTTT
TTTTTVAEKYKHRGEGERKDIVSSSMPRPNREEPVDSRTPVTERVSEF
CTBP1:
(SEQ ID NO: 84)
MGSSHLLNKGLPLGVRPPIMNGPLHPRPLVALLDGRDCTVEMPILKDVATVAFCDAQST
QEIHEKVLNEAVGALMYHTITLTREDLEKFKALRIIVRIGSGFDNIDIKSAGDLGIAVCNVPAASV
EETADSTLCHILNLYRRATWLHQALREGTRVQSVEQIREVASGAARIRGETLGIIGLGRVGQAVA
LRAKAFGFNVLFYDPYLSDGVERALGLQRVSTLQDLLFHSDCVTLHCGLNEHNHHLINDFTVKQ
MRQGAFLVNTARGGLVDEKALAQALKEGRIRGAALDVHESEPFSFSQGPLKDAPNLICTPHAAW
YSEQASIEMREEAAREIRRAITGRIPDSLKNCVNKDHLTAATHWASMDPAVVHPELNGAAYRYP
PGVVGVAPTGIPAAVEGIVPSAMSLSHGLPPVAHPPHAPSPGQTVKPEADRDHASDQL
ZNF283:
(SEQ ID NO: 85)
MESRSVAQAGVQWCDLGSLQAPPPGFTLFSCLSLLSSWDYSSGFSGFCASPIEESHGALIS
SCNSRTMTDGLVTFRDVAIDFSQEEWECLDPAQRDLYVDVMLENYSNLVSLDLESKTYETKKIF
SENDIFEINFSQWEMKDKSKTLGLEASIFRNNWKCKSIFEGLKGHQEGYFSQMIISYEKIPSYRKS
KSLTPHQRIHNTE
ZNF283 + B:
(SEQ ID NO: 86)
MESRSVAQAGVQWCDLGSLQAPPPGFTLFSCLSLLSSWDYSSGFSGFCASPIEESHGALIS
SCNSRTMTDGLVTFRDVAIDFSQEEWECLDPAQRDLYVDVMLENYSNLVSLGYQLTKPDVILRL
EKGEEPIFRNNWKCKSIFEGLKGHQEGYFSQMIISYEKIPSYRKSKSLTPHQRIHNTE
ZNF133:
(SEQ ID NO: 87)
MAFRDVAVDFTQDEWRLLSPAQRTLYREVMLENYSNLVSLGISFSKPELITQLEQGKET
WREEKKCSPATCPDPEPELYLDPFCPPGFSSQKFPMQHVLCNHPPWIFTCLCAEGNIQPGDPGPG
DQEKQQQASEGRPWSDQAEGPEGEGAMPLFGRTKKRTLGAFSRPPQRQPVSSRNGLRGVELEAS
PAQSGNPEETDKLLKRIEVLGFGTV
ZNF140:
(SEQ ID NO: 88)
MSQGSVTFRDVAIDFSQEEWKWLQPAQRDLYRCVMLENYGHLVSLGLSISKPDVVSLLE
QGKEPWLGKREVKRDLFSVSESSGEIKDFSPKNVIYDDSSQYLIMERILSQGPVYSSFKGGWKCK
DHTEMLQENQGCIRKVTVSHQEALAQHMNISTVERP
ZNF45:
(SEQ ID NO: 89)
MTKSKEAVTFKDVAVVFSEEELQLLDLAQRKLYRDVMLENFRNVVSVGHQSTPDGLPQ
LEREEKLWMMKMATQRDNSSGAKNLKEMETLQEVGLRYLPHEELFCSQIWQQITRELIKYQDS
VVNIQRTGCQLEKRDDLHYKDEGFSNQSSHLQVHRVHTGEKP
ZNF274:
(SEQ ID NO: 90)
MASRLPTAWSCEPVTFEDVTLGFTPEEWGLLDLKQKSLYREVMLENYRNLVSVEHQLS
KPDVVSQLEEAEDFWPVERGIPQDTIPEYPELQLDPKLDPLPAESPLMNIEVVEVLTLNQEVAGPR
NAQIQALYAEDGSLSADAPSEQVQQQGKHPGDPEAARQRFRQFRYKDMTGPREALDQLRELCH
QWLQPKARSKEQILELLVLEQFLGALPVKLRTWVESQHPENCQEVVALVEGVTWMSEEEVLPA
GQPAEGTTCCLEVTAQQEEKQEDAAICPVTVLPEEPVTFQDVAVDFSREEWGLLGPTQRTEYRD
VMLETFGHLVSVGWETTLENKELAPNSDIPEEEPAPSLKVQESSRDCALSSTLEDTLQGGVQEVQ
DTVLKQMESAQEKDLPQKKHFDNRESQANSGALDTNQVSLQKIDNPESQANSGALDTNQVLLH
KIPPRKRLRKRDSQVKSMKHNSRVKIHQKSCERQKAKEGNGCRKTFSRSTKQITFIRIHKGSQV
TRIM28D:
(SEQ ID NO: 91)
GVKRSRSGEGEVSGLMRKVPRVSLERLDLDLTADSQPPVFKVFPGSTTEDYNLIVIERGA
AAAATGQPGTAPAGTPGAPPLAGMAIVKEEETEAAIGAPPTATEGPETKPVLMALAEGPGAEGP
RLASPSGSTSSGLEVVAPEGTSAPGGGPGTLDDSATICRVCQKPGDLVMCNQCEFCFHLDCHLPA
LQDVPGEEWSCSLCHVLPDLKEEDGSLSLDGADSTGVVAKLSPANQRKCERVLLALFCHEPCRP
LHQLATDSTFSLDQPGGTLDLTLIRARLQEKLSPPYSSPQEFAQDVGRMFKQFNKLTEDKADVQS
IIGLQRFFETRMNEAFGDTKFSAVLVEPPPMSLPGAGLSSQELSGGPGDGPGVKRSRSGEGEVSGL
MRKVPRVSLERLDLDLTADSQPPVFKVFPGSTTEDYNLIVIERGAAAAATGQPGTAPAGTPGAPP
LAGMAIVKEEETEAAIGAPPTATEGPETKPVLMALAEGPGAEGPRLASPSGSTSSGLEVVAPEGTS
APGGGPGTLDDSATICRVCQKPGDLVMCNQCEFCFHLDCHLPALQDVPGEEWSCSLCHVLPDLK
EEDGSLSLDGADSTGVVAKLSPANQRKCERVLLALFCHEPCRPLHQLATDSTFSLDQPGGTLDLT
LIRARLQEKLSPPYSSPQEFAQDVGRMFKQFNKLTEDKADVQSIIGLQRFFETRMNEAFGDTKFS
AVLVEPPPMSLPGAGLSSQELSGGPGDGP
CBX5-phos:
(SEQ ID NO: 92)
MGKKTKRTADDDDDEDEEEYVVEKVLDRRVVKGQVEYLLKWKGFSEEHNTWEPEKNL
DCPELISEFMKKYKKMKEGENNKPREKSESNKRKSNFSNSADDIKSKKKREQSNDIARGFERGLE
PEKIIGATDSCGDLMFLMKWKDTDEADLVLAKEANVKCPQIVIAFYEERLTWHAYPEDAENKEK
ETAKS
CBX5:
(SEQ ID NO: 93)
MGKKTKRTADSSSSEDEEEYVVEKVLDRRVVKGQVEYLLKWKGFSEEHNTWEPEKNL
DCPELISEFMKKYKKMKEGENNKPREKSESNKRKSNFSNSADDIKSKKKREQSNDIARGFERGLE
PEKIIGATDSCGDLMFLMKWKDTDEADLVLAKEANVKCPQIVIAFYEERLTWHAYPEDAENKEK
ETAKS
SUV39H2:
(SEQ ID NO: 94)
MAAVGAEARGAWCVPCLVSLDTLQELCRKEKLTCKSIGITKRNLNNYEVEYLCDYKVV
KDMEYYLVKWKGWPDSTNTWEPLQNLKCPLLLQQFSNDKHNYLSQVKKGKAITPKDNNKTLK
PAIAEYIVKKAKQRIALQRWQDELNRRKNHKGMIFVENTVDLEGPPSDFYYINEYKPAPGISLVN
EATFGCSCTDCFFQKCCPAEAGVLLAYNKNQQIKIPPGTPIYECNSRCQCGPDCPNRIVQKGTQYS
LCIFRTSNGRGWGVKTLVKIKRMSFVMEYVGEVITSEEAERRGQFYDNKGITYLFDLDYESDEFT
VDAARYGNVSHFVNHSCDPNLQVFNVFIDNLDTRLPRIALFSTRTINAGEELTFDYQMKGSGDIS
SDSIDHSPAKKRVRTVCKCGAVTCRGYLN
IKZF:
(SEQ ID NO: 95)
MNYLESMGLPGTLYPVIKEETNHSEMAEDLCKIGSERSLVLDRLASNVAKRKSSMPQKF
LGDKGLSDTPYDSSASYEKENEMMKSHVMDQAINNAINYLGAESLRPLVQTPPGGSEVVPVISP
MYQLHKPLAEGTPRSNHSAQDSAVENLLLLSKAKLVPSEREASPSNSCQDSTDTESNNEEQRSGL
IYLTNHIAPHARNGLSLKEEHRAYDLLRAASENSQDALRVVSTSGEQMKVYKCEHCRVLFLDHV
MYTIHMGCHGFRDPFECNMCGYHSQDRYEFSSHITRGEHRFHMS
ATF7IP:
(SEQ ID NO: 96)
RSKSEDMDNVQSKRRRYMEEEYEAEFQVKITAKGDINQKLQKVIQWLLEEKLCALQCA
VFDKTLAELKTRVEKIECNKRHKTVLTELQAKIARLTKRFEAAKEDLKKRHEHPPNPPVSPGKTV
NDVNSNNNMSYRNAGTVRQMLESKRNVSESAPPSFQTPVNTVSSTNLVTPPAVVSSQPKLQTPV
TSGSLTATSVLPAPNTATVVATTQVPSGNPQPTISLQPLPVILHVPVAVSSQPQLLQSHPGTLVTN
QPSGNVEFISVQSPPTVSGLTKNPVSLPSLPNPTKPNNVPSVPSPSIQRNPTASAAPLGTTLAVQAV
PTAHSIVQATRTSLPTVGPSGLYSPSTNRGPIQMKIPISAFSTSSAAEQNSNTTPRIENQTNKTIDAS
VSKKAADSTSQCGKATGSDSSGVIDLTMDDEESGASQDPKKLNHTPVSTMSSSQPVSRPLQPIQP
APPLQPSGVPTSGPSQTTIHLLPTAPTTVNVTHRPVTQVTTRLPVPRAPANHQVVYTTLPAPPAQA
PLRGTVMQAPAVRQVNPQNSVTVRVPQTTTYVVNNGLTLGSTGPQLTVHHRPPQVHTEPPRPV
HPAPLPEAPQPQRLPPEAASTSLPQKPHLKLARVQSQNGIVLSWSVLEVDRSCATVDSYHLYAYH
EEPSATVPSQWKKIGEVKALPLPMACTLTQFVSGSKYYFAVRAKDIYGRFGPFCDPQSTDVISST
QSS
DNMT3A-DNMT3L:
(SEQ ID NO: 97)
IRVLSLFDGIATGLLVLKDLGIQVDRYIASEVCEDSITVGMVRHQGKIMYVGDVRSVTQK
HIQEWGPFDLVIGGSPCNDLSIVNPARKGLYEGTGRLFFEFYRLLHDARPKEGDDRPFFWLFENV
VAMGVSDKRDISRFLESNPVMIDAKEVSAAHRARYFWGNLPGMNRPLASTVNDKLELQECLEH
GRIAKFSKVRTITTRSNSIKQGKDQHFPVFMNEKEDILWCTEMERVFGFPVHYTDVSNMSRLARQ
RLLGRSWSVPVIRHLFAPLKEYFACVSSGNSNANSRGPSFSSGLVPLSLRGSHNPLEMFETVPVW
RRQPVRVLSLFEDIKKELTSLGFLESGSDPGQLKHVVDVTDTVRKDVEEWGPFDLVYGATPPLG
HTCDRPPSWYLFQFHRLLQYARPKPGSPRPFFWMFVDNLVLNKEDLDVASRFLEMEPVTIPDVH
GGSLQNAVRVWSNIPAIRSSRHWALVSEEELSLLAQNKQSSKLAAKWPTKLVKNCFLPLREYFK
YFSTELTSSL
DNMT3B:
(SEQ ID NO: 98)
MKGDTRHLNGEEDAGGREDSILVNGACSDQSSDSPPILEAIRTPEIRGRRSSSRLSKREVSS
LLSYTQDLTGDGDGEDGDGSDTPVMPKLFRETRTRSESPAVRTRNNNSVSSRERHRPSPRSTRGR
QGRNHVDESPVEFPATRSLRRRATASAGTPWPSPPSSYLTIDLTDDTEDTHGTPQSSSTPYARLAQ
DSQQGGMESPQVEADSGDGDSSEYQDGKEFGIGDLVWGKIKGFSWWPAMVVSWKATSKRQA
MSGMRWVQWFGDGKFSEVSADKLVALGLFSQHFNLATFNKLVSYRKAMYHALEKARVRAGK
TFPSSPGDSLEDQLKPMLEWAHGGFKPTGIEGLKPNNTQPVVNKSKVRRAGSRKLESRKYENKT
RRRTADDSATSDYCPAPKRLKTNCYNNGKDRGDEDQSREQMASDVANNKSSLEDGCLSCGRK
NPVSFHPLFEGGLCQTCRDRFLELFYMYDDDGYQSYCTVCCEGRELLLCSNTSCCRCFCVECLE
VLVGTGTAAEAKLQEPWSCYMCLPQRCHGVLRRRKDWNVRLQAFFTSDTGLEYEAPKLYPAIP
AARRRPIRVLSLFDGIATGYLVLKELGIKVGKYVASEVCEESIAVGTVKHEGNIKYVNDVRNITK
KNIEEWGPFDLVIGGSPCNDLSNVNPARKGLYEGTGRLFFEFYHLLNYSRPKEGDDRPFFWMFE
NVVAMKVGDKRDISRFLECNPVMIDAIKVSAAHRARYFWGNLPGMNRPVIASKNDKLELQDCL
EYNRIAKLKKVQTITTKSNSIKQGKNQLFPVVMNGKEDVLWCTELERIFGFPVHYTDVSNMGRG
ARQKLLGRSWSVPVIRHLFAPLKDYFACE
ZNF-657-Krab:
(SEQ ID NO: 99)
SQGRVTFEDVTVNFTQGEWQRLNPEQRNLYRDVMLENYSNLVSVGQGETTKPDVILRL
EQGKEPWLEEEEVLGSGRAE
ZNF-554-Krab:
(SEQ ID NO: 100)
SQELVTFEDVSMDFSQEEWELLEPAQKNLYREVMLENYRNVVSLEALKNQCTDVGIKE
GPLSPAQTSQVTSLSSWTGYLLFQPVASSHLEQREALWIEEKGTPQASCS
ZNF-324-Krab:
(SEQ ID NO: 101)
MAFEDVAVYFSQEEWGLLDTAQRALYRRVMLDNFALVASLGLSTSRPRVVIQLERGEEP
WVPSGTDTTLSRTTYRRRNPGSWSLTEDRDVS

### Example 7

### Split Transcriptional Repressors

Modularity is a hallmark of transcription factors. Split encoding of DNA targeting and functional activities on separate molecules, as exemplified in RNA-guided systems such as Cas/CRISPR, offers substantial potential for flexibility and scale. We reasoned that if synthetic repressors could be decomposed into separately delivered T-DBDs (TALE-DBDs) and repressor domains that assembled in situ, it would be possible to screen large numbers of functional alternatives to KRAB by delivering them to the same target site. It would also open new avenues for implementing complex combinatorial cell engineering programs.

Orthogonal protein heterodimer pairs (Z. Chen et al., Nature 565, 106-111, 2019) offer an attractive system for ordered protein-protein pairing. However, the ability of such pairs to function in the complex environment of human cells is unknown. We first tested whether T-DBD and KRAB domains could be split and efficiently assembled following electroporation as separate molecules. We designed modified synthetic repressors that incorporated one half of an orthogonal protein heterodimer pair (see Table 14) after the C-terminal residue of the PD-1 synthetic repressor T-DBD. On a separately encoded molecule, we engineered its cognate half upstream of the N-terminal residue of KRAB. Introduction of either the separately encoded T-DBD/heterodimer or heterodimer/KRAB proteins alone showed no effects on PD-1 gene expression (FIG. 25, left). By contrast, parallel electroporation of separate mRNAs encoding each molecule produced potent repression nearly indistinguishable from that of the same T-DBD/KRAB synthetic repressor encoded by a single chain polypeptide (FIG. 25, right). As such, an obligate heterodimer pair can enable the DNA binding and functional domains of synthetic transcription factors to be split and separately delivered in a flexible, and potentially highly scalable, manner.

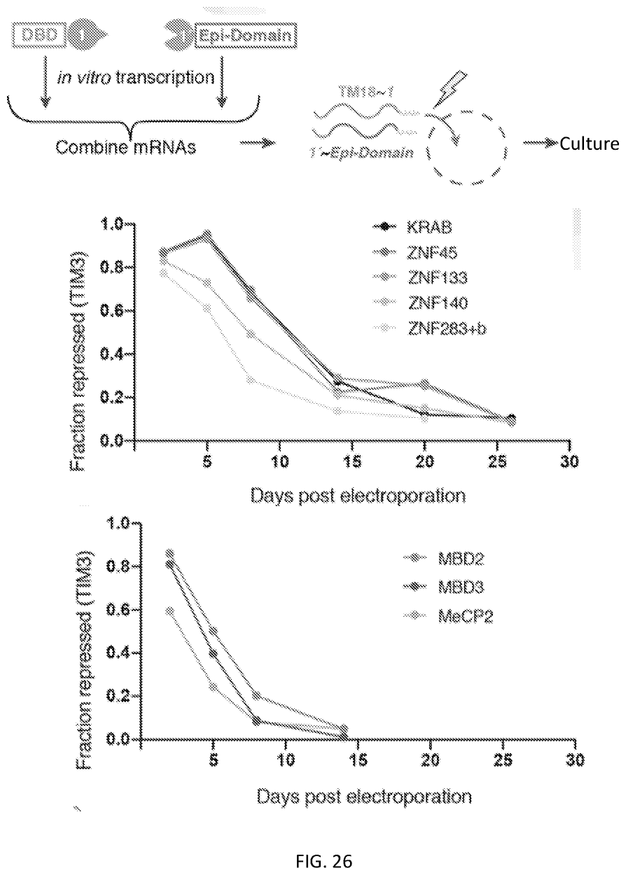

Next, we leveraged synthetic split TFs (SSTFs) to explore the functional impacts on both the potency and the kinetics expression of a wide range of candidate repressor domains extracted from native human TFs by delivering them to a target site in the TIM3 promoter targeted by the DBD of TL8188 (FIG. 26, Top panel). We co-delivered TL8188-DBD-1 mRNA into primary human T-cells together with mRNA encoding each (separately) of 77 candidate repressive domains listed in Table 13, fused to the 37B heterodimer (FIG. 26, Top panel), and assayed TIM3 expression by flow cytometry over a 26 day interval. We identified numerous highly active repressive domains that differed chiefly in their temporal kinetics of repression (FIG. 26, middle and bottom panels). Some SSTFs displayed an immediate sharp decline in repression at 5 days and complete loss by 2 weeks (FIG. 26, bottom panel). In contrast, different KRAB domain homologs from human zinc finger proteins exhibited a relatively slow kinetic profile of de-repression that extended to at least 26 days (FIG. 26, medium panel). The relative potency of different domains was similar but not identical across genes. Further, the spatial presentation of functional domains, whether fused to the heterodimer at the C or N-terminus, altered the repressive efficacy of at least one domain (MBD2), but not others (KRAB, CTBP1, and MECP2) (data not shown). Notably, we observed only modest repressive activity for the DNMT3A-3L dual domain when combined with the DNA binding domain of TL8188, pAL043, or TL8222.

FIG. 26. Large-scale analysis of functional domains enabled by split encoding of DNA targeting and functional activities. Top panel. The DNA binding domain of the TIM3 repressor TL8188 was selected to screen additional functional domains. TL8188-DBD was fused to heterodimer 37A, and a plurality of nucleic acids of functional domains was fused to heterodimer 37B. Both constructs were transiently expressed in primary human T cells by RNA electroporation, and fraction of cells with TIM3 repressed (% TIM3 negative cells in TL8188-treated cells relative to no RNA control) was evaluated periodically for 26 days by cell surface antibody staining and flow cytometry. Cells with greater fluorescence intensity than unstained control were considered TIM3+. Middle panel. Domains containing KRAB showed more durable repression, or relatively slow kinetics of decay, for several different KRAB domains. Bottom panel. Domains from methyl-DNA binding proteins showed less durable repression, or relatively fast kinetics of decay.

The above results thus show that SSTFs can be used to deliver different functional activities to the same keyhole site (or any other targeted site) at scale, and indicate that different classes of repressive domains encoded within native TFs may confer different functions that are reflected chiefly in the kinetics of repression as a function of cell proliferation time.

TABLE 13
List of genes from which candidate
repressor domains were selected for screening.
Domains Tested

ATF7IP
CBX5 (HP1a)
CHD4
COBB (E. coli)
CTBP1
DNMTA43
EED
EZH2
G9a
GFI1
GLP
HDAC1
HDAC3
HDAC9
HDT1
HP1a (mut)
HST2
IKZF1 (C-term)
IKZF1 (C-term)
IKZF1 (N-term)
KMT5A
MBD1
MBD2
MBD3
MBD4
MeCP2 (mouse)
MeCP2 (human)
MTA2
NIPP1 (PPP1R8)
PATZ1 (N-term)
PEDLS pentamer
PLDLS pentamer
PRDM1
PVDLT pentamer
RB1 (mut)
RBBP4
RBBP7 (RbAp46)
RCOR1
RUNX1
RUNX3
SAP18
SAP30
SET-TAF1B
SET8 (T. gondii)
SETD2
SETDB1 (C-term)
SIN3A
SIRT1
SUV39H1
SUV39H2
SUV39H2 (mut)
SUZ12
TLE1
TRIM28
TRIM28 (dup)
YY1
ZBTB16 (N-term)
ZBTB33
ZBTB7B (N-term)
ZNF10
ZNF133
ZNF140
ZNF274
ZNF281
ZNF283
ZNF283 + KRAB B
ZNF45

### Example 8

### Cognate and Non-Cognate Heterodimer Pairs

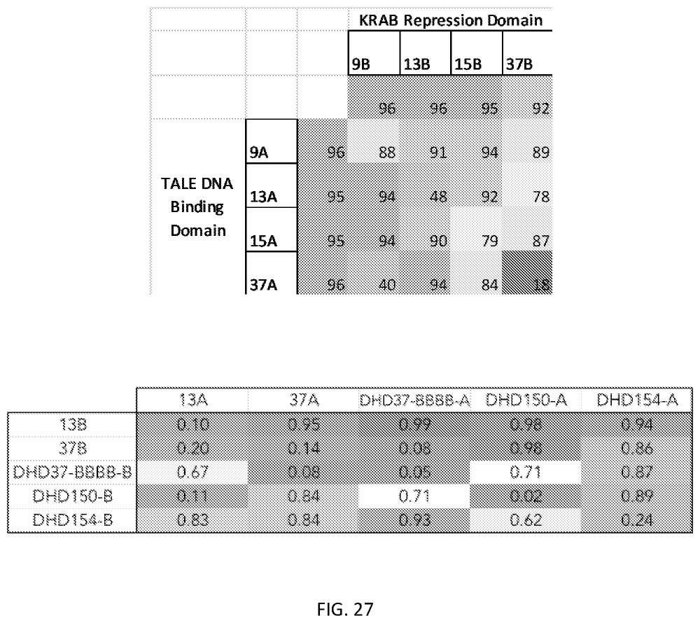

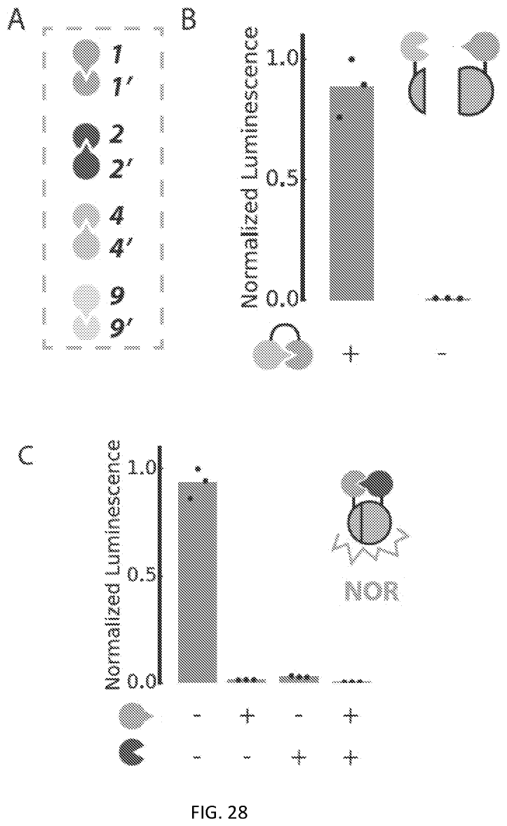

TIM3 expression was assayed using flow cytometry and plotted as % TIM3+ cells at Day 2 post-transfection with an mRNA encoding TIM3 targeting DBD (from TL8188) fused to one member of a heterodimer pair and an mRNA encoding another member of the heterodimer pair fused to a KRAB domain. Cognate pairs: 13A, 13B; 37A, 37B; DHD37-BBB-A, DHD37-BBB-B; DHD150A, DHD150B; DHD154A, DHD154B mediate dimerization and repression. Non-cognate pairs 13, 37; 13, DHD150; 37, DHD37-BBB; and 37, DHD150 also mediated dimerization and repression. See FIG. 27.

Integration of CIPHR logic gates with T cell transcriptional repressors. Engineered T cell therapies are promising therapeutic modalities, but their efficacy for treating solid tumors is limited at least in part by T cell exhaustion. Immune checkpoint genes including PD-1, CTLA4, LAG3, and TIM3 are believed to play critical roles in modulating T cell exhaustion. To put the transcription of such proteins under the control of the CIPHR logic gates, we took advantage of potent and selective transcriptional repressors of immune checkpoint genes in primary T cells that combine sequence-specific transcription activator-like effector (TALE) DNA binding domains with the Krüppel-associated box (KRAB) repressor domain; this repression activity is preserved in split systems pairing a DNA recognition domain fused with a monomer of a heterodimer pair with a functional domain fused to the complementary monomer of the heterodimer pair.

We reasoned that this system could be exploited to engineer programmable therapeutic devices by placing the coupling of separate TALE and KRAB polypeptides fused to monomers (and hence the repression function of the combined molecule) under control of CIPHR gates, such that their proximity could be controlled by logic operations. Use of a repressive domain effectively reverses the logic of CIPHR gates when expression level of the target gene is measured as the output.

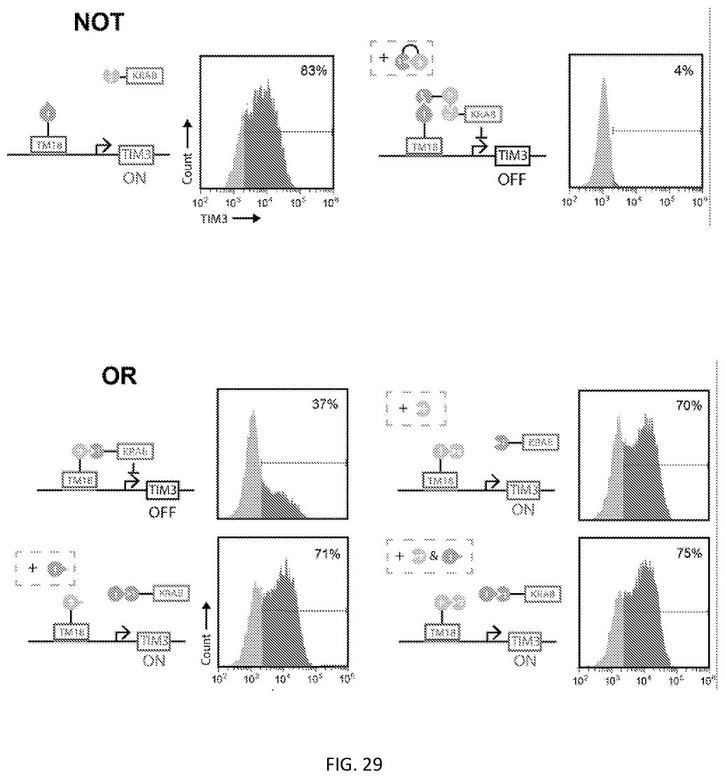

To test the feasibility of this concept, we used a TALE-KRAB fusion engineered to repress TIM3, and thus potentially attenuate T cell exhaustion. We used the all-by-all interaction specificity of a set of four heterodimer pairs (1-1′, 2-2′, 4-4′, and 9-9′) in this TALE-KRAB setting to design a NOT gate, with 1 fused to TALE, 9′ fused to KRAB, and the 1′-9 linker protein as the input. In this scheme, 1′-9 brings KRAB to the promoter region bound by the TALE, therefore triggering repression of TIM3 (FIG. 29, Top panel). Taking advantage of the interaction between 9 and 1′, we built an OR gate with 9-TALE and 1′-KRAB fusions; TIM3 is repressed in the absence of inputs, but upon addition of either 9′ or 1, the weaker 9:1′ interaction is outcompeted in favor of the stronger 9:9′ and 1:1′ interactions, restoring TIM3 expression (FIG. 29, Bottom panel). These results suggest that the combination of CIPHR and TALE-KRAB systems could be directly applied to add signal processing capabilities to adoptive T cell therapy.

TABLE 14
Sequences of the heterodimer members.

| Heterodimer | |
|---|---|
| member | |
| (Alternate | |
| Name) | Sequence |
| 1 (37A) | DSDEHLKKLKTFLENLRRHLDRLDKHIKQLRD |
| | ILSENPEDERVKDVIDLSERSVRIVKTVIKIF |
| | EDSVRKKE (SEQ ID NO: 473) |
| 1′ (37B) | GSDDKELDKLLDTLEKILQTATKIIDDANKLL |
| | EKLRRSERKDPKVVETYVELLKRHEKAVKELL |
| | EIAKTHAKKVE (SEQ ID NO: 474) |
| 9 (13A) | GTKEDILERQRKIIERAQEIHRRQQEILEELE |
| | RIIRKPGSSEEAMKRMLKLLEESLRLLKELLE |
| | LSEESAQLLYEQR (SEQ ID NO: 475) |
| 9′ (13B) | GTEKRLLEEAERAHREQKEIIKKAQELHRRLE |
| | EIVRQSGSSEEAKKEAKKILEEIRELSKRSLE |
| | LLREILYLSQEQKGSLVPR (SEQ ID |
| | NO: 476) |
| DHD37- | DEEDHLKKLKTHLEKLERHLKLLEDHAKKLED |
| BBB-A | ILKERPEDSAVKESIDELRRSIELVRESIEIF |
| | RQSVEEEE (SEQ ID NO: 477) |
| DHD37- | GDVKELTKILDTLTKILETATKVIKDATKLLE |
| BBB-B | EHRKSDKPDPRLIETHKKLVEEHETLVRQHKE |
| | LAEEHLKRTR (SEQ ID NO: 478) |
| DHD150-A | PTDEVIEVLKELLRIHRENLRVNEEIVEVNER |
| | ASRVTDREELERLLRRSNELIKRSRELNEESK |
| | KLIEKLERLAT (SEQ ID NO: 483) |
| DHD150-B | DNEEIIKEARRVVEEYKKAVDRLEELVRRAEN |
| | AKHASEKELKDIVREILRISKELNKVSERLIE |
| | LWERSQERAR (SEQ ID NO: 479) |
| DHD-154-A | TAEELLEVHKKSDRVTKEHLRVSEEILKVVEV |
| | LLTRGEVSSEVLKRVLRKEELTDKLRRVTEEQ |
| | RRVVEKLN (SEQ ID NO: 480) |
| DHD-154-B | DLEDLLRRLRRLVDEQRRLVEELERVSRRLEK |
| | AVRDNEDERELARLSREHSDIQDKHDKLAREI |
| | LEVLKRLLERTE (SEQ ID NO: 481) |

While specific embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

For reasons of completeness, certain aspects of the polypeptides, composition, and methods of the present disclosure are set out in the following numbered clauses:

1. A recombinant polypeptide comprising:
a DNA binding domain (DBD) and a transcriptional repressor domain,
the DBD comprising a plurality of repeat units (RUs) ordered from N-terminus to C-terminus of the DBD to bind to a nucleic acid sequence of the PDCD1 gene, wherein the nucleic acid sequence is present within the sequence:

(SEQ ID NO: 1)
TGGTGGGGCTGCTCCAGGCATGCAGATCCCACAGGCGCCCTGG

wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

2. The recombinant polypeptide of clause 1, wherein the RUs are ordered from N-terminus to the C-terminus to bind to the sequence: GGGGCTGCTCC (SEQ ID NO:2), wherein the first RU at the N-terminus binds to the G at the 5′ end of the sequence and the last RU at the C-terminus binds to the C at the 3′ end of the sequence.

3. The recombinant polypeptide of clause 2, wherein the X₁₂X₁₃ in the RUs from N-terminus to C-terminus are NH, NH, NH, NH, HD, NG, NH, HD, NG, HD, and HD.

4. The recombinant polypeptide of clause 2 or 3, wherein the DBD comprises at least an additional RU at the N-terminus such that the DBD binds to the nucleic acid sequence TGGGGCTGCTCC (SEQ ID NO:3), wherein X₁₂X₁₃ in the additional RU is NG, HG, KG, or RG for recognition of the T.

5. The recombinant polypeptide of clause 1, wherein the RUs are ordered from N-terminus to the C-terminus to bind to the sequence: GGTGGGGCTGCTCC (SEQ ID NO:4), wherein the first RU at the N-terminus binds to the G at the 5′ end of the sequence and the last RU at the C-terminus binds to the C at the 3′ end of the sequence.

6. The recombinant polypeptide of clause 5, wherein the DBD comprises at least fourteen RUs, wherein X₁₂X₁₃ in the RUs from N-terminus to C-terminus are NH, NH, NG, NH, NH, NH, NH, HD, NG, NH, HD, NG, HD, and HD.

7. The recombinant polypeptide of clause 5 or 6, wherein the DBD comprises three additional RU at the N-terminus such that the DBD binds to the nucleic acid sequence TGGTGGGGCTGCTCC (SEQ ID NO:5).

8. The recombinant polypeptide of clause 5, wherein the DBD comprises three additional RUs at the C-terminus such that the DBD binds to the sequence GGTGGGGCTGCTCCAGG (SEQ ID NO:6).

9. The recombinant polypeptide of clause 1, wherein the RUs are arranged from N-terminus to C-terminus to bind to the sequence: GCAGATCCCACAGGCGC (SEQ ID NO:7).

10. The recombinant polypeptide of clause 1, wherein the RUs are arranged from N-terminus to C-terminus to bind to the sequence: CCCACAGGCGCCCTGG (SEQ ID NO:8).

11. The recombinant polypeptide of clause 1, wherein the RUs are arranged from N-terminus to C-terminus to bind to the sequence: GGGGCTGCTCCAGGCATGC (SEQ ID NO:9).

12. A recombinant polypeptide comprising:
a DNA binding domain (DBD) and a transcriptional repressor,
the DBD comprising a plurality of repeat units (RUs) ordered from N-terminus to C-terminus of the DBD to bind to a nucleic acid sequence of the PDCD1 gene, wherein the nucleic acid sequence is present within the sequence:

(SEQ ID NO: 10)
CCTCCCCCAGCACTGCCTCTGTCACTCTCGCCCACGTGGATGTGG,

wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of PD1 receptor encoded by the PDCD1 gene.

13. The recombinant polypeptide of clause 12, wherein the RUs are ordered from N-terminus to C-terminus of the DBD to bind to the nucleic acid sequence TCTGTCACTCTCG (SEQ ID NO: 11).

14. The recombinant polypeptide of clause 13, wherein the DBD comprises at least thirteen RUs, wherein X₁₂X₁₃ in the RUs from N-terminus to C-terminus are NG, HD, NG, NH, NG, HD, NI, HD, NG, HD, NG, HD, and NH.

15. The recombinant polypeptide of clause 13 or 14, wherein the DBD further comprises three additional RUs at the N-terminus such that the DBD binds to the nucleic acid sequence GCCTCTGTCACTCTCG (SEQ ID NO: 12).

16. The recombinant polypeptide of clause 15, wherein the DBD further comprises three additional RUs at the C-terminus such that the DBD binds to the nucleic acid sequence GCCTCTGTCACTCTCGCCC (SEQ ID NO: 13).

17. The recombinant polypeptide of clause 16, wherein the DBD comprises at least nineteen RUs, wherein X₁₂X₁₃ in the RUs from N-terminus to C-terminus are NH, HD, HD, NG, HD, NG, NH, NG, HD, NI, HD, NG, HD, NG, HD, NH, HD, HD, and HD.

18. The recombinant polypeptide of clause 13 or 14, wherein the DBD further comprises five additional RUs at the C-terminus such that the DBD binds to the nucleic acid sequence TCTGTCACTCTCGCCCAC (SEQ ID NO: 14).

19. The recombinant polypeptide of clause 18, wherein the DBD comprises at least eighteen RUs, wherein X₁₂X₁₃ in the RUs from N-terminus to C-terminus are NG, HD, NG, NH, NG, HD, NI, HD, NG, HD, NG, HD, NG, NH, HD, HD, HD, NI, and HD.

20. The recombinant polypeptide of clause 12, wherein the DBD comprises thirteen RUs ordered from N-terminus to C-terminus of the DBD to bind to the nucleic acid sequence: CCCCCAGCACTGC (SEQ ID NO: 15).

21. The recombinant polypeptide of clause 20, wherein the DBD further comprises three additional RUs at the N-terminus such that the DBD binds to the nucleic acid sequence:

| | |
|---|---|
| CCTCCCCCAGCACTGC. | (SEQ ID NO: 16) |

22. The recombinant polypeptide of clause 21, wherein the DBD further comprises an additional RU at the C-terminus such that the DBD binds to the nucleic acid sequence:

| | |
|---|---|
| CCTCCCCCAGCACTGCC. | (SEQ ID NO: 17) |

23. A recombinant polypeptide comprising:

a DNA binding domain (DBD) and a transcriptional repressor,

the DBD comprising at least nine repeat units (RUs) ordered from N-terminus to C-terminus of the DBD to bind to a nucleic acid sequence of the PDCD1 gene, wherein the nucleic acid sequence is present within the sequence:

CCCAGGTCAGGTTGAAG (SEQ ID NO: 18), wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of PD1 receptor encoded by the PDCD1 gene.

24. A recombinant polypeptide comprising:

a DNA binding domain (DBD) and a transcriptional repressor,

the DBD comprising at least nine repeat units (RUs) ordered from N-terminus to C-terminus of the DBD to bind to a nucleic acid sequence of the PDCD1 gene, wherein the nucleic acid sequence is present within the sequence:

(SEQ ID NO: 19)
CCCTTCAACCTGACCTGGGACAGTTTCCCTTCCGCTCACCTCCGCCTGA,

wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of PD1 receptor encoded by the PDCD1 gene.

25. The recombinant polypeptide of clause 24, wherein the DBD comprises ten RUs ordered from N-terminus to C-terminus to bind to the nucleic acid sequence: TCCGCTCACC (SEQ ID NO:20).

26. The recombinant polypeptide of clause 25, wherein the DBD comprises nine additional RUs at the C-terminus such that the DBD binds to the nucleic acid sequence:

| | |
|---|---|
| TCCGCTCACCTCCGCCTGA. | (SEQ ID NO: 21) |

27. The recombinant polypeptide of clause 25, wherein the DBD comprises four additional RUs at the N-terminus such that the DBD binds to the nucleic acid sequence: CCCTTCCGCTCACC (SEQ ID NO:22).

28. The recombinant polypeptide of clause 27, wherein the DBD comprises five additional RUs at the C-terminus such that the DBD binds to the nucleic acid sequence:

| | |
|---|---|
| CCCTTCCGCTCACCTCCGC. | (SEQ ID NO: 23) |

29. The recombinant polypeptide of clause 27, wherein the DBD comprises two additional RUs at the N-terminus such that the DBD binds to the nucleic acid sequence: TTCCCTTCCGCTCACC (SEQ ID NO:24).

30. The recombinant polypeptide of clause 24, wherein the DBD comprises twelve RUs ordered from N-terminus to C-terminus to bind to the nucleic acid sequence: GGGACAGTTTCC (SEQ ID NO:25).

31. The recombinant polypeptide of clause 30, wherein the DBD further comprises four additional RUs at the C-terminus such that the DBD binds to the nucleic acid sequence: GGGACAGTTTCCCTTC (SEQ ID NO:26).

| | |
|---|---|
| GGGACAGTTTCCCTTC. | (SEQ ID NO: 26) |

32. The recombinant polypeptide of clause 30, wherein the DBD further comprises five additional RUs at the N-terminus such that the DBD binds to the nucleic acid sequence:

(SEQ ID NO: 27)
GACCTGGGACAGTTTCC.

33. The recombinant polypeptide of clause 24, wherein the DBD comprises eleven RUs ordered from N-terminus to C-terminus to bind to the nucleic acid sequence: CAACCTGACCT (SEQ ID NO:28).

34. The recombinant polypeptide of clause 33, wherein the DBD comprises nine additional RUs at the C-terminus such that the DBD binds to the nucleic acid sequence:

(SEQ ID NO: 29)
CAACCTGACCTGGGACAGTT.

35. The recombinant polypeptide of clause 33, wherein the DBD comprises five additional RUs at the N-terminus such that the DBD binds to the nucleic acid sequence: CCCTTCAACCTGACCT (SEQ ID NO:30).

36. A recombinant polypeptide comprising:

a DNA binding domain (DBD) and a transcriptional repressor,

the DBD comprising at least nine repeat units (RUs) ordered from N-terminus to C-terminus of the DBD to bind to a nucleic acid sequence of the PDCD1 gene, wherein the nucleic acid sequence is present within the sequence: GCCGCCTTCTCCACTGCTCAGGCGGAGGT (SEQ ID NO:31), wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of PD1 receptor encoded by the PDCD1 gene.

37. The recombinant polypeptide of clause 36, wherein the DBD comprises RUs arranged from N-terminus to C-terminus such that the DBD binds to the nucleic acid sequence: GCCGCCTTCTCCACT (SEQ ID NO:32).

(SEQ ID NO: 32)
GCCGCCTTCTCCACT.

38. The recombinant polypeptide of clause 36, wherein the DBD comprises RUs arranged from N-terminus to C-terminus such that the DBD binds to the nucleic acid sequence:

(SEQ ID NO: 33)
CCACTGCTCAGGCG.

39. The recombinant polypeptide of clause 38, wherein the DBD further comprises three additional RUs at the N-terminus such that the DBD binds to the nucleic acid sequence:

(SEQ ID NO: 34)
TCTCCACTGCTCAGGCG.

40. The recombinant polypeptide of clause 38, wherein the DBD further comprises five additional RUs at the C-terminus such that the DBD binds to the nucleic acid sequence:

(SEQ ID NO: 35)
CCACTGCTCAGGCGGAGGT.

41. A recombinant polypeptide comprising:

a DNA binding domain (DBD) and a transcriptional repressor,

the DBD comprising at least nine repeat units (RUs) ordered from N-terminus to C-terminus of the DBD to bind to a nucleic acid sequence of the PDCD1 gene, wherein the nucleic acid sequence is present within the sequence: GGCCAGGGCGCCTGT (SEQ ID NO:36);

CTGCATGCCTGGAGCAG (SEQ ID NO:37); GCTCCCGCCCCCTCTTCCT (SEQ ID NO:38); CTTCCTCCACATCCACG (SEQ ID NO:39); or CCTCCACATCCACGTGGGC (SEQ ID NO:40), wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of PD1 receptor encoded by the PDCD1 gene.

42. The recombinant polypeptide of any one of clauses 1-41, wherein the DBD comprises at least 11 RUs.

43. The recombinant polypeptide of any one of clauses 1-41, wherein the DBD comprises at least 13 RUs.

44. The recombinant polypeptide of any one of clauses 1-41, wherein the DBD comprises at least 15 RUs.

45. The recombinant polypeptide of any one of clauses 1-41, wherein the DBD comprises at least 17 RUs.

46. The recombinant polypeptide of any one of the preceding clauses, wherein the DBD comprises up to 40 RUs.

47. The recombinant polypeptide of any one of the preceding clauses, wherein the DBD comprises additional RUs at the N-terminus that bind to the nucleotides present upstream of the nucleic acid sequence.

48. The recombinant polypeptide of any one of the preceding clauses, wherein the DBD comprises additional RUs at the C-terminus that bind to the nucleotides present downstream of the nucleic acid sequence.

49. A recombinant polypeptide comprising: a DNA binding domain (DBD) and a transcriptional repressor, the DBD comprising a plurality of repeat units (RUs) ordered from N-terminus to C-terminus of the DBD to bind to a nucleic acid sequence of the TIM3 gene, wherein the nucleic acid sequence is present within the sequence: GGCAGTGTTACTATAAGAATCACTGGCAATCAGACACCCGGGTG (SEQ ID NO:41) or a complement thereof, wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of TIM3 encoded by the TIM3 gene.

50. The recombinant polypeptide of clause 49, wherein the DBD comprises RUs that bind to the nucleic acid sequence TGTTACTATA (SEQ ID NO:42).

51. The recombinant polypeptide of clause 50, wherein the DBD comprises an additional RU at the C-terminus such that the DBD binds to the nucleic acid sequence TGTTACTATAA (SEQ ID NO:43).

52. The recombinant polypeptide of clause 50 or 51, wherein the DBD comprises three additional RUs at the N-terminus such that the DBD binds to the nucleic acid sequence CAGTGTTACTATAA (SEQ ID NO:44).

53. The recombinant polypeptide of clause 52, wherein the DBD comprises two additional RUs at the N-terminus such that the DBD binds to the nucleic acid sequence GGCAGTGTTACTATAA (SEQ ID NO:45).

54. The recombinant polypeptide of clause 49, wherein the DBD comprises RUs that bind to the nucleic acid sequence TCAGACACCCGGGTG (SEQ ID NO:46).

55. The recombinant polypeptide of clause 54, wherein the DBD comprises three additional RUs at the N-terminus such that the DBD binds to the nucleic acid sequence CAATCAGACACCCGGGTG (SEQ ID NO:47).

56. The recombinant polypeptide of clause 54, wherein the DBD comprises three additional RUs at the N-terminus such that the DBD binds to the nucleic acid sequence TGGCAATCAGACACCCGGGTG (SEQ ID NO:48).

57. A recombinant polypeptide comprising:

a DNA binding domain (DBD) and a transcriptional repressor, the DBD comprising a plurality of repeat units (RUs) ordered from N-terminus to C-terminus of the DBD to bind a nucleic acid sequence of the TIM3 gene, wherein the nucleic acid sequence is present within the sequence:

TGTCTGATTGCCAGTGATTCTTATAGT (SEQ ID NO:49), wherein each of the repeat unit comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of TIM3 encoded by the TIM3 gene.

58. The recombinant polypeptide of clause 57, wherein the DBD comprises RUs that are ordered to bind to the sequence TGCCAGTGATT (SEQ ID NO:50).

59. The recombinant polypeptide of clause 58, wherein the DBD comprises eight additional RUs at the C-terminus such that the DBD binds to the sequence TGCCAGTGATTCTTATAGT (SEQ ID NO:51).

60. The recombinant polypeptide of clause 57, wherein the DBD comprises RUs that are ordered to binds to the sequence TGATTGCCAGTGATT (SEQ ID NO:52).

61. The recombinant polypeptide of clause 60, wherein the DBD comprises four additional RUs at the N-terminus such that the DBD binds to the sequence TGTCTGATTGCCAGTGATT (SEQ ID NO:53).

62. A recombinant polypeptide comprising: a DNA binding domain (DBD) and a transcriptional repressor, the DBD comprising a plurality of repeat units (RUs) ordered from N-terminus to C-terminus of the DBD to bind to a nucleic acid sequence of TIM3 gene, wherein the nucleic acid sequence is: TACACACAT (SEQ ID NO:54), wherein each of the repeat unit comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of TIM3 encoded by the TIM3 gene.

63. The recombinant polypeptide of clause 62, wherein the DBD comprises four additional RUs at the N-terminus such that the DBD binds to the sequence ACACTACACACAT (SEQ ID NO:55).

64. The recombinant polypeptide of clause 63, wherein the DBD comprises four additional RUs at the N-terminus such that the DBD binds to the sequence TGCCACACTACACACAT (SEQ ID NO:56).

65. A recombinant polypeptide comprising: a DNA binding domain (DBD) and a transcriptional repressor, the DBD comprising at least nine repeat units (RUs) ordered from N-terminus to C-terminus of the DBD to bind to a nucleic acid sequence of the LAG3 gene, wherein the nucleic acid sequence is present within the sequence:

GCCGTTCTGCTGGTCTCTGGGCCTTCACCCCTGTGCCCGGCCTTCC (SEQ ID NO:57), wherein each of the RU comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from:

(a) NH, HH, KH, NK, NQ, RH, RN, SS, NN, SN, or KN for recognition of guanine (G);

(b) NI, KI, RI, HI, or SI for recognition of adenine (A);

(c) NG, HG, KG, or RG for recognition of thymine (T);

(d) HD, RD, SD, ND, KD, or YG for recognition of cytosine (C); and

(e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of LAG3 encoded by the LAG3 gene.

66. The recombinant polypeptide of clause 65, wherein the DBD comprises RUs that bind to the sequence TCTGCTGGTCT (SEQ ID NO:58).

67. The recombinant polypeptide of clause 66, wherein the DBD comprises five additional RUs at the N-terminus such that the DBD binds to the sequence GCCGTTCTGCTGGTCT (SEQ ID NO:59).

68. The recombinant polypeptide of clause 67, wherein the DBD comprises two additional RUs at the C-terminus such that the DBD binds to the sequence GCCGTTCTGCTGGTCTCT (SEQ ID NO:60).

69. The recombinant polypeptide of clause 66, wherein the DBD comprises four additional RUs at the C-terminus such that the DBD binds to the sequence TCTGCTGGTCTGGGC (SEQ ID NO: 61).

70. The recombinant polypeptide of clause 69, wherein the DBD comprises an additional RUs at the C-terminus such that the DBD binds to the sequence TCTGCTGGTCTGGGCC (SEQ ID NO: 62).

71. The recombinant polypeptide of clause 70, wherein the DBD comprises three additional RUs at the C-terminus such that the DBD binds to the sequence TCTGCTGGTCTGGGCCTTC (SEQ ID NO:63).

72. The recombinant polypeptide of clause 65, wherein the DBD comprises RUs that bind to the sequence TCTCTGGGCCTTCA (SEQ ID NO:64).

73. The recombinant polypeptide of clause 72, wherein the DBD comprises two additional RUs at the N-terminus such that the DBD binds the sequence GGTCTCTGGGCCTTCA (SEQ ID NO:65).

74. The recombinant polypeptide of clause 73, wherein the DBD comprises three additional RUs at the C-terminus such that the DBD binds the sequence GGTCTCTGGGCCTTCACCC (SEQ ID NO:66).

75. The recombinant polypeptide of clause 74, wherein the DBD comprises an additional RUs at the N-terminus such that the DBD binds the sequence TGGTCTCTGGGCCTTCACC (SEQ ID NO:67).

76. The recombinant polypeptide of clause 65, wherein the DBD comprises RUs that bind to the sequence TTCACCCCTGTG (SEQ ID NO:68).

77. The recombinant polypeptide of clause 76, wherein the DBD comprises four additional RUs at the C-terminus such that the DBD binds to the sequence TTCACCCCTGTGCCCG (SEQ ID NO:69).

78. The recombinant polypeptide of clause 77, wherein the DBD comprises four additional RUs at the C-terminus such that the DBD binds to the sequence TTCACCCCTGTGCCCGGCCT (SEQ ID NO:70).

79. The recombinant polypeptide of clause 78, wherein the DBD comprises three additional RUs at the C-terminus such that the DBD binds to the sequence TTCACCCCTGTGCCCGGCCTTCC (SEQ ID NO:71).

80. A recombinant polypeptide comprising: a DNA binding domain (DBD) and a transcriptional repressor, the DBD comprising a plurality of repeat units (RUs) ordered from N-terminus to C-terminus of the DBD to bind to a nucleic acid sequence of LAG3 gene, wherein the nucleic acid sequence is: TGCTCTGTCTGC (SEQ ID NO:72), wherein each of the repeat unit comprises the sequence X₁₋₁₁X₁₂X₁₃X_{14-33, 34, or 35} (SEQ ID NO: 455), wherein: X₁₋₁₁ is a chain of 11 contiguous amino acids, X_{14-33 or 34 or 35} is a chain of 20, 21 or 22 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of LAG3 encoded by the LAG3 gene.

81. The recombinant polypeptide of clause 80, wherein the DBD comprises two additional RUs at the C-terminus such that the DBD binds to the sequence TGCTCTGTCTGCTC (SEQ ID NO:73).

82. The recombinant polypeptide of clause 81, wherein the DBD comprises two additional RUs at the N-terminus such that the DBD binds to the sequence TTTGCTCTGTCTGCTC (SEQ ID NO:74).

83. A recombinant polypeptide comprising: a DNA binding domain (DBD) and a transcriptional repressor, the DBD comprising at least nine repeat units (RUs) ordered from N-terminus to C-terminus of the DBD to bind to a nucleic acid sequence of the CTLA4 gene, wherein the nucleic acid sequence is:

| | |
|---|---|
| ACATATCTGGGATCAAAGCT, | (SEQ ID NO: 75) |
| ATATAAAGTCCTTGAT, | (SEQ ID NO: 76) |
| or | |
| TTCTATTCAAGTGCC, | (SEQ ID NO: 77) |

wherein each of the RU comprises the sequence X₁₋₁₁X₁₂X₁₃X_{14-33, 34, or 35} (SEQ ID NO: 455), wherein:

X₁₋₁₁ is a chain of 11 contiguous amino acids,

X_{14-33 or 34 or 35} is a chain of 20, 21 or 22 contiguous amino acids,

X₁₂X₁₃ is selected from:

(a) NH, HH, KH, NK, NQ, RH, RN, SS, NN, SN, or KN for recognition of guanine (G);

(b) NI, KI, RI, HI, or SI for recognition of adenine (A);

(c) NG, HG, KG, or RG for recognition of thymine (T);

(d) HD, RD, SD, ND, KD, or YG for recognition of cytosine (C); and

(e) NV or HN for recognition of A or G; and (f) H*, HA, KA, N*, NA, NC, NS, RA, or S* for recognition of A or T or G or C, wherein (*) means that the amino acid at X₁₃ is absent, and wherein the transcriptional repressor domain suppresses expression of CTLA4 encoded by the CTLA4 gene.

84. The recombinant polypeptide of any one of the preceding clauses, wherein the DBD comprises up to 40 RUs.

85. The recombinant polypeptide of any one of the preceding clauses, wherein the DBD comprises up to 35 RUs.

86. The recombinant polypeptide of any one of the preceding clauses, wherein the DBD comprises up to 30 RUs.

87. The recombinant polypeptide of any one of the preceding clauses, wherein the DBD comprises up to 25 RUs.

88. The recombinant polypeptide of any one of the preceding clauses, wherein the DBD comprises up to 20 RUs.

89. The recombinant polypeptide of any one of the preceding clauses, wherein the DBD comprises additional RUs at the N-terminus that bind to the nucleotides present upstream of the nucleic acid sequence.

90. The recombinant polypeptide of any one of the preceding clauses, wherein the DBD comprises additional RUs at the C-terminus that bind to the nucleotides present downstream of the nucleic acid sequence.

91. The recombinant polypeptide of any one of the preceding clauses, wherein the transcriptional repressor domain is conjugated to the C-terminus of the DBD.

92. The recombinant polypeptide of any one of the preceding clauses, wherein the chain of 11 contiguous amino acids is at least 80% identical to LTPDQVVAIAS (SEQ ID NO:78).

93. The recombinant polypeptide of any one of the preceding clauses, wherein the chain of 20, 21, or 22 contiguous amino acids is at least 80% identical to GGKQALETVQRLLPVLCQDHG (SEQ ID NO:79).

94. The recombinant polypeptide of any one of the preceding clauses, wherein the DBD comprises a N-cap region comprising an amino acid sequence at least 80% identical to the amino acid sequence set for the in SEQ ID NO:339.

95. The recombinant polypeptide of any one of the preceding clauses, wherein the DBD comprises a C-cap region comprising an amino acid sequence at least 80% identical to the amino acid sequence set forth in SEQ ID NO: 452, wherein the recombinant polypeptide comprises from N-terminus to C-terminus: the N-cap region, the plurality of RUs, and the C-cap region.

96. The recombinant polypeptide of any one of the preceding clauses, wherein the DBD comprises a half-repeat comprising the amino acid sequence X₁₋₁₁X₁₂X₁₃X_{14-19, 20, or 21} (SEQ ID NO: 471), wherein: X₁₋₁₁ is a chain of 11 contiguous amino acids, X_{14-20 or 21 or 22} is a chain of 7, 8 or 9 contiguous amino acids, X₁₂X₁₃ is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S* for recognition of A or T or G or C, wherein (*) means that the amino acid at X₁₃ is absent.

97. The recombinant polypeptide of clause 96, wherein X₁₋₁₁ is at least 80% identical to LTPEQVVAIAS (SEQ ID NO:458).

98. The recombinant polypeptide of clause 96 or 97, wherein X_{14-20 or 21 or 22} is at least 80% identical to GGRPALE (SEQ ID NO:472).

99. A nucleic acid encoding the recombinant polypeptide of any of clauses 1-98.

100. The nucleic acid of clause 99, wherein the nucleic acid is operably linked to a promoter sequence that confers expression of the polypeptide.

101. The nucleic acid of clause 99 or 100, wherein the sequence of the nucleic acid is codon optimized for expression of the polypeptide in a human cell.

102. The nucleic acid of any one of clauses 99-101, wherein the nucleic acid is a deoxyribonucleic acid (DNA).

103. The nucleic acid of any one of clauses 99-101, wherein the nucleic acid is a ribonucleic acid (RNA).

104. A vector comprising the nucleic acid of any of clauses 99-103.

105. The vector of clause 104, wherein the vector is a viral vector.

106. A host cell comprising the nucleic acid of any of clauses 99-103 or the vector of clause 104 or 105.

107. A host cell that expresses the polypeptide of any of clauses 1-98.

108. A pharmaceutical polypeptide comprising the polypeptide of any of clauses 1-98 and a pharmaceutically acceptable excipient.

109. A pharmaceutical polypeptide comprising the nucleic acid of any of clauses 99-103 or the vector of clause 104 or 105 and a pharmaceutically acceptable excipient.

110. A method of suppressing expression of PDCD-1 gene in a cell, the method comprising:
introducing into the cell the recombinant polypeptide of any one of clauses 1-48,
wherein the recombinant polypeptide binds to a target nucleic acid sequence present in the PDCD-1 gene and the transcriptional repressor domain suppresses expression of the PDCD-1 gene.

111. A method of suppressing expression of TIM3 gene in a cell, the method comprising:

introducing into the cell the recombinant polypeptide of any one of clauses 49-64,

wherein the recombinant polypeptide binds to a target nucleic acid sequence present in the TIM3 gene and the transcriptional repressor domain suppresses expression of the TIM3 gene.

112. A method of suppressing expression of LAG3 gene in a cell, the method comprising:

introducing into the cell the recombinant polypeptide of any one of clauses 65-82,

wherein the recombinant polypeptide binds to a target nucleic acid sequence present in the LAG3 gene and the transcriptional repressor domain suppresses expression of the LAG3 gene.

113. A method of suppressing expression of CTLA4 gene in a cell, the method comprising:

introducing into the cell the recombinant polypeptide of any one of clause 83,

wherein the recombinant polypeptide binds to a target nucleic acid sequence present in the CTLA4 gene and the transcriptional repressor domain suppresses expression of the CTLA4 gene.

114. The method of any one of clauses 110-113, wherein the polypeptide is introduced as a nucleic acid encoding the polypeptide.

115. The method of clause 114, wherein the nucleic acid is a deoxyribonucleic acid (DNA).

116. The method of clause 114, wherein the nucleic acid is a ribonucleic acid (RNA).

117. The method of any of clauses 110-116, wherein the sequence of the nucleic acid is codon optimized for expression in a human cell.

118. The method of any of clauses 110-116, wherein the transcriptional repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

119. The method of any one of clauses 110-118, wherein the cell is an animal cell.

120. The method of any one of clauses 110-118, wherein the cell is a human cell.

121. The method of any one of clauses 110-120, wherein the cell is a cancer cell.

122. The method of any one of clauses 110-121, wherein the cell is an ex vivo cell.

123. The method of any one of clauses 110-121, wherein the introducing comprises administering the polypeptide or a nucleic acid encoding the polypeptide to a subject.

124. The method of clause 123, wherein the administering comprises parenteral administration.

125. The method of clause 123, wherein the administering comprises intravenous, intramuscular, intrathecal, or subcutaneous administration.

126. The method of clause 123, wherein the administering comprises direct injection into a site in a subject.

127. The method of any of clause 123, wherein the administering comprises direct injection into a tumor.

128. A recombinant polypeptide comprising a DNA binding domain and a transcriptional repressor domain, wherein the DNA binding domain and the transcriptional repressor domain are heterologous, wherein the transcriptional repressor domain comprises an amino acid sequence at least 80% identical to any one of the sequences set out in SEQ ID NOs: 84-101.

129. The recombinant polypeptide of clause 128, wherein the transcriptional repressor domain comprises an amino acid sequence at least 85% identical to any one of the sequences set out in SEQ ID NOs: 84-101.

130. The recombinant polypeptide of clause 128, wherein the transcriptional repressor domain comprises an amino acid sequence at least 90% identical to any one of the sequences set out in SEQ ID NOs: 84-101.

131. The recombinant polypeptide of clause 128, wherein the transcriptional repressor domain comprises an amino acid sequence at least 95% identical to any one of the sequences set out in SEQ ID NOs: 84-101.

132. The recombinant polypeptide of any one of clauses 128-131, wherein the DNA binding domain comprises zinc finger protein (ZFP), a transcription activator-like effector (TALE), or a guide RNA.

133. The recombinant polypeptide of any one of clauses 128-132, wherein the DNA binding domain binds to a target nucleic acid sequence in a gene and optionally, wherein the DNA binding domain is the DBD of any one of clauses 1-98.

134. The recombinant polypeptide of clause 133, wherein the target nucleic acid sequence is in a PDCD 1 gene, a CTLA4 gene, a LAG3 gene, a TET2 gene, a ETLA gene, a HAVCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.

135. A nucleic acid encoding the recombinant polypeptide of any of clauses 128-134.

136. The nucleic acid of clause 135, wherein the nucleic acid is operably linked to a promoter sequence that confers expression of the polypeptide.

137. The nucleic acid of clause 135 or 136, wherein the sequence of the nucleic acid is codon optimized for expression of the polypeptide in a human cell.

138. The nucleic acid of any one of clauses 135-137, wherein the nucleic acid is a deoxyribonucleic acid (DNA).

139. The nucleic acid of any one of clauses 135-137, wherein the nucleic acid is a ribonucleic acid (RNA).

140. A vector comprising the nucleic acid of any of clauses 135-138.

141. The vector of clause 140, wherein the vector is a viral vector.

142. A host cell comprising the nucleic acid of any of clauses 135-139 or the vector of clause

140 or 141.

143. A host cell comprising the polypeptide of any of clauses 128-134.

144. A host cell that expresses the polypeptide of any of clauses 128-134.

145. A pharmaceutical composition comprising the polypeptide of any of clauses 128-134 and a pharmaceutically acceptable excipient.

146. A pharmaceutical composition comprising the nucleic acid of any of clauses 135-139 or the vector of clause 140 or 141 and a pharmaceutically acceptable excipient.

147. A method of suppressing expression of an endogenous gene in a cell, the method comprising:
introducing into the cell the recombinant polypeptide of any one of clauses 128-134,
wherein the DBD of the polypeptide binds to a target nucleic acid sequence present in the endogenous gene and the heterologous transcriptional repressor domain suppresses expression of the endogenous gene.

148. The method of clause 147, wherein the recombinant polypeptide is introduced as a nucleic acid encoding the polypeptide.

149. The method of clause 148, wherein the nucleic acid is a deoxyribonucleic acid (DNA).

150. The method of clause 148, wherein the nucleic acid is a ribonucleic acid (RNA).

151. The method of any of clauses 148-150, wherein the sequence of the nucleic acid is codon optimized for expression in a human cell.

152. The method of any of clauses 147-151, wherein the gene is a PDCD 1 gene, a CTLA4 gene, a LAG3 gene, a TET2 gene, a ETLA gene, a HAVCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

153. The method of any one of clauses 147-152, wherein the cell is an animal cell.

154. The method of any one of clauses 147-152, wherein the cell is a human cell.

155. The method of any one of clauses 147-152, wherein the cell is a cancer cell.

156. The method of any one of clauses 147-152, wherein the cell is an ex vivo cell.

157. The method of any one of clauses 147-155, wherein the introducing comprises administering the polypeptide or a nucleic acid encoding the polypeptide to a subject.

158. The method of clause 157, wherein the administering comprises parenteral administration.

159. The method of clause 157, wherein the administering comprises intravenous, intramuscular, intrathecal, or subcutaneous administration.

160. The method of clause 157, wherein the administering comprises direct injection into a site in a subject.

161. The method of any of clause 157, wherein the administering comprises direct injection into a tumor.

162. A plurality of nucleic acids encoding:

(i) polypeptides that dimerize via direct dimerization, comprising:
(A) a DNA binding domain (DBD) fused to a first member of a heterodimer pair and a functional domain fused to a second member of the heterodimer pair, or
(B) a DNA binding domain (DBD) fused to a second member of a heterodimer pair and a functional domain fused to a first member of the heterodimer pair,
wherein the first and second members of the heterodimer pair bind to each other thereby directly dimerizing the DBD and the functional domain,
wherein the heterodimer pair is selected from one of the following heterodimer pairs:
37A, 37B;
13A, 13B;
DHD37-BBB-A, DHD37-BBB-B;
DHD150-A, DHD150-B;
DHD154-A, DHD-154B;
37A, 9B;
13A, 37B;
13A, DHD150-B;
37A, DHD37-BBB-B; and
DHD37-BBB-A, 37B; or

(ii) polypeptides that dimerize indirectly via a bridging construct, comprising:
(A) a DNA binding domain (DBD) fused to a first member of a first heterodimer pair; a bridging construct comprising a second member of the first heterodimer pair fused to a first member of a second heterodimer pair; and a functional domain fused to a second member of the second heterodimer pair; or
(B) a DNA binding domain (DBD) fused to a second member of a first heterodimer pair; a bridging construct comprising a first member of the first heterodimer pair fused to a first member of a second heterodimer pair; and a functional domain fused to a second member of the second heterodimer pair; or
(C) a DNA binding domain (DBD) fused to a second member of a first heterodimer pair; a bridging construct comprising a first member of the first heterodimer pair fused to a second member of a second heterodimer pair; and a functional domain fused to a first member of the second heterodimer pair,

wherein the DBD and the functional domain dimerize indirectly via the bridging construct,

wherein the first and second heterodimer pairs are different and are selected from the following heterodimer pairs:
37A, 37B;
13A, 13B;
DHD37-BBB-A, DHD37-BBB-B;
DHD150-A, DHD150-B;
DHD154-A, DHD-154B;
37A, 9B;
13A, 37B;
13A, DHD150-B;
37A, DHD37-BBB-B; and
DHD37-BBB-A, 37B.

163. The plurality of nucleic acids of clause 162, wherein the DBD in (i) (A) or (i) (B) is fused to a first member of a first heterodimer pair and the functional domain is a first functional domain fused a second member of the first heterodimer pair and to a first member of a second heterodimer pair, the system further comprising a second functional domain fused to a second member of the second heterodimer pair, wherein the members of the first heterodimer pair mediate dimerization of the DBD and the first functional domain and members of the second heterodimer pair mediate dimerization of the first functional domain and the second functional domain.

164. The plurality of nucleic acids of clause 163, wherein the DBD is fused to a first member of a first heterodimer pair and to a first member of a second heterodimer pair, and the functional domain is fused a second member of the first heterodimer pair the system further comprising a second functional domain fused to a second member of the second heterodimer pair, wherein the members of the first heterodimer pair mediate assembly of the DBD and the first functional domain and members of the second heterodimer pair mediate assembly of the DBD and the second functional domain.

165. The plurality of nucleic acids of any one of clauses 162-164, wherein the DBD binds to a target nucleic acid sequence present in an endogenous gene in a cell.

166. The plurality of nucleic acids of any one of clauses 162-165, wherein the functional domain comprises an enzyme, a transcriptional activator, a transcriptional repressor, or a DNA nucleotide modifier.

167. The plurality of nucleic acids of clause 166, wherein the enzyme is a nuclease, a DNA modifying protein, or a chromatin modifying protein.

168. The plurality of nucleic acids of clause 167, wherein the nuclease is a cleavage domain or a half-cleavage domain.

169. The plurality of nucleic acids of clause 168, wherein the cleavage domain or half-cleavage domain comprises a type IIS restriction enzyme.

170. The plurality of nucleic acids of clause 169, wherein the type IIS restriction enzyme comprises FokI or Bfil.

171. The plurality of nucleic acids of clause 167, wherein the chromatin modifying protein is lysine-specific histone demethylase 1 (LSD1).

172. The plurality of nucleic acids of clause 166, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

173. The plurality of nucleic acids of clause 168, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, MeCP2, or a transcriptional repressor provided in clauses 128-134.

174. The plurality of nucleic acids of clause 166, wherein the DNA nucleotide modifier is adenosine deaminase.

175. The plurality of nucleic acids of any of clauses 165-174, wherein the target nucleic acid sequence is within a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

176. The plurality of nucleic acids of any of clauses 162-175, wherein the DBD comprises a transcription activator-like effector (TALE).

177. The plurality of nucleic acids of any of clauses 162-176, wherein the DBD comprises a DBD as set out in any one of clauses 1-98.

178. A DNA binding domain and a functional domain or a DNA binding domain, a functional domain and a bridging construct encoded by the plurality of nucleic acids of nucleic acids of any one of clauses 162-177.

179. A DNA binding domain and a functional domain as set forth in clause 162 (i)(A); or (i)(B); or a DNA binding domain, a bridging construct, and a functional domain as set forth in clause 162 (ii)(A), (ii)(B), or (ii)(C).

180. A host cell comprising: (a) nucleic acids encoding the polypeptides as set forth in clause 162 (i)(A) or (i)(B); or (b) nucleic acids encoding the polypeptides as set forth in clause 162 (ii)(A), (ii)(B), or (ii)(C).

181. A host cell comprising: (a) the polypeptides as set forth in clause 162 (i)(A) or (i)(B); or (b) the polypeptides as set forth in clause 162 (ii)(A), (ii)(B), or (ii)(C).

182. A kit comprising:

(a) nucleic acids encoding the polypeptides as set forth in clause 162 (i)(A) or (i)(B); or

(b) nucleic acids encoding the polypeptides as set forth in clause 162 (ii)(A), (ii)(B), or (ii)(C).

183. A kit comprising:

(a) a first vector comprising a nucleic acid encoding the DBD set forth in clause 162 (i)(A); and

(b) a second vector comprising a nucleic acid encoding the functional domain set forth in clause 162 (i)(A); or

(a) a first vector comprising a nucleic acid encoding the DBD set forth in clause 162 (i)(B); and

(b) a second vector comprising a nucleic acid encoding the functional domain set forth in clause 162 (i)(B).

184. A kit comprising:

(a) a first vector comprising a nucleic acid encoding the DBD set forth in clause 162 (ii)(A);

(b) a second vector comprising a nucleic acid encoding the bridging construct set forth in clause 162 (ii)(A); and

(c) a third vector comprising a nucleic acid encoding the functional domain set forth in clause 162 (ii)(A); or

(a) a first vector comprising a nucleic acid encoding the DBD set forth in clause 162 (ii)(B);

(b) a second vector comprising a nucleic acid encoding the bridging construct set forth in clause 162 (ii)(B); and

(c) a third vector comprising a nucleic acid encoding the functional domain set forth in clause 162 (ii)(B); or

(a) a first vector comprising a nucleic acid encoding the DBD set forth in clause 162 (ii)(C);

(b) a second vector comprising a nucleic acid encoding the bridging construct set forth in clause 162 (ii)(C); and

(c) a third vector comprising a nucleic acid encoding the functional domain set forth in clause 162 (ii)(C).

185. A pharmaceutical composition comprising:

(a) nucleic acids encoding the polypeptides as set forth in clause 162 (i)(A) or (i)(B); or

(b) nucleic acids encoding the polypeptides as set forth in clause 162 (ii)(A), (ii)(B), or (ii)(C). 186. A pharmaceutical composition comprising:

(a) a first vector comprising a nucleic acid encoding the DBD set forth in clause 162 (i)(A); and

(b) a second vector comprising a nucleic acid encoding the functional domain set forth in clause 162 (i)(A); or

(a) a first vector comprising a nucleic acid encoding the DBD set forth in clause 162 (i)(B); and

(b) a second vector comprising a nucleic acid encoding the functional domain set forth in clause 162 (i)(B).

187. A pharmaceutical composition comprising:

(a) a first vector comprising a nucleic acid encoding the DBD set forth in clause 162 (ii)(A);

(b) a second vector comprising a nucleic acid encoding the bridging construct set forth in clause 162 (ii)(A); and

(c) a third vector comprising a nucleic acid encoding the functional domain set forth in clause 162 (ii)(A); or

(a) a first vector comprising a nucleic acid encoding the DBD set forth in clause 162 (ii)(B);

(b) a second vector comprising a nucleic acid encoding the bridging construct set forth in clause 162 (ii)(B); and

(c) a third vector comprising a nucleic acid encoding the functional domain set forth in clause 162 (ii)(B); or

(a) a first vector comprising a nucleic acid encoding the DBD set forth in clause 162 (ii)(C);

(b) a second vector comprising a nucleic acid encoding the bridging construct set forth in clause 162 (ii)(C); and

(c) a third vector comprising a nucleic acid encoding the functional domain set forth in clause 162 (ii)(C).

188. A pharmaceutical composition comprising the DBD and a functional domain or a DNA binding domain, a functional domain and a bridging construct of clause 178 and a pharmaceutically acceptable excipient.

189. A pharmaceutical composition comprising the host cell of clause 180 or 181 and a pharmaceutically acceptable excipient.

190. A method for modulating expression from a target gene in a cell, the method comprising:

(i) introducing into the cell a first nucleic acid encoding a DNA binding domain fused to a first member of a heterodimer pair and a second nucleic acid encoding a functional domain fused to a second member of the heterodimer pair; or

(ii) introducing into the cell a first nucleic acid encoding a DNA binding domain fused to a second member of a heterodimer pair and a second nucleic acid encoding a functional domain fused to a first member of the heterodimer pair; or

(iii) introducing into the cell a DNA binding domain fused to a first member of a heterodimer pair and a functional domain fused to a second member of the heterodimer pair; or

(iv) introducing into the cell a DNA binding domain fused to a second member of a heterodimer pair and a functional domain fused to a first member of the heterodimer pair, wherein the heterodimer pair is selected from one of the following heterodimer pairs:

37A, 37B;

13A, 13B;

DHD37-BBB-A, DHD37-BBB-B;

DHD150-A, DHD150-B;

DHD154-A, DHD-154B;

37A, 9B;

13A, 37B;

13A, DHD150-B;

37A, DHD37-BBB-B; and

DHD37-BBB-A, 37B,

wherein the DNA binding domain (DBD) dimerizes with the functional domain via dimerization of the members of the heterodimer pair and wherein binding of the DBD to a target nucleic acid sequence in the target gene results in modulation of expression of the target gene via the functional domain dimerized to the DBD.

191. A method of modulating expression of a target gene in a cell, the method comprising:

(i) introducing into a cell expressing a DNA binding domain (DBD) fused to a first member of a first heterodimer pair and a functional domain fused to a second member of a second heterodimer pair, a bridging construct comprising a second member of the first heterodimer pair fused to a first member of the second heterodimer pair or a nucleic acid encoding the bridging construct; or

(ii) introducing into a cell expressing a DNA binding domain (DBD) fused to a second member of a first heterodimer pair and a functional domain fused to a second member of a second heterodimer pair, a bridging construct comprising a first member of the first heterodimer pair fused to a first member of the second heterodimer pair or a nucleic acid encoding the bridging construct; or

(iii) introducing into a cell expressing a DNA binding domain (DBD) fused to a first member of a first heterodimer pair and a functional domain fused to a first member of a second heterodimer pair, a bridging construct comprising a second member of the first heterodimer pair fused to a second member of the second heterodimer pair or a nucleic acid encoding the bridging construct, wherein the DBD and the functional domain dimerize indirectly via the bridging construct, wherein binding of the DBD to a target nucleic acid sequence in a target gene in the cell results in in modulation of expression of the target gene via the functional domain dimerized to the DBD via the bridging construct, wherein the first and second heterodimer pairs are different and are selected from the following heterodimer pairs:

37A, 37B;

13A, 13B;

DHD37-BBB-A, DHD37-BBB-B;

DHD150-A, DHD150-B;

DHD154-A, DHD-154B;

37A, 9B;

13A, 37B;

13A, DHD150-B;

37A, DHD37-BBB-B; and

DHD37-BBB-A, 37B.

192. A method of reversing modulation of expression of a target gene in a cell expressing a DNA binding domain (DBD) fused to a first member of a non-cognate heterodimer pair and a functional domain fused to a second member of the non-cognate heterodimer pair, wherein the DBD binds to a target nucleic acid sequence in a target gene and the functional domain dimerized to the DBD via dimerization of the members of the heterodimer pair modulates expression of the target gene, the method comprising introducing into the cell a disruptor which binds to either the first member or the second member with a higher binding affinity than the binding affinity between the first and second members, wherein non-cognate heterodimer pairs and the corresponding disruptor are selected from one of the following combinations:

| Combination | Non-Cognate Heterodimer Pair | Disruptor |
|---|---|---|
| 1 | 37A, 9B; | 37B or 9A |
| 2 | 13A, 37B; | 13B or 37A |
| 3 | 13A, DHD150-B; | 13B or DHD150-A |
| 4 | 37A, DHD37-BBB-B; | 37B or DHD37-BBB-A |
| 5 | DHD37-BBB-A, 37B | DHD37-BBB-B or 37A |

What is claimed is:

1. A recombinant polypeptide comprising:
a DNA binding domain (DBD) and a transcriptional repressor domain,
the DBD comprising a plurality of repeat units (RUs) ordered from N-terminus to C-terminus of the DBD to bind to a nucleic acid sequence of the PDCD1 gene, wherein the nucleic acid sequence is present within the sequence:
(SEQ ID NO: 1)
TGGTGGGGCTGCTCCAGGCATGCAGATCCCACAGGCGCCCTGG
wherein each of the RU comprises the sequence X₁₋₁₁X₁₂X₁₃X_{14-33, 34, or 35} (SEQ ID NO: 455) wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

2. The recombinant polypeptide of claim 1, wherein the RUs are ordered from N-terminus to the C-terminus to bind to the sequence: GGGGCTGCTCC (SEQ ID NO:2), wherein the first RU at the N-terminus binds to the G at the 5′ end of the sequence and the last RU at the C-terminus binds to the C at the 3′ end of the sequence.

3. The recombinant polypeptide of claim 2, wherein the X₁₂X₁₃ in the RUs from N-terminus to C-terminus are NH, NH, NH, NH, HD, NG, NH, HD, NG, HD, and HD.

4. The recombinant polypeptide of claim 2 or 3, wherein the DBD comprises at least an additional RU at the N-terminus such that the DBD binds to the nucleic acid sequence TGGGGCTGCTCC (SEQ ID NO:3), wherein X₁₂X₁₃ in the additional RU is NG, HG, KG, or RG for recognition of the T.

5. The recombinant polypeptide of claim 1, wherein the RUs are ordered from N-terminus to the C-terminus to bind to the sequence: GGTGGGGCTGCTCC (SEQ ID NO:4), wherein the first RU at the N-terminus binds to the G at the 5′ end of the sequence and the last RU at the C-terminus binds to the C at the 3′ end of the sequence.

6. The recombinant polypeptide of claim 5, wherein the DBD comprises at least fourteen RUs, wherein X₁₂X₁₃ in the RUs from N-terminus to C-terminus are NH, NH, NG, NH, NH, NH, NH, HD, NG, NH, HD, NG, HD, and HD.

7. The recombinant polypeptide of claim 5 or 6, wherein the DBD comprises three additional RU at the N-terminus such that the DBD binds to the nucleic acid sequence TGGTGGGGCTGCTCC (SEQ ID NO:5).

8. The recombinant polypeptide of claim 5, wherein the DBD comprises three additional RUs at the C-terminus such that the DBD binds to the sequence GGTGGGGCTGCTCCAGG (SEQ ID NO:6).

9. The recombinant polypeptide of claim 1, wherein the RUs are arranged from N-terminus to C-terminus to bind to the sequence: GCAGATCCCACAGGCGC (SEQ ID NO:7).

10. The recombinant polypeptide of claim 1, wherein the RUs are arranged from N-terminus to C-terminus to bind to the sequence: CCCACAGGCGCCCTGG (SEQ ID NO:8).

11. The recombinant polypeptide of claim 1, wherein the RUs are arranged from N-terminus to C-terminus to bind to the sequence: GGGGCTGCTCCAGGCATGC (SEQ ID NO:9).

12. A recombinant polypeptide comprising:
a DNA binding domain (DBD) and a transcriptional repressor,
the DBD comprising a plurality of repeat units (RUs) ordered from N-terminus to C-terminus of the DBD to bind to a nucleic acid sequence of the PDCD1 gene, wherein the nucleic acid sequence is present within the sequence:
(SEQ ID NO: 10)
CCTCCCCCAGCACTGCCTCTGTCACTCTCGCCCACGTGGATGTGG
wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

13. The recombinant polypeptide of claim 12, wherein the RUs are ordered from N-terminus to C-terminus of the DBD to bind to the nucleic acid sequence TCTGTCACTCTCG (SEQ ID NO: 11).

14. The recombinant polypeptide of claim 13, wherein the DBD comprises at least thirteen RUs, wherein X₁₂X₁₃ in the RUs from N-terminus to C-terminus are NG, HD, NG, NH, NG, HD, NI, HD, NG, HD, NG, HD, and NH.

15. The recombinant polypeptide of claim 13 or 14, wherein the DBD further comprises three additional RUs at the N-terminus such that the DBD binds to the nucleic acid sequence GCCTCTGTCACTCTCG (SEQ ID NO: 12).

16. The recombinant polypeptide of claim 15, wherein the DBD further comprises three additional RUs at the C-terminus such that the DBD binds to the nucleic acid sequence GCCTCTGTCACTCTCGCCC (SEQ ID NO: 13).

17. The recombinant polypeptide of claim 16, wherein the DBD comprises at least nineteen RUs, wherein X₁₂X₁₃ in the RUs from N-terminus to C-terminus are NH, HD, HD, NG, HD, NG, NH, NG, HD, NI, HD, NG, HD, NG, HD, NH, HD, HD, and HD.

18. The recombinant polypeptide of claim 13 or 14, wherein the DBD further comprises five additional RUs at the C-terminus such that the DBD binds to the nucleic acid sequence TCTGTCACTCTCGCCCAC (SEQ ID NO: 14).

19. The recombinant polypeptide of claim 18, wherein the DBD comprises at least eighteen RUs, wherein X₁₂X₁₃ in the RUs from N-terminus to C-terminus are NG, HD, NG, NH, NG, HD, NI, HD, NG, HD, NG, HD, NG, NH, HD, HD, HD, NI, and HD.

20. The recombinant polypeptide of claim 12, wherein the DBD comprises thirteen RUs ordered from N-terminus to C-terminus of the DBD to bind to the nucleic acid sequence:
(SEQ ID NO: 15)
CCCCCAGCACTGC.

21. The recombinant polypeptide of claim 20, wherein the DBD further comprises three additional RUs at the N-terminus such that the DBD binds to the nucleic acid sequence:
(SEQ ID NO: 16)
CCTCCCCCAGCACTGC.

22. The recombinant polypeptide of claim 21, wherein the DBD further comprises an additional RU at the C-terminus such that the DBD binds to the nucleic acid sequence:
(SEQ ID NO: 17)
CCTCCCCCAGCACTGCC.

23. A recombinant polypeptide comprising:
a DNA binding domain (DBD) and a transcriptional repressor,
the DBD comprising at least nine repeat units (RUs) ordered from N-terminus to C-terminus of the DBD to bind to a nucleic acid sequence of the PDCD1 gene, wherein the nucleic acid sequence is present within the sequence:
(SEQ ID NO: 18)
CCCAGGTCAGGTTGAAG,
wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

24. A recombinant polypeptide comprising:
a DNA binding domain (DBD) and a transcriptional repressor,
the DBD comprising at least nine repeat units (RUs) ordered from N-terminus to C-terminus of the DBD to bind to a nucleic acid sequence of the PDCD1 gene, wherein the nucleic acid sequence is present within the sequence:
(SEQ ID NO: 19)
CCCTTCAACCTGACCTGGGACAGTTTCCCTTCCGCTCACCTCCGCCTGA,
wherein each of the RU comprises the sequence X₁₋₁₁X₁₂X₁₃X_{14-33, 34, or 35} (SEQ ID NO: 455), wherein:
X₁₋₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

25. The recombinant polypeptide of claim 24, wherein the DBD comprises ten RUs ordered from N-terminus to C-terminus to bind to the nucleic acid sequence: TCCGCTCACC (SEQ ID NO:20).

26. The recombinant polypeptide of claim 25, wherein the DBD comprises nine additional RUs at the C-terminus such that the DBD binds to the nucleic acid sequence:
(SEQ ID NO: 21)
TCCGCTCACCTCCGCCTGA.

27. The recombinant polypeptide of claim 25, wherein the DBD comprises four additional RUs at the N-terminus such that the DBD binds to the nucleic acid sequence: CCCTTCCGCTCACC (SEQ ID NO:22).

28. The recombinant polypeptide of claim 27, wherein the DBD comprises five additional RUs at the C-terminus such that the DBD binds to the nucleic acid sequence:
(SEQ ID NO: 23)
CCCTTCCGCTCACCTCCGC.

29. The recombinant polypeptide of claim 27, wherein the DBD comprises two additional RUs at the N-terminus such that the DBD binds to the nucleic acid sequence: TTCCCTTCCGCTCACC (SEQ ID NO:24).

30. The recombinant polypeptide of claim 24, wherein the DBD comprises twelve RUs ordered from N-terminus to C-terminus to bind to the nucleic acid sequence: GGGACAGTTTCC (SEQ ID NO:25).

31. The recombinant polypeptide of claim 30, wherein the DBD further comprises four additional RUs at the C-terminus such that the DBD binds to the nucleic acid sequence:
(SEQ ID NO: 26)
GGGACAGTTTCCCTTC.

32. The recombinant polypeptide of claim 30, wherein the DBD further comprises five additional RUs at the N-terminus such that the DBD binds to the nucleic acid sequence:
(SEQ ID NO: 27)
GACCTGGGACAGTTTCC.

33. The recombinant polypeptide of claim 24, wherein the DBD comprises eleven RUs ordered from N-terminus to C-terminus to bind to the nucleic acid sequence: CAACCTGACCT (SEQ ID NO:28).

34. The recombinant polypeptide of claim 33, wherein the DBD comprises nine additional RUs at the C-terminus such that the DBD binds to the nucleic acid sequence:
(SEQ ID NO: 29)
CAACCTGACCTGGGACAGTT.

35. The recombinant polypeptide of claim 33, wherein the DBD comprises five additional RUs at the N-terminus such that the DBD binds to the nucleic acid sequence: CCCTTCAACCTGACCT (SEQ ID NO:30).

36. A recombinant polypeptide comprising:
a DNA binding domain (DBD) and a transcriptional repressor,
the DBD comprising at least nine repeat units (RUs) ordered from N-terminus to C-terminus of the DBD to bind to a nucleic acid sequence of the PDCD1 gene, wherein the nucleic acid sequence is present within the sequence: GCCGCCTTCTCCACTGCTCAGGCGGAGGT (SEQ ID NO:31), wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

37. The recombinant polypeptide of claim 36, wherein the DBD comprises RUs arranged from N-terminus to C-terminus such that the DBD binds to the nucleic acid sequence:
(SEQ ID NO: 32)
GCCGCCTTCTCCACT.

38. The recombinant polypeptide of claim 36, wherein the DBD comprises RUs arranged from N-terminus to C-terminus such that the DBD binds to the nucleic acid sequence:
(SEQ ID NO: 33)
CCACTGCTCAGGCG.

39. The recombinant polypeptide of claim 38, wherein the DBD further comprises three additional RUs at the N-terminus such that the DBD binds to the nucleic acid sequence:
(SEQ ID NO: 34)
TCTCCACTGCTCAGGCG.

40. The recombinant polypeptide of claim 38, wherein the DBD further comprises five additional RUs at the C-terminus such that the DBD binds to the nucleic acid sequence:
(SEQ ID NO: 35)
CCACTGCTCAGGCGGAGGT.

41. A recombinant polypeptide comprising:
a DNA binding domain (DBD) and a transcriptional repressor,
the DBD comprising at least nine repeat units (RUs) ordered from N-terminus to C-terminus of the DBD to bind to a nucleic acid sequence of the PDCD1 gene, wherein the nucleic acid sequence is present within the sequence:
(SEQ ID NO: 36)
GGCCAGGGCGCCTGT;
(SEQ ID NO: 37)
CTGCATGCCTGGAGCAG;
(SEQ ID NO: 38)
GCTCCCGCCCCCTCTTCCT;
(SEQ ID NO: 39)
CTTCCTCCACATCCACG;
or
(SEQ ID NO: 40)
CCTCCACATCCACGTGGGC,
wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of PD1 receptor encoded by the PDCD1 gene.

42. The recombinant polypeptide of any one of claims 1-41, wherein the DBD comprises at least 11 RUs.

43. The recombinant polypeptide of any one of claims 1-41, wherein the DBD comprises at least 13 RUs.

44. The recombinant polypeptide of any one of claims 1-41, wherein the DBD comprises at least 15 RUs.

45. The recombinant polypeptide of any one of claims 1-41, wherein the DBD comprises at least 17 RUs.

46. The recombinant polypeptide of any one of the preceding claims, wherein the DBD comprises up to 40 RUs.

47. The recombinant polypeptide of any one of the preceding claims, wherein the DBD comprises additional RUs at the N-terminus that bind to the nucleotides present upstream of the nucleic acid sequence.

48. The recombinant polypeptide of any one of the preceding claims, wherein the DBD comprises additional RUs at the C-terminus that bind to the nucleotides present downstream of the nucleic acid sequence.

49. A recombinant polypeptide comprising:
a DNA binding domain (DBD) and a transcriptional repressor,
the DBD comprising a plurality of repeat units (RUs) ordered from N-terminus to C-terminus of the DBD to bind to a nucleic acid sequence of the TIM3 gene, wherein the nucleic acid sequence is present within the sequence: GGCAGTGTTACTATAAGAATCACTGGCAATCAGACACCCGGGTG (SEQ ID NO:41) or a complement thereof, wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of TIM3 encoded by the TIM3 gene.

50. The recombinant polypeptide of claim 49, wherein the DBD comprises RUs that bind to the nucleic acid sequence TGTTACTATA (SEQ ID NO:42).

51. The recombinant polypeptide of claim 50, wherein the DBD comprises an additional RU at the C-terminus such that the DBD binds to the nucleic acid sequence TGTTACTATAA (SEQ ID NO:43).

52. The recombinant polypeptide of claim 50 or 51, wherein the DBD comprises three additional RUs at the N-terminus such that the DBD binds to the nucleic acid sequence CAGTGTTACTATAA (SEQ ID NO:44).

53. The recombinant polypeptide of claim 52, wherein the DBD comprises two additional RUs at the N-terminus such that the DBD binds to the nucleic acid sequence GGCAGTGTTACTATAA (SEQ ID NO:45).

54. The recombinant polypeptide of claim 49, wherein the DBD comprises RUs that bind to the nucleic acid sequence TCAGACACCCGGGTG (SEQ ID NO:46).

55. The recombinant polypeptide of claim 54, wherein the DBD comprises three additional RUs at the N-terminus such that the DBD binds to the nucleic acid sequence CAATCAGACACCCGGGTG (SEQ ID NO:47).

56. The recombinant polypeptide of claim 54, wherein the DBD comprises three additional RUs at the N-terminus such that the DBD binds to the nucleic acid sequence TGGCAATCAGACACCCGGGTG (SEQ ID NO:48).

57. A recombinant polypeptide comprising:
a DNA binding domain (DBD) and a transcriptional repressor,
the DBD comprising a plurality of repeat units (RUs) ordered from N-terminus to C-terminus of the DBD to bind a nucleic acid sequence of the TIM3 gene, wherein the nucleic acid sequence is present within the sequence:
(SEQ ID NO: 49)
TGTCTGATTGCCAGTGATTCTTATAGT.
wherein each of the repeat unit comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of TIM3 encoded by the TIM3 gene.

58. The recombinant polypeptide of claim 57, wherein the DBD comprises RUs that are ordered to bind to the sequence TGCCAGTGATT (SEQ ID NO:50).

59. The recombinant polypeptide of claim 58, wherein the DBD comprises eight additional RUs at the C-terminus such that the DBD binds to the sequence TGCCAGTGATTCTTATAGT (SEQ ID NO:51).

60. The recombinant polypeptide of claim 57, wherein the DBD comprises RUs that are ordered to binds to the sequence TGATTGCCAGTGATT (SEQ ID NO:52).

61. The recombinant polypeptide of claim 60, wherein the DBD comprises four additional RUs at the N-terminus such that the DBD binds to the sequence TGTCTGATTGCCAGTGATT (SEQ ID NO:53).

62. A recombinant polypeptide comprising:
a DNA binding domain (DBD) and a transcriptional repressor,
the DBD comprising a plurality of repeat units (RUs) ordered from N-terminus to C-terminus of the DBD to bind to a nucleic acid sequence of TIM3 gene, wherein the nucleic acid sequence is:
TACACACAT (SEQ ID NO:54),
wherein each of the repeat unit comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of TIM3 encoded by the TIM3 gene.

63. The recombinant polypeptide of claim 62, wherein the DBD comprises four additional RUs at the N-terminus such that the DBD binds to the sequence ACACTACACACAT (SEQ ID NO:55).

64. The recombinant polypeptide of claim 63, wherein the DBD comprises four additional RUs at the N-terminus such that the DBD binds to the sequence TGCCACACTACACACAT (SEQ ID NO:56).

65. A recombinant polypeptide comprising:
a DNA binding domain (DBD) and a transcriptional repressor,
the DBD comprising at least nine repeat units (RUs) ordered from N-terminus to C-terminus of the DBD to bind to a nucleic acid sequence of the LAG3 gene, wherein the nucleic acid sequence is present within the sequence: GCCGTTCTGCTGGTCTCTGGGCCTTCACCCCTGTGCCCGGCCTTCC (SEQ ID NO:57), wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of LAG3 encoded by the LAG3 gene.

66. The recombinant polypeptide of claim 65, wherein the DBD comprises RUs that bind to the sequence TCTGCTGGTCT (SEQ ID NO:58).

67. The recombinant polypeptide of claim 66, wherein the DBD comprises five additional RUs at the N-terminus such that the DBD binds to the sequence GCCGTTCTGCTGGTCT (SEQ ID NO:59).

68. The recombinant polypeptide of claim 67, wherein the DBD comprises two additional RUs at the C-terminus such that the DBD binds to the sequence GCCGTTCTGCTGGTCTCT (SEQ ID NO:60).

69. The recombinant polypeptide of claim 66, wherein the DBD comprises four additional RUs at the C-terminus such that the DBD binds to the sequence TCTGCTGGTCTGGGC (SEQ ID NO: 61).

70. The recombinant polypeptide of claim 69, wherein the DBD comprises an additional RUs at the C-terminus such that the DBD binds to the sequence TCTGCTGGTCTGGGCC (SEQ ID NO: 62).

71. The recombinant polypeptide of claim 70, wherein the DBD comprises three additional RUs at the C-terminus such that the DBD binds to the sequence TCTGCTGGTCTGGGCCTTC (SEQ ID NO:63).

72. The recombinant polypeptide of claim 65, wherein the DBD comprises RUs that bind to the sequence TCTCTGGGCCTTCA (SEQ ID NO:64).

73. The recombinant polypeptide of claim 72, wherein the DBD comprises two additional RUs at the N-terminus such that the DBD binds the sequence GGTCTCTGGGCCTTCA (SEQ ID NO:65).

74. The recombinant polypeptide of claim 73, wherein the DBD comprises three additional RUs at the C-terminus such that the DBD binds the sequence GGTCTCTGGGCCTTCACCC (SEQ ID NO:66).

75. The recombinant polypeptide of claim 74, wherein the DBD comprises an additional RUs at the N-terminus such that the DBD binds the sequence TGGTCTCTGGGCCTTCACC (SEQ ID NO:67).

76. The recombinant polypeptide of claim 65, wherein the DBD comprises RUs that bind to the sequence TTCACCCCTGTG (SEQ ID NO:68).

77. The recombinant polypeptide of claim 76, wherein the DBD comprises four additional RUs at the C-terminus such that the DBD binds to the sequence TTCACCCCTGTGCCCG (SEQ ID NO:69).

78. The recombinant polypeptide of claim 77, wherein the DBD comprises four additional RUs at the C-terminus such that the DBD binds to the sequence TTCACCCCTGTGCCCGGCCT (SEQ ID NO:70).

79. The recombinant polypeptide of claim 78, wherein the DBD comprises three additional RUs at the C-terminus such that the DBD binds to the sequence TTCACCCCTGTGCCCGGCCTTCC (SEQ ID NO:71).

80. A recombinant polypeptide comprising:
a DNA binding domain (DBD) and a transcriptional repressor,
the DBD comprising a plurality of repeat units (RUs) ordered from N-terminus to C-terminus of the DBD to bind to a nucleic acid sequence of LAG3 gene, wherein the nucleic acid sequence is:
(SEQ ID NO: 72)
TGCTCTGTCTGC,
wherein each of the repeat unit comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of LAG3 encoded by the LAG3 gene.

81. The recombinant polypeptide of claim 80, wherein the DBD comprises two additional RUs at the C-terminus such that the DBD binds to the sequence TGCTCTGTCTGCTC (SEQ ID NO:73).

82. The recombinant polypeptide of claim 81, wherein the DBD comprises two additional RUs at the N-terminus such that the DBD binds to the sequence TTTGCTCTGTCTGCTC (SEQ ID NO:74).

83. A recombinant polypeptide comprising:
a DNA binding domain (DBD) and a transcriptional repressor,
the DBD comprising at least nine repeat units (RUs) ordered from N-terminus to C-terminus of the DBD to bind to a nucleic acid sequence of the CTLA4 gene, wherein the nucleic acid sequence is:

| | |
|---|---|
| ACATATCTGGGATCAAAGCT, | (SEQ ID NO: 75) |
| ATATAAAGTCCTTGAT, | (SEQ ID NO: 76) |
| or | |
| TTCTATTCAAGTGCC, | (SEQ ID NO: 77) |

wherein each of the RU comprises the sequence X₁₋₁₁X₁₂X₁₃X_{14-33, 34, or 35} (SEQ ID NO: 455), wherein:
X₁₋₁₁ is a chain of 11 contiguous amino acids,
X_{14-33 or 34 or 35} is a chain of 20, 21 or 22 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent, and
wherein the transcriptional repressor domain suppresses expression of CTLA4 encoded by the CTLA4 gene.

84. The recombinant polypeptide of any one of the preceding claims, wherein the DBD comprises up to 40 RUs.

85. The recombinant polypeptide of any one of the preceding claims, wherein the DBD comprises up to 35 RUs.

86. The recombinant polypeptide of any one of the preceding claims, wherein the DBD comprises up to 30 RUs.

87. The recombinant polypeptide of any one of the preceding claims, wherein the DBD comprises up to 25 RUs.

88. The recombinant polypeptide of any one of the preceding claims, wherein the DBD comprises up to 20 RUs.

89. The recombinant polypeptide of any one of the preceding claims, wherein the DBD comprises additional RUs at the N-terminus that bind to the nucleotides present upstream of the nucleic acid sequence.

90. The recombinant polypeptide of any one of the preceding claims, wherein the DBD comprises additional RUs at the C-terminus that bind to the nucleotides present downstream of the nucleic acid sequence.

91. The recombinant polypeptide of any one of the preceding claims, wherein the transcriptional repressor domain is conjugated to the C-terminus of the DBD.

92. The recombinant polypeptide of any one of the preceding claims, wherein the chain of 11 contiguous amino acids is at least 80% identical to LTPDQVVAIAS (SEQ ID NO:78).

93. The recombinant polypeptide of any one of the preceding claims, wherein the chain of 20, 21, or 22 contiguous amino acids is at least 80% identical to GGKQALETVQRLLPVLCQDHG (SEQ ID NO:79).

94. The recombinant polypeptide of any one of the preceding claims, wherein the DBD comprises a N-cap region comprising an amino acid sequence at least 80% identical to the amino acid sequence set for the in SEQ ID NO:339.

95. The recombinant polypeptide of any one of the preceding claims, wherein the DBD comprises a C-cap region comprising an amino acid sequence at least 80% identical to the amino acid sequence set forth in SEQ ID NO: 452, wherein the recombinant polypeptide comprises from N-terminus to C-terminus: the N-cap region, the plurality of RUs, and the C-cap region.

96. The recombinant polypeptide of any one of the preceding claims, wherein the DBD comprises a half-repeat comprising the amino acid sequence X₁₋₁₁X₁₂X₁₃X_{14-19, 20, or 21} (SEQ ID NO: 471), wherein:
X₁₋₁₁ is a chain of 11 contiguous amino acids,
X_{14-20 or 21 or 22} is a chain of 7, 8 or 9 contiguous amino acids,
X₁₂X₁₃ is selected from:
(a) NH, HH, KH, NK, NQ, RH, RN, SS, NN, SN, or KN for recognition of guanine (G);
(b) NI, KI, RI, HI, or SI for recognition of adenine (A);
(c) NG, HG, KG, or RG for recognition of thymine (T);
(d) HD, RD, SD, ND, KD, or YG for recognition of cytosine (C); and
(e) NV or HN for recognition of A or G; and (f) H*, HA, KA, N*, NA, NC, NS, RA, or S* for recognition of A or T or G or C, wherein (*) means that the amino acid at X₁₃ is absent.

97. The recombinant polypeptide of claim 96, wherein X₁₋₁₁ is at least 80% identical to LTPEQVVAIAS (SEQ ID NO:458).

98. The recombinant polypeptide of claim 96 or 97, wherein X_{14-20 or 21 or 22} is at least 80% identical to GGRPALE (SEQ ID NO:472).

99. A nucleic acid encoding the recombinant polypeptide of any of claims 1-98.

100. The nucleic acid of claim 99, wherein the nucleic acid is operably linked to a promoter sequence that confers expression of the polypeptide.

101. The nucleic acid of claim 99 or 100, wherein the sequence of the nucleic acid is codon optimized for expression of the polypeptide in a human cell.

102. The nucleic acid of any one of claims 99-101, wherein the nucleic acid is a deoxyribonucleic acid (DNA).

103. The nucleic acid of any one of claims 99-101, wherein the nucleic acid is a ribonucleic acid (RNA).

104. A vector comprising the nucleic acid of any of claims 99-103.

105. The vector of claim 104, wherein the vector is a viral vector.

106. A host cell comprising the nucleic acid of any of claims 99-103 or the vector of claim 104 or 105.

107. A host cell that expresses the polypeptide of any of claims 1-98.

108. A pharmaceutical composition comprising the polypeptide of any of claims 1-98 and a pharmaceutically acceptable excipient.

109. A pharmaceutical composition comprising the nucleic acid of any of claims 99-103 or the vector of claim 104 or 105 and a pharmaceutically acceptable excipient.

110. A method of suppressing expression of PDCD-1 gene in a cell, the method comprising:
introducing into the cell the recombinant polypeptide of any one of claims 1-48,
wherein the recombinant polypeptide binds to a target nucleic acid sequence present in the PDCD-1 gene and the transcriptional repressor domain suppresses expression of the PDCD-1 gene.

111. A method of suppressing expression of TIM3 gene in a cell, the method comprising:
introducing into the cell the recombinant polypeptide of any one of claims 49-64,
wherein the recombinant polypeptide binds to a target nucleic acid sequence present in the TIM3 gene and the transcriptional repressor domain suppresses expression of the TIM3 gene.

112. A method of suppressing expression of LAG3 gene in a cell, the method comprising:
introducing into the cell the recombinant polypeptide of any one of claims 65-82,
wherein the recombinant polypeptide binds to a target nucleic acid sequence present in the LAG3 gene and the transcriptional repressor domain suppresses expression of the LAG3 gene.

113. A method of suppressing expression of CTLA4 gene in a cell, the method comprising:
introducing into the cell the recombinant polypeptide of any one of claim 83,
wherein the recombinant polypeptide binds to a target nucleic acid sequence present in the CTLA4 gene and the transcriptional repressor domain suppresses expression of the CTLA4 gene.

114. The method of any one of claims 110-113, wherein the polypeptide is introduced as a nucleic acid encoding the polypeptide.

115. The method of claim 114, wherein the nucleic acid is a deoxyribonucleic acid (DNA).

116. The method of claim 114, wherein the nucleic acid is a ribonucleic acid (RNA).

117. The method of any of claims 110-116, wherein the sequence of the nucleic acid is codon optimized for expression in a human cell.

118. The method of any of claims 110-116, wherein the transcriptional repressor domain comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

119. The method of any one of claims 110-118, wherein the cell is an animal cell.

120. The method of any one of claims 110-118, wherein the cell is a human cell.

121. The method of any one of claims 110-120, wherein the cell is a cancer cell.

122. The method of any one of claims 110-121, wherein the cell is an ex vivo cell.

123. The method of any one of claims 110-121, wherein the introducing comprises administering the polypeptide or a nucleic acid encoding the polypeptide to a subject.

124. The method of claim 123, wherein the administering comprises parenteral administration.

125. The method of claim 123, wherein the administering comprises intravenous, intramuscular, intrathecal, or subcutaneous administration.

126. The method of claim 123, wherein the administering comprises direct injection into a site in a subject.

127. The method of any of claim 123, wherein the administering comprises direct injection into a tumor.

128. A recombinant polypeptide comprising a DNA binding domain and a transcriptional repressor domain, wherein the DNA binding domain and the transcriptional repressor domain are heterologous, wherein the transcriptional repressor domain comprises an amino acid sequence at least 80% identical to any one of the sequences set out in SEQ ID NOs: 84-101.

129. The recombinant polypeptide of claim 128, wherein the transcriptional repressor domain comprises an amino acid sequence at least 85% identical to any one of the sequences set out in SEQ ID NOs: 84-101.

130. The recombinant polypeptide of claim 128, wherein the transcriptional repressor domain comprises an amino acid sequence at least 90% identical to any one of the sequences set out in SEQ ID NOs: 84-101.

131. The recombinant polypeptide of claim 128, wherein the transcriptional repressor domain comprises an amino acid sequence at least 95% identical to any one of the sequences set out in SEQ ID NOs: 84-101.

132. The recombinant polypeptide of any one of claims 128-131, wherein the DNA binding domain comprises zinc finger protein (ZFP), a transcription activator-like effector (TALE), or a guide RNA.

133. The recombinant polypeptide of any one of claims 128-132, wherein the DNA binding domain binds to a target nucleic acid sequence in a gene and optionally, wherein the DNA binding domain is the DBD of any one of claims 1-98.

134. The recombinant polypeptide of claim 133, wherein the target nucleic acid sequence is in a PDCD 1 gene, a CTLA4 gene, a LAG3 gene, a TET2 gene, a ETLA gene, a HAVCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.

135. A nucleic acid encoding the recombinant polypeptide of any of claims 128-134.

136. The nucleic acid of claim 135, wherein the nucleic acid is operably linked to a promoter sequence that confers expression of the polypeptide.

137. The nucleic acid of claim 135 or 136, wherein the sequence of the nucleic acid is codon optimized for expression of the polypeptide in a human cell.

138. The nucleic acid of any one of claims 135-137, wherein the nucleic acid is a deoxyribonucleic acid (DNA).

139. The nucleic acid of any one of claims 135-137, wherein the nucleic acid is a ribonucleic acid (RNA).

140. A vector comprising the nucleic acid of any of claims 135-138.

141. The vector of claim 140, wherein the vector is a viral vector.

142. A host cell comprising the nucleic acid of any of claims 135-139 or the vector of claim 140 or 141.

143. A host cell comprising the polypeptide of any of claims 128-134.

144. A host cell that expresses the polypeptide of any of claims 128-134.

145. A pharmaceutical composition comprising the polypeptide of any of claims 128-134 and a pharmaceutically acceptable excipient.

146. A pharmaceutical composition comprising the nucleic acid of any of claims 135-139 or the vector of claim 140 or 141 and a pharmaceutically acceptable excipient.

147. A method of suppressing expression of an endogenous gene in a cell, the method comprising:
introducing into the cell the recombinant polypeptide of any one of claims 128-134,
wherein the DBD of the polypeptide binds to a target nucleic acid sequence present in the endogenous gene and the heterologous transcriptional repressor domain suppresses expression of the endogenous gene.

148. The method of claim 147, wherein the recombinant polypeptide is introduced as a nucleic acid encoding the polypeptide.

149. The method of claim 148, wherein the nucleic acid is a deoxyribonucleic acid (DNA).

150. The method of claim 148, wherein the nucleic acid is a ribonucleic acid (RNA).

151. The method of any of claims 148-150, wherein the sequence of the nucleic acid is codon optimized for expression in a human cell.

152. The method of any of claims 147-151, wherein the gene is a PDCD 1 gene, a CTLA4 gene, a LAG3 gene, a TET2 gene, a ETLA gene, a HAVCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

153. The method of any one of claims 147-152, wherein the cell is an animal cell.

154. The method of any one of claims 147-152, wherein the cell is a human cell.

155. The method of any one of claims 147-152, wherein the cell is a cancer cell.

156. The method of any one of claims 147-152, wherein the cell is an ex vivo cell.

157. The method of any one of claims 147-155, wherein the introducing comprises administering the polypeptide or a nucleic acid encoding the polypeptide to a subject.

158. The method of claim 157, wherein the administering comprises parenteral administration.

159. The method of claim 157, wherein the administering comprises intravenous, intramuscular, intrathecal, or subcutaneous administration.

160. The method of claim 157, wherein the administering comprises direct injection into a site in a subject.

161. The method of any of claim 157, wherein the administering comprises direct injection into a tumor.

162. A plurality of nucleic acids encoding:
(i) polypeptides that dimerize via direct dimerization, comprising:
(A) a DNA binding domain (DBD) fused to a first member of a heterodimer pair and a functional domain fused to a second member of the heterodimer pair, or
(B) a DNA binding domain (DBD) fused to a second member of a heterodimer pair and a functional domain fused to a first member of the heterodimer pair,
wherein the first and second members of the heterodimer pair bind to each other thereby directly dimerizing the DBD and the functional domain,
wherein the heterodimer pair is selected from one of the following heterodimer pairs:
37A, 37B;
13A, 13B;
DHD37-BBB-A, DHD37-BBB-B;
DHD150-A, DHD150-B;
DHD154-A, DHD-154B;
37A, 9B;
13A, 37B;
13A, DHD150-B;
37A, DHD37-BBB-B; and
DHD37-BBB-A, 37B; or
(ii) polypeptides that dimerize indirectly via a bridging construct, comprising:
(A) a DNA binding domain (DBD) fused to a first member of a first heterodimer pair;
a bridging construct comprising a second member of the first heterodimer pair fused to a first member of a second heterodimer pair; and
a functional domain fused to a second member of the second heterodimer pair; or
(B) a DNA binding domain (DBD) fused to a second member of a first heterodimer pair;
a bridging construct comprising a first member of the first heterodimer pair fused to a first member of a second heterodimer pair; and
a functional domain fused to a second member of the second heterodimer pair; or
(C) a DNA binding domain (DBD) fused to a second member of a first heterodimer pair;
a bridging construct comprising a first member of the first heterodimer pair fused to a second member of a second heterodimer pair; and
a functional domain fused to a first member of the second heterodimer pair,
wherein the DBD and the functional domain dimerize indirectly via the bridging construct,
wherein the first and second heterodimer pairs are different and are selected from the following heterodimer pairs:
37A, 37B;
13A, 13B;
DHD37-BBB-A, DHD37-BBB-B;
DHD150-A, DHD150-B;
DHD154-A, DHD-154B;
37A, 9B;
13A, 37B;
13A, DHD150-B;
37A, DHD37-BBB-B; and
DHD37-BBB-A, 37B.

163. The plurality of nucleic acids of claim 162, wherein the DBD in (i) (A) or (i) (B) is fused to a first member of a first heterodimer pair and the functional domain is a first functional domain fused a second member of the first heterodimer pair and to a first member of a second heterodimer pair, the system further comprising a second functional domain fused to a second member of the second heterodimer pair, wherein the members of the first heterodimer pair mediate dimerization of the DBD and the first functional domain and members of the second heterodimer pair mediate dimerization of the first functional domain and the second functional domain.

164. The plurality of nucleic acids of claim 163, wherein the DBD is fused to a first member of a first heterodimer pair and to a first member of a second heterodimer pair, and the functional domain is fused a second member of the first heterodimer pair the system further comprising a second functional domain fused to a second member of the second heterodimer pair, wherein the members of the first heterodimer pair mediate assembly of the DBD and the first functional domain and members of the second heterodimer pair mediate assembly of the DBD and the second functional domain.

165. The plurality of nucleic acids of any one of claims 162-164, wherein the DBD binds to a target nucleic acid sequence present in an endogenous gene in a cell.

166. The plurality of nucleic acids of any one of claims 162-165, wherein the functional domain comprises an enzyme, a transcriptional activator, a transcriptional repressor, or a DNA nucleotide modifier.

167. The plurality of nucleic acids of claim 166, wherein the enzyme is a nuclease, a DNA modifying protein, or a chromatin modifying protein.

168. The plurality of nucleic acids of claim 167, wherein the nuclease is a cleavage domain or a half-cleavage domain.

169. The plurality of nucleic acids of claim 168, wherein the cleavage domain or half-cleavage domain comprises a type IIS restriction enzyme.

170. The plurality of nucleic acids of claim 169, wherein the type IIS restriction enzyme comprises FokI or Bfil.

171. The plurality of nucleic acids of claim 167, wherein the chromatin modifying protein is lysine-specific histone demethylase 1 (LSD1).

172. The plurality of nucleic acids of claim 166, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

173. The plurality of nucleic acids of claim 168, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, MeCP2, or a transcriptional repressor provided in claims 128-134.

174. The plurality of nucleic acids of claim 166, wherein the DNA nucleotide modifier is adenosine deaminase.

175. The plurality of nucleic acids of any of claims 165-174, wherein the target nucleic acid sequence is within a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

176. The plurality of nucleic acids of any of claims 162-175, wherein the DBD comprises a transcription activator-like effector (TALE).

177. The plurality of nucleic acids of any of claims 162-176, wherein the DBD comprises a DBD as set out in any one of claims 1-98.

178. A DNA binding domain and a functional domain or a DNA binding domain, a functional domain and a bridging construct encoded by the plurality of nucleic acids of nucleic acids of any one of claims 162-177.

179. A DNA binding domain and a functional domain as set forth in claim 162 (i)(A); or (i)(B); or a DNA binding domain, a bridging construct, and a functional domain as set forth in claim 162 (ii)(A), (ii)(B), or (ii)(C).

180. A host cell comprising: (a) nucleic acids encoding the polypeptides as set forth in claim 162 (i)(A) or (i)(B); or (b) nucleic acids encoding the polypeptides as set forth in claim 162 (ii)(A), (ii)(B), or (ii)(C).

181. A host cell comprising: (a) the polypeptides as set forth in claim 162 (i)(A) or (i)(B); or (b) the polypeptides as set forth in claim 162 (ii)(A), (ii)(B), or (ii)(C).

182. A kit comprising:
(a) nucleic acids encoding the polypeptides as set forth in claim 162 (i)(A) or (i)(B); or
(b) nucleic acids encoding the polypeptides as set forth in claim 162 (ii)(A), (ii)(B), or (ii)(C).

183. A kit comprising:
(a) a first vector comprising a nucleic acid encoding the DBD set forth in claim 162 (i)(A); and
(b) a second vector comprising a nucleic acid encoding the functional domain set forth in claim 162 (i)(A); or
(a) a first vector comprising a nucleic acid encoding the DBD set forth in claim 162 (i)(B); and
(b) a second vector comprising a nucleic acid encoding the functional domain set forth in claim 162 (i)(B).

184. A kit comprising:
(a) a first vector comprising a nucleic acid encoding the DBD set forth in claim 162 (ii)(A);
(b) a second vector comprising a nucleic acid encoding the bridging construct set forth in claim 162 (ii)(A); and
(c) a third vector comprising a nucleic acid encoding the functional domain set forth in claim 162 (ii)(A); or
(a) a first vector comprising a nucleic acid encoding the DBD set forth in claim 162 (ii)(B);
(b) a second vector comprising a nucleic acid encoding the bridging construct set forth in claim 162 (ii)(B); and
(c) a third vector comprising a nucleic acid encoding the functional domain set forth in claim 162 (ii)(B); or
(a) a first vector comprising a nucleic acid encoding the DBD set forth in claim 162 (ii)(C);
(b) a second vector comprising a nucleic acid encoding the bridging construct set forth in claim 162 (ii)(C); and
(c) a third vector comprising a nucleic acid encoding the functional domain set forth in claim 162 (ii)(C).

185. A pharmaceutical composition comprising:
(a) nucleic acids encoding the polypeptides as set forth in claim 162 (i)(A) or (i)(B); or
(b) nucleic acids encoding the polypeptides as set forth in claim 162 (ii)(A), (ii)(B), or (ii)(C).

186. A pharmaceutical composition comprising:
(a) a first vector comprising a nucleic acid encoding the DBD set forth in claim 162 (i)(A); and
(b) a second vector comprising a nucleic acid encoding the functional domain set forth in claim 162 (i)(A); or
(a) a first vector comprising a nucleic acid encoding the DBD set forth in claim 162 (i)(B); and
(b) a second vector comprising a nucleic acid encoding the functional domain set forth in claim 162 (i)(B).

187. A pharmaceutical composition comprising:
(a) a first vector comprising a nucleic acid encoding the DBD set forth in claim 162 (ii)(A);
(b) a second vector comprising a nucleic acid encoding the bridging construct set forth in claim 162 (ii)(A); and
(c) a third vector comprising a nucleic acid encoding the functional domain set forth in claim 162 (ii)(A); or
(a) a first vector comprising a nucleic acid encoding the DBD set forth in claim 162 (ii)(B);
(b) a second vector comprising a nucleic acid encoding the bridging construct set forth in claim 162 (ii)(B); and
(c) a third vector comprising a nucleic acid encoding the functional domain set forth in claim 162 (ii)(B); or
(a) a first vector comprising a nucleic acid encoding the DBD set forth in claim 162 (ii)(C);
(b) a second vector comprising a nucleic acid encoding the bridging construct set forth in claim 162 (ii)(C); and
(c) a third vector comprising a nucleic acid encoding the functional domain set forth in claim 162 (ii)(C).

188. A pharmaceutical composition comprising the DBD and a functional domain or a DNA binding domain, a functional domain and a bridging construct of claim 178 and a pharmaceutically acceptable excipient.

189. A pharmaceutical composition comprising the host cell of claim 180 or 181 and a pharmaceutically acceptable excipient.

190. A method for modulating expression from a target gene in a cell, the method comprising:
(i) introducing into the cell a first nucleic acid encoding a DNA binding domain fused to a first member of a heterodimer pair and a second nucleic acid encoding a functional domain fused to a second member of the heterodimer pair; or
(ii) introducing into the cell a first nucleic acid encoding a DNA binding domain fused to a second member of a heterodimer pair and a second nucleic acid encoding a functional domain fused to a first member of the heterodimer pair; or
(iii) introducing into the cell a DNA binding domain fused to a first member of a heterodimer pair and a functional domain fused to a second member of the heterodimer pair; or
(iv) introducing into the cell a DNA binding domain fused to a second member of a heterodimer pair and a functional domain fused to a first member of the heterodimer pair,
wherein the heterodimer pair is selected from one of the following heterodimer pairs:
37A, 37B;
13A, 13B;
DHD37-BBB-A, DHD37-BBB-B;
DHD150-A, DHD150-B;
DHD154-A, DHD-154B;
37A, 9B;
13A, 37B;
13A, DHD150-B;
37A, DHD37-BBB-B; and
DHD37-BBB-A, 37B,
wherein the DNA binding domain (DBD) dimerizes with the functional domain via dimerization of the members of the heterodimer pair and wherein binding of the DBD to a target nucleic acid sequence in the target gene results in modulation of expression of the target gene via the functional domain dimerized to the DBD.

191. A method of modulating expression of a target gene in a cell, the method comprising:
(i) introducing into a cell expressing a DNA binding domain (DBD) fused to a first member of a first heterodimer pair and a functional domain fused to a second member of a second heterodimer pair, a bridging construct comprising a second member of the first heterodimer pair fused to a first member of the second heterodimer pair or a nucleic acid encoding the bridging construct; or
(ii) introducing into a cell expressing a DNA binding domain (DBD) fused to a second member of a first heterodimer pair and a functional domain fused to a second member of a second heterodimer pair, a bridging construct comprising a first member of the first heterodimer pair fused to a first member of the second heterodimer pair or a nucleic acid encoding the bridging construct; or
(iii) introducing into a cell expressing a DNA binding domain (DBD) fused to a first member of a first heterodimer pair and a functional domain fused to a first member of a second heterodimer pair, a bridging construct comprising a second member of the first heterodimer pair fused to a second member of the second heterodimer pair or a nucleic acid encoding the bridging construct,
wherein the DBD and the functional domain dimerize indirectly via the bridging construct,
wherein binding of the DBD to a target nucleic acid sequence in a target gene in the cell results in in modulation of expression of the target gene via the functional domain dimerized to the DBD via the bridging construct,
wherein the first and second heterodimer pairs are different and are selected from the following heterodimer pairs:
37A, 37B;
13A, 13B;
DHD37-BBB-A, DHD37-BBB-B;
DHD150-A, DHD150-B;
DHD154-A, DHD-154B;
37A, 9B;
13A, 37B;
13A, DHD150-B;
37A, DHD37-BBB-B; and
DHD37-BBB-A, 37B.

192. A method of reversing modulation of expression of a target gene in a cell expressing a DNA binding domain (DBD) fused to a first member of a non-cognate heterodimer pair and a functional domain fused to a second member of the non-cognate heterodimer pair, wherein the DBD binds to a target nucleic acid sequence in a target gene and the functional domain dimerized to the DBD via dimerization of the members of the heterodimer pair modulates expression of the target gene,
the method comprising introducing into the cell a disruptor which binds to either the first member or the second member with a higher binding affinity than the binding affinity between the first and second members, wherein non-cognate heterodimer pairs and the corresponding disruptor are selected from one of the following combinations:

| Combination | Non-Cognate Heterodimer Pair | Disruptor |
|---|---|---|
| 1 | 37A, 9B; | 37B or 9A |
| 2 | 13A, 37B; | 13B or 37A |
| 3 | 13A, DHD150-B; | 13B or DHD150-A |
| 4 | 37A, DHD37-BBB-B; | 37B or DHD37-BBB-A |
| 5 | DHD37-BBB-A, 37B | DHD37-BBB-B or 37A |

193. The method of any one of claims 190-192, wherein the functional domain comprises an enzyme, a transcriptional activator, a transcriptional repressor, or a DNA nucleotide modifier.

194. The method of any one of claims 190-193, wherein the target nucleic acid sequence is within a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

195. The method of any one of claims 190-194, wherein the DBD comprises a transcription activator-like effector (TALE).


SEQUENCE LISTING (483 sequences)

SEQ ID NO: 1 - DNA, 43 bp, Artificial sequence
  synthetic sequence
tggtggggctgctccaggcatgcagatcccacaggcgccctgg

SEQ ID NO: 2 - DNA, 11 bp, Artificial sequence
  synthetic sequence
ggggctgctcc

SEQ ID NO: 3 - DNA, 12 bp, Artificial sequence
  synthetic sequence
tggggctgctcc

SEQ ID NO: 4 - DNA, 14 bp, Artificial sequence
  synthetic sequence
ggtggggctgctcc

SEQ ID NO: 5 - DNA, 15 bp, Artificial sequence
  synthetic sequence
tggtggggctgctcc

SEQ ID NO: 6 - DNA, 17 bp, Artificial sequence
  synthetic sequence
ggtggggctgctccagg

SEQ ID NO: 7 - DNA, 17 bp, Artificial sequence
  synthetic sequence
gcagatcccacaggcgc

SEQ ID NO: 8 - DNA, 16 bp, Artificial sequence
  synthetic sequence
cccacaggcgccctgg

SEQ ID NO: 9 - DNA, 19 bp, Artificial sequence
  synthetic sequence
ggggctgctccaggcatgc

SEQ ID NO: 10 - DNA, 45 bp, Artificial sequence
  synthetic sequence
cctcccccagcactgcctctgtcactctcgcccacgtggatgtgg

SEQ ID NO: 11 - DNA, 13 bp, Artificial sequence
  synthetic sequence
tctgtcactctcg

SEQ ID NO: 12 - DNA, 16 bp, Artificial sequence
  synthetic sequence
gcctctgtcactctcg

SEQ ID NO: 13 - DNA, 19 bp, Artificial sequence
  synthetic sequence
gcctctgtcactctcgccc

SEQ ID NO: 14 - DNA, 18 bp, Artificial sequence
  synthetic sequence
tctgtcactctcgcccac

SEQ ID NO: 15 - DNA, 13 bp, Artificial sequence
  synthetic sequence
cccccagcactgc

SEQ ID NO: 16 - DNA, 16 bp, Artificial sequence
  synthetic sequence
cctcccccagcactgc

SEQ ID NO: 17 - DNA, 17 bp, Artificial sequence
  synthetic sequence
cctcccccagcactgcc

SEQ ID NO: 18 - DNA, 17 bp, Artificial sequence
  synthetic sequence
cccaggtcaggttgaag

SEQ ID NO: 19 - DNA, 49 bp, Artificial sequence
  synthetic sequence
cccttcaacctgacctgggacagtttcccttccgctcacctccgcctga

SEQ ID NO: 20 - DNA, 10 bp, Artificial sequence
  synthetic sequence
tccgctcacc

SEQ ID NO: 21 - DNA, 19 bp, Artificial sequence
  synthetic sequence
tccgctcacctccgcctga

SEQ ID NO: 22 - DNA, 14 bp, Artificial sequence
  synthetic sequence
cccttccgctcacc

SEQ ID NO: 23 - DNA, 19 bp, Artificial sequence
  synthetic sequence
cccttccgctcacctccgc

SEQ ID NO: 24 - DNA, 16 bp, Artificial sequence
  synthetic sequence
ttcccttccgctcacc

SEQ ID NO: 25 - DNA, 12 bp, Artificial sequence
  synthetic sequence
gggacagtttcc

SEQ ID NO: 26 - DNA, 16 bp, Artificial sequence
  synthetic sequence
gggacagtttcccttc

SEQ ID NO: 27 - DNA, 17 bp, Artificial sequence
  synthetic sequence
gacctgggacagtttcc

SEQ ID NO: 28 - DNA, 11 bp, Artificial sequence
  synthetic sequence
caacctgacct

SEQ ID NO: 29 - DNA, 20 bp, Artificial sequence
  synthetic sequence
caacctgacctgggacagtt

SEQ ID NO: 30 - DNA, 16 bp, Artificial sequence
  synthetic sequence
cccttcaacctgacct

SEQ ID NO: 31 - DNA, 29 bp, Artificial sequence
  synthetic sequence
gccgccttctccactgctcaggcggaggt

SEQ ID NO: 32 - DNA, 15 bp, Artificial sequence
  synthetic sequence
gccgccttctccact

SEQ ID NO: 33 - DNA, 14 bp, Artificial sequence
  synthetic sequence
ccactgctcaggcg

SEQ ID NO: 34 - DNA, 17 bp, Artificial sequence
  synthetic sequence
tctccactgctcaggcg

SEQ ID NO: 35 - DNA, 19 bp, Artificial sequence
  synthetic sequence
ccactgctcaggcggaggt

SEQ ID NO: 36 - DNA, 15 bp, Artificial sequence
  synthetic sequence
ggccagggcgcctgt

SEQ ID NO: 37 - DNA, 17 bp, Artificial sequence
  synthetic sequence
ctgcatgcctggagcag

SEQ ID NO: 38 - DNA, 19 bp, Artificial sequence
  synthetic sequence
gctcccgccccctcttcct

SEQ ID NO: 39 - DNA, 17 bp, Artificial sequence
  synthetic sequence
cttcctccacatccacg

SEQ ID NO: 40 - DNA, 19 bp, Artificial sequence
  synthetic sequence
cctccacatccacgtgggc

SEQ ID NO: 41 - DNA, 44 bp, Artificial sequence
  synthetic sequence
ggcagtgttactataagaatcactggcaatcagacacccgggtg

SEQ ID NO: 42 - DNA, 10 bp, Artificial sequence
  synthetic sequence
tgttactata

SEQ ID NO: 43 - DNA, 11 bp, Artificial sequence
  synthetic sequence
tgttactataa

SEQ ID NO: 44 - DNA, 14 bp, Artificial sequence
  synthetic sequence
cagtgttactataa

SEQ ID NO: 45 - DNA, 16 bp, Artificial sequence
  synthetic sequence
ggcagtgttactataa

SEQ ID NO: 46 - DNA, 15 bp, Artificial sequence
  synthetic sequence
tcagacacccgggtg

SEQ ID NO: 47 - DNA, 18 bp, Artificial sequence
  synthetic sequence
caatcagacacccgggtg

SEQ ID NO: 48 - DNA, 21 bp, Artificial sequence
  synthetic sequence
tggcaatcagacacccgggtg

SEQ ID NO: 49 - DNA, 27 bp, Artificial sequence
  synthetic sequence
tgtctgattgccagtgattcttatagt

SEQ ID NO: 50 - DNA, 11 bp, Artificial sequence
  synthetic sequence
tgccagtgatt

SEQ ID NO: 51 - DNA, 19 bp, Artificial sequence
  synthetic sequence
tgccagtgattcttatagt

SEQ ID NO: 52 - DNA, 15 bp, Artificial sequence
  synthetic sequence
tgattgccagtgatt

SEQ ID NO: 53 - DNA, 19 bp, Artificial sequence
  synthetic sequence
tgtctgattgccagtgatt

SEQ ID NO: 54 - DNA, 9 bp, Artificial sequence
  synthetic sequence
tacacacat

SEQ ID NO: 55 - DNA, 13 bp, Artificial sequence
  synthetic sequence
acactacacacat

SEQ ID NO: 56 - DNA, 17 bp, Artificial sequence
  synthetic sequence
tgccacactacacacat

SEQ ID NO: 57 - DNA, 46 bp, Artificial sequence
  synthetic sequence
gccgttctgctggtctctgggccttcacccctgtgcccggccttcc

SEQ ID NO: 58 - DNA, 11 bp, Artificial sequence
  synthetic sequence
tctgctggtct

SEQ ID NO: 59 - DNA, 16 bp, Artificial sequence
  synthetic sequence
gccgttctgctggtct

SEQ ID NO: 60 - DNA, 18 bp, Artificial sequence
  synthetic sequence
gccgttctgctggtctct

SEQ ID NO: 61 - DNA, 15 bp, Artificial sequence
  synthetic sequence
tctgctggtctgggc

SEQ ID NO: 62 - DNA, 16 bp, Artificial sequence
  synthetic sequence
tctgctggtctgggcc

SEQ ID NO: 63 - DNA, 19 bp, Artificial sequence
  synthetic sequence
tctgctggtctgggccttc

SEQ ID NO: 64 - DNA, 14 bp, Artificial sequence
  synthetic sequence
tctctgggccttca

SEQ ID NO: 65 - DNA, 16 bp, Artificial sequence
  synthetic sequence
ggtctctgggccttca

SEQ ID NO: 66 - DNA, 19 bp, Artificial sequence
  synthetic sequence
ggtctctgggccttcaccc

SEQ ID NO: 67 - DNA, 19 bp, Artificial sequence
  synthetic sequence
tggtctctgggccttcacc

SEQ ID NO: 68 - DNA, 12 bp, Artificial sequence
  synthetic sequence
ttcacccctgtg

SEQ ID NO: 69 - DNA, 16 bp, Artificial sequence
  synthetic sequence
ttcacccctgtgcccg

SEQ ID NO: 70 - DNA, 20 bp, Artificial sequence
  synthetic sequence
ttcacccctgtgcccggcct

SEQ ID NO: 71 - DNA, 23 bp, Artificial sequence
  synthetic sequence
ttcacccctgtgcccggccttcc

SEQ ID NO: 72 - DNA, 12 bp, Artificial sequence
  synthetic sequence
tgctctgtctgc

SEQ ID NO: 73 - DNA, 14 bp, Artificial sequence
  synthetic sequence
tgctctgtctgctc

SEQ ID NO: 74 - DNA, 16 bp, Artificial sequence
  synthetic sequence
tttgctctgtctgctc

SEQ ID NO: 75 - DNA, 20 bp, Artificial sequence
  synthetic sequence
acatatctgggatcaaagct

SEQ ID NO: 76 - DNA, 16 bp, Artificial sequence
  synthetic sequence
atataaagtccttgat

SEQ ID NO: 77 - DNA, 15 bp, Artificial sequence
  synthetic sequence
ttctattcaagtgcc

SEQ ID NO: 78 - protein, 11 aa, Artificial sequence
  synthetic sequence
LTPDQVVAIAS

SEQ ID NO: 79 - protein, 21 aa, Artificial sequence
  synthetic sequence
GGKQALETVQRLLPVLCQDHG

SEQ ID NO: 80 - protein, 14 aa, Artificial sequence
  synthetic sequence
LTPEQVVAIASREF

SEQ ID NO: 81 - protein, 346 aa, Artificial sequence
  synthetic sequence
MRAHPGGGRCCPEQEEGESAAGGSGAGGDSAIEQGGQGSALAPSPVSGVRREGARGGGRGRGRWKQAGRGGGVCGRGRGRGRGRGRGRGRGRGRGRPPSGGSGLGGDGGGCGGGGSGGGGAPRREPVPFPSGSAGPGPRGPRATESGKRMSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKKLEEALMADILSRAADTEEMDIEMDSGDEA

SEQ ID NO: 82 - protein, 213 aa, Artificial sequence
  synthetic sequence
MRVRYDSSNQVKGKPDLNTALPVRQTASIFKQPVTKITNHPSNKVKSDPQKAVDQPRQLFWEKKLSGLNAFDIAEELVKTMDLPKGLQGVGPGCTDETLLSAIASALHTSTMPITGQLSAAVEKNPGVWLNTTQPLCKAFMVTDEDIRKQEELVQQVRKRLEEALMADMLAHVEELARDGEAPLDKACAEDDDEEDEEEEEEEPDPDPEMEHV

SEQ ID NO: 83 - protein, 302 aa, Artificial sequence
  synthetic sequence
MASSPKKKRKVEASVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVSEF

SEQ ID NO: 84 - protein, 440 aa, Artificial sequence
  synthetic sequence
MGSSHLLNKGLPLGVRPPIMNGPLHPRPLVALLDGRDCTVEMPILKDVATVAFCDAQSTQEIHEKVLNEAVGALMYHTITLTREDLEKFKALRIIVRIGSGFDNIDIKSAGDLGIAVCNVPAASVEETADSTLCHILNLYRRATWLHQALREGTRVQSVEQIREVASGAARIRGETLGIIGLGRVGQAVALRAKAFGFNVLFYDPYLSDGVERALGLQRVSTLQDLLFHSDCVTLHCGLNEHNHHLINDFTVKQMRQGAFLVNTARGGLVDEKALAQALKEGRIRGAALDVHESEPFSFSQGPLKDAPNLICTPHAAWYSEQASIEMREEAAREIRRAITGRIPDSLKNCVNKDHLTAATHWASMDPAVVHPELNGAAYRYPPGVVGVAPTGIPAAVEGIVPSAMSLSHGLPPVAHPPHAPSPGQTVKPEADRDHASDQL

SEQ ID NO: 85 - protein, 204 aa, Artificial sequence
  synthetic sequence
MESRSVAQAGVQWCDLGSLQAPPPGFTLFSCLSLLSSWDYSSGFSGFCASPIEESHGALISSCNSRTMTDGLVTFRDVAIDFSQEEWECLDPAQRDLYVDVMLENYSNLVSLDLESKTYETKKIFSENDIFEINFSQWEMKDKSKTLGLEASIFRNNWKCKSIFEGLKGHQEGYFSQMIISYEKIPSYRKSKSLTPHQRIHNTE

SEQ ID NO: 86 - protein, 183 aa, Artificial sequence
  synthetic sequence
MESRSVAQAGVQWCDLGSLQAPPPGFTLFSCLSLLSSWDYSSGFSGFCASPIEESHGALISSCNSRTMTDGLVTFRDVAIDFSQEEWECLDPAQRDLYVDVMLENYSNLVSLGYQLTKPDVILRLEKGEEPIFRNNWKCKSIFEGLKGHQEGYFSQMIISYEKIPSYRKSKSLTPHQRIHNTE

SEQ ID NO: 87 - protein, 213 aa, Artificial sequence
  synthetic sequence
MAFRDVAVDFTQDEWRLLSPAQRTLYREVMLENYSNLVSLGISFSKPELITQLEQGKETWREEKKCSPATCPDPEPELYLDPFCPPGFSSQKFPMQHVLCNHPPWIFTCLCAEGNIQPGDPGPGDQEKQQQASEGRPWSDQAEGPEGEGAMPLFGRTKKRTLGAFSRPPQRQPVSSRNGLRGVELEASPAQSGNPEETDKLLKRIEVLGFGTV

SEQ ID NO: 88 - protein, 160 aa, Artificial sequence
  synthetic sequence
MSQGSVTFRDVAIDFSQEEWKWLQPAQRDLYRCVMLENYGHLVSLGLSISKPDVVSLLEQGKEPWLGKREVKRDLFSVSESSGEIKDFSPKNVIYDDSSQYLIMERILSQGPVYSSFKGGWKCKDHTEMLQENQGCIRKVTVSHQEALAQHMNISTVERP

SEQ ID NO: 89 - protein, 163 aa, Artificial sequence
  synthetic sequence
MTKSKEAVTFKDVAVVFSEEELQLLDLAQRKLYRDVMLENFRNVVSVGHQSTPDGLPQLEREEKLWMMKMATQRDNSSGAKNLKEMETLQEVGLRYLPHEELFCSQIWQQITRELIKYQDSVVNIQRTGCQLEKRDDLHYKDEGFSNQSSHLQVHRVHTGEKP

SEQ ID NO: 90 - protein, 506 aa, Artificial sequence
  synthetic sequence
MASRLPTAWSCEPVTFEDVTLGFTPEEWGLLDLKQKSLYREVMLENYRNLVSVEHQLSKPDVVSQLEEAEDFWPVERGIPQDTIPEYPELQLDPKLDPLPAESPLMNIEVVEVLTLNQEVAGPRNAQIQALYAEDGSLSADAPSEQVQQQGKHPGDPEAARQRFRQFRYKDMTGPREALDQLRELCHQWLQPKARSKEQILELLVLEQFLGALPVKLRTWVESQHPENCQEVVALVEGVTWMSEEEVLPAGQPAEGTTCCLEVTAQQEEKQEDAAICPVTVLPEEPVTFQDVAVDFSREEWGLLGPTQRTEYRDVMLETFGHLVSVGWETTLENKELAPNSDIPEEEPAPSLKVQESSRDCALSSTLEDTLQGGVQEVQDTVLKQMESAQEKDLPQKKHFDNRESQANSGALDTNQVSLQKIDNPESQANSGALDTNQVLLHKIPPRKRLRKRDSQVKSMKHNSRVKIHQKSCERQKAKEGNGCRKTFSRSTKQITFIRIHKGSQV

SEQ ID NO: 91 - protein, 738 aa, Artificial sequence
  synthetic sequence
GVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGPGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 92 - protein, 191 aa, Artificial sequence
  synthetic sequence
MGKKTKRTADDDDDEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 93 - protein, 191 aa, Artificial sequence
  synthetic sequence
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 94 - protein, 410 aa, Artificial sequence
  synthetic sequence
MAAVGAEARGAWCVPCLVSLDTLQELCRKEKLTCKSIGITKRNLNNYEVEYLCDYKVVKDMEYYLVKWKGWPDSTNTWEPLQNLKCPLLLQQFSNDKHNYLSQVKKGKAITPKDNNKTLKPAIAEYIVKKAKQRIALQRWQDELNRRKNHKGMIFVENTVDLEGPPSDFYYINEYKPAPGISLVNEATFGCSCTDCFFQKCCPAEAGVLLAYNKNQQIKIPPGTPIYECNSRCQCGPDCPNRIVQKGTQYSLCIFRTSNGRGWGVKTLVKIKRMSFVMEYVGEVITSEEAERRGQFYDNKGITYLFDLDYESDEFTVDAARYGNVSHFVNHSCDPNLQVFNVFIDNLDTRLPRIALFSTRTINAGEELTFDYQMKGSGDISSDSIDHSPAKKRVRTVCKCGAVTCRGYLN

SEQ ID NO: 95 - protein, 296 aa, Artificial sequence
  synthetic sequence
MNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 96 - protein, 715 aa, Artificial sequence
  synthetic sequence
RSKSEDMDNVQSKRRRYMEEEYEAEFQVKITAKGDINQKLQKVIQWLLEEKLCALQCAVFDKTLAELKTRVEKIECNKRHKTVLTELQAKIARLTKRFEAAKEDLKKRHEHPPNPPVSPGKTVNDVNSNNNMSYRNAGTVRQMLESKRNVSESAPPSFQTPVNTVSSTNLVTPPAVVSSQPKLQTPVTSGSLTATSVLPAPNTATVVATTQVPSGNPQPTISLQPLPVILHVPVAVSSQPQLLQSHPGTLVTNQPSGNVEFISVQSPPTVSGLTKNPVSLPSLPNPTKPNNVPSVPSPSIQRNPTASAAPLGTTLAVQAVPTAHSIVQATRTSLPTVGPSGLYSPSTNRGPIQMKIPISAFSTSSAAEQNSNTTPRIENQTNKTIDASVSKKAADSTSQCGKATGSDSSGVIDLTMDDEESGASQDPKKLNHTPVSTMSSSQPVSRPLQPIQPAPPLQPSGVPTSGPSQTTIHLLPTAPTTVNVTHRPVTQVTTRLPVPRAPANHQVVYTTLPAPPAQAPLRGTVMQAPAVRQVNPQNSVTVRVPQTTTYVVNNGLTLGSTGPQLTVHHRPPQVHTEPPRPVHPAPLPEAPQPQRLPPEAASTSLPQKPHLKLARVQSQNGIVLSWSVLEVDRSCATVDSYHLYAYHEEPSATVPSQWKKIGEVKALPLPMACTLTQFVSGSKYYFAVRAKDIYGRFGPFCDPQSTDVISSTQSS

SEQ ID NO: 97 - protein, 520 aa, Artificial sequence
  synthetic sequence
IRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSSRHWALVSEEELSLLAQNKQSSKLAAKWPTKLVKNCFLPLREYFKYFSTELTSSL

SEQ ID NO: 98 - protein, 853 aa, Artificial sequence
  synthetic sequence
MKGDTRHLNGEEDAGGREDSILVNGACSDQSSDSPPILEAIRTPEIRGRRSSSRLSKREVSSLLSYTQDLTGDGDGEDGDGSDTPVMPKLFRETRTRSESPAVRTRNNNSVSSRERHRPSPRSTRGRQGRNHVDESPVEFPATRSLRRRATASAGTPWPSPPSSYLTIDLTDDTEDTHGTPQSSSTPYARLAQDSQQGGMESPQVEADSGDGDSSEYQDGKEFGIGDLVWGKIKGFSWWPAMVVSWKATSKRQAMSGMRWVQWFGDGKFSEVSADKLVALGLFSQHFNLATFNKLVSYRKAMYHALEKARVRAGKTFPSSPGDSLEDQLKPMLEWAHGGFKPTGIEGLKPNNTQPVVNKSKVRRAGSRKLESRKYENKTRRRTADDSATSDYCPAPKRLKTNCYNNGKDRGDEDQSREQMASDVANNKSSLEDGCLSCGRKNPVSFHPLFEGGLCQTCRDRFLELFYMYDDDGYQSYCTVCCEGRELLLCSNTSCCRCFCVECLEVLVGTGTAAEAKLQEPWSCYMCLPQRCHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 99 - protein, 78 aa, Artificial sequence
  synthetic sequence
SQGRVTFEDVTVNFTQGEWQRLNPEQRNLYRDVMLENYSNLVSVGQGETTKPDVILRLEQGKEPWLEEEEVLGSGRAE

SEQ ID NO: 100 - protein, 108 aa, Artificial sequence
  synthetic sequence
SQELVTFEDVSMDFSQEEWELLEPAQKNLYREVMLENYRNVVSLEALKNQCTDVGIKEGPLSPAQTSQVTSLSSWTGYLLFQPVASSHLEQREALWIEEKGTPQASCS

SEQ ID NO: 101 - protein, 91 aa, Artificial sequence
  synthetic sequence
MAFEDVAVYFSQEEWGLLDTAQRALYRRVMLDNFALVASLGLSTSRPRVVIQLERGEEPWVPSGTDTTLSRTTYRRRNPGSWSLTEDRDVS

SEQ ID NO: 102 - protein, 8 aa, Artificial sequence
  synthetic sequence
  misc_feature (1)..(8): Xaa can be any naturally occurring amino acid
XXXXXXXX

SEQ ID NO: 103 - protein, 8 aa, Artificial sequence
  synthetic sequence
  misc_feature (1)..(8): Xaa can be any naturally occurring amino acid
XXXXXXXX

SEQ ID NO: 104 - protein, 8 aa, Artificial sequence
  synthetic sequence
  misc_feature (1)..(8): Xaa can be any naturally occurring amino acid
XXXXXXXX

SEQ ID NO: 105 - DNA, 11 bp, Artificial sequence
  synthetic sequence
ggagcagcccc

SEQ ID NO: 106 - DNA, 19 bp, Artificial sequence
  synthetic sequence
ggagcagccccaccagagt

SEQ ID NO: 107 - protein, 137 aa, Artificial sequence
  synthetic sequence
MVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 108 - protein, 278 aa, Artificial sequence
  synthetic sequence
SIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPTHEGDQRRASSRKRSRSDRAVTGPSAQQSFEVRAPEQRDALHLPLSWRVKRPRTSIGGGLPDPGTPTAADLAASSTVMREQDEDPFAGAADDFPAFNEEELAWLMELLPQ

SEQ ID NO: 109 - protein, 15 aa, Artificial sequence
  synthetic sequence
GGGGGMDAKSLTAWS

SEQ ID NO: 110 - protein, 35 aa, Artificial sequence
  synthetic sequence
LDTEQVVAIASHNGGKQALEAVKADLLDLLGAPYV

SEQ ID NO: 111 - protein, 35 aa, Artificial sequence
  synthetic sequence
LDTEQVVAIASHNGGKQALEAVKADLLDLRGAPYA

SEQ ID NO: 112 - protein, 35 aa, Artificial sequence
  synthetic sequence
LDTEQVVAIASHNGGKQALEAVKADLLELRGAPYA

SEQ ID NO: 113 - protein, 35 aa, Artificial sequence
  synthetic sequence
LDTEQVVAIASHNGGKQALEAVKAHLLDLRGAPYA

SEQ ID NO: 114 - protein, 35 aa, Artificial sequence
  synthetic sequence
LNTEQVVAIASHNGGKQALEAVKADLLDLRGAPYA

SEQ ID NO: 115 - protein, 35 aa, Artificial sequence
  synthetic sequence
LNTEQVVAIASNNGGKQALEAVKTHLLDLRGARYA

SEQ ID NO: 116 - protein, 35 aa, Artificial sequence
  synthetic sequence
LNTEQVVAIASNPGGKQALEAVRALFPDLRAAPYA

SEQ ID NO: 117 - protein, 35 aa, Artificial sequence
  synthetic sequence
LNTEQVVAIASSHGGKQALEAVRALFPDLRAAPYA

SEQ ID NO: 118 - protein, 35 aa, Artificial sequence
  synthetic sequence
LNTEQVVAVASNKGGKQALEAVGAQLLALRAVPYA

SEQ ID NO: 119 - protein, 35 aa, Artificial sequence
  synthetic sequence
LNTEQVVAVASNKGGKQALEAVGAQLLALRAVPYE

SEQ ID NO: 120 - protein, 35 aa, Artificial sequence
  synthetic sequence
LSAAQVVAIASHDGGKQALEAVGTQLVALRAAPYA

SEQ ID NO: 121 - protein, 35 aa, Artificial sequence
  synthetic sequence
LSIAQVVAVASRSGGKQALEAVRAQLLALRAAPYG

SEQ ID NO: 122 - protein, 35 aa, Artificial sequence
  synthetic sequence
LSPEQVVAIASNHGGKQALEAVRALFRGLRAAPYG

SEQ ID NO: 123 - protein, 35 aa, Artificial sequence
  synthetic sequence
LSPEQVVAIASNNGGKQALEAVKAQLLELRAAPYE

SEQ ID NO: 124 - protein, 35 aa, Artificial sequence
  synthetic sequence
LSTAQLVAIASNPGGKQALEAIRALFRELRAAPYA

SEQ ID NO: 125 - protein, 35 aa, Artificial sequence
  synthetic sequence
LSTAQLVAIASNPGGKQALEAVRALFRELRAAPYA

SEQ ID NO: 126 - protein, 35 aa, Artificial sequence
  synthetic sequence
LSTAQLVAIASNPGGKQALEAVRAPFREVRAAPYA

SEQ ID NO: 127 - protein, 35 aa, Artificial sequence
  synthetic sequence
LSTAQLVSIASNPGGKQALEAVRALFRELRAAPYA

SEQ ID NO: 128 - protein, 35 aa, Artificial sequence
  synthetic sequence
LSTAQVAAIASHDGGKQALEAVGTQLVVLRAAPYA

SEQ ID NO: 129 - protein, 35 aa, Artificial sequence
  synthetic sequence
LSTAQVATIASSIGGRQALEALKVQLPVLRAAPYG

SEQ ID NO: 130 - protein, 35 aa, Artificial sequence
  synthetic sequence
LSTAQVATIASSIGGRQALEAVKVQLPVLRAAPYG

SEQ ID NO: 131 - protein, 35 aa, Artificial sequence
  synthetic sequence
LSTAQVVAIAANNGGKQALEAVRALLPVLRVAPYE

SEQ ID NO: 132 - protein, 35 aa, Artificial sequence
  synthetic sequence
LSTAQVVAIAGNGGGKQALEGIGEQLLKLRTAPYG

SEQ ID NO: 133 - protein, 35 aa, Artificial sequence
  synthetic sequence
LSTAQVVAIASHDGGKQALEAAGTQLVALRAAPYA

SEQ ID NO: 134 - protein, 35 aa, Artificial sequence
  synthetic sequence
LSTAQVVAIASHDGGKQALEAVGAQLVELRAAPYA

SEQ ID NO: 135 - protein, 35 aa, Artificial sequence
  synthetic sequence
LSTAQVVAIASHDGGKQALEAVGTQLVALRAAPYA

SEQ ID NO: 136 - protein, 35 aa, Artificial sequence
  synthetic sequence
LSTAQVVAIASHDGGNQALEAVGTQLVALRAAPYA

SEQ ID NO: 137 - protein, 35 aa, Artificial sequence
  synthetic sequence
LSTAQVVAIASHNGGKQALEAVKAQLLDLRGAPYA

SEQ ID NO: 138 - protein, 35 aa, Artificial sequence
  synthetic sequence
LSTAQVVAIASNDGGKQALEEVEAQLLALRAAPYE

SEQ ID NO: 139 - protein, 35 aa, Artificial sequence
  synthetic sequence
LSTAQVVAIASNGGGKQALEGIGEQLLKLRTAPYG

SEQ ID NO: 140 - protein, 35 aa, Artificial sequence
  synthetic sequence
LSTAQVVAIASNGGGKQALEGIGEQLRKLRTAPYG

SEQ ID NO: 141 - protein, 35 aa, Artificial sequence
  synthetic sequence
LSTAQVVAIASNPGGKQALEAVRALFRELRAAPYA

SEQ ID NO: 142 - protein, 35 aa, Artificial sequence
  synthetic sequence
LSTAQVVAIASQNGGKQALEAVKAQLLDLRGAPYA

SEQ ID NO: 143 - protein, 35 aa, Artificial sequence
  synthetic sequence
LSTAQVVAIASSHGGKQALEAVRALFRELRAAPYG

SEQ ID NO: 144 - protein, 35 aa, Artificial sequence
  synthetic sequence
LSTAQVVAIASSNGGKQALEAVWALLPVLRATPYD

SEQ ID NO: 145 - protein, 35 aa, Artificial sequence
  synthetic sequence
LSTAQVVAIATRSGGKQALEAVRAQLLDLRAAPYG

SEQ ID NO: 146 - protein, 35 aa, Artificial sequence
  synthetic sequence
LSTAQVVAVAGRNGGKQALEAVRAQLPALRAAPYG

SEQ ID NO: 147 - protein, 35 aa, Artificial sequence
  synthetic sequence
LSTAQVVAVASSNGGKQALEAVWALLPVLRATPYD

SEQ ID NO: 148 - protein, 35 aa, Artificial sequence
  synthetic sequence
LSTAQVVTIASSNGGKQALEAVWALLPVLRATPYD

SEQ ID NO: 149 - protein, 35 aa, Artificial sequence
  synthetic sequence
LSTEQVVAIAGHDGGKQALEAVGAQLVALRAAPYA

SEQ ID NO: 150 - protein, 35 aa, Artificial sequence
  synthetic sequence
LSTEQVVAIASHDGGKQALEAVGAQLVALLAAPYA

SEQ ID NO: 151 - protein, 35 aa, Artificial sequence
  synthetic sequence
LSTEQVVAIASHDGGKQALEAVGAQLVALRAAPYA

SEQ ID NO: 152 - protein, 35 aa, Artificial sequence
  synthetic sequence
LSTEQVVAIASHDGGKQALEAVGGQLVALRAAPYA

SEQ ID NO: 153 - protein, 35 aa, Artificial sequence
  synthetic sequence
LSTEQVVAIASHDGGKQALEAVGTQLVALRAAPYA

SEQ ID NO: 154 - protein, 35 aa, Artificial sequence
  synthetic sequence
LSTEQVVAIASHDGGKQALEAVGVQLVALRAAPYA

SEQ ID NO: 155 - protein, 35 aa, Artificial sequence
  synthetic sequence
LSTEQVVAIASHDGGKQALEAVVAQLVALRAAPYA

SEQ ID NO: 156 - protein, 35 aa, Artificial sequence
  synthetic sequence
LSTEQVVAIASHDGGKQPLEAVGAQLVALRAAPYA

SEQ ID NO: 157 - protein, 35 aa, Artificial sequence
  synthetic sequence
LSTEQVVAIASHGGGKQVLEGIGEQLLKLRAAPYG

SEQ ID NO: 158 - protein, 35 aa, Artificial sequence
  synthetic sequence
LSTEQVVAIASHKGGKQALEGIGEQLLKLRAAPYG

SEQ ID NO: 159 - protein, 35 aa, Artificial sequence
  synthetic sequence
LSTEQVVAIASHNGGKQALEAVKADLLDLRGAPYA

SEQ ID NO: 160 - protein, 35 aa, Artificial sequence
  synthetic sequence
LSTEQVVAIASHNGGKQALEAVKADLLELRGAPYA

SEQ ID NO: 161 - protein, 35 aa, Artificial sequence
  synthetic sequence
LSTEQVVAIASHNGGKQALEAVKAHLLDLRGAPYA

SEQ ID NO: 162 - protein, 35 aa, Artificial sequence
  synthetic sequence
LSTEQVVAIASHNGGKQALEAVKAHLLDLRGVPYA

SEQ ID NO: 163 - protein, 35 aa, Artificial sequence
  synthetic sequence
LSTEQVVAIASHNGGKQALEAVKAHLLELRGAPYA

SEQ ID NO: 164 - protein, 35 aa, Artificial sequence
  synthetic sequence
LSTEQVVAIASHNGGKQALEAVKAQLLDLRGAPYA

SEQ ID NO: 165 - protein, 35 aa, Artificial sequence
  synthetic sequence
LSTEQVVAIASHNGGKQALEAVKAQLLELRGAPYA

SEQ ID NO: 166 - protein, 35 aa, Artificial sequence
  synthetic sequence
LSTEQVVAIASHNGGKQALEAVKAQLPVLRRAPYG

SEQ ID NO: 167 - protein, 35 aa, Artificial sequence
  synthetic sequence
LSTEQVVAIASHNGGKQALEAVKTQLLELRGAPYA

SEQ ID NO: 168 - protein, 35 aa, Artificial sequence
  synthetic sequence
LSTEQVVAIASHNGGKQALEAVRAQLPALRAAPYG

SEQ ID NO: 169 - protein, 35 aa, Artificial sequence
  synthetic sequence
LSTEQVVAIASHNGSKQALEAVKAQLLDLRGAPYA

SEQ ID NO: 170 - protein, 35 aa, Artificial sequence
  synthetic sequence
LSTEQVVAIASNGGGKQALEGIGKQLQELRAAPHG

SEQ ID NO: 171 - protein, 35 aa, Artificial sequence
  synthetic sequence
LSTEQVVAIASNGGGKQALEGIGKQLQELRAAPYG

SEQ ID NO: 172 - protein, 35 aa, Artificial sequence
  synthetic sequence
LSTEQVVAIASNHGGKQALEAVRALFRELRAAPYA

SEQ ID NO: 173 - protein, 35 aa, Artificial sequence
  synthetic sequence
LSTEQVVAIASNHGGKQALEAVRALFRGLRAAPYG

SEQ ID NO: 174 - protein, 35 aa, Artificial sequence
  synthetic sequence
LSTEQVVAIASNKGGKQALEAVKADLLDLRGAPYV

SEQ ID NO: 175 - protein, 35 aa, Artificial sequence
  synthetic sequence
LSTEQVVAIASNKGGKQALEAVKAHLLDLLGAPYV

SEQ ID NO: 176 - protein, 35 aa, Artificial sequence
  synthetic sequence
LSTEQVVAIASNKGGKQALEAVKAQLLALRAAPYA

SEQ ID NO: 177 - protein, 35 aa, Artificial sequence
  synthetic sequence
LSTEQVVAIASNKGGKQALEAVKAQLLELRGAPYA

SEQ ID NO: 178 - protein, 35 aa, Artificial sequence
  synthetic sequence
LSTEQVVAIASNNGGKQALEAVKALLLELRAAPYE

SEQ ID NO: 179 - protein, 35 aa, Artificial sequence
  synthetic sequence
LSTEQVVAIASNNGGKQALEAVKAQLLALRAAPYE

SEQ ID NO: 180 - protein, 35 aa, Artificial sequence
  synthetic sequence
LSTEQVVAIASNNGGKQALEAVKAQLLDLRGAPYA

SEQ ID NO: 181 - protein, 35 aa, Artificial sequence
  synthetic sequence
LSTEQVVAIASNNGGKQALEAVKAQLLVLRAAPYG

SEQ ID NO: 182 - protein, 35 aa, Artificial sequence
  synthetic sequence
LSTEQVVAIASNNGGKQALEAVKAQLPALRAAPYE

SEQ ID NO: 183 - protein, 35 aa, Artificial sequence
  synthetic sequence
LSTEQVVAIASNNGGKQALEAVKAQLPVLRRAPCG

SEQ ID NO: 184 - protein, 35 aa, Artificial sequence
  synthetic sequence
LSTEQVVAIASNNGGKQALEAVKAQLPVLRRAPYG

SEQ ID NO: 185 - protein, 35 aa, Artificial sequence
  synthetic sequence
LSTEQVVAIASNNGGKQALEAVKARLLDLRGAPYA

SEQ ID NO: 186 - protein, 35 aa, Artificial sequence
  synthetic sequence
LSTEQVVAIASNNGGKQALEAVKTQLLALRTAPYE

SEQ ID NO: 187 - protein, 35 aa, Artificial sequence
  synthetic sequence
LSTEQVVAIASNPGGKQALEAVRALFPDLRAAPYA

SEQ ID NO: 188 - protein, 35 aa, Artificial sequence
  synthetic sequence
LSTEQVVAIASSHGGKQALEAVRALFPDLRAAPYA

SEQ ID NO: 189 - protein, 35 aa, Artificial sequence
  synthetic sequence
LSTEQVVAIASSHGGKQALEAVRALLPVLRATPYD

SEQ ID NO: 190 - protein, 35 aa, Artificial sequence
  synthetic sequence
LSTEQVVAVASHNGGKQALEAVRAQLLDLRAAPYE

SEQ ID NO: 191 - protein, 35 aa, Artificial sequence
  synthetic sequence
LSTEQVVAVASNKGGKQALAAVEAQLLRLRAAPYE

SEQ ID NO: 192 - protein, 35 aa, Artificial sequence
  synthetic sequence
LSTEQVVAVASNKGGKQALEEVEAQLLRLRAAPYE

SEQ ID NO: 193 - protein, 35 aa, Artificial sequence
  synthetic sequence
LSTEQVVAVASNKGGKQVLEAVGAQLLALRAVPYE

SEQ ID NO: 194 - protein, 35 aa, Artificial sequence
  synthetic sequence
LSTEQVVAVASNNGGKQALKAVKAQLLALRAAPYE

SEQ ID NO: 195 - protein, 35 aa, Artificial sequence
  synthetic sequence
LSTEQVVVIANSIGGKQALEAVKVQLPVLRAAPYE

SEQ ID NO: 196 - protein, 35 aa, Artificial sequence
  synthetic sequence
LSTGQVVAIASNGGGRQALEAVREQLLALRAVPYE

SEQ ID NO: 197 - protein, 35 aa, Artificial sequence
  synthetic sequence
LSVAQVVTIASHNGGKQALEAVRAQLLALRAAPYG

SEQ ID NO: 198 - protein, 35 aa, Artificial sequence
  synthetic sequence
LTIAQVVAVASHNGGKQALEAIGAQLLALRAAPYA

SEQ ID NO: 199 - protein, 35 aa, Artificial sequence
  synthetic sequence
LTIAQVVAVASHNGGKQALEVIGAQLLALRAAPYA

SEQ ID NO: 200 - protein, 35 aa, Artificial sequence
  synthetic sequence
LTPQQVVAIAANTGGKQALGAITTQLPILRAAPYE

SEQ ID NO: 201 - protein, 35 aa, Artificial sequence
  synthetic sequence
LTPQQVVAIASNTGGKQALEAVTVQLRVLRGARYG

SEQ ID NO: 202 - protein, 35 aa, Artificial sequence
  synthetic sequence
LTPQQVVAIASNTGGKRALEAVCVQLPVLRAAPYR

SEQ ID NO: 203 - protein, 35 aa, Artificial sequence
  synthetic sequence
LTPQQVVAIASNTGGKRALEAVRVQLPVLRAAPYE

SEQ ID NO: 204 - protein, 35 aa, Artificial sequence
  synthetic sequence
LTTAQVVAIASNDGGKQALEAVGAQLLVLRAVPYE

SEQ ID NO: 205 - protein, 35 aa, Artificial sequence
  synthetic sequence
LTTAQVVAIASNDGGKQTLEVAGAQLLALRAVPYE

SEQ ID NO: 206 - protein, 35 aa, Artificial sequence
  synthetic sequence
LSTAQVVAVASGSGGKPALEAVRAQLLALRAAPYG

SEQ ID NO: 207 - protein, 35 aa, Artificial sequence; identical to SEQ ID NO: 206
  synthetic sequence

SEQ ID NO: 208 - protein, 35 aa, Artificial sequence
  synthetic sequence
LNTAQIVAIASHDGGKPALEAVWAKLPVLRGAPYA

SEQ ID NO: 209 - protein, 35 aa, Artificial sequence
  synthetic sequence
LNTAQVVAIASHDGGKPALEAVRAKLPVLRGVPYA

SEQ ID NO: 210 - protein, 35 aa, Artificial sequence
  synthetic sequence
LNTAQVVAIASHDGGKPALEAVWAKLPVLRGVPYA

SEQ ID NO: 211 - protein, 35 aa, Artificial sequence
  synthetic sequence
LNTAQVVAIASHDGGKPALEAVWAKLPVLRGVPYE

SEQ ID NO: 212 - protein, 35 aa, Artificial sequence
  synthetic sequence
LSTAQVVAIASHDGGKPALEAVWAKLPVLRGAPYA

SEQ ID NO: 213 - protein, 35 aa, Artificial sequence
  synthetic sequence
LSTAQVVAVASHDGGKPALEAVRKQLPVLRGVPHQ

SEQ ID NO: 214 - protein, 35 aa, Artificial sequence; identical to SEQ ID NO: 213
  synthetic sequence

SEQ ID NO: 215 - protein, 35 aa, Artificial sequence; identical to SEQ ID NO: 210
  synthetic sequence

SEQ ID NO: 216 - protein, 35 aa, Artificial sequence
  synthetic sequence
LSTEQVVAIASHNGGKLALEAVKAHLLDLRGAPYA

SEQ ID NO: 217 - protein, 35 aa, Artificial sequence
  synthetic sequence
LSTEQVVAIASHNGGKPALEAVKAHLLALRAAPYA

SEQ ID NO: 218 - protein, 35 aa, Artificial sequence
  synthetic sequence
LNTAQVVAIASHYGGKPALEAVWAKLPVLRGVPYA

SEQ ID NO: 219 - protein, 35 aa, Artificial sequence
  synthetic sequence
LNTEQVVAIASNNGGKPALEAVKAQLLELRAAPYE

SEQ ID NO: 220 - protein, 35 aa, Artificial sequence
  synthetic sequence
LSPEQVVAIASNNGGKPALEAVKALLLALRAAPYE

SEQ ID NO: 221 - protein, 35 aa, Artificial sequence
  synthetic sequence
LSPEQVVAIASNNGGKPALEAVKAQLLELRAAPYE

SEQ ID NO: 222 - protein, 35 aa, Artificial sequence
  synthetic sequence
LSTEQVVAIASNNGGKPALEAVKALLLALRAAPYE

SEQ ID NO: 223 - protein, 35 aa, Artificial sequence
  synthetic sequence
LSTEQVVAIASNNGGKPALEAVKALLLELRAAPYE

SEQ ID NO: 224 - protein, 35 aa, Artificial sequence; identical to SEQ ID NO: 220
  synthetic sequence

SEQ ID NO: 225 - protein, 35 aa, Artificial sequence; identical to SEQ ID NO: 221
  synthetic sequence

SEQ ID NO: 226 - protein, 35 aa, Artificial sequence; identical to SEQ ID NO: 223
  synthetic sequence

SEQ ID NO: 227 - protein, 137 aa, Artificial sequence
  synthetic sequence
FGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLH

SEQ ID NO: 228 - protein, 120 aa, Artificial sequence
  synthetic sequence
KQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLH

SEQ ID NO: 229 - protein, 19 aa, Artificial sequence
  synthetic sequence
LSTAQVVAIACISGQQALE

SEQ ID NO: 230 - protein, 62 aa, Artificial sequence
  synthetic sequence
AIEAHMPTLRQASHSLSPERVAAIACIGGRSAVEAVRQGLPVKAIRRIRREKAPVAGPPPAS

SEQ ID NO: 231 - protein, 115 aa, Artificial sequence
  synthetic sequence
SEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLH

SEQ ID NO: 232 - protein, 33 aa, Artificial sequence
  synthetic sequence
FSSQQIIRMVSHAGGANNLKAVTANHDDLQNMG

SEQ ID NO: 233 - protein, 33 aa, Artificial sequence
  synthetic sequence
FNVEQIVRMVSHNGGSKNLKAVTDNHDDLKNMG

SEQ ID NO: 234 - protein, 33 aa, Artificial sequence
  synthetic sequence
FNAEQIVRMVSHGGGSKNLKAVTDNHDDLKNMG

SEQ ID NO: 235 - protein, 33 aa, Artificial sequence
  synthetic sequence
FNAEQIVSMVSNNGGSKNLKAVTDNHDDLKNMG

SEQ ID NO: 236 - protein, 33 aa, Artificial sequence
  synthetic sequence
FNAEQIVSMVSNGGGSLNLKAVKKYHDALKDRG

SEQ ID NO: 237 - protein, 33 aa, Artificial sequence
  synthetic sequence
FNTEQIVRMVSHDGGSLNLKAVKKYHDALRERK

SEQ ID NO: 238 - protein, 33 aa, Artificial sequence
  synthetic sequence
FNVEQIVSIVSHGGGSLNLKAVKKYHDVLKDRE

SEQ ID NO: 239 - protein, 33 aa, Artificial sequence
  synthetic sequence
FNAEQIVRMVSHDGGSLNLKAVTDNHDDLKNMG

SEQ ID NO: 240 - protein, 33 aa, Artificial sequence
  synthetic sequence
FSAEQIVRIAAHDGGSRNIEAVQQAQHVLKELG

SEQ ID NO: 241 - protein, 33 aa, Artificial sequence
  synthetic sequence
FSAEQIVSIVAHDGGSRNIEAVQQAQHILKELG

SEQ ID NO: 242 - protein, 33 aa, Artificial sequence
  synthetic sequence
LDRQQILRIASHDGGSKNIAAVQKFLPKLMNFG

SEQ ID NO: 243 - protein, 33 aa, Artificial sequence
  synthetic sequence
FSAEQIVRIAAHDGGSLNIDAVQQAQQALKELG

SEQ ID NO: 244 - protein, 33 aa, Artificial sequence
  synthetic sequence
FSTEQIVCIAGHGGGSLNIKAVLLAQQALKDLG

SEQ ID NO: 245 - protein, 33 aa, Artificial sequence
  synthetic sequence
YSSEQIVRVAAHGGGSLNIKAVLQAHQALKELD

SEQ ID NO: 246 - protein, 33 aa, Artificial sequence
  synthetic sequence
FSAEQIVHIAAHGGGSLNIKAILQAHQTLKELN

SEQ ID NO: 247 - protein, 33 aa, Artificial sequence
  synthetic sequence
FSAEQIVRIAAHIGGSRNIEAIQQAHHALKELG

SEQ ID NO: 248 - protein, 33 aa, Artificial sequence
  synthetic sequence
FSAEQIVRIAAHIGGSHNLKAVLQAQQALKELD

SEQ ID NO: 249 - protein, 33 aa, Artificial sequence
  synthetic sequence
FSAKHIVRIAAHIGGSLNIKAVQQAQQALKELG

SEQ ID NO: 250 - protein, 33 aa, Artificial sequence
  synthetic sequence
FNAEQIVRMVSHKGGSKNLALVKEYFPVFSSFH

SEQ ID NO: 251 - protein, 33 aa, Artificial sequence
  synthetic sequence
FSADQIVRIAAHKGGSHNIVAVQQAQQALKELD

SEQ ID NO: 252 - protein, 33 aa, Artificial sequence
  synthetic sequence
FSAEQIVSIAAHVGGSHNIEAVQKAHQALKELD

SEQ ID NO: 253 - protein, 33 aa, Artificial sequence
  synthetic sequence
FSSGETVGATVGAGGTETVAQGGTASNTTVSSG

SEQ ID NO: 254 - protein, 33 aa, Artificial sequence
  synthetic sequence
FSGGMATSTTVGSGGTQDVLAGGAAVGGTVGTG

SEQ ID NO: 255 - protein, 33 aa, Artificial sequence
  synthetic sequence
FSAADIVKIAGKIGGAQALQAFITHRAALIQAG

SEQ ID NO: 256 - protein, 33 aa, Artificial sequence
  synthetic sequence
FNPTDIVKIAGNDGGAQALQAVLELEPALRERG

SEQ ID NO: 257 - protein, 33 aa, Artificial sequence
  synthetic sequence
FNPTDIVRMAGNDGGAQALQAVFELEPAFRERS

SEQ ID NO: 258 - protein, 33 aa, Artificial sequence
  synthetic sequence
FNPTDIVRMAGNDGGAQALQAVLELEPAFRERG

SEQ ID NO: 259 - protein, 33 aa, Artificial sequence
  synthetic sequence
FSQVDIVKIASNDGGAQALYSVLDVEPTFRERG

SEQ ID NO: 260 - protein, 33 aa, Artificial sequence
  synthetic sequence
FSRADIVKIAGNDGGAQALYSVLDVEPPLRERG

SEQ ID NO: 261 - protein, 33 aa, Artificial sequence
  synthetic sequence
FSRGDIVKIAGNDGGAQALYSVLDVEPPLRERG

SEQ ID NO: 262 - protein, 33 aa, Artificial sequence
  synthetic sequence
FNRADIVRIAGNGGGAQALYSVRDAGPTLGKRG

SEQ ID NO: 263 - protein, 33 aa, Artificial sequence
  synthetic sequence
FRQADIVKIASNGGSAQALNAVIKLGPTLRQRG

SEQ ID NO: 264 - protein, 33 aa, Artificial sequence
  synthetic sequence
FRQADIVKMASNGGSAQALNAVIKLGPTLRQRG

SEQ ID NO: 265 - protein, 33 aa, Artificial sequence
  synthetic sequence
FSRADIVKIAGNGGGAQALQAVLELEPTFRERG

SEQ ID NO: 266 - protein, 33 aa, Artificial sequence
  synthetic sequence
FSRADIVRIAGNGGGAQALYSVLDVGPTLGKRG

SEQ ID NO: 267 - protein, 33 aa, Artificial sequence
  synthetic sequence
FSRGDIVRIAGNGGGAQALQAVLELEPTLGERG

SEQ ID NO: 268 - protein, 33 aa, Artificial sequence
  synthetic sequence
FSRADIVKIAGNGGGAQALQAVITHRAALTQAG

SEQ ID NO: 269 - protein, 33 aa, Artificial sequence
  synthetic sequence
FSRGDTVKIAGNIGGAQALQAVLELEPTLRERG

SEQ ID NO: 270 - protein, 33 aa, Artificial sequence
  synthetic sequence
FNPTDIVKIAGNIGGAQALQAVLELEPAFRERG

SEQ ID NO: 271 - protein, 33 aa, Artificial sequence
  synthetic sequence
FSAADIVKIAGNIGGAQALQAIFTHRAALIQAG

SEQ ID NO: 272 - protein, 33 aa, Artificial sequence
  synthetic sequence
FSAADIVKIAGNIGGAQALQAVITHRATLTQAG

SEQ ID NO: 273 - protein, 33 aa, Artificial sequence
  synthetic sequence
FSATDIVKIASNIGGAQALQAVISRRAALIQAG

SEQ ID NO: 274 - protein, 33 aa, Artificial sequence
  synthetic sequence
FSQPDIVKIAGNIGGAQALQAVLELEPAFRERG

SEQ ID NO: 275 - protein, 33 aa, Artificial sequence
  synthetic sequence
FSRADIVKIAGNIGGAQALQAVLELESTFRERS

SEQ ID NO: 276 - protein, 33 aa, Artificial sequence
  synthetic sequence
FSRADIVKIAGNIGGAQALQAVLELESTLRERS

SEQ ID NO: 277 - protein, 33 aa, Artificial sequence
  synthetic sequence
FSRGDIVKMAGNIGGAQALQAGLELEPAFRERG

SEQ ID NO: 278 - protein, 33 aa, Artificial sequence
  synthetic sequence
FSRGDIVKMAGNIGGAQALQAVLELEPAFHERS

SEQ ID NO: 279 - protein, 33 aa, Artificial sequence
  synthetic sequence
FTLTDIVKMAGNIGGAQALKAVLEHGPTLRQRD

SEQ ID NO: 280 - protein, 33 aa, Artificial sequence
  synthetic sequence
FTLTDIVKMAGNIGGAQALKVVLEHGPTLRQRD

SEQ ID NO: 281 - protein, 33 aa, Artificial sequence
  synthetic sequence
FNPTDIVKIAGNNGGAQALQAVLELEPALRERG

SEQ ID NO: 282 - protein, 33 aa, Artificial sequence
  synthetic sequence
FNPTDIVKIAGNNGGAQALQAVLELEPALRERS

SEQ ID NO: 283 - protein, 33 aa, Artificial sequence
  synthetic sequence
FNPTDMVKIAGNNGGAQALQAVLELEPALRERG

SEQ ID NO: 284 - protein, 33 aa, Artificial sequence
  synthetic sequence
FSAADIVKIASNNGGAQALQALIDHWSTLSGKT

SEQ ID NO: 285 - protein, 33 aa, Artificial sequence
  synthetic sequence
FSAADIVKIASNNGGAQALQAVISRRAALIQAG

SEQ ID NO: 286 - protein, 33 aa, Artificial sequence
  synthetic sequence
FSAADIVKIASNNGGAQALQAVITHRAALAQAG

SEQ ID NO: 287 - protein, 33 aa, Artificial sequence
  synthetic sequence
FSAADIVKIASNNGGARALQALIDHWSTLSGKT

SEQ ID NO: 288 - protein, 33 aa, Artificial sequence
  synthetic sequence
FTLTDIVEMAGNNGGAQALKAVLEHGSTLDERG

SEQ ID NO: 289 - protein, 33 aa, Artificial sequence
  synthetic sequence
FTLTDIVKMAGNNGGAQALKAVLEHGPTLDERG

SEQ ID NO: 290 - protein, 33 aa, Artificial sequence
  synthetic sequence
FTLTDIVKMAGNNGGAQALKVVLEHGPTLRQRG

SEQ ID NO: 291 - protein, 33 aa, Artificial sequence
  synthetic sequence
FTLTDIVKMASNNGGAQALKAVLEHGPTLDERG

SEQ ID NO: 292 - protein, 33 aa, Artificial sequence
  synthetic sequence
FSAADIVKIAGNSGGAQALQAVISHRAALTQAG

SEQ ID NO: 293 - protein, 33 aa, Artificial sequence
  synthetic sequence
FSGGDAVSTVVRSGGAQSVASGGTASGTTVSAG

SEQ ID NO: 294 - protein, 33 aa, Artificial sequence
  synthetic sequence
FRQTDIVKMAGSGGSAQALNAVIKHGPTLRQRG

SEQ ID NO: 295 - protein, 33 aa, Artificial sequence
  synthetic sequence
FSLIDIVEIASNGGAQALKAVLKYGPVLTQAGR

SEQ ID NO: 296 - protein, 33 aa, Artificial sequence
  synthetic sequence
FSGGDAAGTVVSSGGAQNVTGGLASGTTVASGG

SEQ ID NO: 297 - protein, 33 aa, Artificial sequence
  synthetic sequence
FNLTDIVEMAANSGGAQALKAVLEHGPTLRQRG

SEQ ID NO: 298 - protein, 33 aa, Artificial sequence
  synthetic sequence
FNRASIVKIAGNSGGAQALQAVLKHGPTLDERG

SEQ ID NO: 299 - protein, 33 aa, Artificial sequence
  synthetic sequence
FSQANIVKMAGNSGGAQALQAVLDLELVFRERG

SEQ ID NO: 300 - protein, 33 aa, Artificial sequence
  synthetic sequence
FSQPDIVKMAGNSGGAQALQAVLDLELAFRERG

SEQ ID NO: 301 - protein, 33 aa, Artificial sequence
  synthetic sequence
FSLIDIVEIASNGGAQALKAVLKYGPVLMQAGR

SEQ ID NO: 302 - protein, 33 aa, Artificial sequence
  synthetic sequence
YKSEDIIRLASHDGGSVNLEAVLRLHSQLTRLG

SEQ ID NO: 303 - protein, 33 aa, Artificial sequence
  synthetic sequence
YKPEDIIRLASHGGGSVNLEAVLRLNPQLIGLG

SEQ ID NO: 304 - protein, 33 aa, Artificial sequence
  synthetic sequence
YKSEDIIRLASHGGGSVNLEAVLRLHSQLTRLG

SEQ ID NO: 305 - protein, 33 aa, Artificial sequence
  synthetic sequence
YKSEDIIRLASHGGGSVNLEAVLRLNPQLIGLG

SEQ ID NO: 306 - protein, 33 aa, Artificial sequence
  synthetic sequence
LGHKELIKIAARNGGGNNLIAVLSCYAKLKEMG

SEQ ID NO: 307 - protein, 33 aa, Artificial sequence
  synthetic sequence
FNLTDIVEMAGKGGGAQALKAVLEHGPTLRQRG

SEQ ID NO: 308 - protein, 33 aa, Artificial sequence
  synthetic sequence
FRQADIIKIAGNDGGAQALQAVIEHGPTLRQHG

SEQ ID NO: 309 - protein, 33 aa, Artificial sequence
  synthetic sequence
FSQADIVKIAGNDGGTQALHAVLDLERMLGERG

SEQ ID NO: 310 - protein, 33 aa, Artificial sequence
  synthetic sequence
FSRADIVKIAGNGGGAQALKAVLEHEATLDERG

SEQ ID NO: 311 - protein, 33 aa, Artificial sequence
  synthetic sequence
FSRADIVRIAGNGGGAQALYSVLDVEPTLGKRG

SEQ ID NO: 312 - protein, 33 aa, Artificial sequence
  synthetic sequence
FSQPDIVKMASNIGGAQALQAVLELEPALRERG

SEQ ID NO: 313 - protein, 33 aa, Artificial sequence
  synthetic sequence
FSQPDIVKMAGNIGGAQALQAVLSLGPALRERG

SEQ ID NO: 314 - protein, 33 aa, Artificial sequence
  synthetic sequence
FSQPEIVKIAGNIGGAQALHTVLELEPTLHKRG

SEQ ID NO: 315 - protein, 33 aa, Artificial sequence
  synthetic sequence
FSQSDIVKIAGNIGGAQALQAVLDLESMLGKRG

SEQ ID NO: 316 - protein, 33 aa, Artificial sequence
  synthetic sequence
FSQSDIVKIAGNIGGAQALQAVLELEPTLRESD

SEQ ID NO: 317 - protein, 33 aa, Artificial sequence
  synthetic sequence
FNPTDIVKIAGNKGGAQALQAVLELEPALRERG

SEQ ID NO: 318 - protein, 33 aa, Artificial sequence
  synthetic sequence
FSPTDIIKIAGNNGGAQALQAVLDLELMLRERG

SEQ ID NO: 319 - protein, 33 aa, Artificial sequence
  synthetic sequence
FSQADIVKIAGNNGGAQALYSVLDVEPTLGKRG

SEQ ID NO: 320 - protein, 33 aa, Artificial sequence
  synthetic sequence
FSRGDIVTIAGNNGGAQALQAVLELEPTLRERG

SEQ ID NO: 321 - protein, 33 aa, Artificial sequence
  synthetic sequence
FSRIDIVKIAANNGGAQALHAVLDLGPTLRECG

SEQ ID NO: 322 - protein, 33 aa, Artificial sequence
  synthetic sequence
FSQADIVKIVGNNGGAQALQAVFELEPTLRERG

SEQ ID NO: 323 - protein, 33 aa, Artificial sequence
  synthetic sequence
FSQPDIVRITGNRGGAQALQAVLALELTLRERG

SEQ ID NO: 324 - protein, 33 aa, Artificial sequence
  synthetic sequence
FKADDAVRIACRTGGSHNLKAVHKNYERLRARG

SEQ ID NO: 325 - protein, 33 aa, Artificial sequence
  synthetic sequence
FNADQVIKIVGHDGGSNNIDVVQQFFPELKAFG

SEQ ID NO: 326 - protein, 33 aa, Artificial sequence
  synthetic sequence
FSAEQIVRIAAHIGGSRNIEATIKHYAMLTQPP

SEQ ID NO: 327 - protein, 33 aa, Artificial sequence
  synthetic sequence
YKSEDIIRLASHDGGSVNLEAVLRLNPQLIGLG

SEQ ID NO: 328 - protein, 33 aa, Artificial sequence
  synthetic sequence
YKSEDIIRLASHDGGSINLEAVLRLNPQLIGLG

SEQ ID NO: 329 - protein, 33 aa, Artificial sequence
  synthetic sequence
YKSEDIIRLASSNGGSVNLEAVLRLNPQLIGLG

SEQ ID NO: 330 - protein, 33 aa, Artificial sequence
  synthetic sequence
YKSEDIIRLASSNGGSVNLEAVIAVHKALHSNG

SEQ ID NO: 331 - protein, 33 aa, Artificial sequence
  synthetic sequence
FSADQVVKIAGHSGGSNNIAVMLAVFPRLRDFG

SEQ ID NO: 332 - protein, 33 aa, Artificial sequence
  synthetic sequence
YKINHCVNLLKLNHDGFMLKNLIPYDSKLTGLG

SEQ ID NO: 333 - protein, 19 aa, Artificial sequence
  synthetic sequence
  MISC_FEATURE (12)..(13): The residues may include base contacting residues (BCR) as listed in the table 8 (e.g., NI, NK, NN, NR, RT, HD, HI, SN, HS, LN) and may be chosen based upon the target nucleic acid sequence.
FNAEQIVRMVSXXGGSKNL

SEQ ID NO: 334 - protein, 16 aa, Artificial sequence
  synthetic sequence
  MISC_FEATURE (12)..(13): The residues may include base contacting residues (BCR) as listed in the table 8 (e.g., NI, NK, NN, NR, RT, HD, HI, SN, HS, LN) and may be chosen based upon the target nucleic acid sequence.
YNKKQIVLIASXXSGG

SEQ ID NO: 335 - protein, 143 aa, Artificial sequence
  synthetic sequence
MPDLELNFAIPLHLFDDETVFTHDATNDNSQASSSYSSKSSPASANARKRTSRKEMSGPPSKEPANTKSRRANSQNNKLSLADRLTKYNIDEEFYQTRSDSLLSLNYTKKQIERLILYKGRTSAVQQLLCKHEELLNLISPDG

SEQ ID NO: 336 - protein, 176 aa, Artificial sequence
  synthetic sequence
ALVKEYFPVFSSFHFTADQIVALICQSKQCFRNLKKNHQQWKNKGLSAEQIVDLILQETPPKPNFNNTSSSTPSPSAPSFFQGPSTPIPTPVLDNSPAPIFSNPVCFFSSRSENNTEQYLQDSTLDLDSQLGDPTKNFNVNNFWSLFPFDDVGYHPHSNDVGYHLHSDEESPFFDF

SEQ ID NO: 337 - protein, 63 aa, Artificial sequence
  synthetic sequence
ALVKEYFPVFSSFHFTADQIVALICQSKQCFRNLKKNHQQWKNKGLSAEQIVDLILQETPPKP

SEQ ID NO: 338 - protein, 62 aa, Artificial sequence
  synthetic sequence
RTLVTFKDVFVDFTREEWKLLDTAQQIVYRNVMLENYKNLVSLGYQLTKPDVILRLEKGEEP

SEQ ID NO: 339 - protein, 178 aa, Artificial sequence
  synthetic sequence
DYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLETPN

SEQ ID NO: 340 - protein, 34 aa, Artificial sequence
  synthetic sequence
  MISC_FEATURE (12)..(13): The residues may be NH for binding G; NG for binding T; NI for binding A; and HD for binding C
LTPDQVVAIASXXGGKQALETVQRLLPVLCQDHG

SEQ ID NO: 341 - protein, 15 aa, Artificial sequence
  synthetic sequence
  MISC_FEATURE (12)..(13): The residues may be NH for binding G; NG for binding T; NI for binding A; and HD for binding C
LTPEQVVAIASXXGG

SEQ ID NO: 342 - protein, 68 aa, Artificial sequence
  synthetic sequence
RPALESIVAQLSRPDPALAALTNDHLVALACLGGRPALDAVKKGLPHAPALIKRTNRRIPERTSHRVA

SEQ ID NO: 343 - protein, 17 aa, Artificial sequence
  synthetic sequence
GAGGGGGMDAKSLTAWS

SEQ ID NO: 344 - DNA, 17 bp, Artificial sequence
  synthetic sequence
gctcaggcggaggtgag

SEQ ID NO: 345 - DNA, 19 bp, Artificial sequence
  synthetic sequence
ctcgcccacgtggatgtgg

SEQ ID NO: 346 - DNA, 19 bp, Artificial sequence
  synthetic sequence
cactctcgcccacgtggat

SEQ ID NO: 347 - DNA, 19 bp, Artificial sequence
  synthetic sequence
ctgtcactctcgcccacgt

SEQ ID NO: 348 - DNA, 17 bp, Artificial sequence
  synthetic sequence
gacagaggcagtgctgg

SEQ ID NO: 349 - DNA, 17 bp, Artificial sequence
  synthetic sequence
cccccagcactgcctct

SEQ ID NO: 350 - DNA, 16 bp, Artificial sequence
  synthetic sequence
ggccagggcgcctgtg

SEQ ID NO: 351 - DNA, 15 bp, Artificial sequence
  synthetic sequence
gtgggatctgcatgc

SEQ ID NO: 352 - DNA, 19 bp, Artificial sequence
  synthetic sequence
gggatctgcatgcctggag

SEQ ID NO: 353 - DNA, 20 bp, Artificial sequence
  synthetic sequence
ggagcagccccaccagagtg

SEQ ID NO: 354 - DNA, 20 bp, Artificial sequence
  synthetic sequence
ggagaaggcggcactctggt

SEQ ID NO: 355 - DNA, 19 bp, Artificial sequence
  synthetic sequence
ggagaaggcggcactctgg

SEQ ID NO: 356 - DNA, 17 bp, Artificial sequence
  synthetic sequence
gagcagtggagaaggcg

SEQ ID NO: 357 - DNA, 20 bp, Artificial sequence
  synthetic sequence
gagcggaagggaaactgtcc

SEQ ID NO: 358 - DNA, 19 bp, Artificial sequence
  synthetic sequence
caggttgaagggagggtgc

SEQ ID NO: 359 - DNA, 20 bp, Artificial sequence
  synthetic sequence
gaagggagggtgcccgcccc

SEQ ID NO: 360 - DNA, 15 bp, Artificial sequence
  synthetic sequence
gcccgccccttgctc

SEQ ID NO: 361 - DNA, 17 bp, Artificial sequence
  synthetic sequence
gcccgccccttgctccc

SEQ ID NO: 362 - DNA, 15 bp, Artificial sequence
  synthetic sequence
tgctcccgccccctc

SEQ ID NO: 363 - DNA, 17 bp, Artificial sequence
  synthetic sequence
ggaggaagagggggcgg

SEQ ID NO: 364 - DNA, 20 bp, Artificial sequence
  synthetic sequence
ggatgtggaggaagaggggg

SEQ ID NO: 365 - DNA, 17 bp, Artificial sequence
  synthetic sequence
tgaagggagggtgcccg

SEQ ID NO: 366 - DNA, 19 bp, Artificial sequence
  synthetic sequence
gggggcggctactgctcat

SEQ ID NO: 367 - DNA, 18 bp, Artificial sequence
  synthetic sequence
gtgctgagctagcactca

SEQ ID NO: 368 - DNA, 18 bp, Artificial sequence
  synthetic sequence
ggcatgacagagaacttt

SEQ ID NO: 369 - DNA, 18 bp, Artificial sequence
  synthetic sequence
atcacaggacagacatca

SEQ ID NO: 370 - DNA, 19 bp, Artificial sequence
  synthetic sequence
cagaatattagaacagaga

SEQ ID NO: 371 - DNA, 19 bp, Artificial sequence
  synthetic sequence
acatgcatggctctctgtt

SEQ ID NO: 372 - DNA, 18 bp, Artificial sequence
  synthetic sequence
tggaagtttgaaggtcaa

SEQ ID NO: 373 - DNA, 19 bp, Artificial sequence
  synthetic sequence
aatattctgactttgacct

SEQ ID NO: 374 - DNA, 19 bp, Artificial sequence
  synthetic sequence
tcaaacttccaactcttca

SEQ ID NO: 375 - DNA, 16 bp, Artificial sequence
  synthetic sequence
gttgccaaaaggaaca

SEQ ID NO: 376 - DNA, 17 bp, Artificial sequence
  synthetic sequence
gggttcaaacacatttc

SEQ ID NO: 377 - DNA, 20 bp, Artificial sequence
  synthetic sequence
agagcaaaacctttcaggat

SEQ ID NO: 378 - DNA, 20 bp, Artificial sequence
  synthetic sequence
tctgtgtgggttcaaacaca

SEQ ID NO: 379 - DNA, 17 bp, Artificial sequence
  synthetic sequence
tgattctgtgtgggttc

SEQ ID NO: 380 - DNA, 17 bp, Artificial sequence
  synthetic sequence
ttcaggatcctgaagct

SEQ ID NO: 381 - DNA, 16 bp, Artificial sequence
  synthetic sequence
tgattctgtgtgggtt

SEQ ID NO: 382 - DNA, 20 bp, Artificial sequence
  synthetic sequence
ttcaggatcctgaagctttg

SEQ ID NO: 383 - DNA, 19 bp, Artificial sequence
  synthetic sequence
aaagtccttgattctgtgt

SEQ ID NO: 384 - DNA, 16 bp, Artificial sequence
  synthetic sequence
cctgaagctttgaaat

SEQ ID NO: 385 - DNA, 20 bp, Artificial sequence
  synthetic sequence
ttgaaatgtgtttgaaccca

SEQ ID NO: 386 - DNA, 19 bp, Artificial sequence
  synthetic sequence
caaagctatctatataaag

SEQ ID NO: 387 - DNA, 17 bp, Artificial sequence
  synthetic sequence
gtgtttgaacccacaca

SEQ ID NO: 388 - DNA, 17 bp, Artificial sequence
  synthetic sequence
ttgaacccacacagaat

SEQ ID NO: 389 - DNA, 20 bp, Artificial sequence
  synthetic sequence
atctgggatcaaagctatct

SEQ ID NO: 390 - DNA, 20 bp, Artificial sequence
  synthetic sequence
ttgaacccacacagaatcaa

SEQ ID NO: 391 - DNA, 20 bp, Artificial sequence
  synthetic sequence
aatacatatctgggatcaaa

SEQ ID NO: 392 - DNA, 20 bp, Artificial sequence
  synthetic sequence
tctgtgtgtgcacatgtgta

SEQ ID NO: 393 - DNA, 17 bp, Artificial sequence
  synthetic sequence
tctgtgtgtgcacatgt

SEQ ID NO: 394 - DNA, 19 bp, Artificial sequence
  synthetic sequence
atagatagctttgatccca

SEQ ID NO: 395 - DNA, 18 bp, Artificial sequence
  synthetic sequence
gccttctgtgtgtgcaca

SEQ ID NO: 396 - DNA, 17 bp, Artificial sequence
  synthetic sequence
agctttgatcccagata

SEQ ID NO: 397 - DNA, 19 bp, Artificial sequence
  synthetic sequence
tcaagtgccttctgtgtgt

SEQ ID NO: 398 - DNA, 17 bp, Artificial sequence
  synthetic sequence
ttgatcccagatatgta

SEQ ID NO: 399 - DNA, 20 bp, Artificial sequence
  synthetic sequence
tctattcaagtgccttctgt

SEQ ID NO: 400 - DNA, 20 bp, Artificial sequence
  synthetic sequence
tgatcccagatatgtattac

SEQ ID NO: 401 - DNA, 17 bp, Artificial sequence
  synthetic sequence
ttctattcaagtgcctt

SEQ ID NO: 402 - DNA, 20 bp, Artificial sequence
  synthetic sequence
aaaaccaaaacaaaaaggct

SEQ ID NO: 403 - DNA, 17 bp, Artificial sequence
  synthetic sequence
cgtaaaaccaaaacaaa

SEQ ID NO: 404 - DNA, 20 bp, Artificial sequence
  synthetic sequence
gcacacacagaaggcacttg

SEQ ID NO: 405 - DNA, 18 bp, Artificial sequence
  synthetic sequence
tgaatagaaagccttttt

SEQ ID NO: 406 - DNA, 20 bp, Artificial sequence
  synthetic sequence
aaacccacggcttcctttct

SEQ ID NO: 407 - DNA, 17 bp, Artificial sequence
  synthetic sequence
aaacccacggcttcctt

SEQ ID NO: 408 - DNA, 20 bp, Artificial sequence
  synthetic sequence
aacagctaaacccacggctt

SEQ ID NO: 409 - DNA, 20 bp, Artificial sequence
  synthetic sequence
cgacgtaacagctaaaccca

SEQ ID NO: 410 - DNA, 20 bp, Artificial sequence
  synthetic sequence
tttcgacgtaacagctaaac

SEQ ID NO: 411 - DNA, 20 bp, Artificial sequence
  synthetic sequence
tgttttggttttacgagaaa

SEQ ID NO: 412 - DNA, 19 bp, Artificial sequence
  synthetic sequence
cttttcgacgtaacagcta

SEQ ID NO: 413 - DNA, 20 bp, Artificial sequence
  synthetic sequence
ttggttttacgagaaaggaa

SEQ ID NO: 414 - DNA, 15 bp, Artificial sequence
  synthetic sequence
cttttcgacgtaaca

SEQ ID NO: 415 - DNA, 18 bp, Artificial sequence
  synthetic sequence
tttacgagaaaggaagcc

SEQ ID NO: 416 - DNA, 20 bp, Artificial sequence
  synthetic sequence
tgaggttgtcttttcgacgt

SEQ ID NO: 417 - DNA, 17 bp, Artificial sequence
  synthetic sequence
gcttgaggttgtctttt

SEQ ID NO: 418 - DNA, 20 bp, Artificial sequence
  synthetic sequence
ttgttcagttgagtgcttga

SEQ ID NO: 419 - DNA, 18 bp, Artificial sequence
  synthetic sequence
gggtttagctgttacgtc

SEQ ID NO: 420 - DNA, 20 bp, Artificial sequence
  synthetic sequence
tgttttgttcagttgagtgc

SEQ ID NO: 421 - DNA, 16 bp, Artificial sequence
  synthetic sequence
tgttttgttcagttga

SEQ ID NO: 422 - DNA, 17 bp, Artificial sequence
  synthetic sequence
gttacgtcgaaaagaca

SEQ ID NO: 423 - DNA, 18 bp, Artificial sequence
  synthetic sequence
tggcttgttttgttcagt

SEQ ID NO: 424 - DNA, 18 bp, Artificial sequence
  synthetic sequence
ccatggattggcttgttt

SEQ ID NO: 425 - DNA, 20 bp, Artificial sequence
  synthetic sequence
cgaaaagacaacctcaagca

SEQ ID NO: 426 - DNA, 19 bp, Artificial sequence
  synthetic sequence
atataaagtccttgattct

SEQ ID NO: 427 - DNA, 17 bp, Artificial sequence
  synthetic sequence
ggggaaggtggagggaa

SEQ ID NO: 428 - DNA, 20 bp, Artificial sequence
  synthetic sequence
ggggaaggtggagggaaggc

SEQ ID NO: 429 - DNA, 17 bp, Artificial sequence
  synthetic sequence
ggagggaaggccgggca

SEQ ID NO: 430 - DNA, 18 bp, Artificial sequence
  synthetic sequence
ggagggaaggccgggcac

SEQ ID NO: 431 - DNA, 19 bp, Artificial sequence
  synthetic sequence
ggagggaaggccgggcaca

SEQ ID NO: 432 - DNA, 17 bp, Artificial sequence
  synthetic sequence
gtcccagggaacagagc

SEQ ID NO: 433 - DNA, 19 bp, Artificial sequence
  synthetic sequence
ctgctctccgccacggccc

SEQ ID NO: 434 - DNA, 20 bp, Artificial sequence
  synthetic sequence
gaggaggtgggggcgggggt

SEQ ID NO: 435 - DNA, 17 bp, Artificial sequence
  synthetic sequence
gaggaggtgggggcggg

SEQ ID NO: 436 - DNA, 16 bp, Artificial sequence
  synthetic sequence
ctgttccctgggacac

SEQ ID NO: 437 - DNA, 18 bp, Artificial sequence
  synthetic sequence
gggcagatcaggcagcct

SEQ ID NO: 438 - protein, 34 aa, Artificial sequence
  synthetic sequence
LTPDQVVAIASNHGGKQALETVQRLLPVLCQDHG

SEQ ID NO: 439 - protein, 34 aa, Artificial sequence
  synthetic sequence
LTPDQVVAIASNGGGKQALETVQRLLPVLCQDHG

SEQ ID NO: 440 - protein, 34 aa, Artificial sequence
  synthetic sequence
LTPDQVVAIASNIGGKQALETVQRLLPVLCQDHG

SEQ ID NO: 441 - protein, 34 aa, Artificial sequence
  synthetic sequence
LTPDQVVAIASHDGGKQALETVQRLLPVLCQDHG

SEQ ID NO: 442 - DNA, 15 bp, Artificial sequence
  synthetic sequence
acttcttccaactgt

SEQ ID NO: 443 - DNA, 18 bp, Artificial sequence
  synthetic sequence
gagaaaattgtattagat

SEQ ID NO: 444 - DNA, 21 bp, Artificial sequence
  synthetic sequence
gcctctgtcactctcgcccac

SEQ ID NO: 445 - DNA, 8 bp, Artificial sequence
  synthetic sequence
tctccact

SEQ ID NO: 446 - DNA, 20 bp, Artificial sequence
  synthetic sequence
tggtctctgggccttcaccc

SEQ ID NO: 447 - protein, 110 aa, Artificial sequence
  synthetic sequence
HHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 448 - DNA, 18 bp, Artificial sequence
  synthetic sequence
tctgctggtctctgggcc

SEQ ID NO: 449 - DNA, 20 bp, Artificial sequence
  synthetic sequence
tggtctctgggccttcaccc

SEQ ID NO: 450 - DNA, 21 bp, Artificial sequence
  synthetic sequence
tctgctggtctctgggccttc

SEQ ID NO: 451 - DNA, 20 bp, Artificial sequence
  synthetic sequence
ctgttccctgggacaccccc

SEQ ID NO: 452 - protein, 63 aa, Artificial sequence
  synthetic sequence
SIVAQLSRPDPALAALTNDHLVALACLGGRPALDAVKKGLPHAPALIKRTNRRIPERTSHRVA

SEQ ID NO: 453 - protein, 35 aa, Artificial sequence
  synthetic sequence
  MISC_FEATURE (1)..(11): The residues include a chain of 11 contiguous amino acids
  MISC_FEATURE (1)..(35): The residues may be repeated 7-40, 7-35, or 7-25 times.
  MISC_FEATURE (12)..(13): The residues may be selected from, e.g., NH, HH, KH, NK, NQ, RH, RN, SS, NN, SN, KN, NI, KI, RI, HI, SI, NG, HG, KG, RG, HD, RD, SD, ND, KD, YG, NV, HN, H*, HA, KA, N*, NA, NC, NS, RA, or S* wherein (*) means that the amino acid at X13 is absent
  MISC_FEATURE (14)..(35): X14-33 or 34 or 35 is a chain of 20, 21 or 22 contiguous amino acids
XXXXXXXXXXXXXXXXXXXXXXXXXXXXXXXXXXX

SEQ ID NO: 454 - DNA, 37 bp, Artificial sequence
  synthetic sequence
cctcccccagcactgcctctgtcactctcgcccacgt

SEQ ID NO: 455 - protein, 35 aa, Artificial sequence; identical to SEQ ID NO: 453
  synthetic sequence
  MISC_FEATURE (1)..(11): X1-11 is a chain of 11 contiguous amino acids
  MISC_FEATURE (12)..(13): The residues may be selected from ,e.g., NH, HH, KH, NK, NQ, RH, RN, SS, NN, SN, KN, NI, KI, RI, HI, SI, NG, HG, KG, RG, HD, RD, SD, ND, KD, YG, NV, HN, H*, HA, KA, N*, NA, NC, NS, RA, or S* wherein (*) means that the amino acid at X13 is absent
  MISC_FEATURE (14)..(35): X14-33 or 34 or 35 is a chain of 20, 21 or 22 contiguous amino acids

SEQ ID NO: 456 - DNA, 21 bp, Artificial sequence
  synthetic sequence
tctgctggtctctgggccttc

SEQ ID NO: 457 - DNA, 18 bp, Artificial sequence
  synthetic sequence
tctgctggtctctgggcc

SEQ ID NO: 458 - protein, 11 aa, Artificial sequence
  synthetic sequence
LTPEQVVAIAS

SEQ ID NO: 459 - protein, 11 aa, Artificial sequence
  synthetic sequence
LTPAQVVAIAS

SEQ ID NO: 460 - protein, 11 aa, Artificial sequence
  synthetic sequence
LTPDQVVAIAN

SEQ ID NO: 461 - protein, 11 aa, Artificial sequence
  synthetic sequence
LTPDQVVAIAS

SEQ ID NO: 462 - protein, 11 aa, Artificial sequence
  synthetic sequence
LTPYQVVAIAS

SEQ ID NO: 463 - protein, 11 aa, Artificial sequence
  synthetic sequence
LTREQVVAIAS

SEQ ID NO: 464 - protein, 11 aa, Artificial sequence
  synthetic sequence
LSTAQVVAIAS

SEQ ID NO: 465 - protein, 22 aa, Artificial sequence
  synthetic sequence
  misc_feature (1)..(22): Xaa can be any naturally occurring amino acid
XXXXXXXXXXXXXXXXXXXXXX

SEQ ID NO: 466 - protein, 21 aa, Artificial sequence
  synthetic sequence
GGKQALETVQRLLPVLCQDHG

SEQ ID NO: 467 - protein, 21 aa, Artificial sequence
  synthetic sequence
GGKQALATVQRLLPVLCQDHG

SEQ ID NO: 468 - protein, 21 aa, Artificial sequence
  synthetic sequence
GGKQALETVQRVLPVLCQDHG

SEQ ID NO: 469 - protein, 21 aa, Artificial sequence
  synthetic sequence
GGKQALETVQRVLPVLCQDHG

SEQ ID NO: 470 - protein, 34 aa, Artificial sequence
  synthetic sequence
  MISC_FEATURE (12)..(13): The residues may be selected from, e.g., NH, HH, KH, NK, NQ, RH, RN, SS, NN, SN, KN, NI, KI, RI, HI, SI, NG, HG, KG, RG, HD, RD, SD, ND, KD, YG, NV, HN, H*, HA, KA, N*, NA, NC, NS, RA, or S* wherein (*) means that the amino acid at X13 is absent
LTPEQVVAIASXXGGKQALETVQRLLPVLCQDHG

SEQ ID NO: 471 - protein, 21 aa, Artificial sequence
  synthetic sequence
  MISC_FEATURE (1)..(11): X1-11 is a chain of 11 contiguous amino acids
  MISC_FEATURE (12)..(13): The residues may be selected from, e.g., NH, HH, KH, NK, NQ, RH, RN, SS, NN, SN, KN, NI, KI, RI, HI, SI, NG, HG, KG, RG, HD, RD, SD, ND, KD, YG, NV, HN, H*, HA, KA, N*, NA, NC, NS, RA, or S* wherein (*) means that the amino acid at X13 is absent
  MISC_FEATURE (14)..(21): X14-19 or 20 or 21 is a chain of 7, 8 or 9 contiguous amino acids
XXXXXXXXXXXXXXXXXXXXX

SEQ ID NO: 472 - protein, 7 aa, Artificial sequence
  synthetic sequence
GGRPALE

SEQ ID NO: 473 - protein, 72 aa, Artificial sequence
  synthetic sequence
DSDEHLKKLKTFLENLRRHLDRLDKHIKQLRDILSENPEDERVKDVIDLSERSVRIVKTVIKIFEDSVRKKE

SEQ ID NO: 474 - protein, 75 aa, Artificial sequence
  synthetic sequence
GSDDKELDKLLDTLEKILQTATKIIDDANKLLEKLRRSERKDPKVVETYVELLKRHEKAVKELLEIAKTHAKKVE

SEQ ID NO: 475 - protein, 77 aa, Artificial sequence
  synthetic sequence
GTKEDILERQRKIIERAQEIHRRQQEILEELERIIRKPGSSEEAMKRMLKLLEESLRLLKELLELSEESAQLLYEQR

SEQ ID NO: 476 - protein, 83 aa, Artificial sequence
  synthetic sequence
GTEKRLLEEAERAHREQKEIIKKAQELHRRLEEIVRQSGSSEEAKKEAKKILEEIRELSKRSLELLREILYLSQEQKGSLVPR

SEQ ID NO: 477 - protein, 72 aa, Artificial sequence
  synthetic sequence
DEEDHLKKLKTHLEKLERHLKLLEDHAKKLEDILKERPEDSAVKESIDELRRSIELVRESIEIFRQSVEEEE

SEQ ID NO: 478 - protein, 74 aa, Artificial sequence
  synthetic sequence
GDVKELTKILDTLTKILETATKVIKDATKLLEEHRKSDKPDPRLIETHKKLVEEHETLVRQHKELAEEHLKRTR

SEQ ID NO: 479 - protein, 74 aa, Artificial sequence
  synthetic sequence
DNEEIIKEARRVVEEYKKAVDRLEELVRRAENAKHASEKELKDIVREILRISKELNKVSERLIELWERSQERAR

SEQ ID NO: 480 - protein, 72 aa, Artificial sequence
  synthetic sequence
TAEELLEVHKKSDRVTKEHLRVSEEILKVVEVLTRGEVSSEVLKRVLRKLEELTDKLRRVTEEQRRVVEKLN

SEQ ID NO: 481 - protein, 76 aa, Artificial sequence
  synthetic sequence
DLEDLLRRLRRLVDEQRRLVEELERVSRRLEKAVRDNEDERELARLSREHSDIQDKHDKLAREILEVLKRLLERTE

SEQ ID NO: 482 - protein, 15 aa, Artificial sequence
  synthetic sequence
GGGGGMDAKSLTAWS

SEQ ID NO: 483 - protein, 75 aa, Artificial sequence
  synthetic sequence
PTDEVIEVLKELLRIHRENLRVNEEIVEVNERASRVTDREELERLLRRSNELIKRSRELNEESKKLIEKLERLAT